(19) United States
(12) Patent Application Publication
(10) Pub. No.: US 2025/0228134 A1
(43) Pub. Date: Jul. 10, 2025

(54) ORGANIC COMPOUND, LIGHT-EMITTING DEVICE INCLUDING THE SAME, AND ELECTRONIC APPARATUS INCLUDING THE LIGHT-EMITTING DEVICE

(71) Applicant: Samsung Display Co., Ltd., Yongin-si (KR)

(72) Inventors: Taeil Kim, Yongin-si (KR); Junha Park, Yongin-si (KR); Minjae Sung, Yongin-si (KR); Munki Sim, Yongin-si (KR); Chihyun Ryoo, Yongin-si (KR); Minjung Jung, Yongin-si (KR); Seonhyoung Hur, Yongin-si (KR)

(21) Appl. No.: 19/012,686

(22) Filed: Jan. 7, 2025

(30) Foreign Application Priority Data
Jan. 8, 2024 (KR) 10-2024-0003122

(51) Int. Cl. H10K 85/60; C09K 11/06; H10K 85/30; H10K 85/40

(52) U.S. Cl. CPC H10K 85/658; C09K 11/06; H10K 85/346; H10K 85/40; H10K 85/653; H10K 85/654; H10K 85/656; H10K 85/6572; H10K 85/6574; H10K 85/6576

(57) ABSTRACT

A light-emitting device including a first electrode, a second electrode opposite to (e.g., facing) the first electrode, and an interlayer arranged between the first electrode and the second electrode is provided. The interlayer includes an emission layer and an organic compound represented by Formula 1.

### CROSS-REFERENCE TO RELATED APPLICATION

This application claims priority to and the benefit of Korean Patent Application No. 10-2024-0003122, filed on Jan. 8, 2024, in the Korean Intellectual Property Office, the entire content of which is incorporated herein by reference.

### BACKGROUND

### 1. Field

One or more aspects of embodiments of the present disclosure relate to an organic compound, a light-emitting device including the same, and an electronic apparatus including the light-emitting device.

### 2. Description of the Related Art

Self-emissive devices (for example, organic light-emitting devices and/or the like) among light-emitting devices have relatively wide viewing angles, high contrast ratios, short response times, and excellent or suitable characteristics in terms of luminance, driving voltage, and response speed.

A light-emitting device may include a first electrode, a hole transport region, an emission layer, an electron transport region, and a second electrode, arranged sequentially. Holes injected from the first electrode may move toward the emission layer through the hole transport region. Electrons injected from the second electrode may move toward the emission layer through the electron transport region. Carriers, such as holes and electrons, may combine in the emission layer to produce excitons that may transition (and/or relax) from an excited state to a ground state, and light may be generated thereby.

### SUMMARY

One or more aspects of embodiments of the present disclosure are directed toward an organic compound having a high photoluminescence quantum yield, a high molar extinction coefficient, and low delayed fluorescence lifespan, and a light-emitting device including the organic compound as a thermally activated delayed fluorescence (i.e., TADF) material and having high luminescence efficiency and long lifespan.

Additional aspects will be set forth in part in the description which follows and, in part, will be apparent from the description, or may be learned by practice of the presented embodiments of the disclosure.

According to one or more embodiments, a light-emitting device includes a first electrode, a second electrode opposite to (e.g., facing) the first electrode, and an interlayer arranged between the first electrode and the second electrode and including an emission layer, wherein the interlayer further includes an organic compound represented by Formula 1.

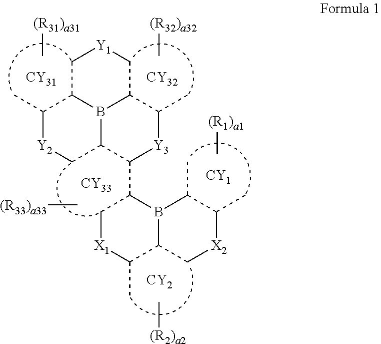

In Formula 1,
X₁ may be selected from among O, S, Se, C(Ar₁₁)(Ar₁₂), Si(Ar₁₁)(Ar₁₂), and N(Ar₁₁),
X₂ may be selected from among O, S, Se, C(Ar₂₁)(Ar₂₂), Si(Ar₂₁)(Ar₂₂), and N(Ar₂₁),
Y₁ may be selected from among O, S, Se, C(Z₁₁)(Z₁₂), Si(Z₁₁)(Z₁₂), and N(Z₁₁),
Y₂ may be selected from among O, S, Se, C(Z₂₁)(Z₂₂), Si(Z₂₁)(Z₂₂), and N(Z₂₁),
Y₃ may be selected from among O, S, Se, C(Z₃₁)(Z₃₂), Si(Z₃₁)(Z₃₂), and N(Z₃₁),
Z₁₁ and Z₁₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or Z₁₁ and Z₁₂ may not be bonded,
Z₂₁ and Z₂₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or Z₂₁ and Z₂₂ may not be bonded,
Z₃₁ and Z₃₂ may (e.g., optionally) be bonded to each other to form a C₃-C₆₀ carbocyclic group that is unsubstituted or substituted with at least one R₁₀ₐ, or a C₁-C₆₀ heterocyclic group that is unsubstituted or substituted with at least one R₁₀ₐ, or Z₃₁ and Z₃₂ may not be bonded,
ring CY₁, ring CY₂, and ring CY₃₁ to ring CY₃₃ may each independently be a C₃-C₆₀ carbocyclic group or a C₁-C₆₀ heterocyclic group,
Ar₁₁, Ar₁₂, Ar₂₁, and Ar₂₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
Z₁₁, Z₁₂, Z₂₁, Z₂₂, Z₃₁, Z₃₂, R₁, R₂, and R₃₁ to R₃₃ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a2, and a31 to a33 may each independently be an integer from 0 to 20,
at least one selected from among Z₁₁ and Z₁₂ may (e.g., optionally) be bonded to ring CY₃₁ or ring CY₃₂ via a single bond, *—O—*′, *—S—*′, *—Se—*′, *—C(T₁₁)(T₁₂)-*′, or *—Si(T₁₁)(T₁₂)-*′, or Z₁₁ and Z₁₂ are not bonded to ring CY₃₁ and ring CY₃₂,
at least one selected from among Z₂₁ and Z₂₂ may (e.g., optionally) be bonded to ring CY₃₁ or ring CY₃₃ via a single bond, *—O—*′, *—S—*′, *—Se—*′, *—C(T₂₁)(T₂₂)-*′, or *—Si(T₂₁)(T₂₂)-*′, or Z₂₁ and Z₂₂ are not bonded to ring CY₃₁ and ring CY₃₃,
at least one selected from among Z₃₁ and Z₃₂ may (e.g., optionally) be bonded to ring CY₃₂ or ring CY₃₃ via a single bond, *—O—*′, *—S—*′, *—Se—*′, *—C(T₃₁)(T₃₂)-*′, or *—Si(T₃₁)(T₃₂)-*′, or Z₃₁ and Z₃₂ are not bonded to ring CY₃₂ and ring CY₃₃,
T₁₁, T₁₂, T₂₁, T₂₂, T₃₁, and T₃₂ may each independently be hydrogen, deuterium, —F, a cyano group, a C₁-C₆₀ alkyl group, a C₁-C₆₀ alkoxy group, a C₆-C₆₀ aryl group, or a C₁-C₆₀ heteroaryl group,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be
hydrogen, deuterium, —F, —Cl, —Br, —I, a hydroxyl group, a cyano group, or a nitro group, or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

According to one or more embodiments, an electronic apparatus includes the light-emitting device and a thin-film transistor electrically connected to the light-emitting device.

According to one or more embodiments, an electronic equipment includes the light-emitting device and may be at least one selected from among a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a 3D display, a virtual or augmented reality display, a vehicle, a video wall including multiple displays tiled together, a theater or stadium screen, a phototherapy device, a sign, and combinations thereof.

According to one or more embodiments, provided is the organic compound represented by Formula 1.

### BRIEF DESCRIPTION OF THE DRAWINGS

The accompanying drawings are included to provide a further understanding of the preceding and other aspects, features, and advantages of certain embodiments of the disclosure are incorporated in and constitute a part of this specification. The drawings illustrate example embodiments and, together with the following description taken in conjunction with the accompanying drawings. In the drawings:

FIG. 1 is a schematic view showing a light-emitting device according to one or more embodiments;

FIG. 2 is a schematic view of an electronic apparatus according to one or more embodiments;

FIG. 3 is a schematic view of an electronic apparatus according to one or more embodiments;

FIG. 4 is a schematic perspective view of electronic equipment including a light-emitting device, according to one or more embodiments;

FIG. 5 is a schematic view of the exterior of a vehicle as electronic equipment including a light-emitting device, according to one or more embodiments; and

FIGS. 6A-6C are each a schematic view showing the interior of the vehicle in FIG. 5.

### DETAILED DESCRIPTION

Reference will now be made in more detail to one or more embodiments, examples of which are illustrated in the accompanying drawings, wherein like reference numerals refer to like elements throughout, and duplicative descriptions thereof may not be provided the specification. In this regard, the present embodiments may have different forms and should not be construed as being limited to the descriptions set forth herein. Accordingly, one or more embodiments are merely described in more detail, by referring to the drawings, to explain aspects of the present description. An aspect and a characteristic of the disclosure, and a method of accomplishing these will be apparent if referring to one or more embodiments described with reference to the drawings. The same or corresponding components will be denoted by the same reference numerals, and thus redundant description thereof will not be provided.

As used herein, the term “and/or” includes any and all combinations of one or more of the associated listed items. Expressions such as “at least one of,” “one of,” “selected from,” and “selected from among,” when preceding a list of elements, modify the entire list of elements and do not modify the individual elements of the list. For example, throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

Unless otherwise defined, all chemical names, technical and scientific terms, and terms defined in common dictionaries should be interpreted as having meanings consistent with the context of the related art, and should not be interpreted in an ideal or overly formal sense. It will be understood that although the terms “first,” “second,” and/or the like may be utilized herein to describe one or more suitable components, these components should not be limited by these terms. These terms are only utilized to distinguish one component from another. Thus, a first element could be termed a second element without departing from the teachings of the present disclosure. Similarly, a second element could be termed a first element. An expression utilized in the singular forms such as “a,” “an,” and “the” are intended to encompass the expression of the plural forms as well, unless it has a clearly different meaning in the context.

It will be further understood that the terms “comprises,” “comprising,” “comprise,” “has,” “have,” “having,” “include,” “includes,” and/or “including,” as utilized herein specify the presence of stated features or elements, but do not preclude the presence or addition of one or more other features or elements.

As used herein, the terms “use,” “using,” and “used” may be considered synonymous with the terms “utilize,” “utilizing,” and “utilized,” respectively.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

In the following embodiments, if one or more components such as layers, films, regions, plates, and/or the like are said to be “connected to,” or “on” another component, this may include not only a case in which other components are “immediately on” the layers, films, regions, or plates, but also a case in which other components may be placed therebetween. Sizes of elements in the drawings may be exaggerated for convenience of explanation. In other words, because sizes and thicknesses of components in the drawings are arbitrarily illustrated for convenience of explanation, the following embodiments are not limited thereto.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

In this context, “consisting essentially of” indicates that any additional components will not materially affect the chemical, physical, optical or electrical properties of the semiconductor film.

Further, in this specification, the phrase “on a plane,” or “plan view,” indicates viewing a target portion from the top, and the phrase “on a cross-section” indicates viewing a cross-section formed by vertically cutting a target portion from the side.

The term “interlayer” as utilized herein refers to a single layer and/or all of a plurality of layers located between the first electrode and the second electrode of the light-emitting device.

Descriptions of features or aspects within each embodiment should typically be considered as available for other similar features or aspects in other embodiments.

### Light-Emitting Device

According to one or more embodiments, a light-emitting device includes a first electrode, a second electrode opposite to (e.g., facing) the first electrode, and an interlayer arranged between the first electrode and the second electrode and including an emission layer, wherein the interlayer further includes an organic compound represented by Formula 1:

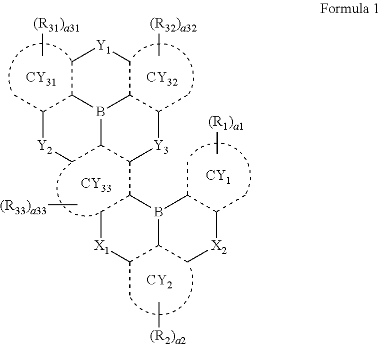

wherein, in Formula 1,
X₁ may be selected from among O, S, Se, C(Ar₁₁)(Ar₁₂), Si(Ar₁₁)(Ar₁₂), and N(Ar₁₁),
X₂ may be selected from among O, S, Se, C(Ar₂₁)(Ar₂₂), Si(Ar₂₁)(Ar₂₂), and N(Ar₂₁),
Y₁ may be selected from among O, S, Se, C(Z₁₁)(Z₁₂), Si(Z₁₁)(Z₁₂), and N(Z₁₁),
Y₂ may be selected from among O, S, Se, C(Z₂₁)(Z₂₂), Si(Z₂₁)(Z₂₂), and N(Z₂₁),
Y₃ may be selected from among O, S, Se, C(Z₃₁)(Z₃₂), Si(Z₃₁)(Z₃₂), and N(Z₃₁),
Z₁₁ and Z₁₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or Z₁₁ and Z₁₂ may not be bonded,
Z₂₁ and Z₂₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or Z₂₁ and Z₂₂ may not be bonded,
Z₃₁ and Z₃₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or Z₃₁ and Z₃₂ may not be bonded,
ring CY₁, ring CY₂, and ring CY₃₁ to ring CY₃₃ may each independently be a C₃-C₆₀ carbocyclic group or a C₁-C₆₀ heterocyclic group,
Ar₁₁, Ar₁₂, Ar₂₁, and Ar₂₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
Z₁₁, Z₁₂, Z₂₁, Z₂₂, Z₃₁, Z₃₂, R₁, R₂, and R₃₁ to R₃₃ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a2, and a31 to a33 may each independently be an integer from 0 to 20,
at least one selected from among Z₁₁ and Z₁₂ may (e.g., optionally) be bonded to ring CY₃₁ or ring CY₃₂ via a single bond, *—O—*′, *—S—*′, *—Se—*′, *—C(T₁₁)(T₁₂)-*′, or *—Si(T₁₁)(T₁₂)-*′, or Z₁₁ and Z₁₂ are not bonded to ring CY₃₁ and ring CY₃₂,
at least one selected from among Z₂₁ and Z₂₂ may (e.g., optionally) be bonded to ring CY₃₁ or ring CY₃₃ via a single bond, *—O—*′, *—S—*′, *—Se—*′, *—C(T₂₁)(T₂₂)-*′, or *—Si(T₂₁)(T₂₂)-*′, or Z₂₁ and Z₂₂ are not bonded to ring CY₃₁ and ring CY₃₃,
at least one selected from among Z₃₁ and Z₃₂ may (e.g., optionally) be bonded to ring CY₃₂ or ring CY₃₃ via a single bond, *—O—*′, *—S—*′, *—Se—*′, *—C(T₃₁)(T₃₂)-*′, or *—Si(T₃₁)(T₃₂)-*′, or Z₃₁ and Z₃₂ are not bonded to ring CY₃₂ and ring CY₃₃,
T₁₁, T₁₂, T₂₁, T₂₂, T₃₁, and T₃₂ may each independently be hydrogen, deuterium, —F, a cyano group, a C₁-C₆₀ alkyl group, a C₁-C₆₀ alkoxy group, a C₆-C₆₀ aryl group, or a C₁-C₆₀ heteroaryl group,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

Because the light-emitting device includes the organic compound, the light-emitting device may have improved luminescence efficiency and improved lifespan characteristics. For example, the organic compound may be included in the emission layer.

In one or more embodiments, the light-emitting device may include a second compound including at least one π electron-deficient nitrogen-containing C₁-C₆₀ heterocyclic group, a third compound including a group represented by Formula 3, a fourth compound including a transition metal, or any combination thereof, wherein the organic compound, the second compound, the third compound, and the fourth compound are different from each other:

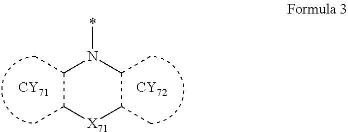

wherein, in Formula 3,
ring CY₇₁ and ring CY₇₂ may each independently be a π electron-rich C₃-C₆₀ cyclic group or a pyridine group,
X₇₁ may be a single bond, or a linking group including O, S, N, B, C, Si, or any combination thereof, and
indicates a binding site to any atom included in a remaining part other than Formula 3 in the third compound (e.g., a binding site to an atom of the third compound excluding an (e.g., any) atom in Formula 3).

Formulae 1 and 3 are each as described in the present specification.

In one or more embodiments, the light-emitting device may include a layer (e.g., an interlayer) including 1) the organic compound and 2) the second compound, the third compound, the fourth compound, or any combination thereof. The layer (e.g., interlayer) may include a mixture including 1) the organic compound and 2) the second compound, the third compound, the fourth compound, or any combination thereof. For example, the layer (e.g., interlayer) is clearly distinct from a double layer including (e.g., consisting of) 1) a first layer including the organic compound and 2) a second layer including the second compound, the third compound, the fourth compound, or any combination thereof. For example, the layer (e.g., interlayer) may be the emission layer.

For example, the interlayer (for example, the emission layer) may include i) the organic compound, ii) the organic compound and the second compound, iii) the organic compound and the third compound, iv) the organic compound and the fourth compound, v) the organic compound, the second compound, and the third compound, vi) the organic compound, the second compound, and the fourth compound, vii) the organic compound, the third compound, and the fourth compound, or viii) the organic compound and the second compound to the fourth compound.

In one or more embodiments, the organic compound and the second compound to the fourth compound may each include at least one deuterium.

In one or more embodiments, the second compound and the third compound may each include at least one silicon.

In one or more embodiments, the second compound and the third compound may form an exciplex.

In one or more embodiments, the emission layer may be to emit blue light.

In one or more embodiments, the maximum emission wavelength of the blue light may be about 430 nanometer (nm) to about 475 nm, about 440 nm to about 475 nm, about 450 nm to about 475 nm, about 430 nm to about 470 nm, about 440 nm to about 470 nm, about 450 nm to about 470 nm, about 430 nm to about 465 nm, about 440 nm to about 465 nm, about 450 nm to about 465 nm, about 430 nm to about 460 nm, about 440 nm to about 460 nm, or about 450 nm to about 460 nm.

In one or more embodiments, the emission full width at half maximum (FWHM) of the blue light may be 40 nm or less, about 5 nm to about 40 nm, about 10 nm to about 40 nm, about 15 nm to about 40 nm, about 20 nm to about 40 nm, about 5 nm to about 37 nm, about 10 nm to about 37 nm, about 15 nm to about 37 nm, or about 20 nm to about 37 nm.

In one or more embodiments, the blue light may be deep blue light.

In one or more embodiments, the CIEx coordinate (for example, the top emission CIEx coordinate) of the blue light may be about 0.125 to about 0.140 or about 0.130 to about 0.140.

In one or more embodiments, the CIEy coordinate (for example, the top emission CIEy coordinate) of the blue light may be about 0.120 to about 0.210. For example, the CIEy coordinate may be measured via Evaluation Example 2 as described in more detail herein.

In one or more embodiments, the second compound may include a pyridine group, a pyrimidine group, a pyrazine group, a pyridazine group, a triazine group, or any combination thereof.

In one or more embodiments, the second compound may include a compound represented by Formula 2:

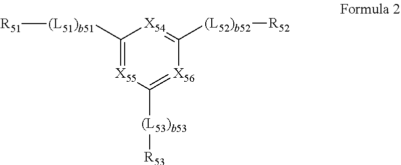

wherein, in Formula 2,
L₅₁ to L₅₃ may each independently be a single bond, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
b51 to b53 may each independently be an integer from 1 to 5,
X₅₄ may be N or C(R₅₄), X₅₅ may be N or C(R₅₅), X₅₆ may be N or C(R₅₆), and at least one selected from among X₅₄ to X₅₆ may be N, and
R₅₁ to R₅₆ are each as described in connection with R₁₀ₐ.

In one or more embodiments, the third compound may include a compound represented by Formula 3-1, a compound represented by Formula 3-2, a compound represented by Formula 3-3, a compound represented by Formula 3-4, a compound represented by Formula 3-5, or any combination thereof:

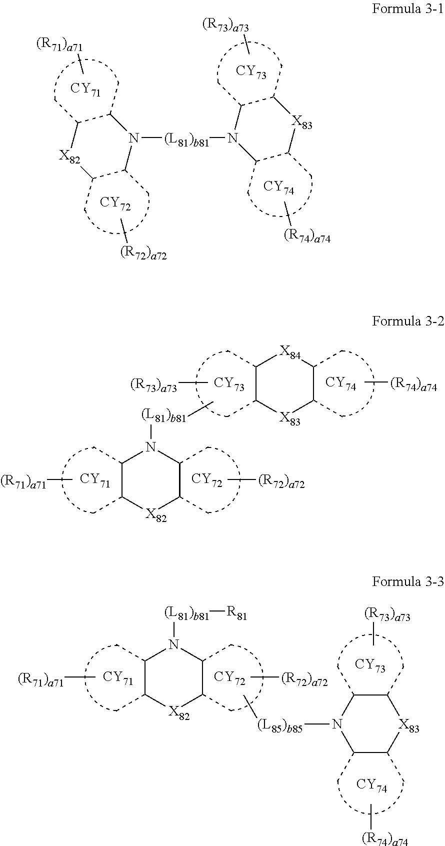

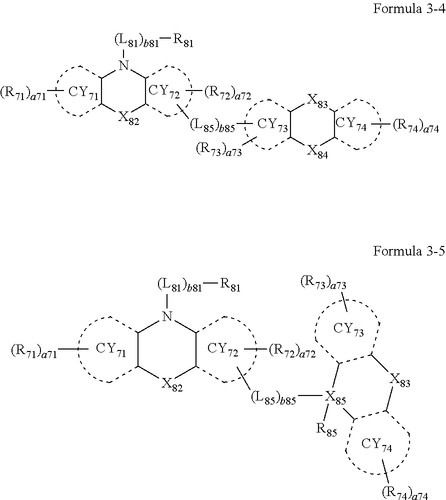

wherein, in Formulae 3-1 to 3-5,
ring CY₇₁ to ring CY₇₄ may each independently be a π electron-rich C₃-C₆₀ cyclic group or a pyridine group,
X₈₂ may be a single bond, O, S, N-[(L₈₂)_{b82}-R₈₂], C(R₈₂ₐ)(R_{82b}), or Si(R₈₂ₐ)(R_{82b}),
X₈₃ may be a single bond, O, S, N-[(L₈₃)_{b83}-R₈₃], C(R₈₃ₐ)(R_{83b}), or Si(R₈₃ₐ)(R_{83b}),
X₈₄ may be O, S, N-[(L₈₄)_{b84}-R₈₄], C(R₈₄ₐ)(R_{84b}), or Si(R₈₄ₐ)(R_{84b}),
X₈₅ may be C or Si,
L₈₁ to L₈₅ may each independently be a single bond, *—C(Q₄)(Q₅)-*′, *—Si(Q₄)(Q₅)-*′, a π electron-rich C₃-C₆₀ cyclic group that is unsubstituted or substituted with at least one R₁₀ₐ or a pyridine group that is unsubstituted or substituted with at least one R₁₀ₐ, wherein Q₄ and Q₅ are each as described in connection with Q₁,
b81 to b85 may each independently be an integer from 1 to 5,
R₇₁ to R₇₄, R₈₁ to R₈₅, R₈₂ₐ, R_{82b}, R₈₃ₐ, R_{83b}, R₈₄ₐ, and R_{84b} are each as described in connection with R₁,
a71 to a74 may each independently be an integer from 0 to 20, and
R₁₀ₐ is as described in the present specification.

In one or more embodiments, the third compound may exclude (e.g., neither be) Compound CBP nor mCBP:

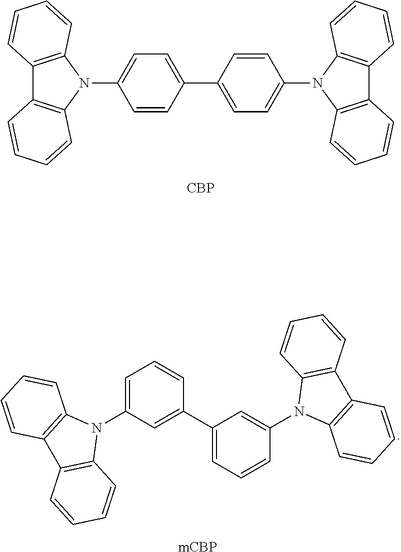 [SMILES: c1ccc2c(c1)c1ccccc1n2-c1ccc(-c2ccc(-n3c4ccccc4c4ccccc43)cc2)cc1 ; "CBP" ; c1cc(-c2cccc(-n3c4ccccc4c4ccccc43)c2)cc(-n2c3ccccc3c3ccccc32)c1 ; "mCBP"]

In one or more embodiments, the fourth compound may be a compound including a transition metal and a ligand. The transition metal may be platinum (Pt). The ligand may include a monodentate ligand, a bidentate ligand, a tridentate ligand, a tetradentate ligand, or any combination thereof.

In one or more embodiments, the fourth compound may be represented by Formula 4:

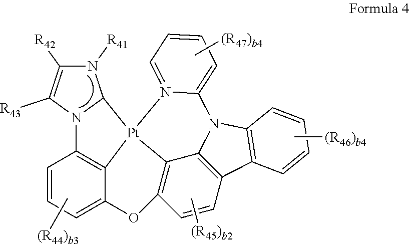

wherein, in Formula 4,
R₄₁ to R₄₇ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, a C₇-C₆₀ arylalkyl group that is unsubstituted or substituted with at least one R₁₀ₐ, a C₂-C₆₀ heteroarylalkyl group that is unsubstituted or substituted with at least one R₁₀ₐ, —C(Q₁)(Q₂)(Q₃), —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₄₂ and R₄₃ may (e.g., optionally) be bonded to each other to form a C₄-C₁₀ carbocyclic group that is unsubstituted or substituted with at least one R₁₀ₐ,
R₁₀ₐ and Q₁ to Q₃ are each as described in the present specification,
b2 may be an integer from 0 to 2,
b3 may be an integer from 0 to 3, and
b4 may be an integer from 0 to 4.

In one or more embodiments, in Formula 4, R₄₂ and R₄₃ may be bonded to each other to form a benzene group that is unsubstituted or substituted with at least one R₁₀ₐ.

In one or more embodiments, the light-emitting device may satisfy at least one selected from among Condition 1 to Condition 4:

lowest unoccupied molecular orbital (LUMO) energy level (eV) of third compound>LUMO energy level (eV) of fourth compound; Condition 1

LUMO energy level (eV) of fourth compound>LUMO energy level (eV) of second compound; Condition 2

highest occupied molecular orbital (HOMO) energy level (eV) of fourth compound>HOMO energy level (eV) of third compound; and Condition 3

HOMO energy level (eV) of third compound>HOMO energy level (eV) of second compound. Condition 4

Each of the highest occupied molecular orbital (HOMO) energy level and the lowest unoccupied molecular orbital (LUMO) energy level of each of the second compound to the fourth compound may be a negative value, and may be measured according to a suitable method.

In one or more embodiments, the absolute value of a difference between the LUMO energy level of the second compound and the LUMO energy level of the fourth compound may be about 0.1 electron volt (eV) to about 1.0 eV, and/or the absolute value of a difference between the LUMO energy level of the third compound and the LUMO energy level of the fourth compound may be about 0.1 eV to about 1.0 eV.

In one or more embodiments, the absolute value of a difference between the HOMO energy level of the second compound and the HOMO energy level of the fourth compound may be 1.25 eV or less (for example, about 0.2 eV to about 1.25 eV), and/or the absolute value of a difference between the HOMO energy level of the third compound and the HOMO energy level of the fourth compound may be 1.25 eV or less (for example, about 0.2 eV to about 1.25 eV).

When the relationships between LUMO energy level and HOMO energy level satisfy the conditions as described herein, the balance between holes and electrons injected into the emission layer may be achieved.

In one or more embodiments, the emission layer may include i) a dopant or an emitter (organic compound), ii) at least one host (second compound and/or third compound), and iii) a sensitizer (fourth compound), and the emission layer may be to emit phosphorescence or fluorescence (for example, delayed fluorescence) emitted from the dopant. For example, the sensitizer may not act as the dopant, but instead as an auxiliary dopant that transfers energy to the dopant. As another example, the sensitizer may act as an auxiliary dopant that transfers energy to the dopant and may also act as a dopant that emits light.

The emitted phosphorescence or fluorescence may be blue phosphorescence or blue fluorescence (for example, blue delayed fluorescence). The blue light may have a maximum emission wavelength of about 390 nm to about 500 nm, about 410 nm to about 490 nm, about 430 nm to about 480 nm, about 440 nm to about 475 nm, or about 455 nm to about 470 nm.

In one or more embodiments, the light-emitting device may further include a capping layer arranged outside (e.g., and on) the first electrode and/or outside (e.g., and on) the second electrode.

For example, the light-emitting device may further include at least one selected from among a first capping layer arranged outside (e.g., and on) the first electrode and a second capping layer arranged outside (e.g., and on) the second electrode.

At least one selected from among the first capping layer and the second capping layer may include the organic compound represented by Formula 1.

In one or more embodiments, the light-emitting device may include:
a first capping layer located outside (e.g., and on) the first electrode and including the organic compound represented by Formula 1;
a second capping layer located outside (e.g., and on) the second electrode and including the organic compound represented by Formula 1; or
both (e.g., simultaneously) the first capping layer located outside (e.g., and on) the first electrode and the second capping layer located outside (e.g., and on) the second electrode, wherein at least one selected from among the first capping layer and the second capping layer may include the organic compound represented by Formula 1.

The expression “(interlayer and/or a capping layer) includes an organic compound represented by Formula 1” as used herein may be to refer to that the (interlayer and/or the capping layer) may include one kind of organic compound represented by Formula 1 or two or more different kinds of organic compounds, each represented by Formula 1.

For example, the interlayer and/or the capping layer may include Compound 1 only as the organic compound. Compound 1 may be included in the emission layer of the light-emitting device.

As another example, the interlayer may include, as the organic compound, Compound 1 and Compound 2 as described in more detail herein. Compound 1 and Compound 2 may be included in a substantially identical layer (for example, both (e.g., simultaneously) Compound 1 and Compound 2 may be included in the emission layer), or may be included in different layers (for example, Compound 1 may be included in the emission layer, and Compound 2 may be included in the electron transport region).

The term “interlayer” as used herein refers to a single layer and/or each (e.g., all) of a plurality of layers arranged between the first electrode and the second electrode of the light-emitting device.

According to one or more embodiments, an electronic apparatus includes: the light-emitting device; and a thin-film transistor electrically connected to the light-emitting device. For example, the thin-film transistor may include a source electrode and a drain electrode, and the first electrode of light-emitting device may be electrically connected to the source electrode or the drain electrode. The electronic apparatus may further include a color filter, a color conversion layer, a touch screen layer, a polarizing layer, or any combination thereof. More details of the electronic apparatus may be referred to the descriptions provided herein.

According to one or more embodiments, an electronic equipment includes the light-emitting device and is at least one selected from among a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a 3D display, a virtual or augmented reality display, a vehicle, a video wall including multiple displays tiled together, a theater or stadium screen, a phototherapy device, a sign, and combinations thereof.

According to one or more embodiments, provided is the organic compound represented by Formula 1. Formula 1 is as described in the present specification.

Synthesis methods of the organic compound may be recognizable by one of ordinary skill in the art by referring to Synthesis Examples and/or Examples provided herein.

### Description of Formula

The organic compound may be represented by Formula 1:

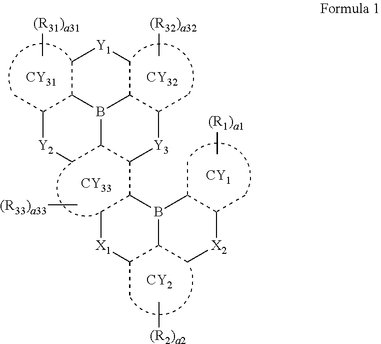

wherein, in Formula 1,
X₁ may be selected from among O, S, Se, C(Ar₁₁)(Ar₁₂), Si(Ar₁₁)(Ar₁₂), and N(Ar₁₁),
X₂ may be selected from among O, S, Se, C(Ar₂₁)(Ar₂₂), Si(Ar₂₁)(Ar₂₂), and N(Ar₂₁),
Y₁ may be selected from among O, S, Se, C(Z₁₁)(Z₁₂), Si(Z₁₁)(Z₁₂), and N(Z₁₁),
Y₂ may be selected from among O, S, Se, C(Z₂₁)(Z₂₂), Si(Z₂₁)(Z₂₂), and N(Z₂₁),
Y₃ may be selected from among O, S, Se, C(Z₃₁)(Z₃₂), Si(Z₃₁)(Z₃₂), and N(Z₃₁),
Z₁₁ and Z₁₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
Z₂₁ and Z₂₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
Z₃₁ and Z₃₂ may (e.g., optionally) be bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
ring CY₁, ring CY₂, and ring CY₃₁ to ring CY₃₃ may each independently be a C₃-C₆₀ carbocyclic group or a C₁-C₆₀ heterocyclic group,
Ar₁₁, Ar₁₂, Ar₂₁, and Ar₂₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
Z₁₁, Z₁₂, Z₂₁, Z₂₂, Z₃₁, Z₃₂, R₁, R₂, and R₃₁ to R₃₃ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a2, and a31 to a33 may each independently be an integer from 0 to 20,
at least one selected from among Z₁₁ and Z₁₂ may (e.g., optionally) be bonded to ring CY₃₁ or ring CY₃₂ via a single bond, *—O—*′, *—S—*′, *—Se—*′, *—C(T₁₁)(T₁₂)-*′, or *—Si(T₁₁)(T₁₂)-*′, or Z₁₁ and Z₁₂ are not bonded to ring CY₃₁ and ring CY₃₂,
at least one selected from among Z₂₁ and Z₂₂ may (e.g., optionally) be bonded to ring CY₃₁ or ring CY₃₃ via a single bond, *—O—*′, *—S—*′, *—Se—*′, *—C(T₂₁)(T₂₂)-*′, or *—Si(T₂₁)(T₂₂)-*′, or Z₂₁ and Z₂₂ are not bonded to ring CY₃₁ and ring CY₃₃,
at least one selected from among Z₃₁ and Z₃₂ may (e.g., optionally) be bonded to ring CY₃₂ or ring CY₃₃ via a single bond, *—O—*′, *—S—*′, *—Se—*′, *—C(T₃₁)(T₃₂)-*′, or *—Si(T₃₁)(T₃₂)-*′, or Z₃₁ and Z₃₂ are not bonded to ring CY₃₂ and ring CY₃₃,
T₁₁, T₁₂, T₂₁, T₂₂, T₃₁, and T₃₂ may each independently be hydrogen, deuterium, —F, a cyano group, a C₁-C₆₀ alkyl group, a C₁-C₆₀ alkoxy group, a C₆-C₆₀ aryl group, or a C₁-C₆₀ heteroaryl group,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

An example in which Y₁ in Formula 1 is C(Z₁₁)(Z₁₂), wherein Z₁₁ and Z₁₂ are bonded to each other to form a C₃-C₆₀ carbocyclic group, may refer to Compound 20:

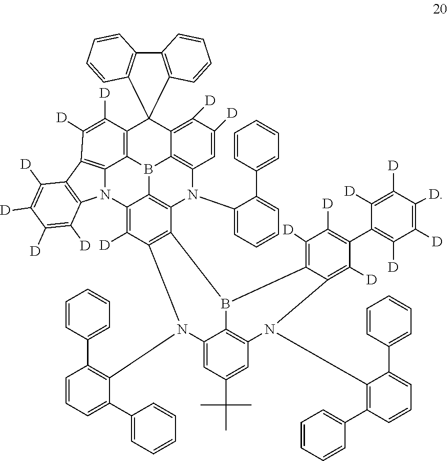

An example in which Y₂ in Formula 1 is N(Z₂₁), wherein Z₂₁ is bonded to ring CY₃₁ via a single bond, may refer to Formula FT2, Compound 5, and/or the like:

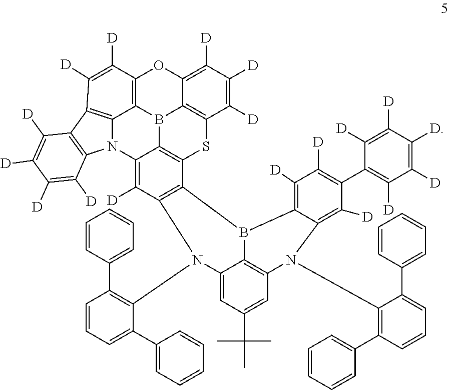

An example in which Y₂ in Formula 1 is N(Z₂₁), wherein Z₂₁ is bonded to ring CY₃₁ via *—O—*′, may refer to Formula FT2, Compound 8, and/or the like:

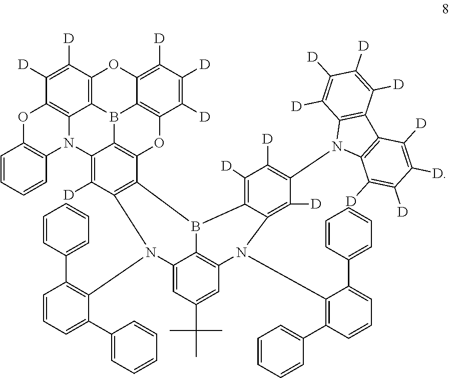

An example in which Y₂ in Formula 1 is N(Z₂₁), wherein Z₂₁ is bonded to ring CY₃₁ via *—S—*′, and Y₃ is N(Z₃₁), wherein Z₃₁ is bonded to ring CY₃₂ via *—S—*′, may refer to Formula FT4, Compound 15, and/or the like:

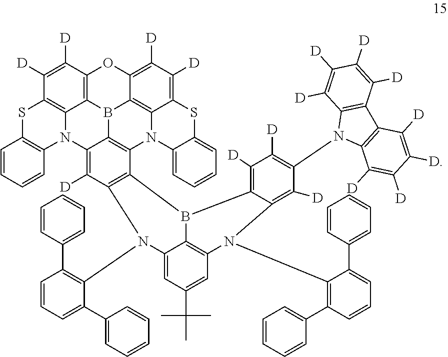

An example in which Y₂ in Formula 1 is N(Z₂₁), wherein Z₂₁ is bonded to ring CY₃₁ via a single bond, and Y₃ is N(Z₃₁), wherein Z₃₁ is bonded to ring CY₃₂ via *—Si(T₃₁)(T₃₂)-*′, may refer to Formula FT4, Compound 14, and/or the like:

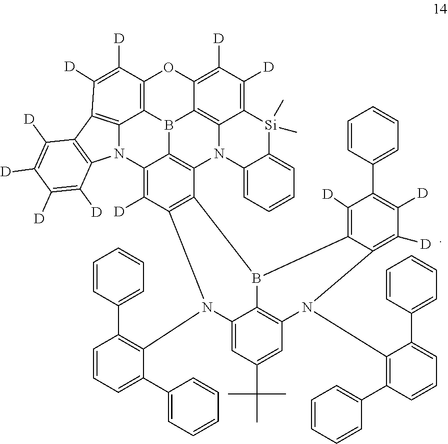 [SMILES: [2H]c1c([2H])c2c3c4c1Oc1c([2H])c([2H])c5c6c([2H])c([2H])c([2H])c([2H])c6n6c5c1B4c1c(c4c(c([2H])c1-6)N(c1c(-c5ccccc5)cccc1-c1ccccc1)c1cc(C(C)(C)C)cc5c1B4c1c([2H])c(-c4ccccc4)c([2H])c([2H])c1N5c1c(-c4ccccc4)cccc1-c1ccccc1)N3c1ccccc1[Si]2(C)C]

In one or more embodiments, the organic compound may include at least one nitrogen atom.

In one or more embodiments, in Formula 1, i) X₁ may be N(Ar₁₁); ii) X₂ may be N(Ar₂₁); or iii) X₁ may be N(Ar₁₁), and concurrently X₂ may be N(Ar₂₁).

In one or more embodiments, Ar₁₁, Ar₁₂, Ar₂₁, and Ar₂₂ may each independently be a biphenyl group that is unsubstituted or substituted with at least one R₁₀ₐ, a terphenyl group that is unsubstituted or substituted with at least one R₁₀ₐ, a fluorenyl group that is unsubstituted or substituted with at least one R₁₀ₐ, a dibenzofuranyl group that is unsubstituted or substituted with at least one R₁₀ₐ, or a dibenzothiophenyl group that is unsubstituted or substituted with at least one R₁₀ₐ.

In one or more embodiments, Ar₁₁, Ar₁₂, Ar₂₁, and Ar₂₂ may each independently be a group represented by any one selected from among Formulae AR1 to AR4:

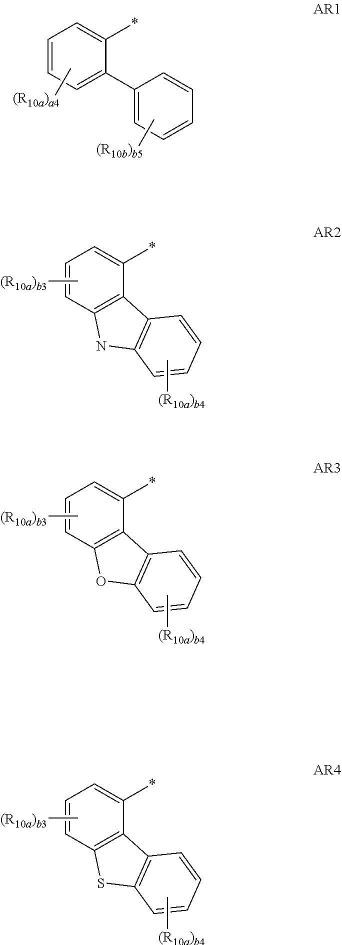

wherein, in Formulae AR1 to AR4,
b3 may be an integer from 0 to 3,
b4 may be an integer from 0 to 4,
b5 may be an integer from 0 to 5,
R_{10b} may be deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), or —P(═O)(Q₂₁)(Q₂₂), wherein Q₂₁, Q₂₂, and Q₂₃ are each independently defined as in Formula 1, and
* indicates a binding site to a neighboring atom.

In one or more embodiments, a group represented by Formula AR1 may be a group represented by any one selected from among Formulae AR11 to AR21:

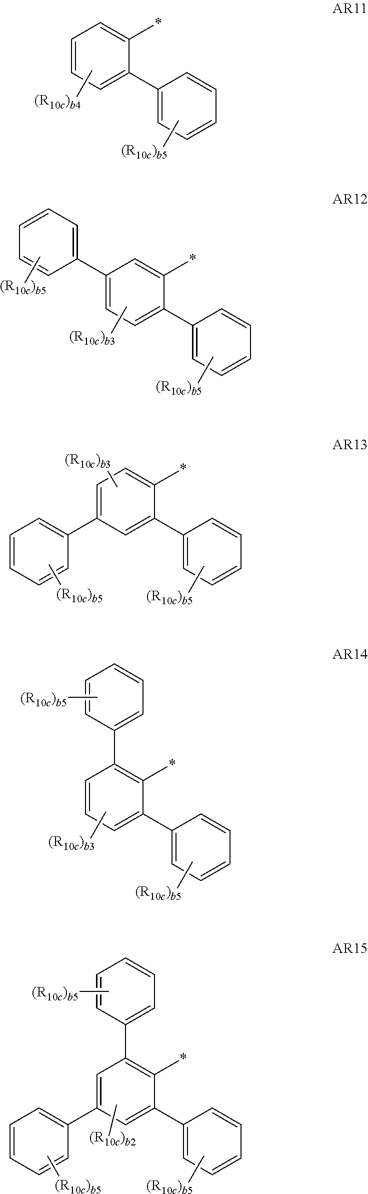

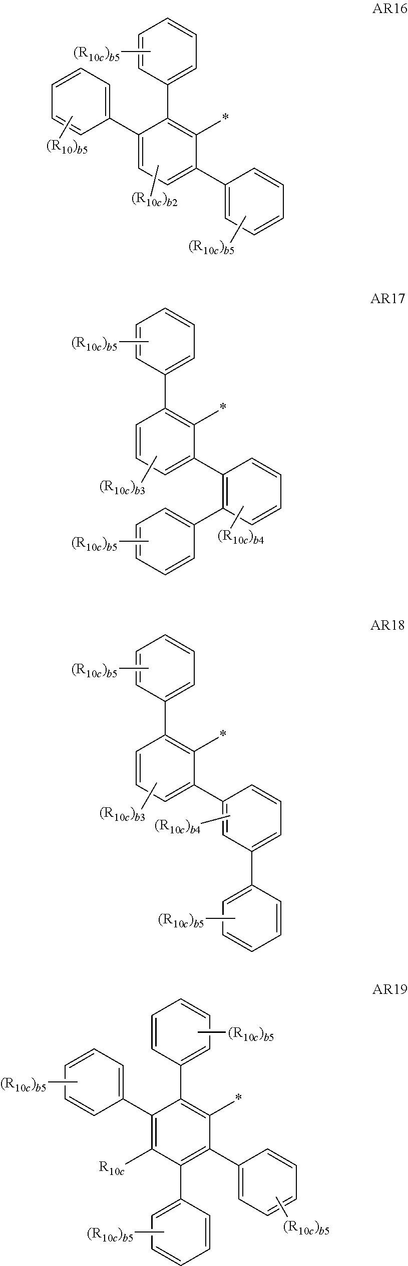

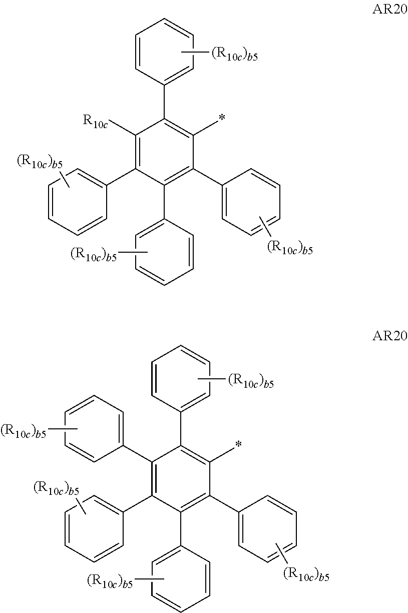

wherein, in Formulae AR11 to AR21,
b2 may be an integer selected from 0 to 2, b3 may be an integer selected from 0 to 3, b4 may be an integer selected from 0 to 4, and b5 may be an integer selected from 0 to 5, and
R_{10c} is as described in connection with R_{10b} and may exclude (e.g., may not be) a phenyl group.

For example, R_{10c} may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, and may not be a phenyl group.

In one or more embodiments, i) Y₂ may be selected from among O, S, Se, and N(Z₂₁), and Z₂₁ may optionally be bonded to ring CY₃₁ via a single bond, *—O—*, *—S—*′, *—Se—*′, *—C(T₂₁)(T₂₂)-*′, or *—Si(T₂₁)(T₂₂)-*′; ii) Y₃ may be selected from among O, S, Se, and N(Z₃₁), and Z₃₁ may optionally be bonded to ring CY₃₂ via a single bond, *—O—*′, *—S—*′, *—Se—*′, *—C(T₃₁)(T₃₂)-*′, or *—Si(T₃₁)(T₃₂)-*′; or iii) Y₂ may be selected from among O, S, Se, and N(Z₂₁), Y₃ may be selected from among O, S, Se, and N(Z₃₁), Z₂₁ may optionally be bonded to ring CY₃₁ via a single bond, *—O—*′, *—S—*′, *—Se—*′, *—C(T₂₁)(T₂₂)-*′, or *—Si(T₂₁)(T₂₂)-*′, and Z₃₁ may optionally be bonded to ring CY₃₂ via a single bond, *—O—*′, *—S—*′, *—Se—*′, *—C(T₃₁)(T₃₂)-*′, or *—Si(T₃₁)(T₃₂)-*′.

In one or more embodiments, Z₂₁, Z₂₂, Z₃₁, and Z₃₂ may each independently be a C₃-C₁₀ cycloalkyl group that is unsubstituted or substituted with at least one R₁₀ₐ, a C₁-C₁₀ heterocycloalkyl group that is unsubstituted or substituted with at least one R₁₀ₐ, a C₃-C₁ cycloalkenyl group that is unsubstituted or substituted with at least one R₁₀ₐ, a C₁-C₁₀ heterocycloalkenyl group that is unsubstituted or substituted with at least one R₁₀ₐ, a C₆-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ.

In one or more embodiments, ring CY₁, ring CY₂, and ring CY₃₁ to ring CY₃₃ may each independently be a benzene group, a naphthalene group, a pyridine group, a pyrimidine group, a triazine group, a quinoline group, or an isoquinoline group. For example, ring CY₁, ring CY₂, and ring CY₃₁ to ring CY₃₃ may each be a benzene group.

In one or more embodiments, ring CY₁, ring CY₂, and ring CY₃₁ to ring CY₃₃ may each independently be a 6-membered ring. In one or more embodiments, ring CY₁, ring CY₂, and ring CY₃₁ to ring CY₃₃ may each independently be a C₆ carbocyclic group.

In one or more embodiments, a group represented by

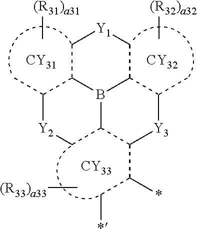

in Formula 1 may be a group represented by any one selected from among Formulae FT1 to FT4:

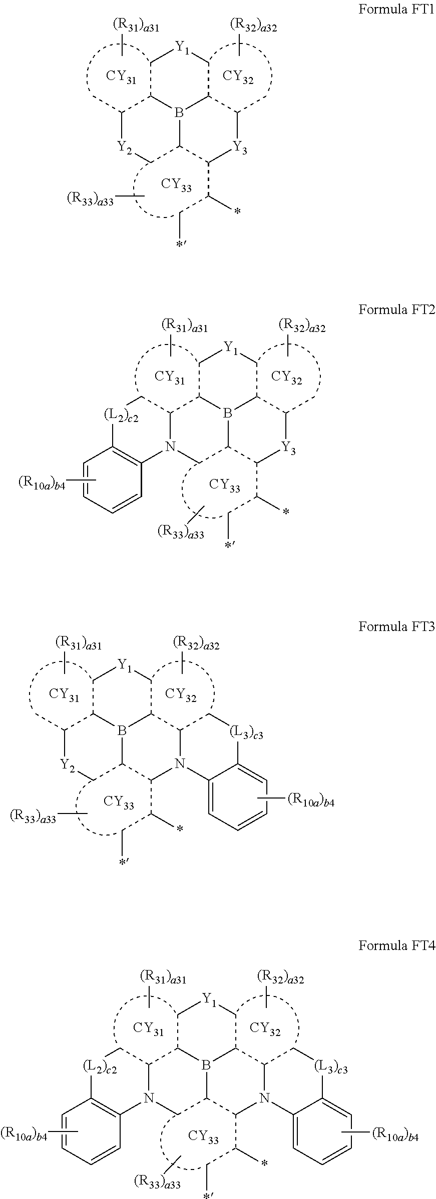

wherein, in Formulae FT1 to FT4, ring CY₃₁, ring CY₃₂, ring CY₃₃, R₃₁, R₃₂, R₃₃, Y₁, a31, a32, and a33 are each independently defined as in Formula 1,
Y₂ and Y₃ may each independently be selected from among O, S, and Se,
b4 may be an integer from 0 to 4,
L₂ may be O, S, Se, C(T₂₁)(T₂₂), or Si(T₂₁)(T₂₂),
L₃ may be O, S, Se, C(T₃₁)(T₃₂), or Si(T₃₁)(T₃₂),
c2 may be 0 or 1, wherein, when c2 is 0, (L₂)_{c2} may be a single bond,
c3 may be 0 or 1, wherein, when c3 is 0, (L₃)_{c3} may be a single bond,
* indicates a binding site to B in Formula 1, and
*′ indicates a binding site to X₁ in Formula 1.

In one or more embodiments, at least one selected from among Y₁ to Y₃ in Formula FT1 may be O or S, at least one selected from among Y₁, L₂, and Y₃ in Formula FT2 may be O or S, at least one selected from among Y₁, Y₂, and L₃ in Formula FT3 may be O or S, and at least one selected from among Y₁, L₂, and L₃ in Formula FT4 may be O or S. For example, the organic compound may include at least one O or at least one S.

In one or more embodiments, the organic compound may include at least one deuterium.

In one or more embodiments, R₁, R₂, and R₃₁ to R₃₃ may each independently be selected from among:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, and a phenyl group;
a C₁-C₆₀ alkyl group, a phenyl group, a naphthyl group, an anthracenyl group, a phenanthrenyl group, a phenalenyl group, a triphenylenyl group, a pyrenyl group, a chrysenyl group, a pyrrolyl group, a furanyl group, a thiophenyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzimidazolyl group, a benzofuranyl group, a dibenzofuranyl group, a benzothiophenyl group, a dibenzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a carbazolyl group, a benzocarbazolyl group, a fluorenyl group, a benzoisothiazolyl group, a benzoxazolyl group, a benzoisoxazolyl group, a phenoxazinyl group, an acridinyl group, and a xanthenyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a phenyl group, or any combination thereof; and
—Si(Q₁)(Q₂)(Q₃) and —N(Q₁)(Q₂).

In one or more embodiments, at least one selected from among R₃₁ to R₃₃ may be deuterium.

In one or more embodiments, the organic compound represented by Formula 1 may be represented by Formula 1-1:

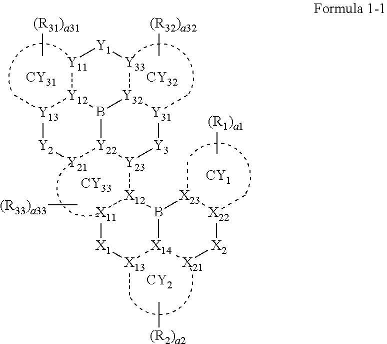

wherein, in Formula 1-1,
X₁₁ to X₁₄, X₂₁ to X₂₃, Y₁₁ to Y₁₃, Y₂₁ to Y₂₃, and Y₃₁ to Y₃₃ may each be a carbon atom (C) or a heteroatom, and the heteroatom may be selected from among a nitrogen atom (N), an oxygen atom (O), a sulfur atom (S), a phosphorus atom (P), a boron atom (B), a selenium atom (Se), and a silicon atom (Si). For example, X₁₁ to X₁₄, X₂₁ to X₂₃, Y₁₁ to Y₁₃, Y₂₁ to Y₂₃, and Y₃₁ to Y₃₃ may each be C, N, or O.

In one or more embodiments, a bond between two neighboring atoms selected from among X₁₁ to X₁₄, X₂₁ to X₂₃, Y₁₁ to Y₁₃, Y₂₁ to Y₂₃, and Y₃₁ to Y₃₃ may be a single bond or a double bond.

In one or more embodiments, the organic compound represented by Formula 1 and/or Formula 1-1 may include i) a 6-membered ring formed by X₁, X₁₁, X₁₂, B, X₁₄, and X₁₃, ii) a 6-membered ring formed by B, X₁₄, X₂₁, X₂, X₂₂, and X₂₃, iii) a 6-membered ring formed by Y₁, Y₁₁, Y₁₂, B, Y₃₂, and Y₃₃, iv) a 6-membered ring formed by B, Y₁₂, Y₁₃, Y₂, Y₂₁, and Y₂₂, and v) a 6-membered ring formed by B, Y₂₂, Y₂₃, Y₃, Y₃₁, and Y₃₂.

In one or more embodiments, ring CY₁, ring CY₂, and ring CY₃₁ to ring CY₃₃ in Formulae 1 and 1-1 may each be a benzene group.

In one or more embodiments, the organic compound represented by Formula 1 may be represented by Formula 1-2:

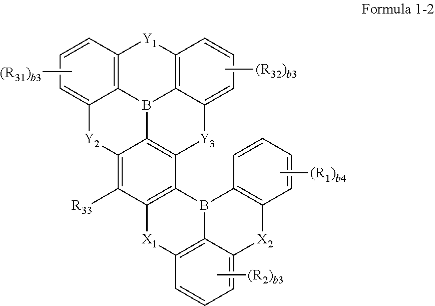

wherein, in Formula 1-2,
X₁, X₂, Y₁ to Y₃, R₁, R₂, and R₃₁ to R₃₃ are each as described in connection with Formula 1,
b3 may be an integer from 0 to 3, and
b4 may be an integer from 0 to 4.

In one or more embodiments, the organic compound represented by Formula 1 may be represented by Formula 1-2a, 1-2b, or 1-2c:

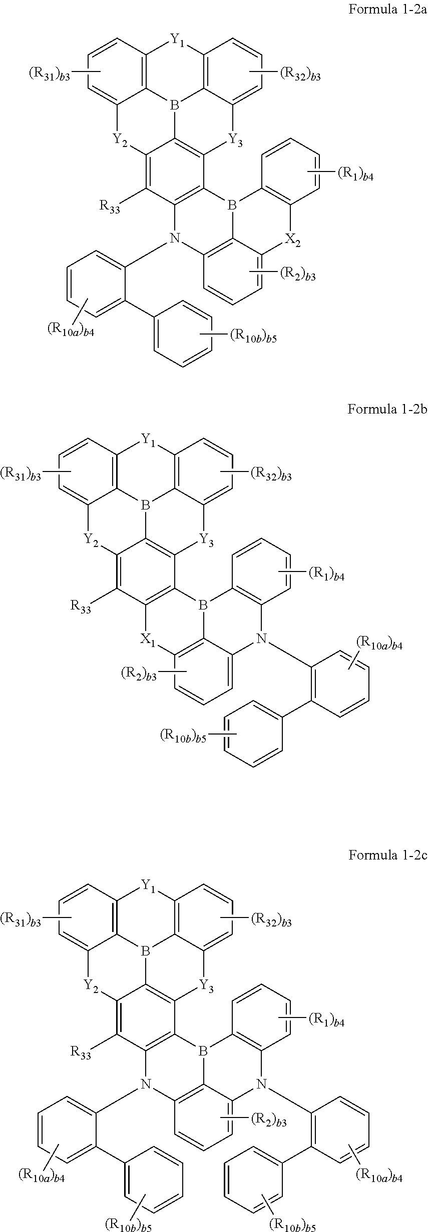

wherein, in Formulae 1-2a, 1-2b, and 1-2c,
X₁, X₂, Y₁ to Y₃, R₁, R₂, R₃₁ to R₃₃, and R₁₀ₐ are each as described in connection with Formula 1,
b3 may be an integer from 0 to 3,
b4 may be an integer from 0 to 4,
b5 may be an integer from 0 to 5, and
R_{10b} may be deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), or —P(═O)(Q₂₁)(Q₂₂) wherein Q₂₁, Q₂₂, and Q₂₃ are each independently defined as in Formula 1.

The organic compound represented by Formula 1, 1-1, 1-2, 1-2a, 1-2b, or 1-2c includes at least two boron atoms (B) at appropriate or suitable positions and includes at least one heteroatom at an appropriate or suitable position such as X₁, X₂, and any of Y₁ to Y₃, and these atoms are connected to each other via a plurality of 6-membered rings, ring CY₁, ring CY₂, and ring CY₃₁ to ring CY₃₃. In some embodiments, ring CY₃₁ and ring CY₃₂ are bonded to each other via a boron atom (B) and Y₁, ring CY₃₂ and ring CY₃₃ are bonded to each other via a boron atom (B) and Y₃, and ring CY₃₃ and ring CY₃₁ are bonded to each other via a boron atom (B) and Y₂. Therefore, the organic compound may undergo enhancement of a multiple-resonance (MR) aspect and reinforcement of structural rigidity, leading to induction of a distortion aspect. As a result, a local excited state (LE state) and a charge transfer state (CT state) are mixed, and thus, an orbital distribution between a lowest excited singlet state (S1) and a lowest excited triplet state (T1) may be changed. For example, spin-orbit coupling is strengthened, and thus, an inhibitory aspect according to the EI-Sayed rule may be offset, and reverse intersystem crossing (RISC) may be induced. Therefore, the organic compound may concurrently (e.g., simultaneously) have a high photoluminescence quantum yield (PLQY), a high molar extinction coefficient, and low delayed fluorescence lifespan, and the luminescence efficiency and lifespan of a light-emitting device including the organic compound may be improved.

### Examples of Compounds

In one or more embodiments, the organic compound may be any one selected from among Compounds 1 to 80:

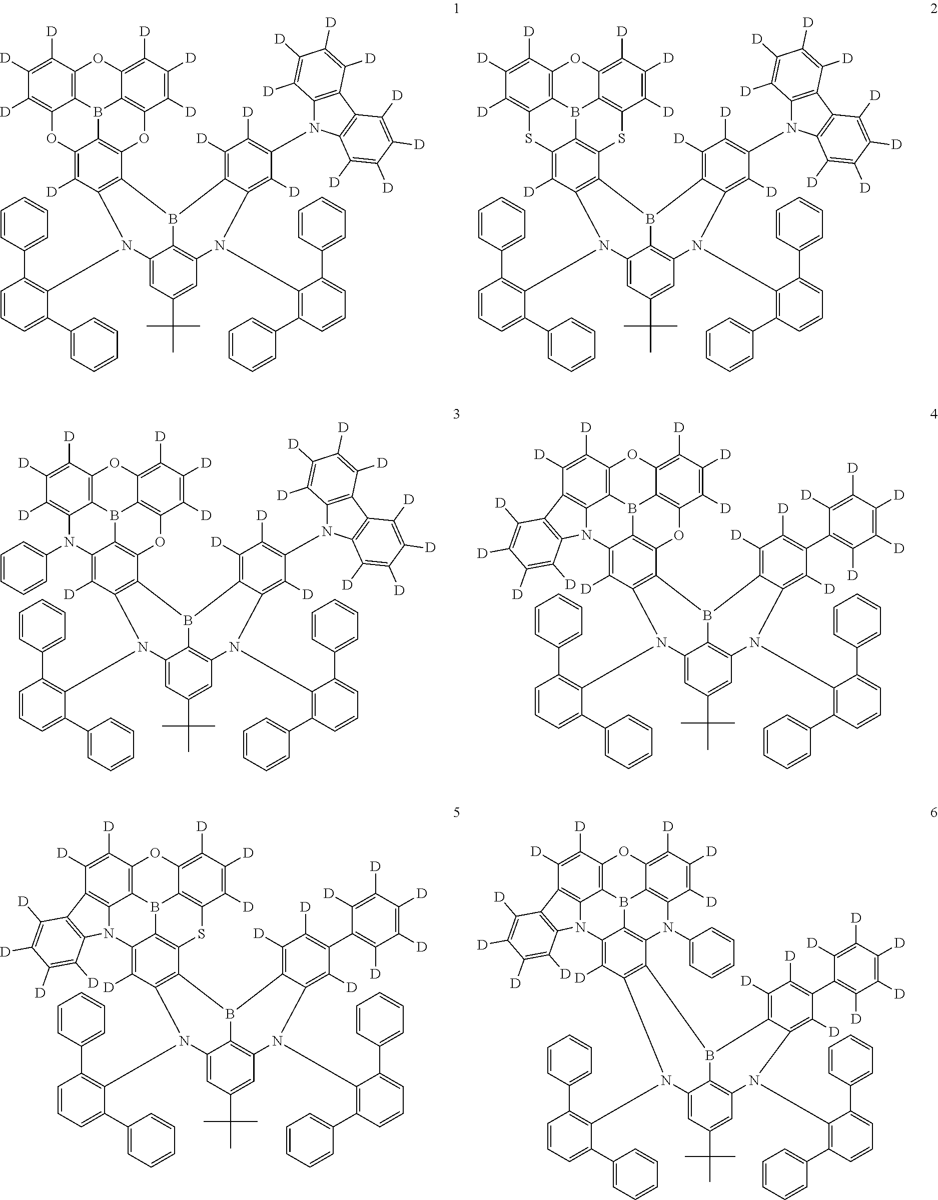

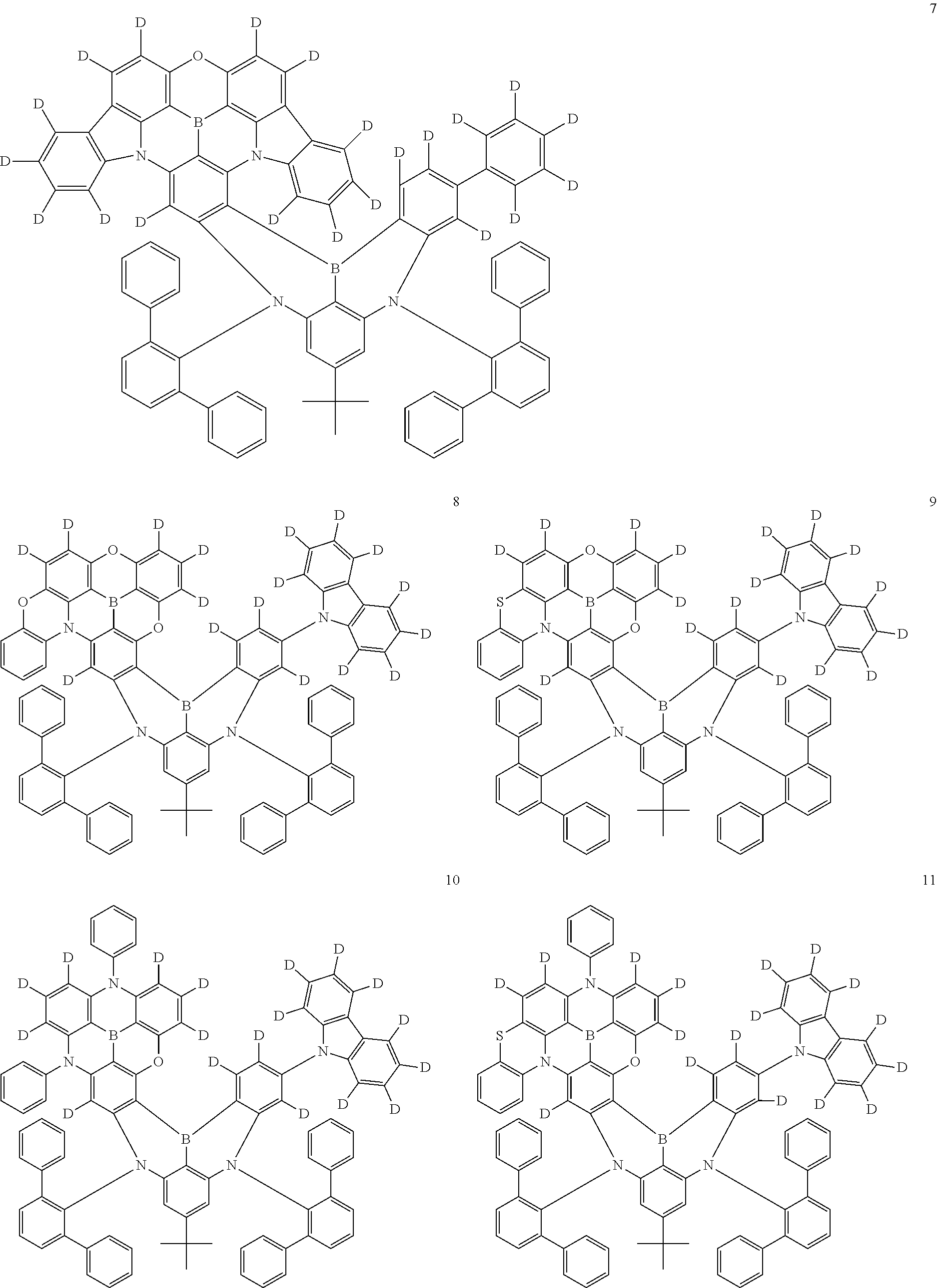

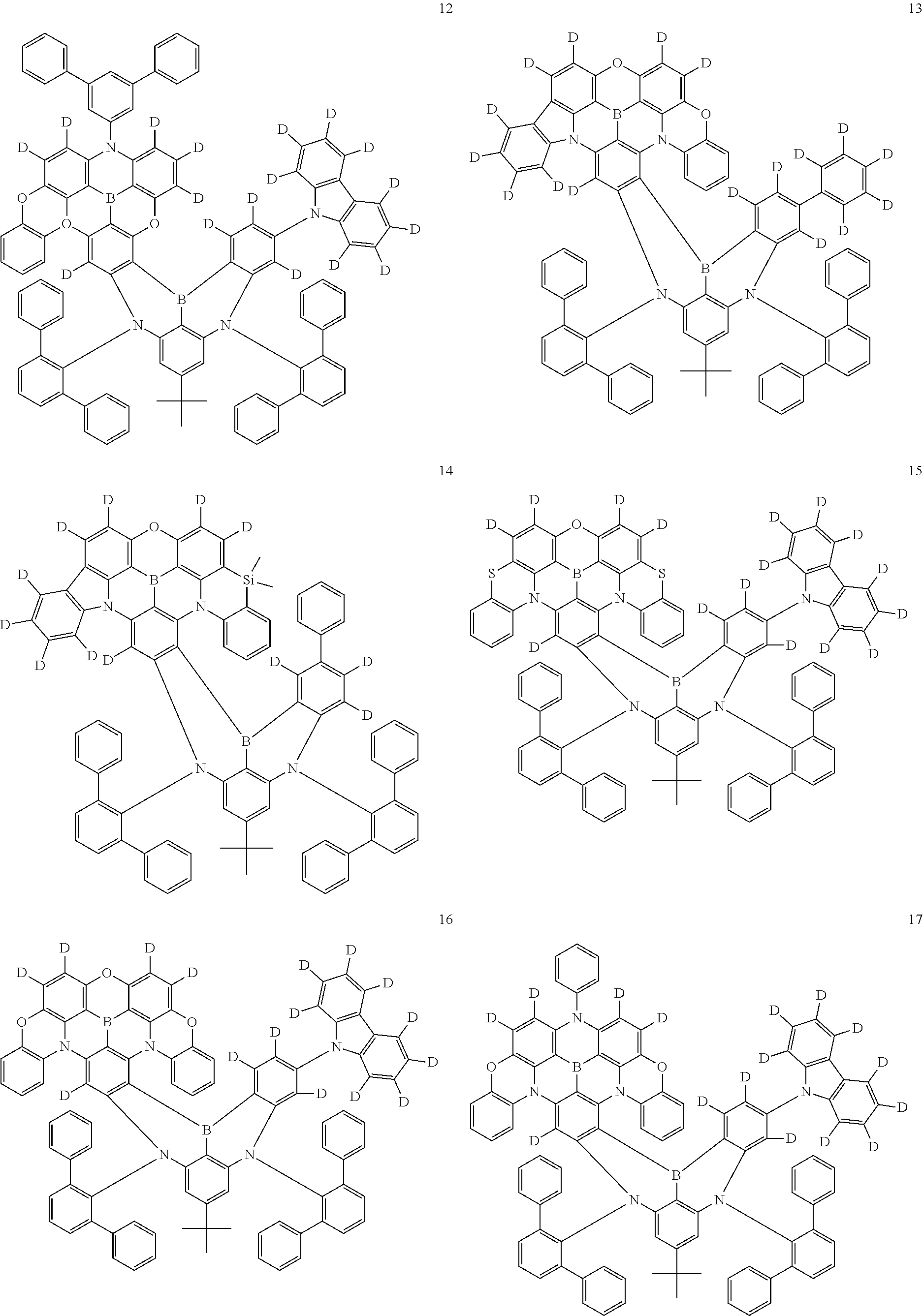

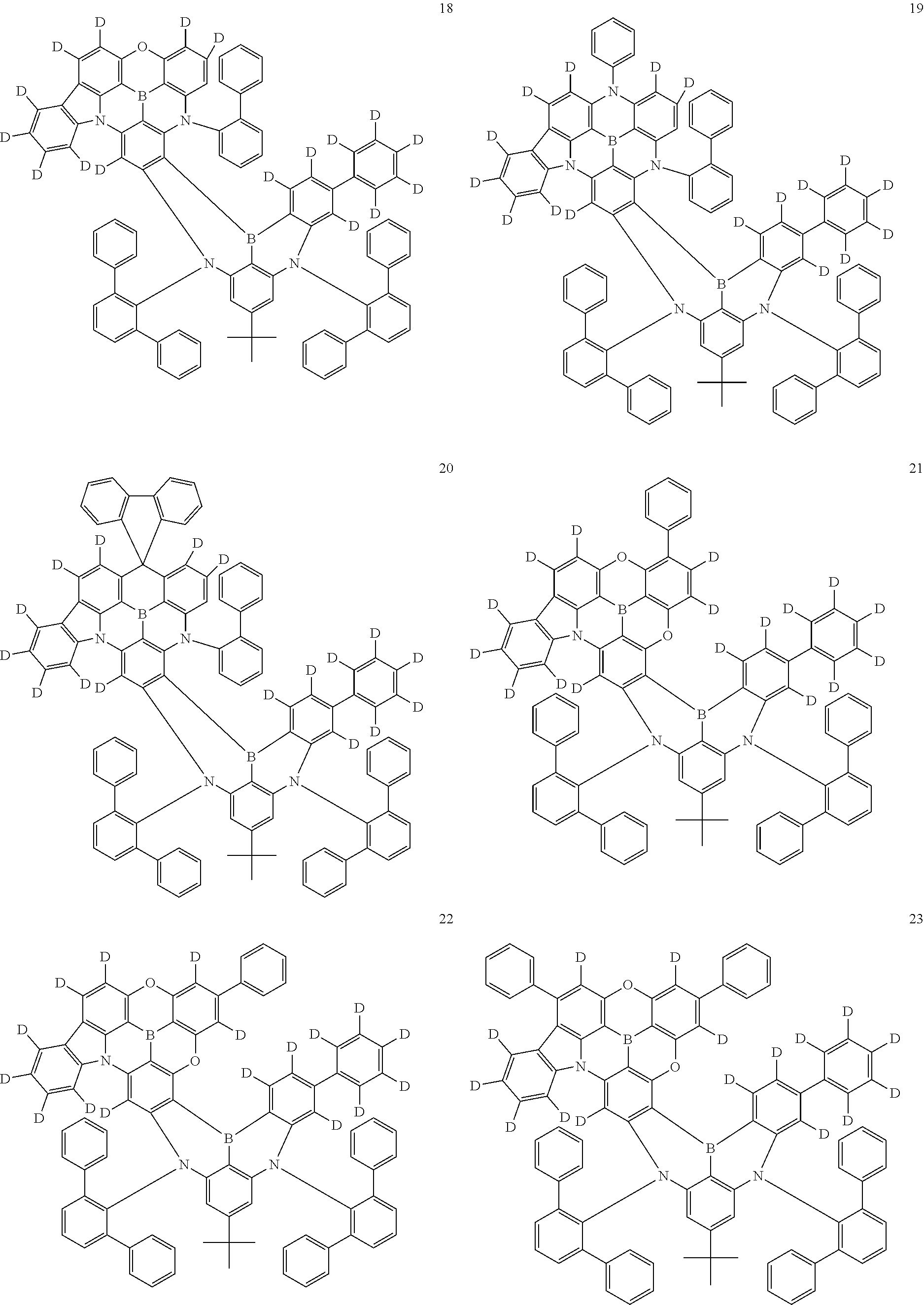

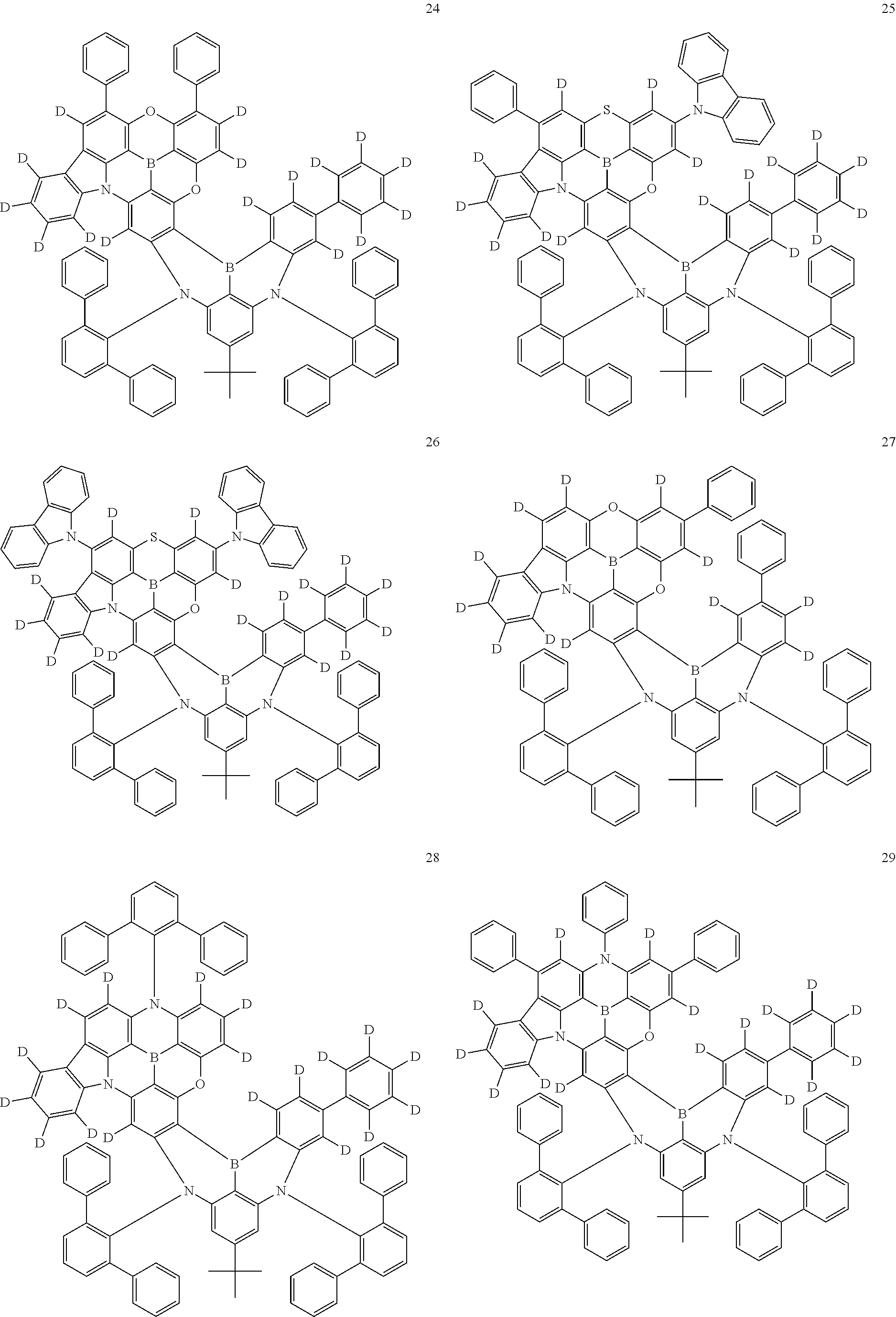

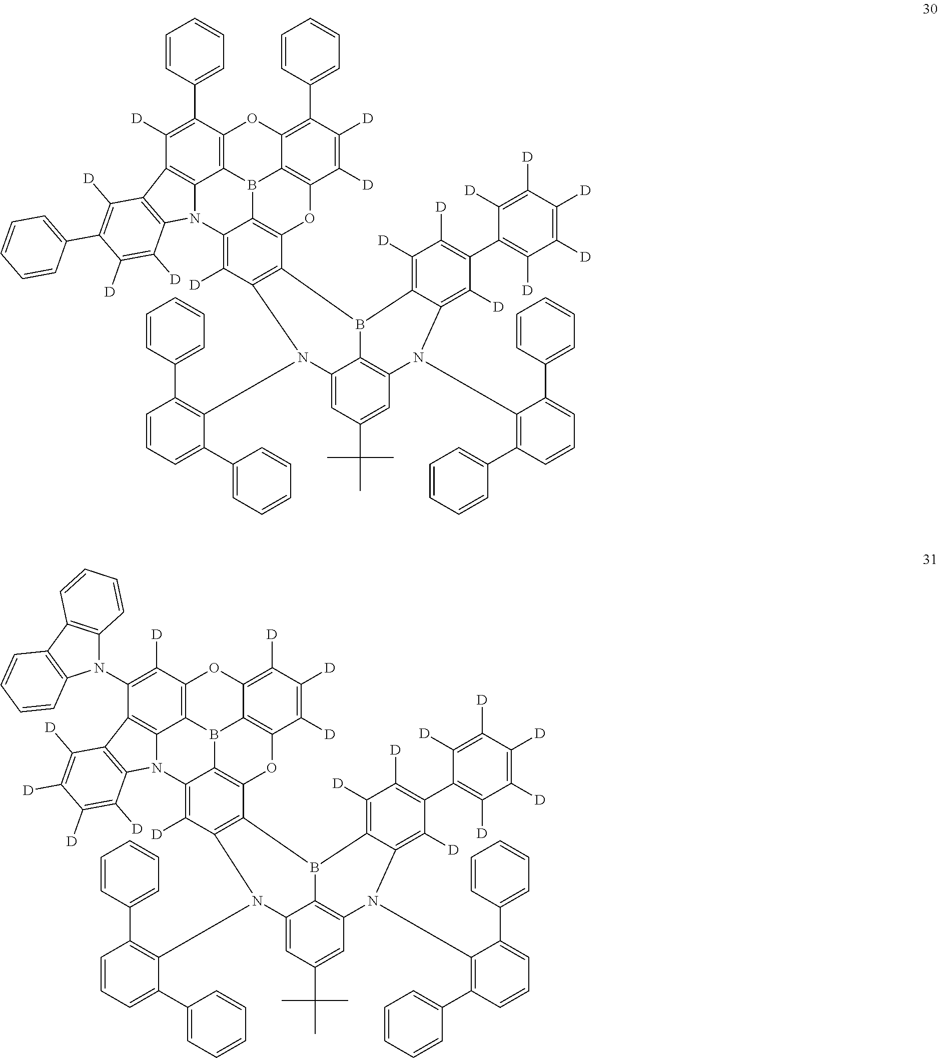

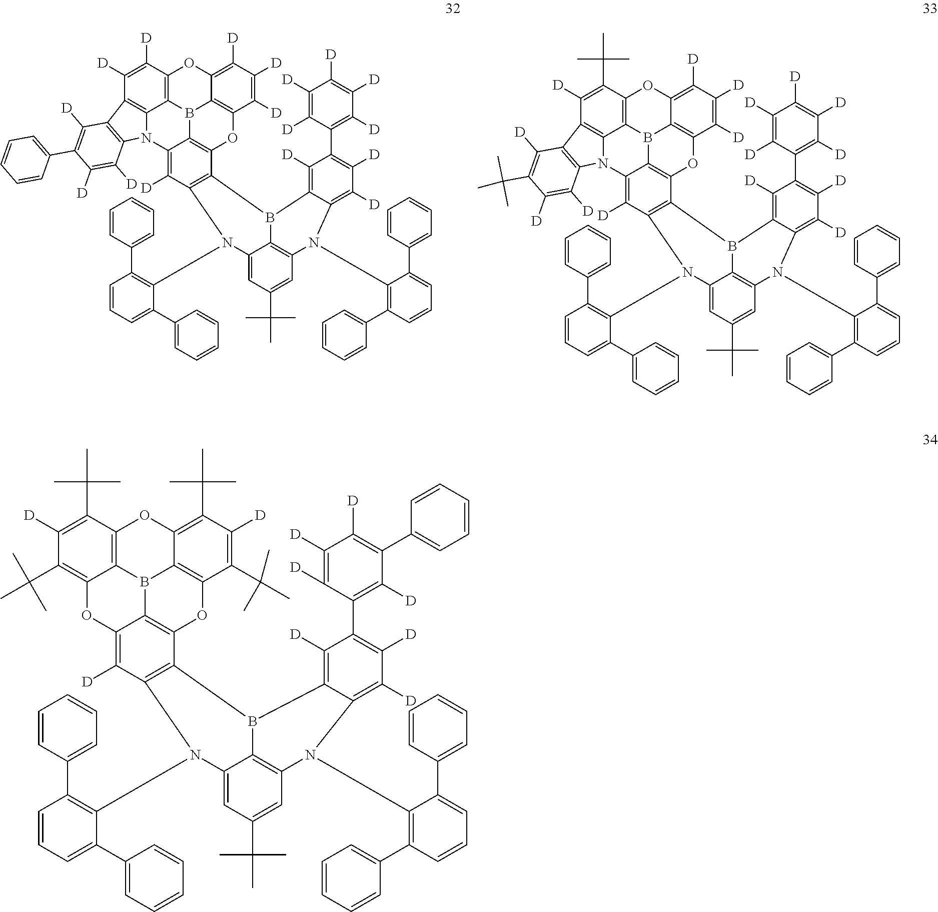

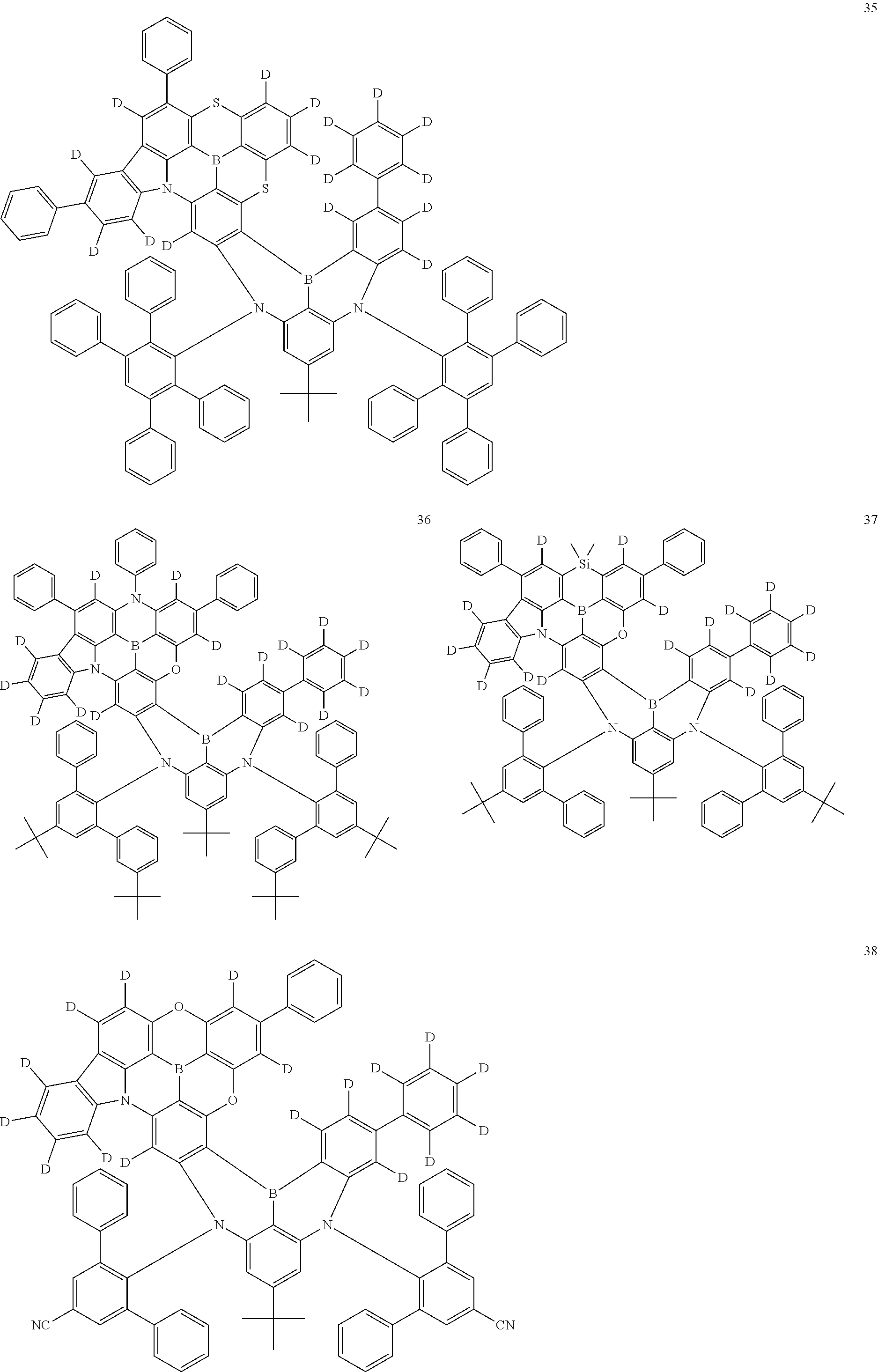

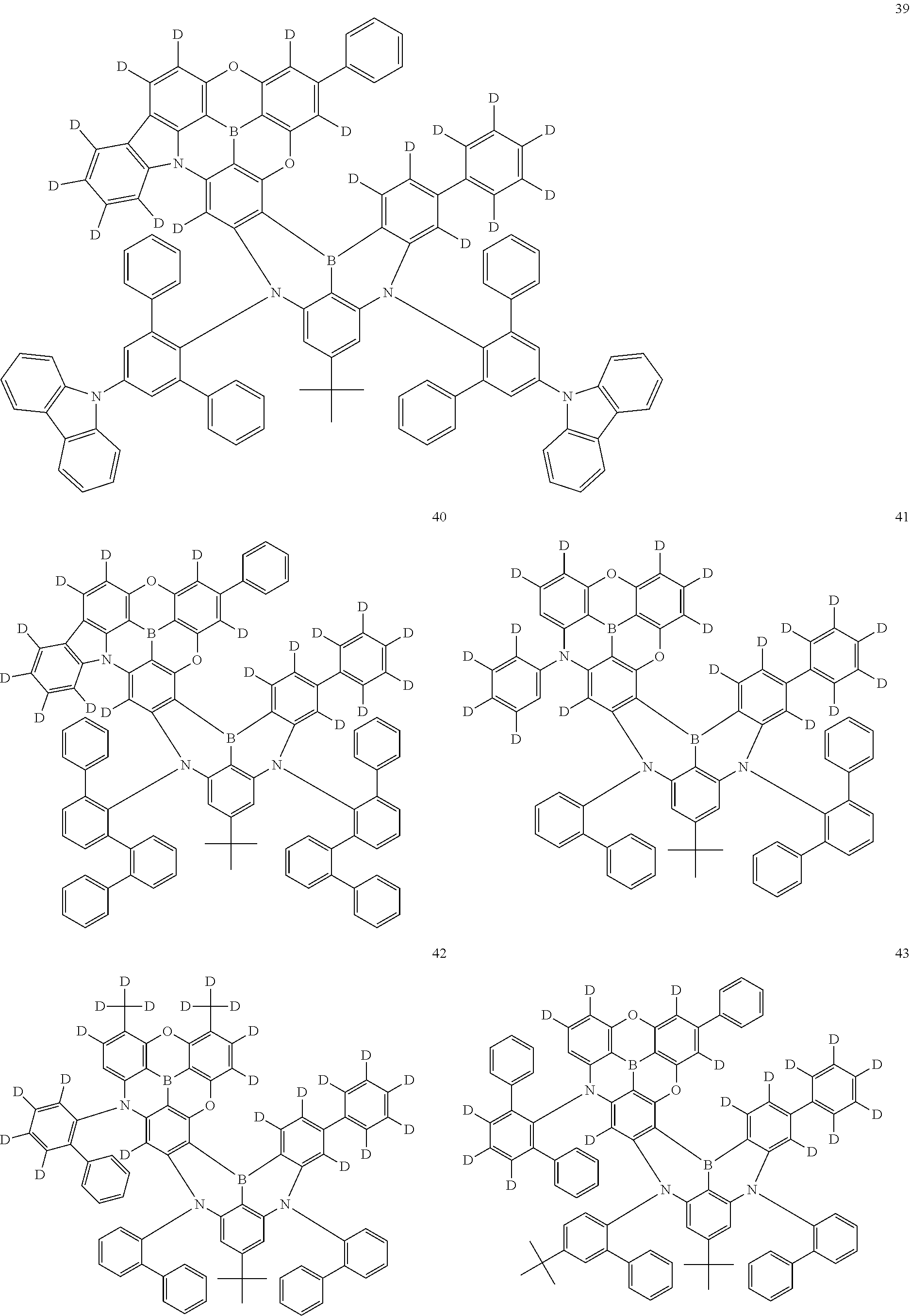

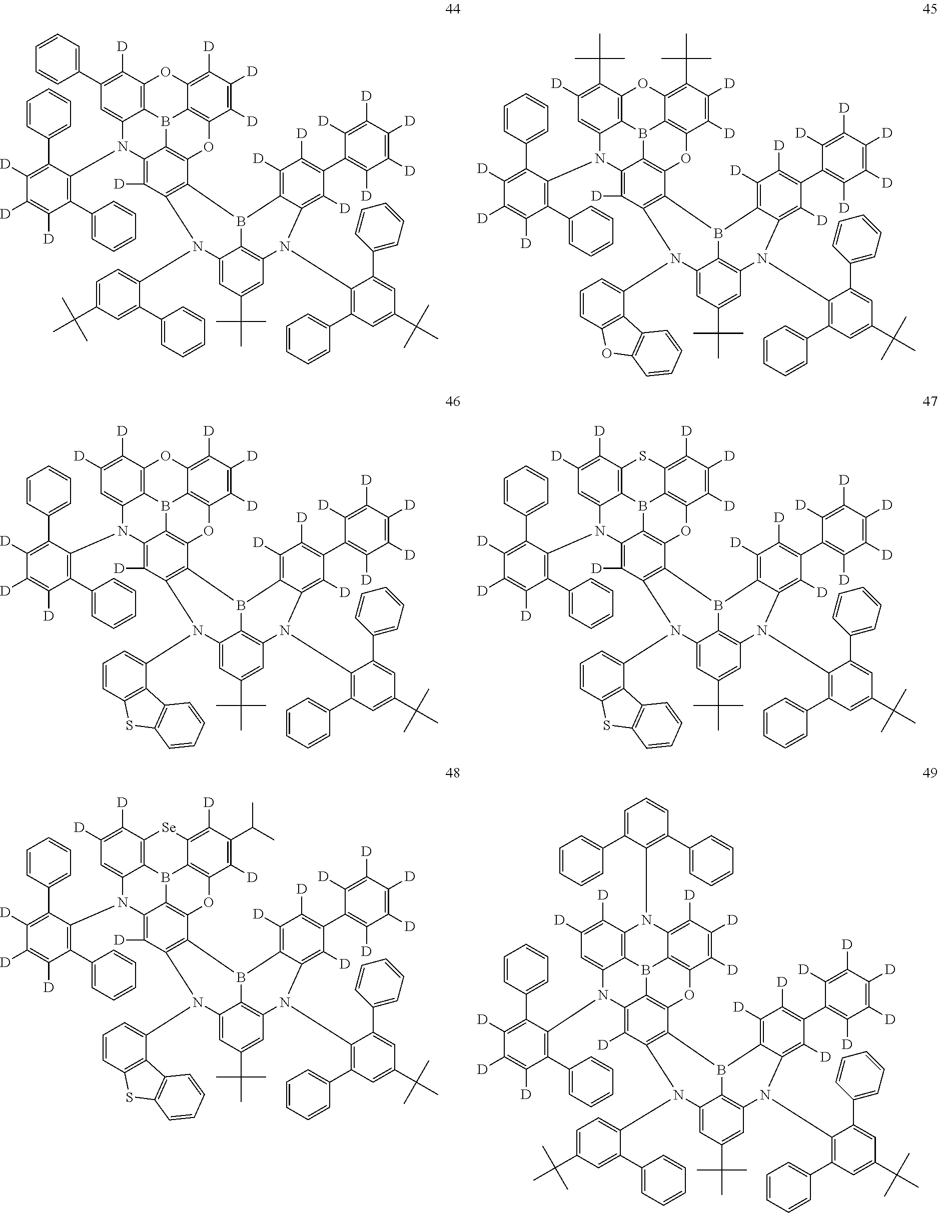

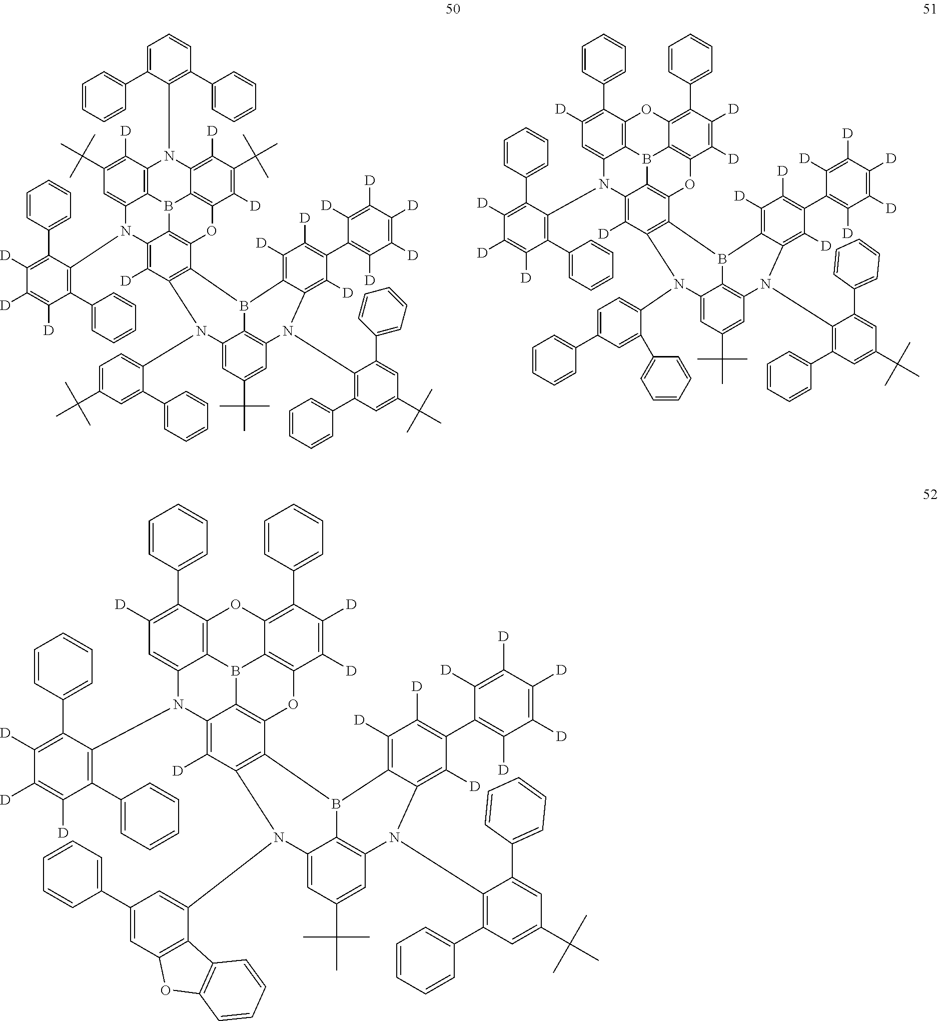

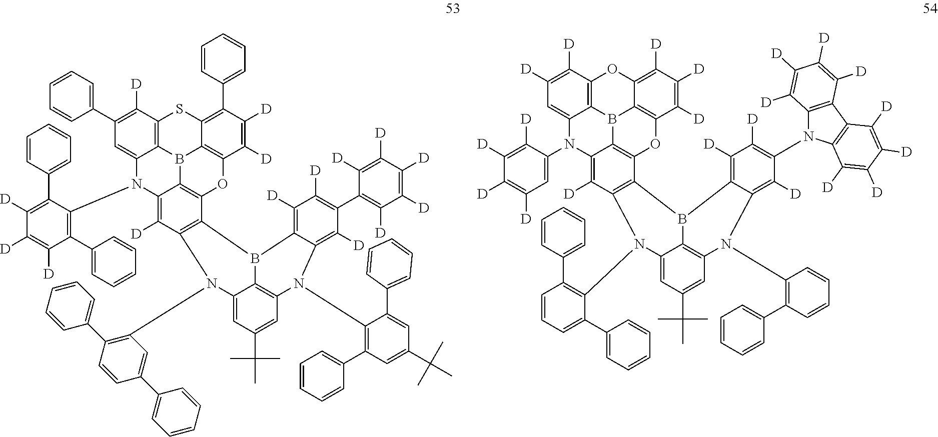

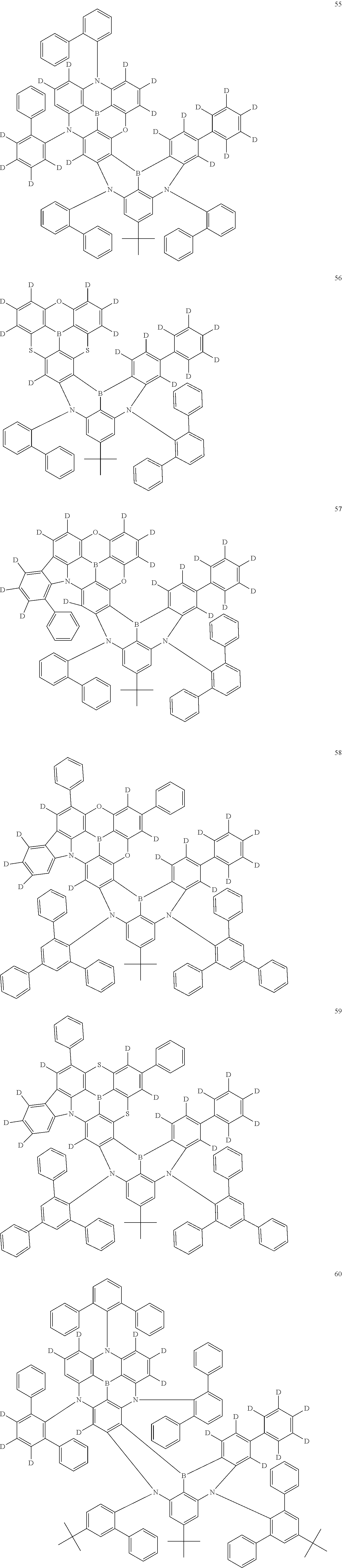

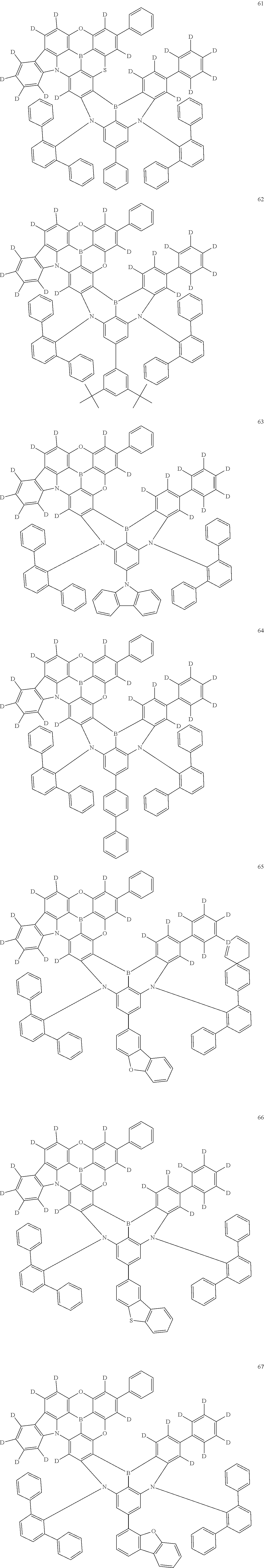

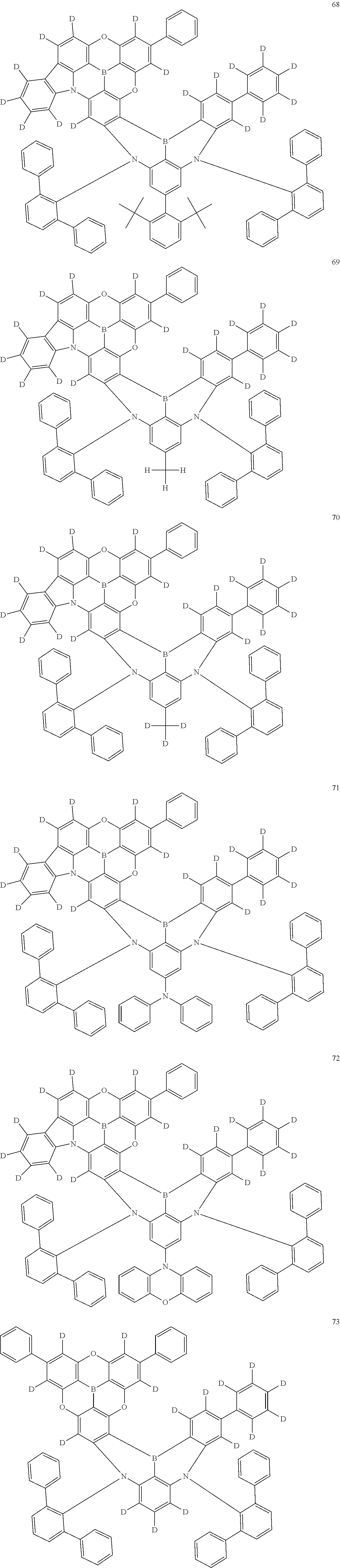

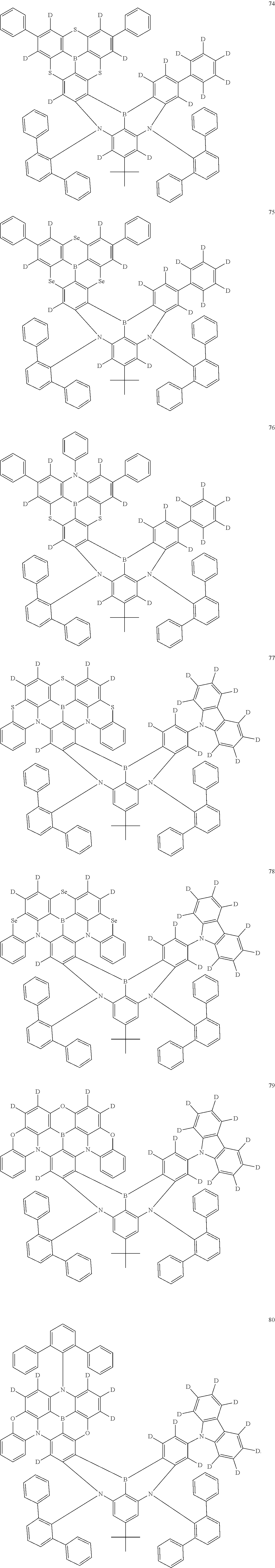

In one or more embodiments, the second compound may be any one selected from among Compounds ETH1 to ETH100.

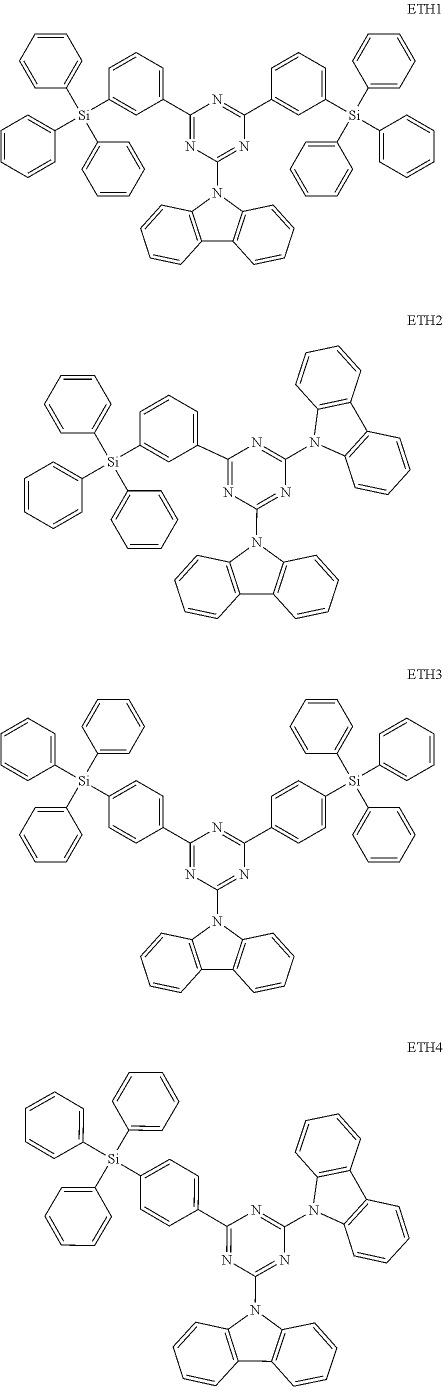 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

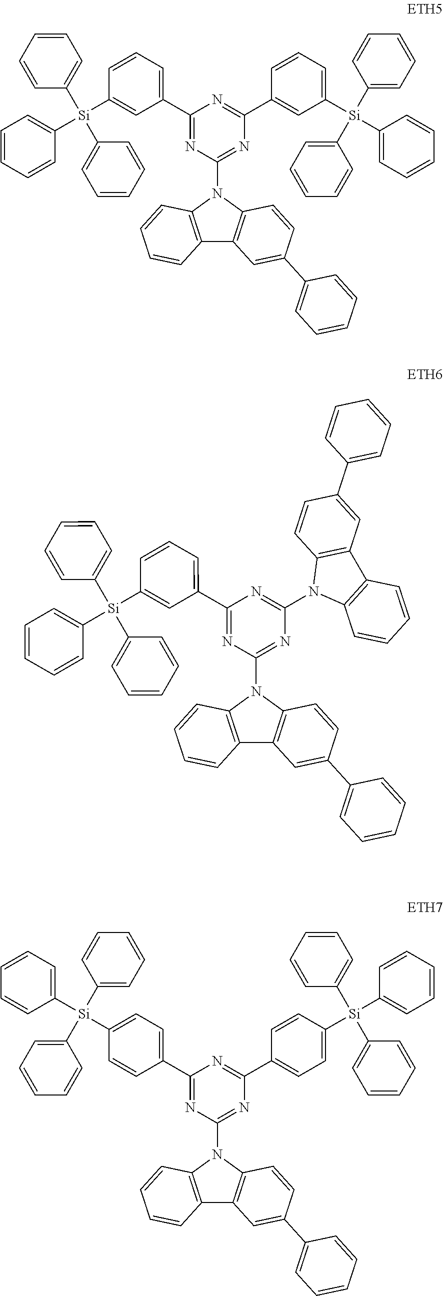 [SMILES: c1ccc(-c2ccc3c(c2)c2ccccc2n3-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; c1ccc(-c2ccc3c(c2)c2ccccc2n3-c2nc(-c3cccc([Si](c4ccccc4)(c4ccccc4)c4ccccc4)c3)nc(-n3c4ccccc4c4cc(-c5ccccc5)ccc43)n2)cc1 ; c1ccc(-c2ccc3c(c2)c2ccccc2n3-c2nc(-c3ccc([Si](c4ccccc4)(c4ccccc4)c4ccccc4)cc3)nc(-c3ccc([Si](c4ccccc4)(c4ccccc4)c4ccccc4)cc3)n2)cc1]

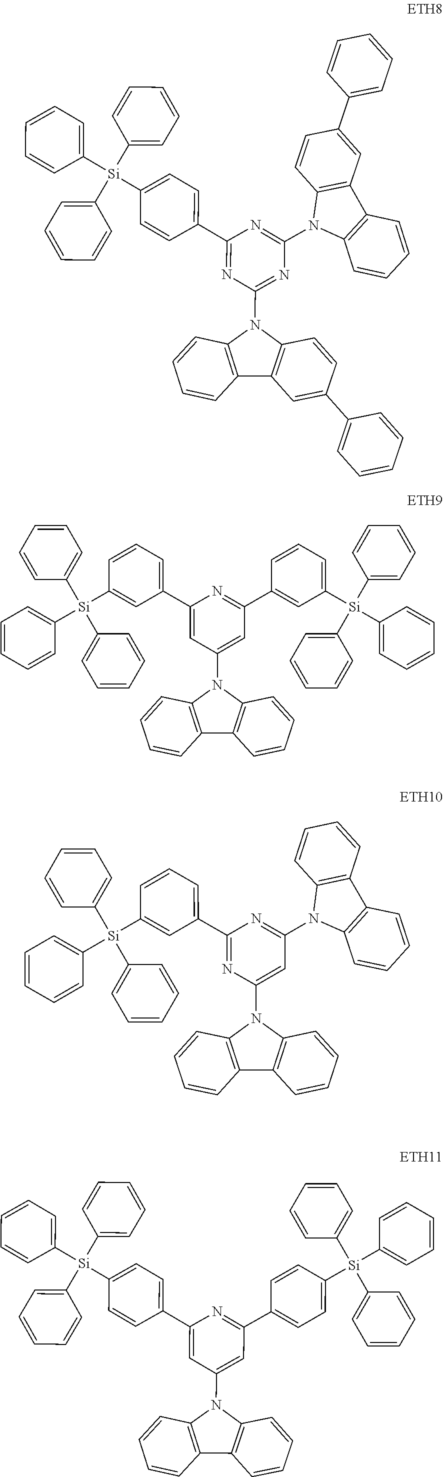 [SMILES: c1ccc(-c2ccc3c(c2)c2ccccc2n3-c2nc(-c3ccc([Si](c4ccccc4)(c4ccccc4)c4ccccc4)cc3)nc(-n3c4ccccc4c4cc(-c5ccccc5)ccc43)n2)cc1 ; c1ccc([Si](c2ccccc2)(c2ccccc2)c2cccc(-c3cc(-n4c5ccccc5c5ccccc54)cc(-c4cccc([Si](c5ccccc5)(c5ccccc5)c5ccccc5)c4)n3)c2)cc1 ; c1ccc([Si](c2ccccc2)(c2ccccc2)c2cccc(-c3nc(-n4c5ccccc5c5ccccc54)cc(-n4c5ccccc5c5ccccc54)n3)c2)cc1 ; c1ccc([Si](c2ccccc2)(c2ccccc2)c2ccc(-c3cc(-n4c5ccccc5c5ccccc54)cc(-c4ccc([Si](c5ccccc5)(c5ccccc5)c5ccccc5)cc4)n3)cc2)cc1]

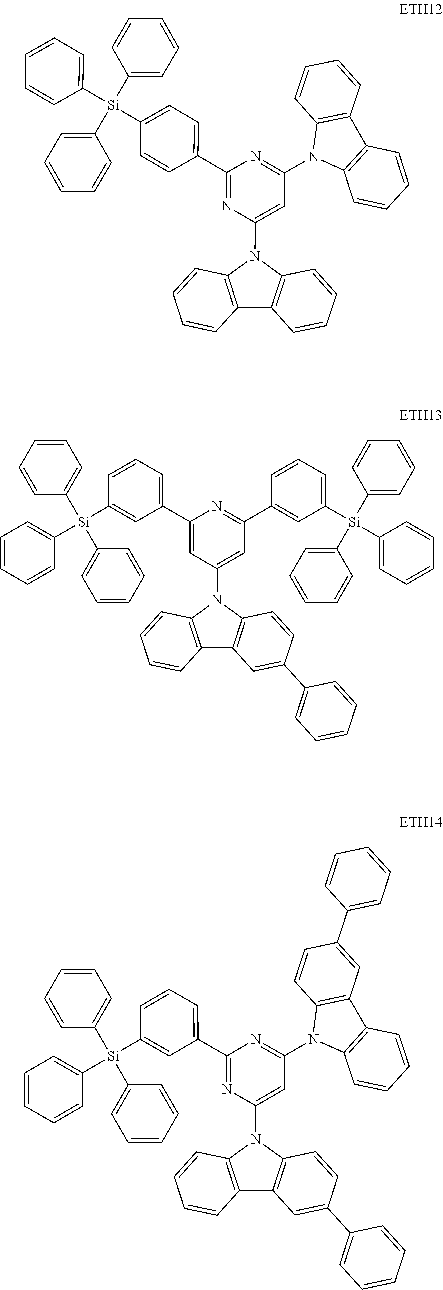 [SMILES: c1ccc([Si](c2ccccc2)(c2ccccc2)c2ccc(-c3nc(-n4c5ccccc5c5ccccc54)cc(-n4c5ccccc5c5ccccc54)n3)cc2)cc1 ; c1ccc(-c2ccc3c(c2)c2ccccc2n3-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; c1ccc(-c2ccc3c(c2)c2ccccc2n3-c2cc(-n3c4ccccc4c4cc(-c5ccccc5)ccc43)nc(-c3cccc([Si](c4ccccc4)(c4ccccc4)c4ccccc4)c3)n2)cc1]

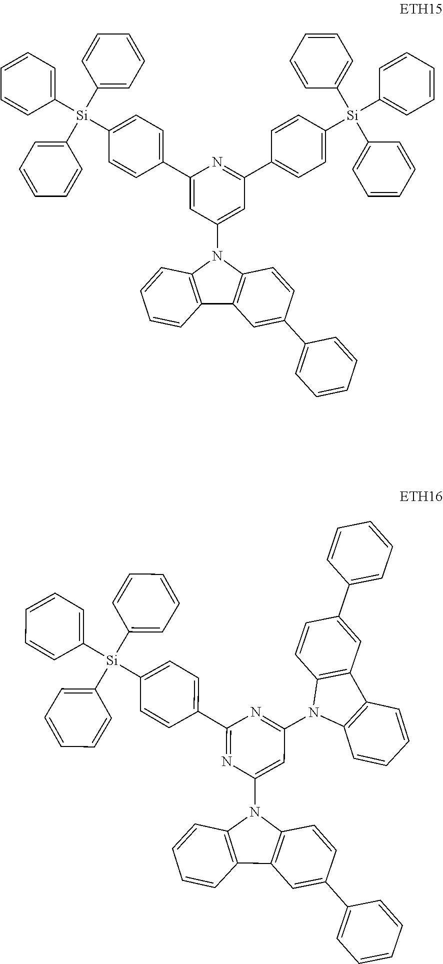 [SMILES: c1ccc(-c2ccc3c(c2)c2ccccc2n3-c2cc(-c3ccc([Si](c4ccccc4)(c4ccccc4)c4ccccc4)cc3)nc(-c3ccc([Si](c4ccccc4)(c4ccccc4)c4ccccc4)cc3)c2)cc1 ; c1ccc(-c2ccc3c(c2)c2ccccc2n3-c2cc(-n3c4ccccc4c4cc(-c5ccccc5)ccc43)nc(-c3ccc([Si](c4ccccc4)(c4ccccc4)c4ccccc4)cc3)n2)cc1]

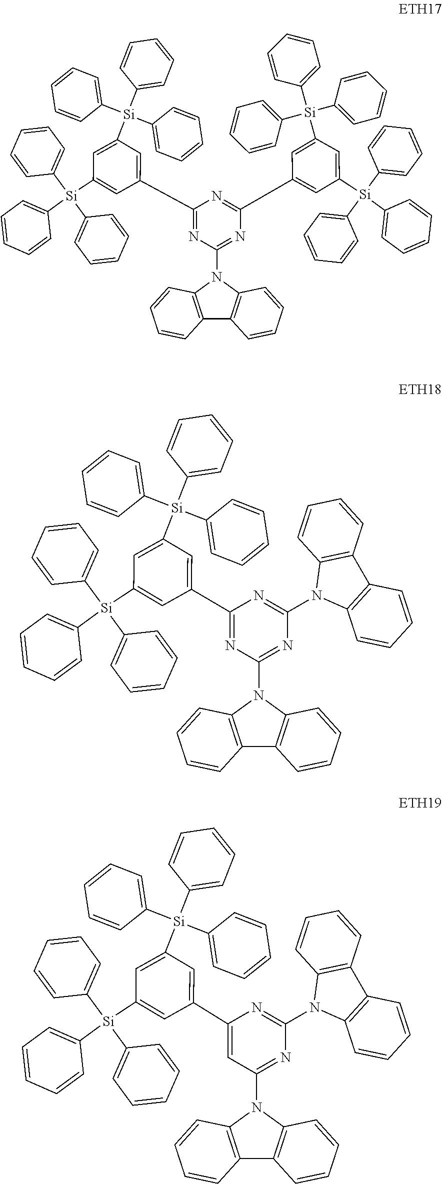 [SMILES: c1ccc([Si](c2ccccc2)(c2ccccc2)c2cc(-c3nc(-c4cc([Si](c5ccccc5)(c5ccccc5)c5ccccc5)cc([Si](c5ccccc5)(c5ccccc5)c5ccccc5)c4)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3cc(-n4c5ccccc5c5ccccc54)nc(-n4c5ccccc5c5ccccc54)n3)cc([Si](c3ccccc3)(c3ccccc3)c3ccccc3)c2)cc1]

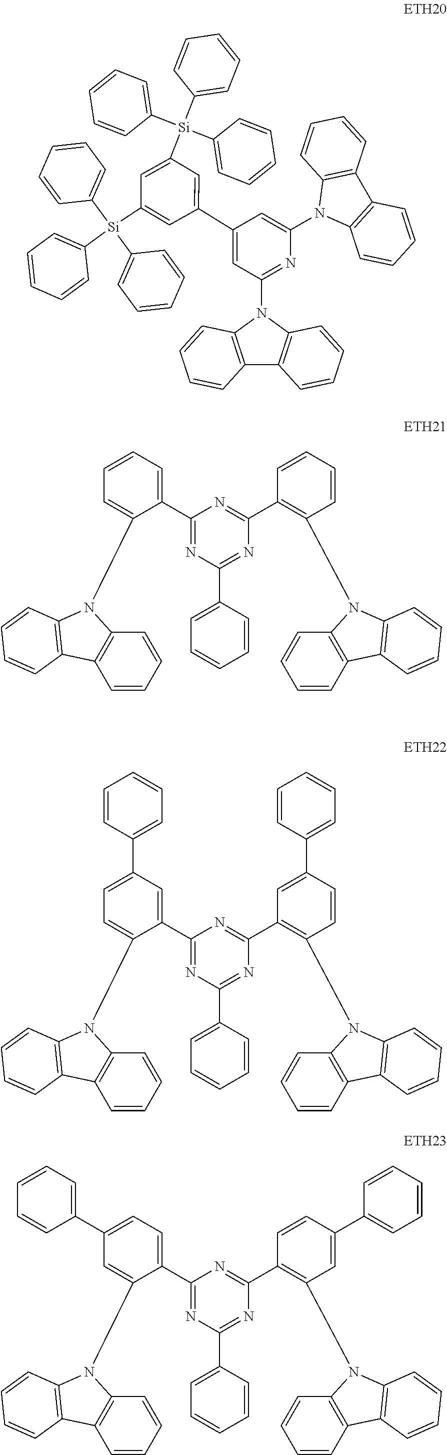 [SMILES: c1ccc([Si](c2ccccc2)(c2ccccc2)c2cc(-c3cc(-n4c5ccccc5c5ccccc54)nc(-n4c5ccccc5c5ccccc54)c3)cc([Si](c3ccccc3)(c3ccccc3)c3ccccc3)c2)cc1 ; c1ccc(-c2nc(-c3ccccc3-n3c4ccccc4c4ccccc43)nc(-c3ccccc3-n3c4ccccc4c4ccccc43)n2)cc1 ; c1ccc(-c2ccc(-n3c4ccccc4c4ccccc43)c(-c3nc(-c4ccccc4)nc(-c4cc(-c5ccccc5)ccc4-n4c5ccccc5c5ccccc54)n3)c2)cc1 ; c1ccc(-c2ccc(-c3nc(-c4ccccc4)nc(-c4ccc(-c5ccccc5)cc4-n4c5ccccc5c5ccccc54)n3)c(-n3c4ccccc4c4ccccc43)c2)cc1]

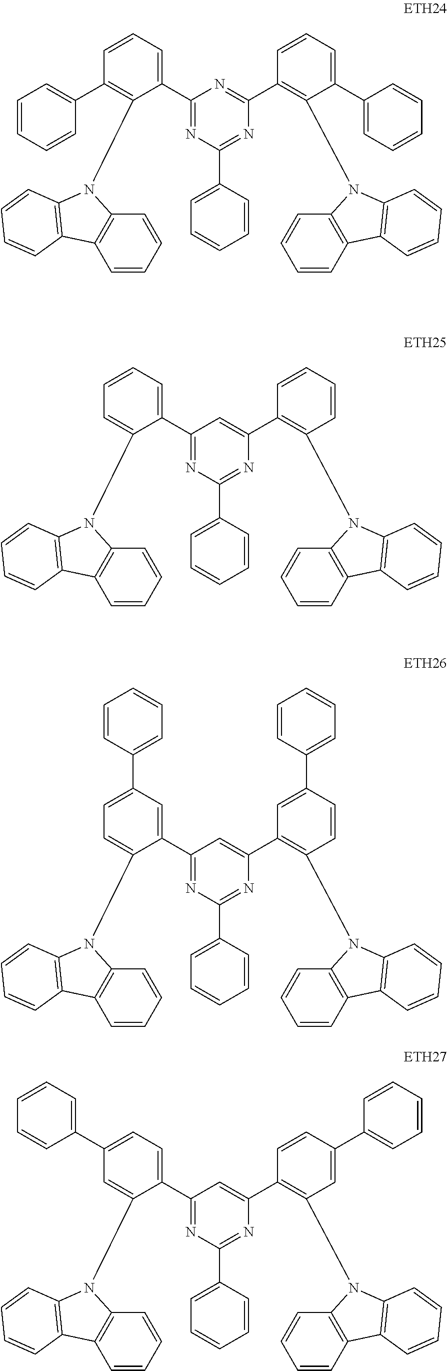 [SMILES: c1ccc(-c2nc(-c3cccc(-c4ccccc4)c3-n3c4ccccc4c4ccccc43)nc(-c3cccc(-c4ccccc4)c3-n3c4ccccc4c4ccccc43)n2)cc1 ; c1ccc(-c2nc(-c3ccccc3-n3c4ccccc4c4ccccc43)cc(-c3ccccc3-n3c4ccccc4c4ccccc43)n2)cc1 ; c1ccc(-c2ccc(-n3c4ccccc4c4ccccc43)c(-c3cc(-c4cc(-c5ccccc5)ccc4-n4c5ccccc5c5ccccc54)nc(-c4ccccc4)n3)c2)cc1 ; c1ccc(-c2ccc(-c3cc(-c4ccc(-c5ccccc5)cc4-n4c5ccccc5c5ccccc54)nc(-c4ccccc4)n3)c(-n3c4ccccc4c4ccccc43)c2)cc1]

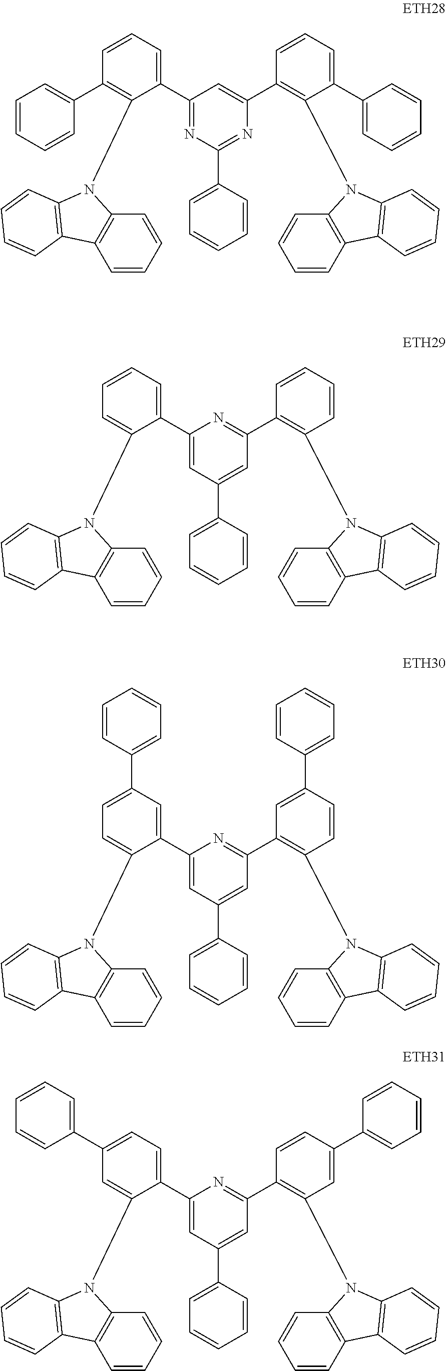 [SMILES: c1ccc(-c2nc(-c3cccc(-c4ccccc4)c3-n3c4ccccc4c4ccccc43)cc(-c3cccc(-c4ccccc4)c3-n3c4ccccc4c4ccccc43)n2)cc1 ; c1ccc(-c2cc(-c3ccccc3-n3c4ccccc4c4ccccc43)nc(-c3ccccc3-n3c4ccccc4c4ccccc43)c2)cc1 ; c1ccc(-c2cc(-c3cc(-c4ccccc4)ccc3-n3c4ccccc4c4ccccc43)nc(-c3cc(-c4ccccc4)ccc3-n3c4ccccc4c4ccccc43)c2)cc1 ; c1ccc(-c2cc(-c3ccc(-c4ccccc4)cc3-n3c4ccccc4c4ccccc43)nc(-c3ccc(-c4ccccc4)cc3-n3c4ccccc4c4ccccc43)c2)cc1]

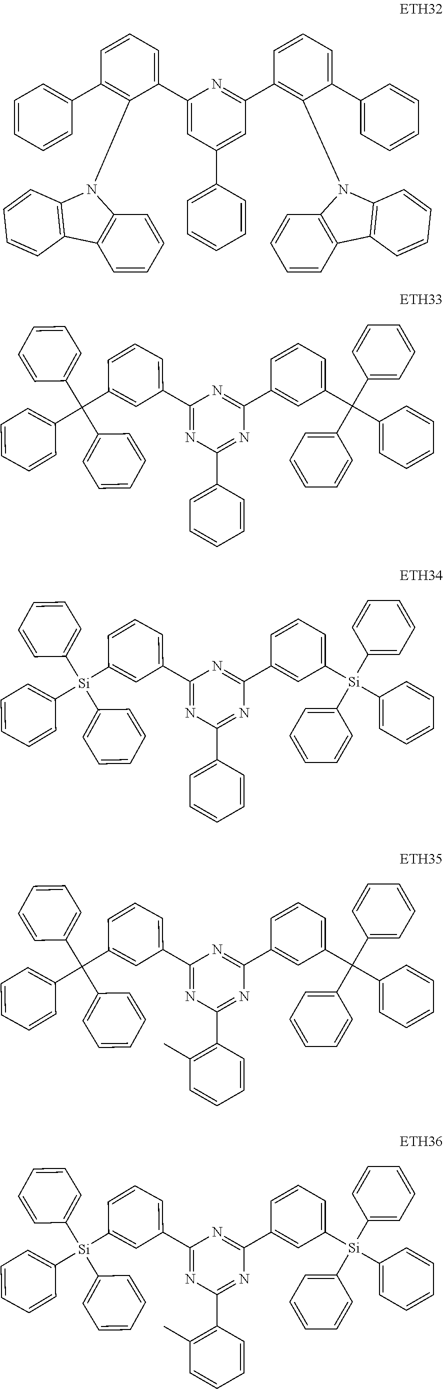

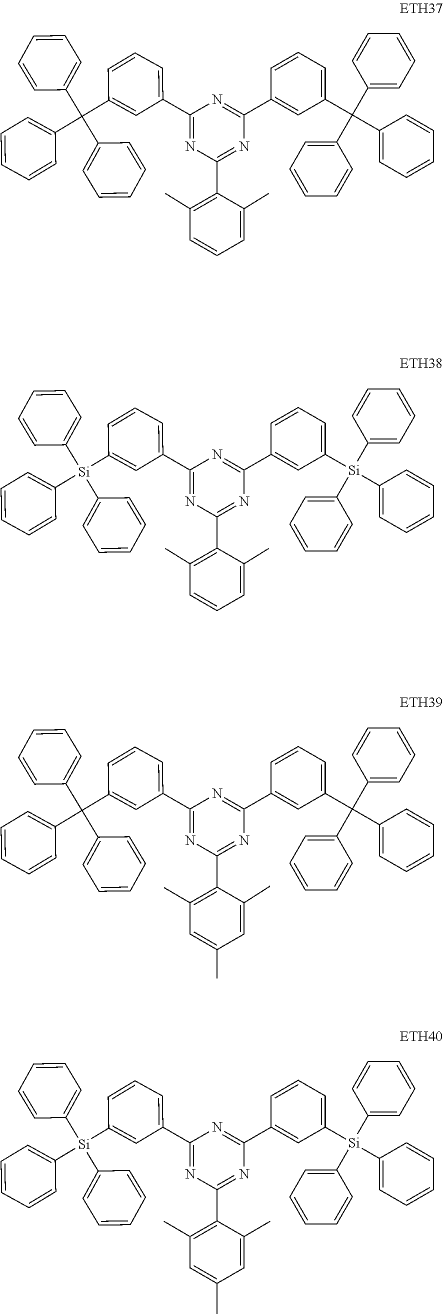 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

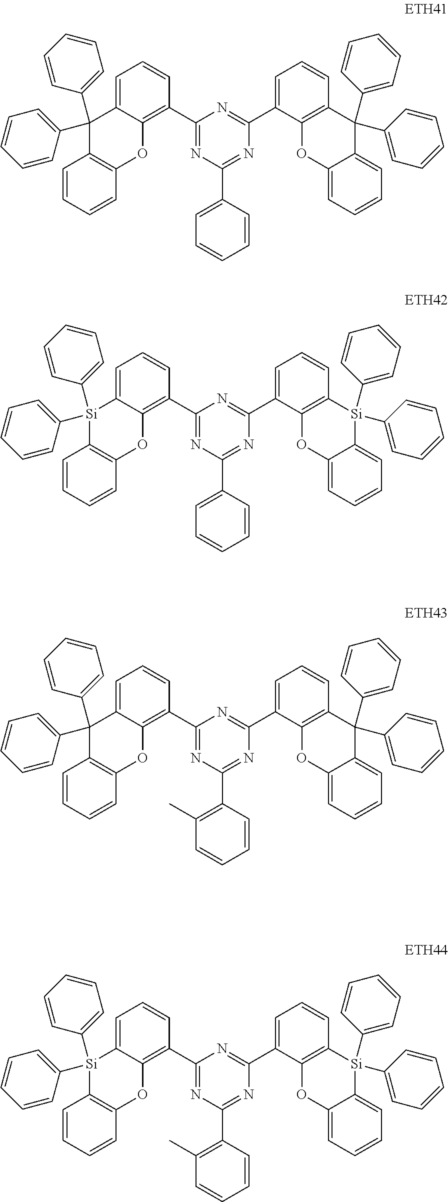 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

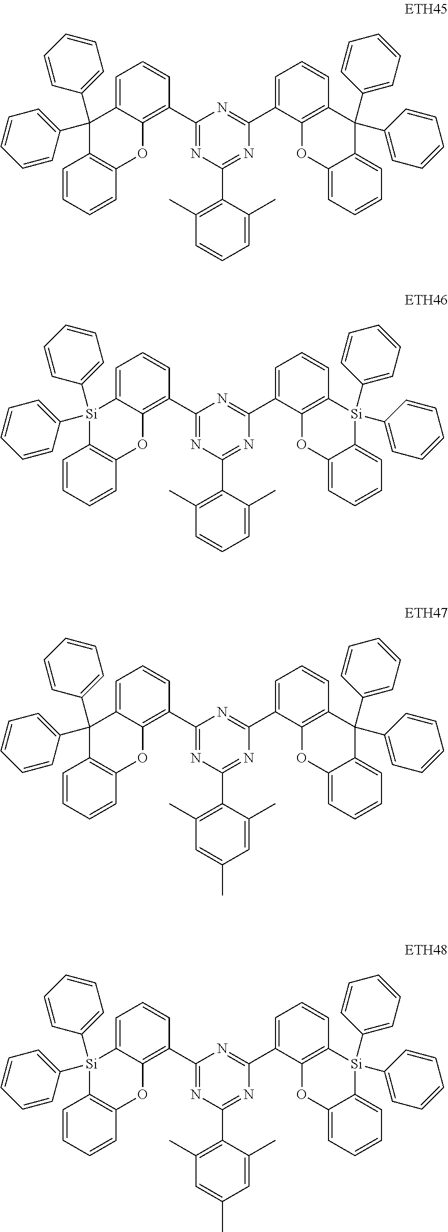 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

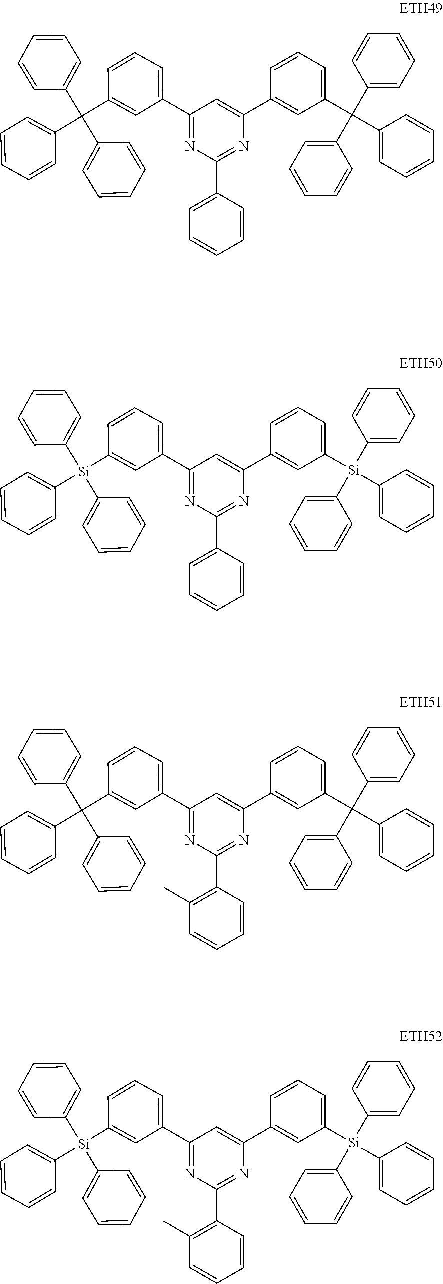 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

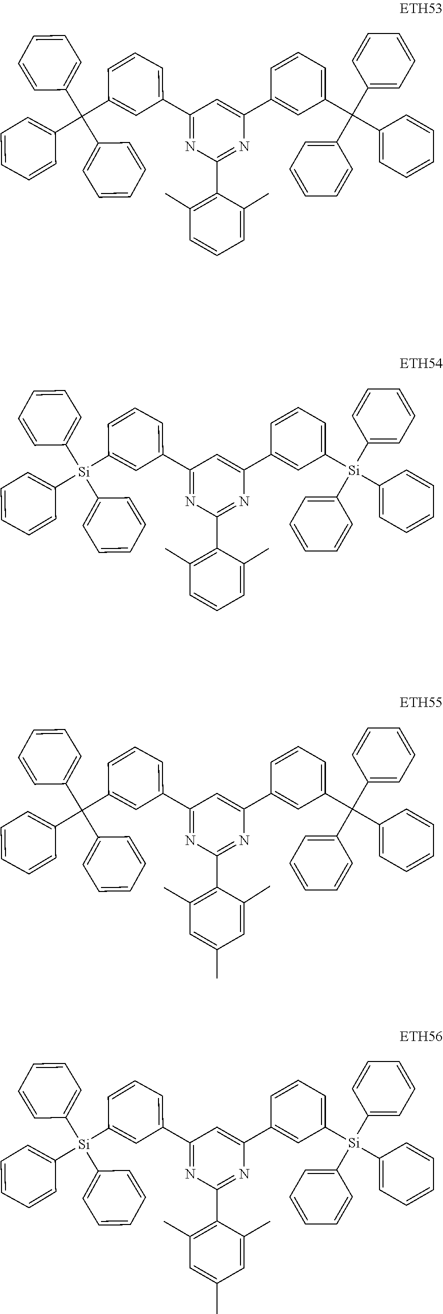 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

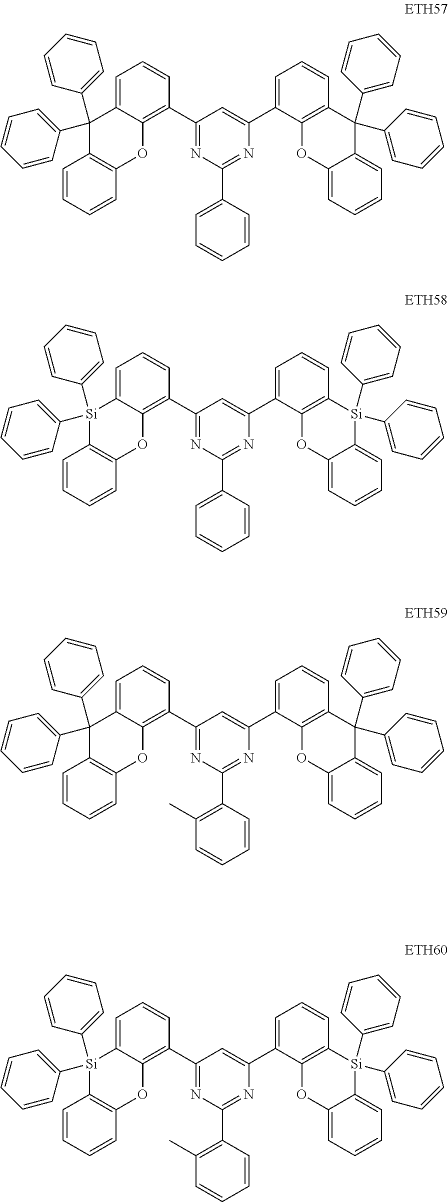 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

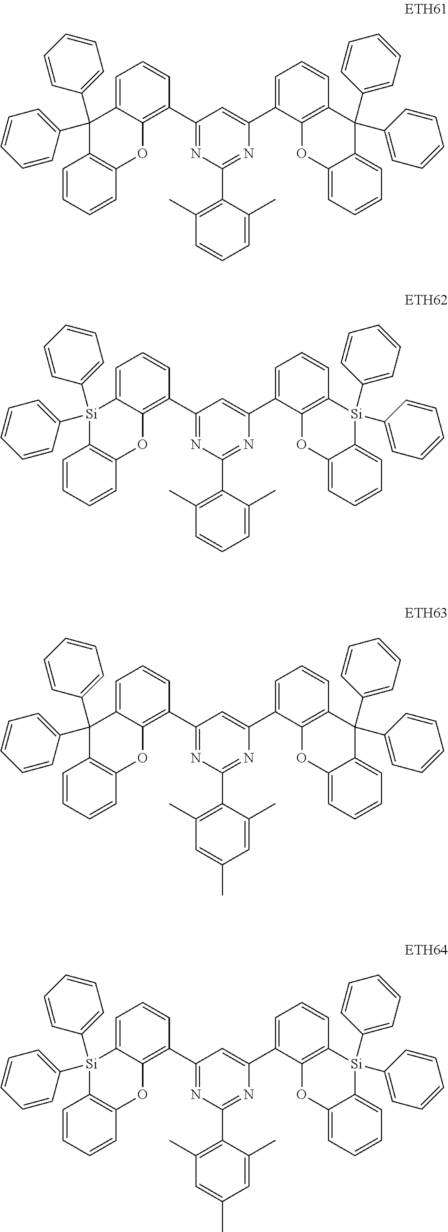 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

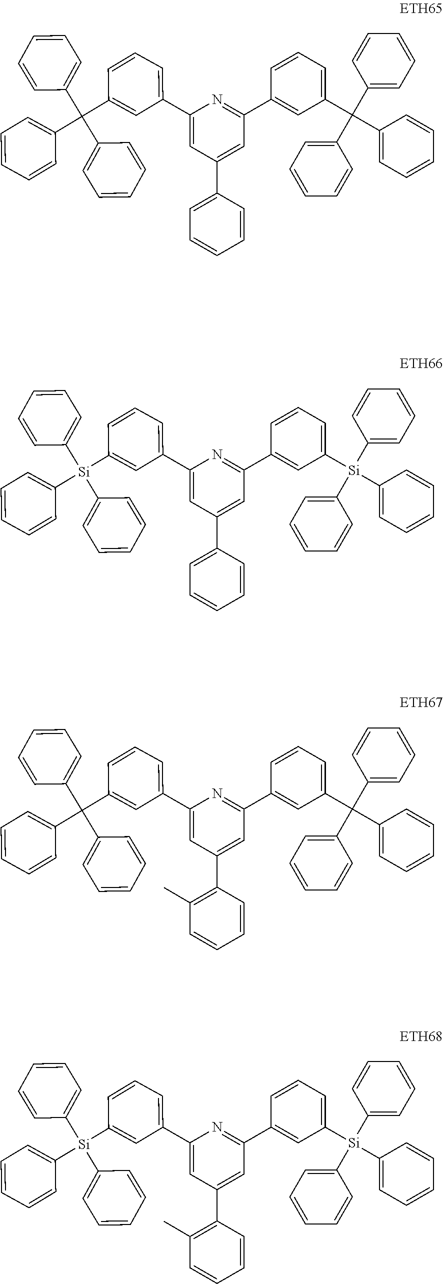 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

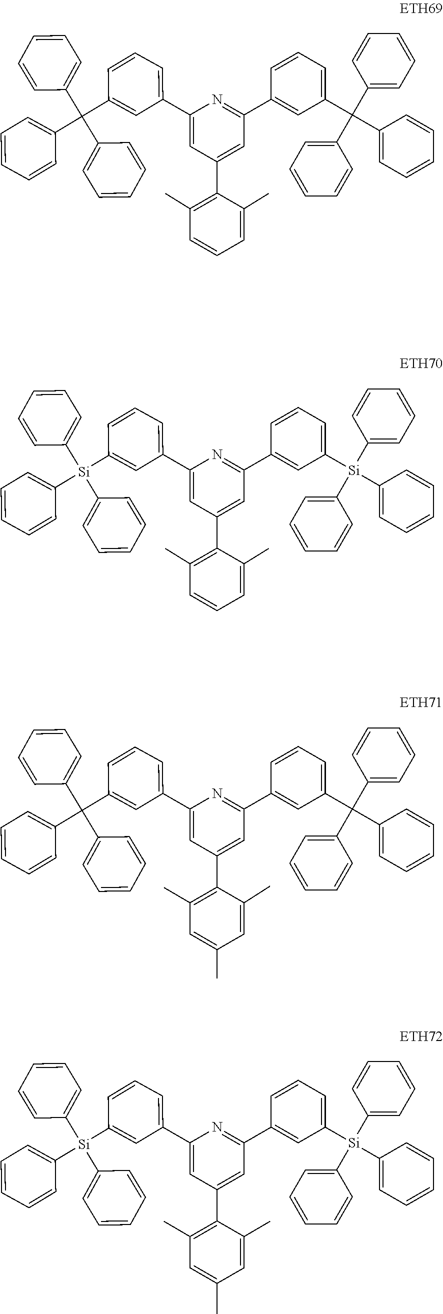 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

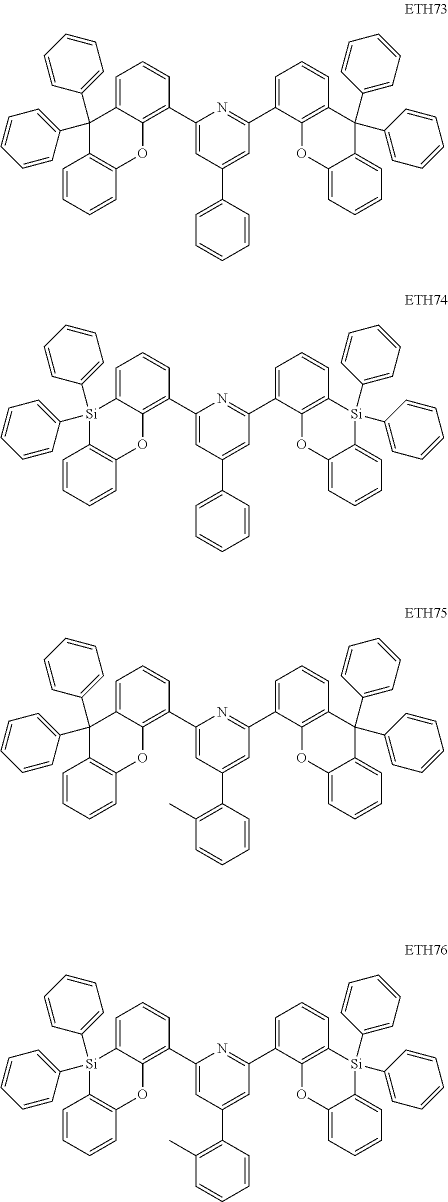 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

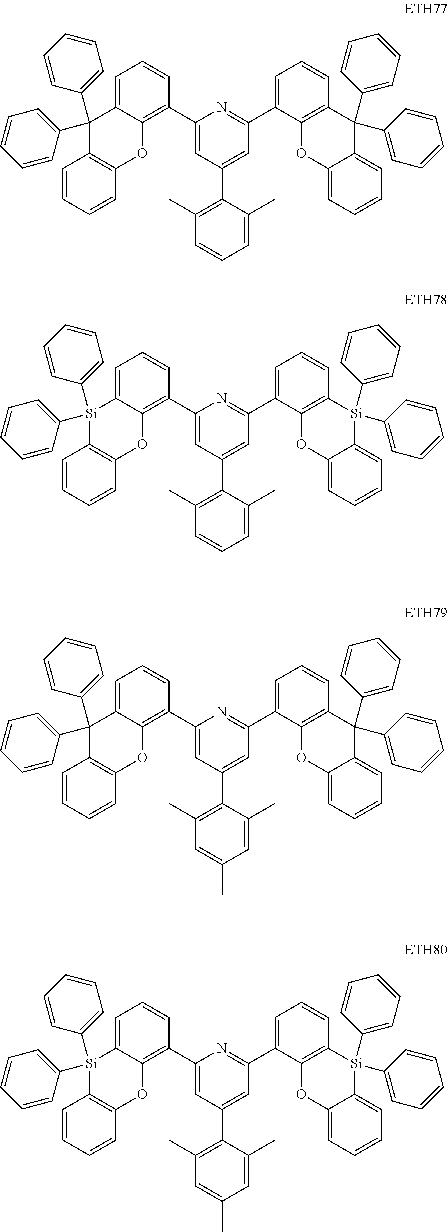 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

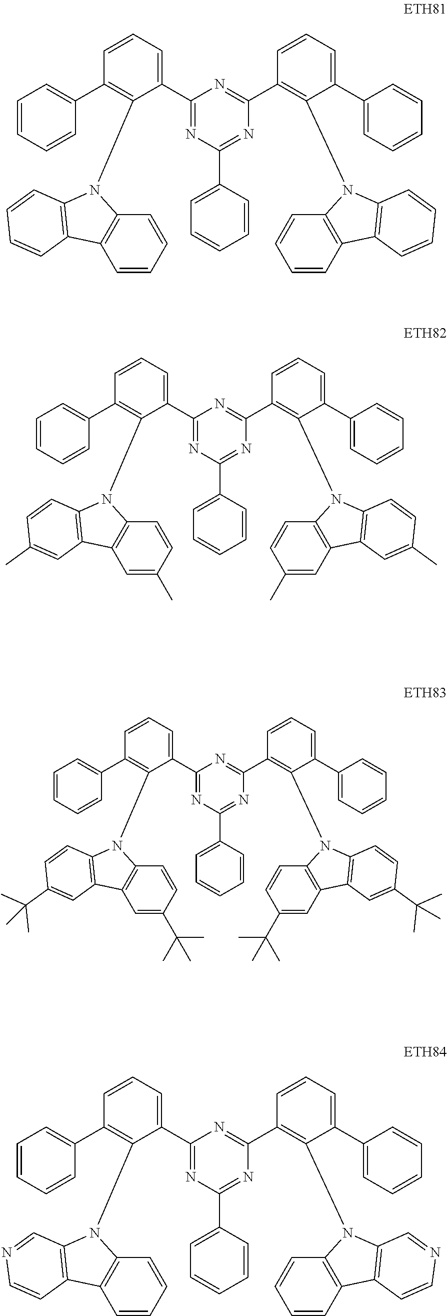

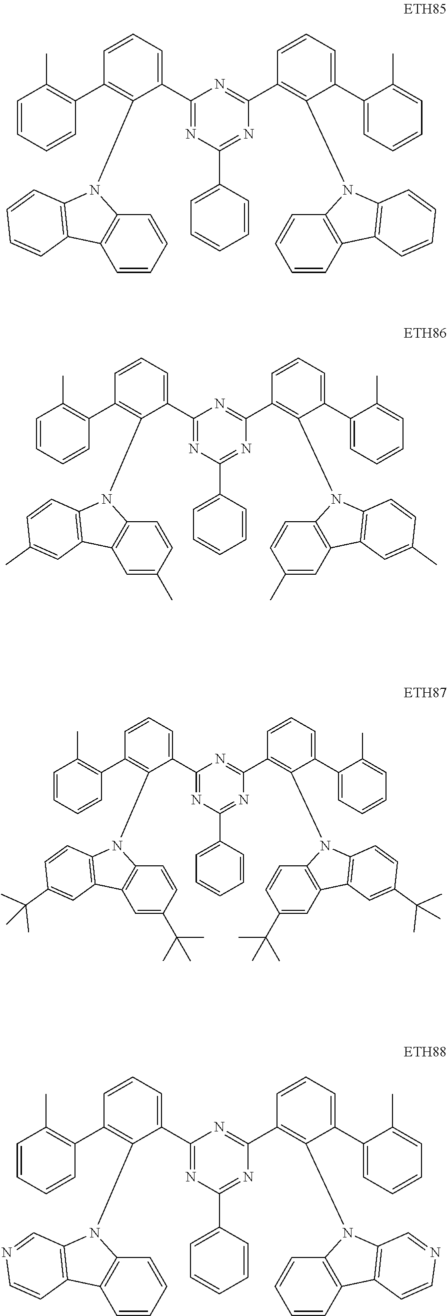

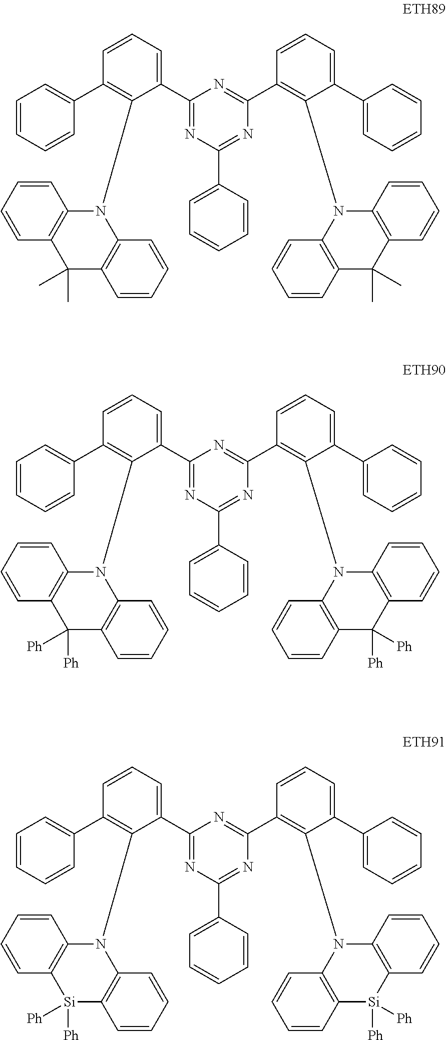 [SMILES: CC1(C)c2ccccc2N(c2c(-c3ccccc3)cccc2-c2nc(-c3ccccc3)nc(-c3cccc(-c4ccccc4)c3N3c4ccccc4C(C)(C)c4ccccc43)n2)c2ccccc21 ; c1ccc(-c2nc(-c3cccc(-c4ccccc4)c3N3c4ccccc4C(c4ccccc4)(c4ccccc4)c4ccccc43)nc(-c3cccc(-c4ccccc4)c3N3c4ccccc4C(c4ccccc4)(c4ccccc4)c4ccccc43)n2)cc1 ; c1ccc(-c2nc(-c3cccc(-c4ccccc4)c3N3c4ccccc4[Si](c4ccccc4)(c4ccccc4)c4ccccc43)nc(-c3cccc(-c4ccccc4)c3N3c4ccccc4[Si](c4ccccc4)(c4ccccc4)c4ccccc43)n2)cc1]

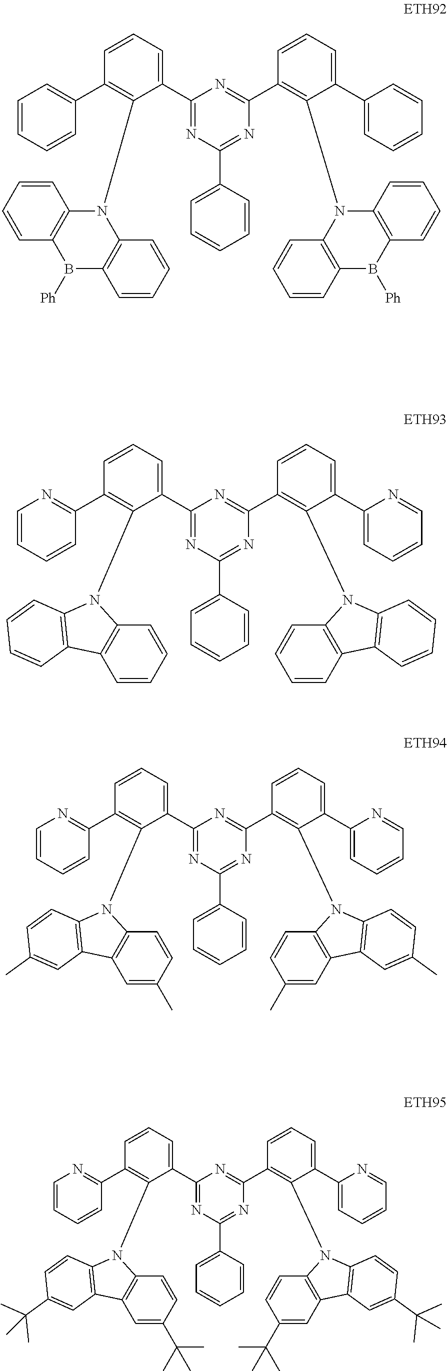

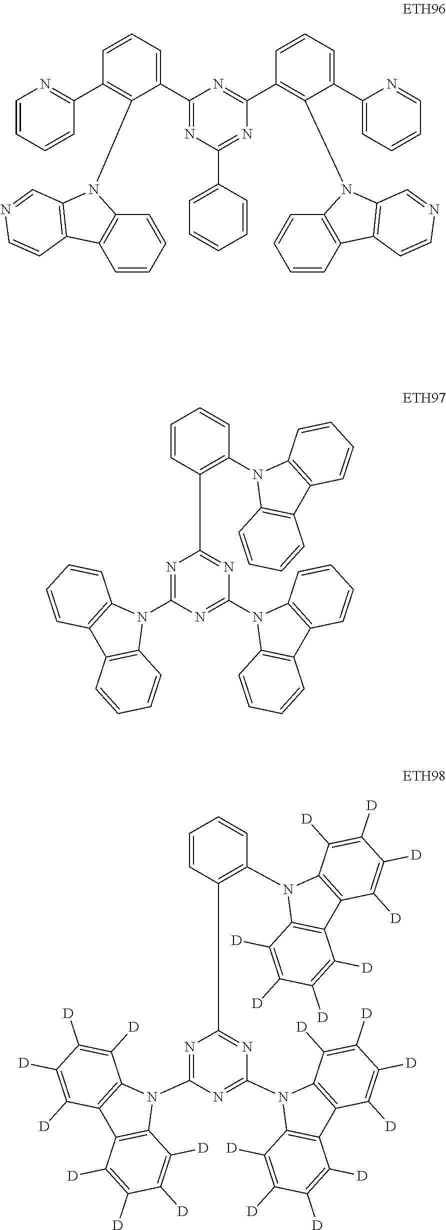 [SMILES: c1ccc(-c2nc(-c3cccc(-c4ccccn4)c3-n3c4ccccc4c4ccncc43)nc(-c3cccc(-c4ccccn4)c3-n3c4ccccc4c4ccncc43)n2)cc1 ; c1ccc(-n2c3ccccc3c3ccccc32)c(-c2nc(-n3c4ccccc4c4ccccc43)nc(-n3c4ccccc4c4ccccc43)n2)c1 ; [2H]c1c([2H])c([2H])c2c(c1[2H])c1c([2H])c([2H])c([2H])c([2H])c1n2-c1nc(-c2ccccc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c([2H])c([2H])c([2H])c32)n1]

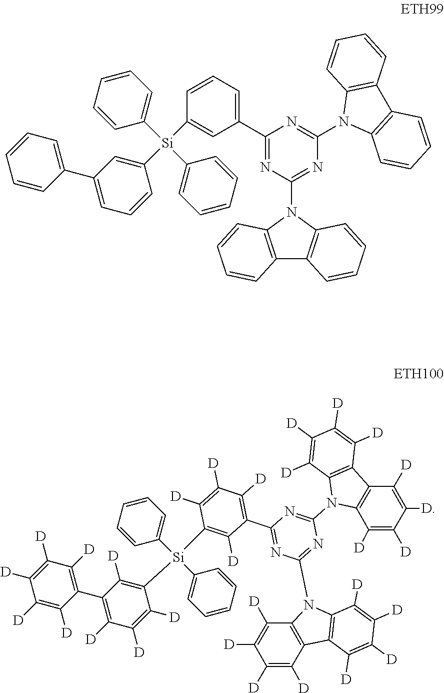

In one or more embodiments, the third compound may be any one selected from among Compounds HTH1 to HTH46 and HT-1:

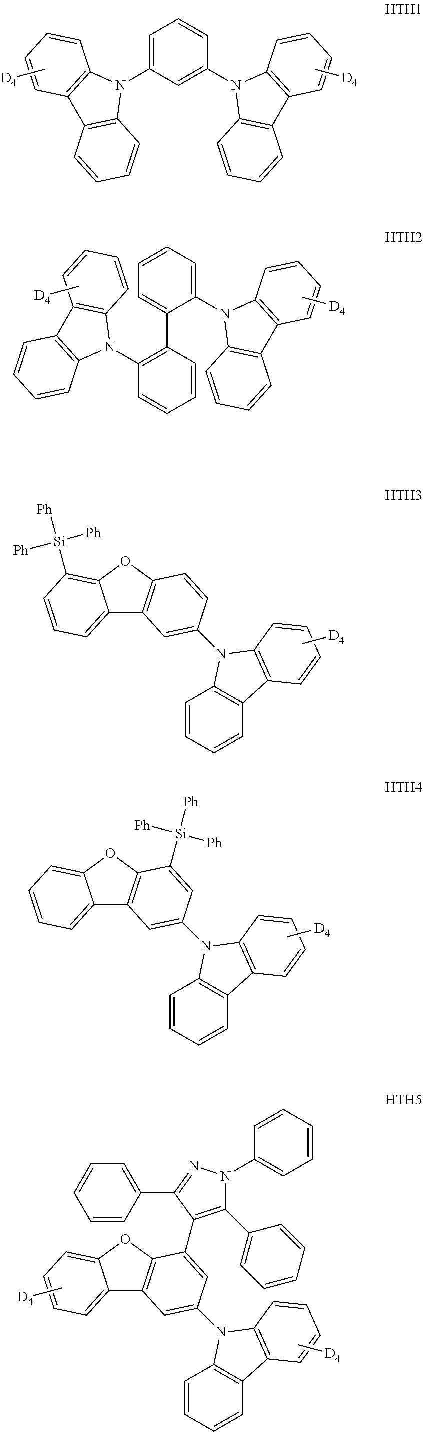

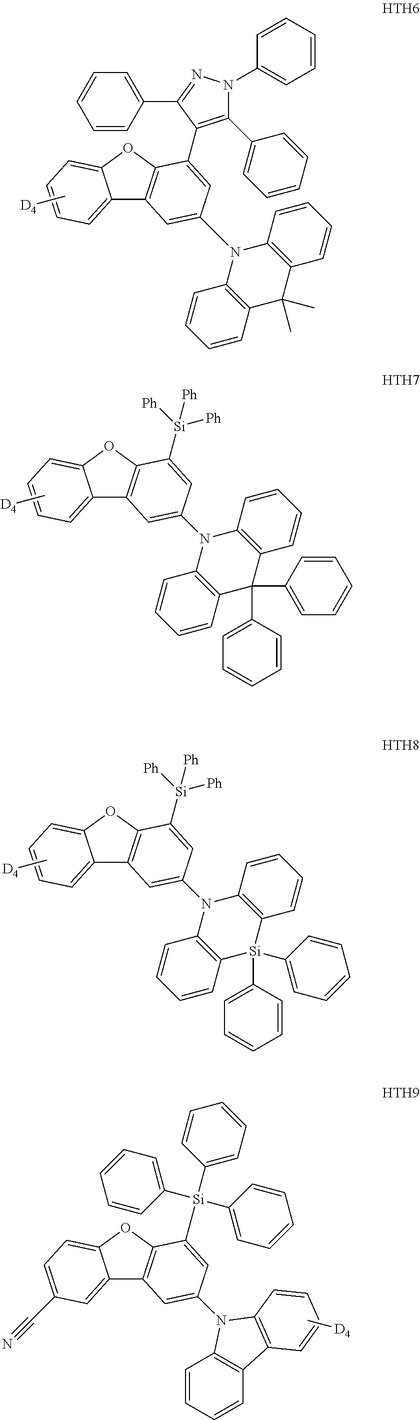

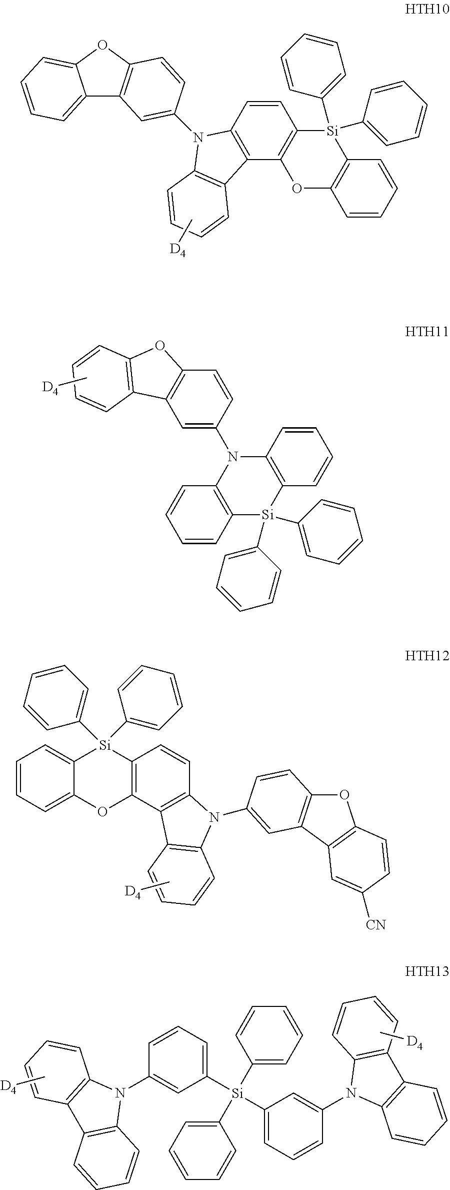

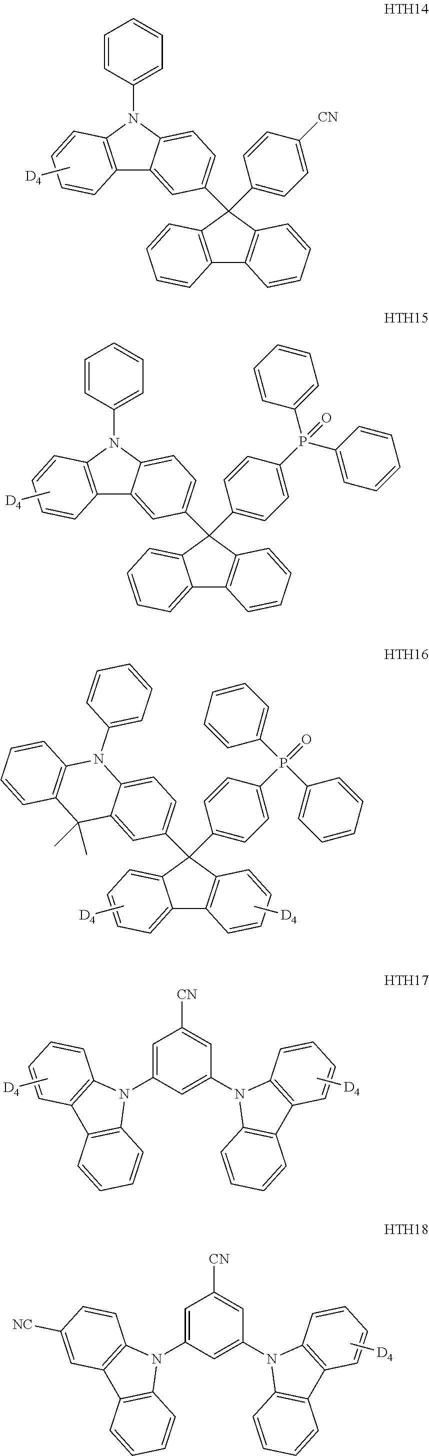

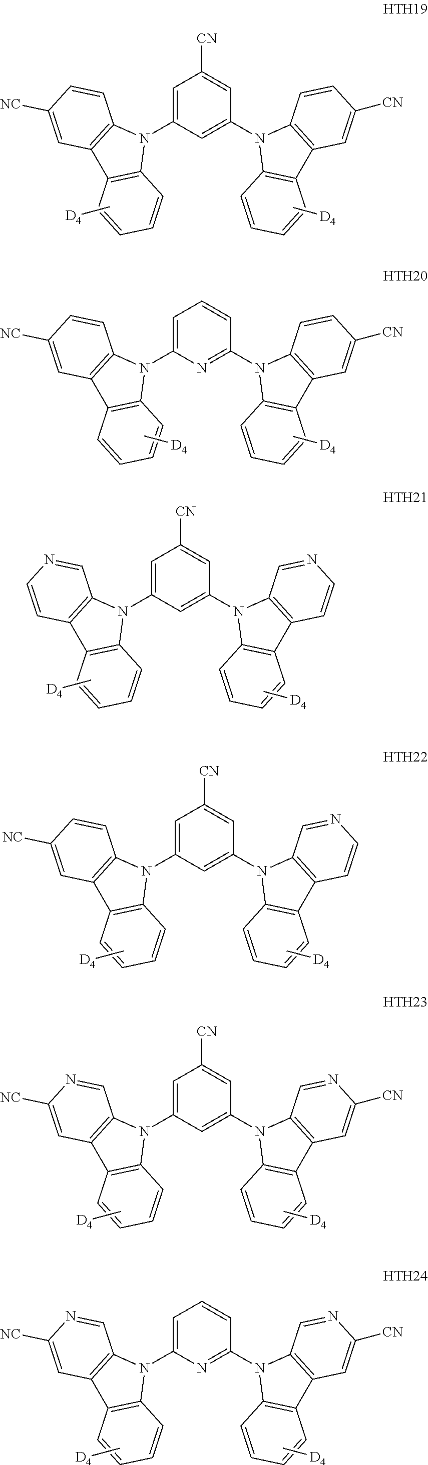

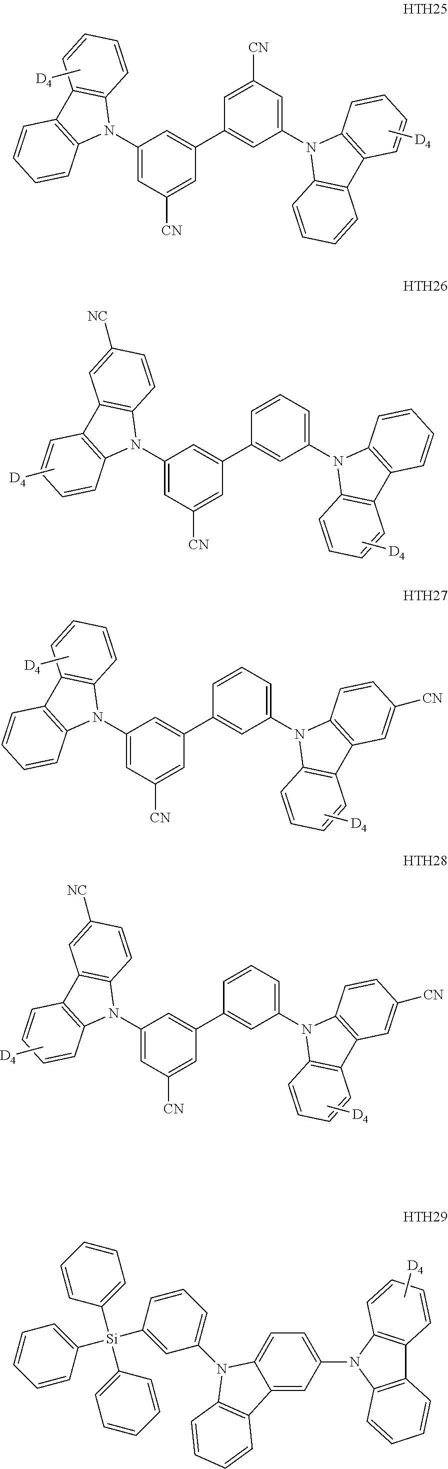

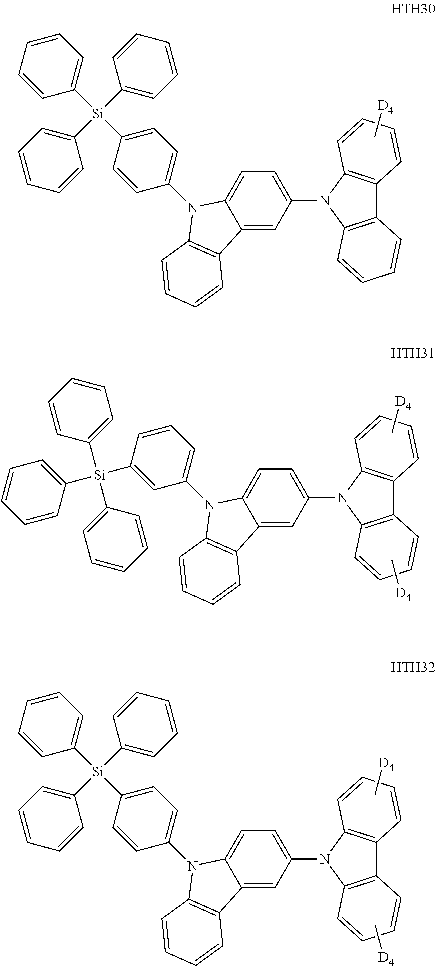

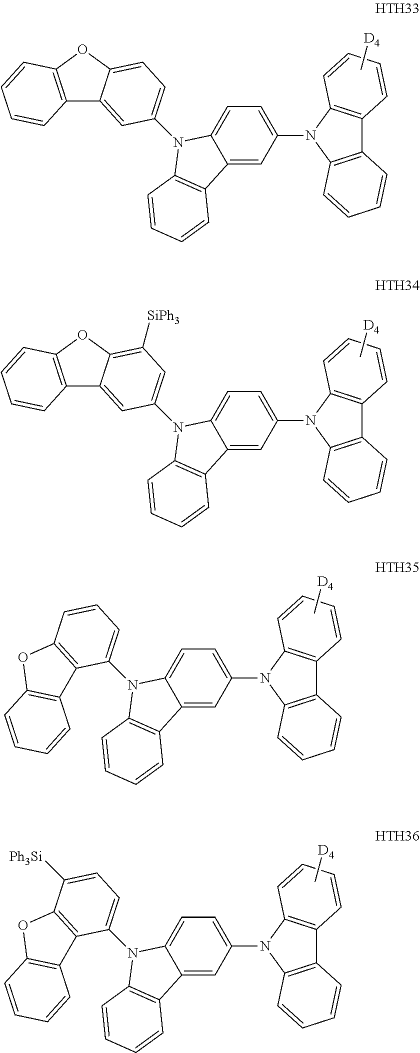

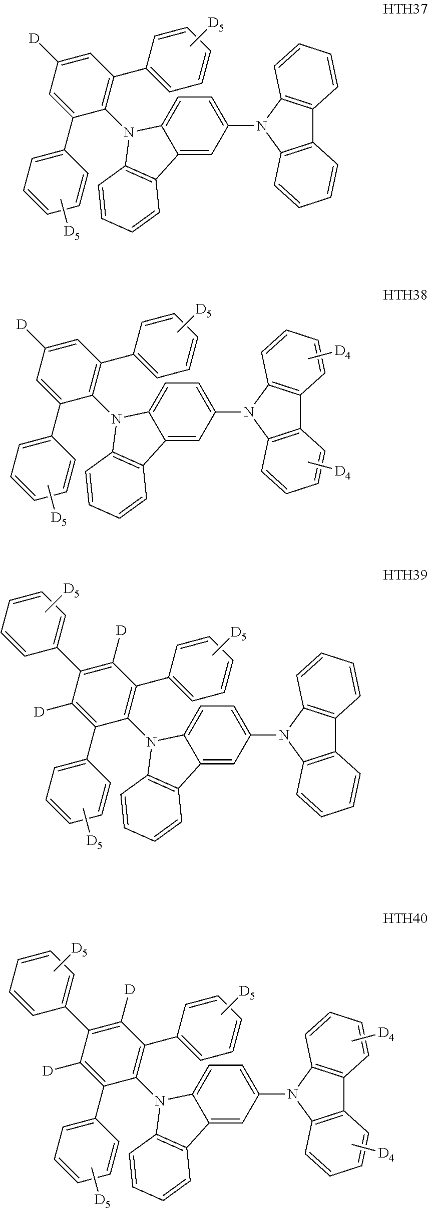

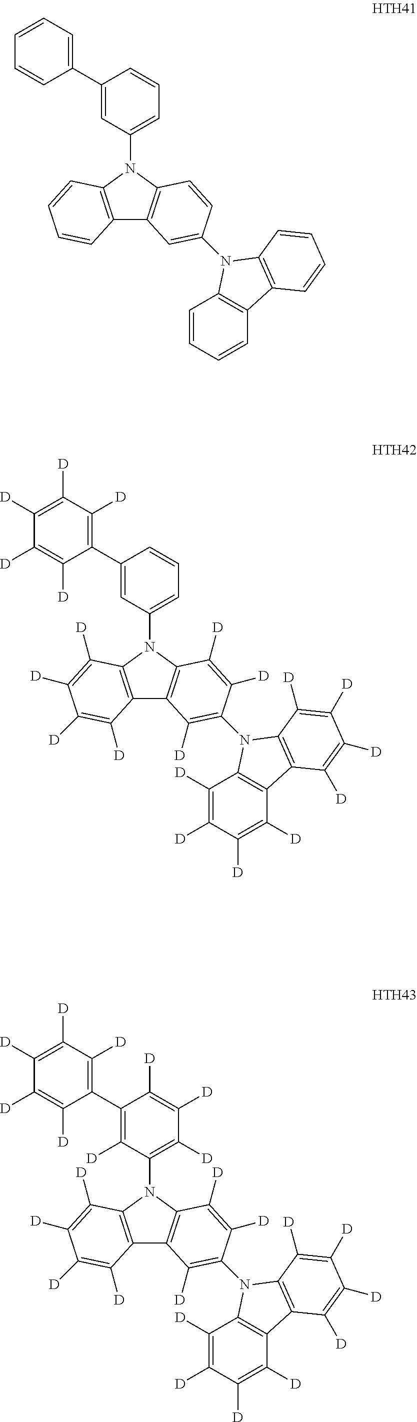 [SMILES: c1ccc(-c2cccc(-n3c4ccccc4c4cc(-n5c6ccccc6c6ccccc65)ccc43)c2)cc1 ; [2H]c1c([2H])c([2H])c(-c2cccc(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c2)c([2H])c1[2H] ; [2H]c1c([2H])c([2H])c(-c2c([2H])c([2H])c([2H])c(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c2[2H])c([2H])c1[2H]]

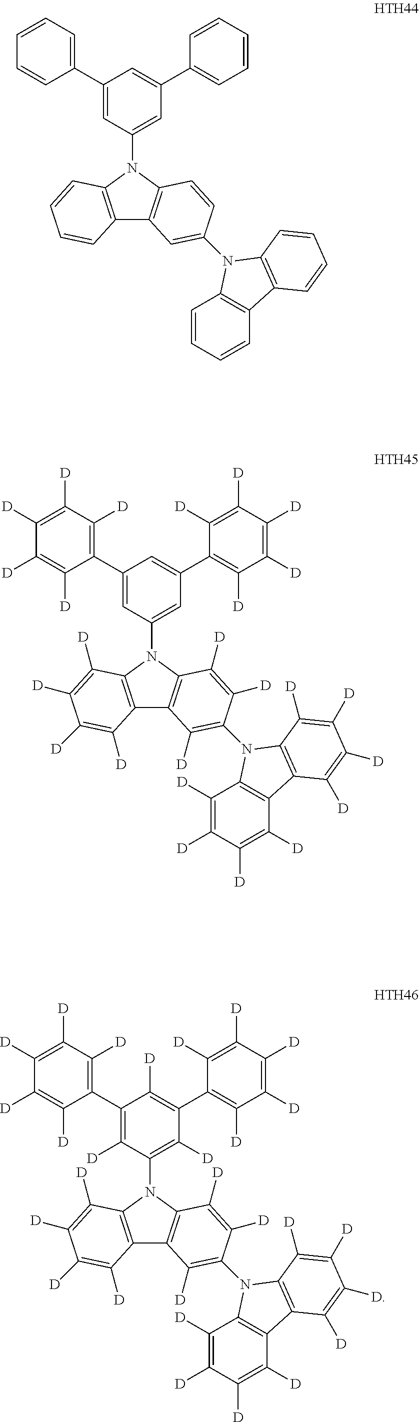

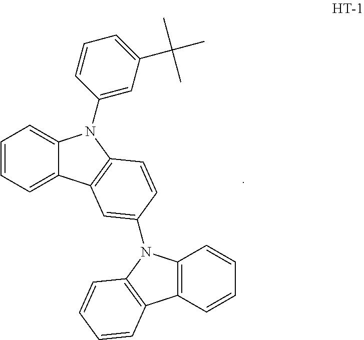 [SMILES: CC(C)(C)c1cccc(-n2c3ccccc3c3cc(-n4c5ccccc5c5ccccc54)ccc32)c1]

In one or more embodiments, the fourth compound may be any one selected from among Compounds D1 to D10 and PS-1, or may be a compound in which at least one hydrogen included in one of Compounds D1 to D10 and PS-1 is substituted with deuterium:

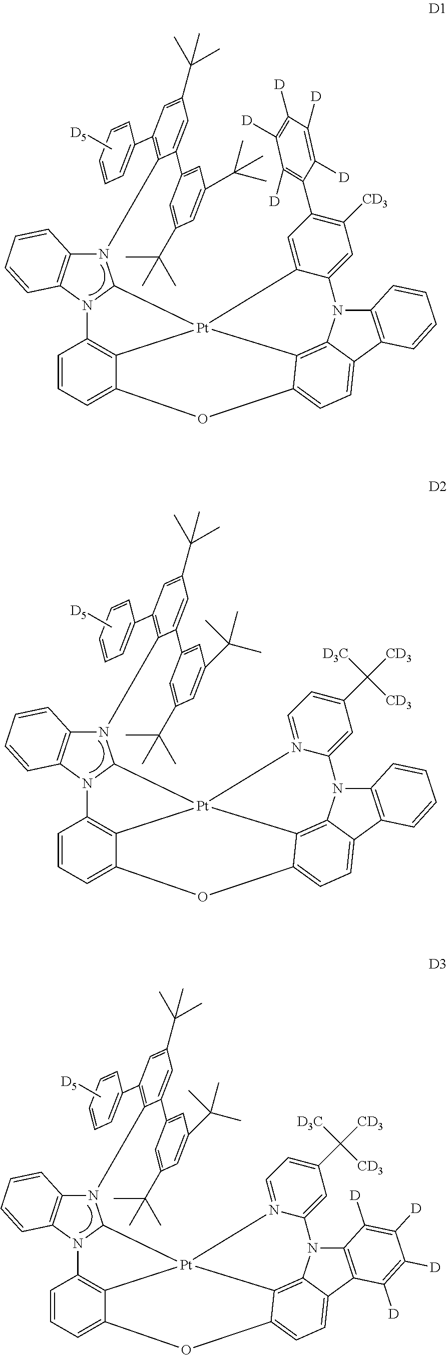

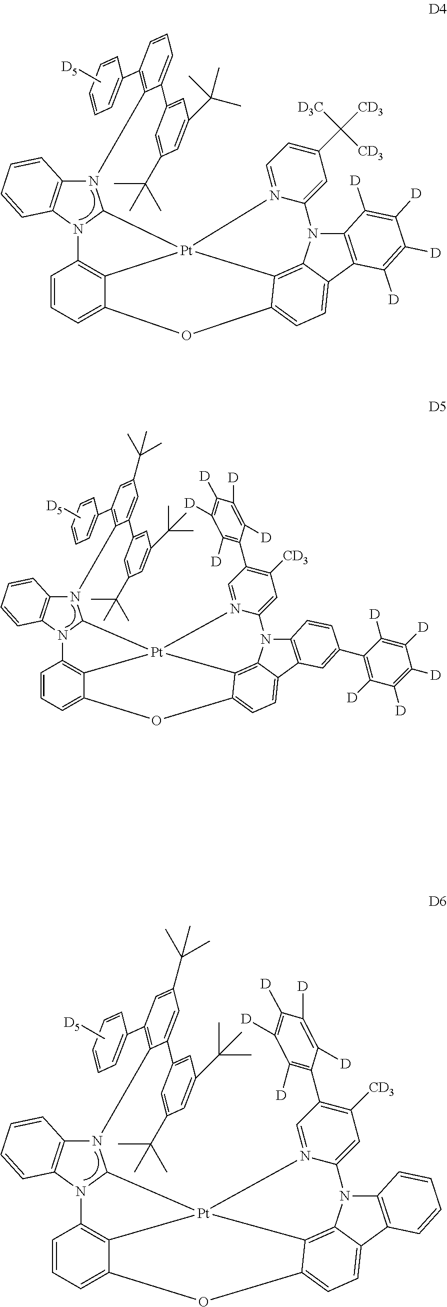

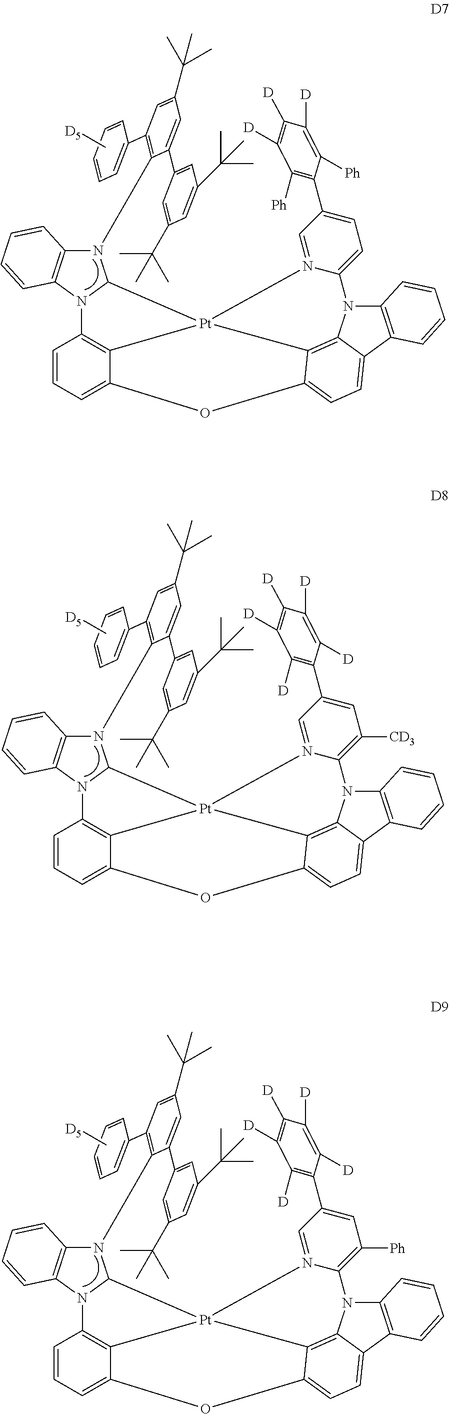

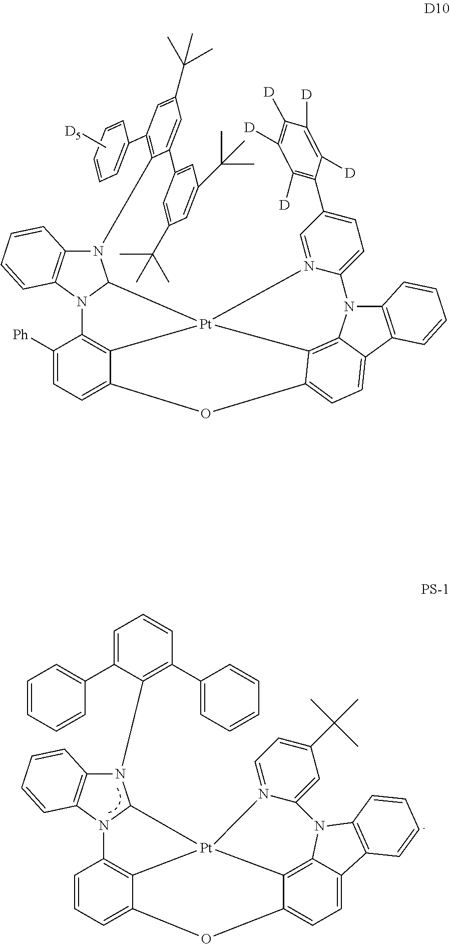

In the preceding compounds, Ph represents a phenyl group, D represents deuterium, D4 represents substitution with four deuterium atoms, and D5 represents substitution with five deuterium atoms. For example, a group represented by

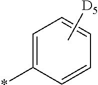

may be substantially identical to a group represented by

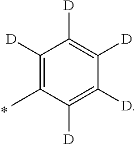

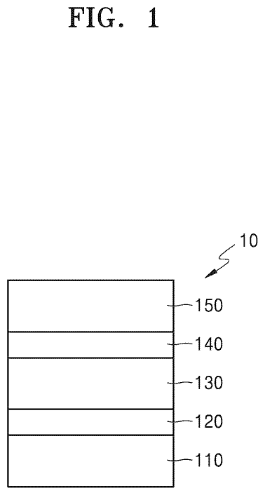

### Description of FIG. 1

FIG. 1 is a schematic cross-sectional view of a light-emitting device 10 according to one or more embodiments. The light-emitting device 10 may include a first electrode 110, an interlayer, and a second electrode 150. The interlayer may include a hole transport region 120, an emission layer 130, and an electron transport region 140.

Hereinafter, the structure and manufacturing method of the light-emitting device 10 according to one or more embodiments are described with reference to FIG. 1.

### First Electrode 110

In FIG. 1, a substrate may be additionally arranged under the first electrode 110 or above the second electrode 150. As the substrate, a glass substrate or a plastic substrate may be used. The substrate may be a flexible substrate. For example, the substrate may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by depositing or sputtering a material for forming the first electrode 110 on the substrate. When the first electrode 110 is an anode, a high-work function material that facilitates injection of holes may be used as a material for forming the first electrode 110.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. When the first electrode 110 is a semi-transmissive electrode or a reflective electrode, magnesium (Mg), silver (Ag), aluminum (Al), aluminum-lithium (Al—Li), calcium (Ca), magnesium-indium (Mg—In), magnesium-silver (Mg—Ag), or any combination thereof may be used as a material for forming the first electrode 110.

The first electrode 110 may have a single-layered structure including (e.g., consisting of) a single layer or a multilayer structure including a plurality of layers. In one or more embodiments, the first electrode 110 may have a three-layer structure of ITO/Ag/ITO.

### Interlayer

The interlayer may be arranged above the first electrode 110. The interlayer may include the hole transport region 120, the emission layer 130, and the electron transport region 140.

The interlayer may include one or more suitable organic materials, a metal-containing compound such as an organometallic compound, an inorganic material such as quantum dots, and/or the like.

In one or more embodiments, the interlayer may include i) at least two emitting units sequentially stacked between the first electrode 110 and the second electrode 150 and ii) a charge generation layer arranged between the two emitting units. When the interlayer includes the emitting units and the charge generation layer as described herein, the light-emitting device 10 may be a tandem light-emitting device.

### Hole Transport Region 120

The hole transport region 120 may have i) a single-layer structure including (e.g., consisting of) a single layer including a single material, ii) a single-layer structure including (e.g., consisting of) a single layer including a plurality of materials that are different from each other, or iii) a multilayer structure including (e.g., consisting of) a plurality of layers including a plurality of materials that are different from each other.

The hole transport region 120 may include a hole injection layer, a hole transport layer, an emission auxiliary layer, an electron blocking layer, or any combination thereof.

For example, the hole transport region 120 may have a multilayer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, wherein constituent layers of each structure are stacked sequentially from the first electrode 110.

The hole transport region 120 may include a compound represented by Formula 201, a compound represented by Formula 202, or any combination thereof:

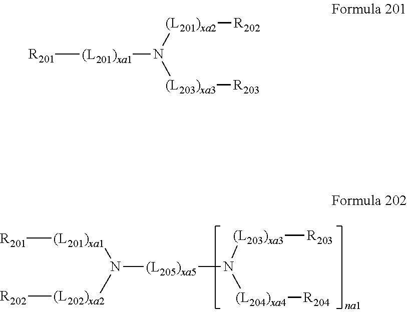

wherein, in Formulae 201 and 202,
L₂₀₁ to L₂₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
L₂₀₅ may be *—O—*′, *—S—*′, *—N(Q₂₀₁)-*′, a C₁-C₂₀ alkylene group that is unsubstituted or substituted with at least one R₁₀ₐ, a C₂-C₂₀ alkenylene group that is unsubstituted or substituted with at least one R₁₀ₐ, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
R₂₀₁ and R₂₀₂ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group (for example, a carbazole group and/or the like) that is unsubstituted or substituted with at least one R₁₀ₐ (for example, Compound HT16 and/or the like),
R₂₀₃ and R₂₀₄ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group that is unsubstituted or substituted with at least one R₁₀ₐ, and
na1 may be an integer from 1 to 4.

In one or more embodiments, each of Formulae 201 and 202 may include at least one of (e.g., selected from among) groups represented by Formulae CY201 to CY217:

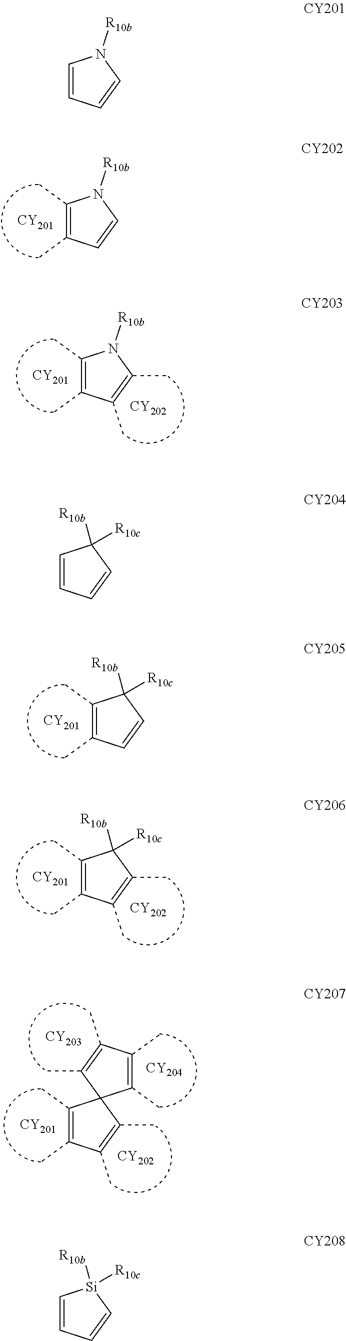

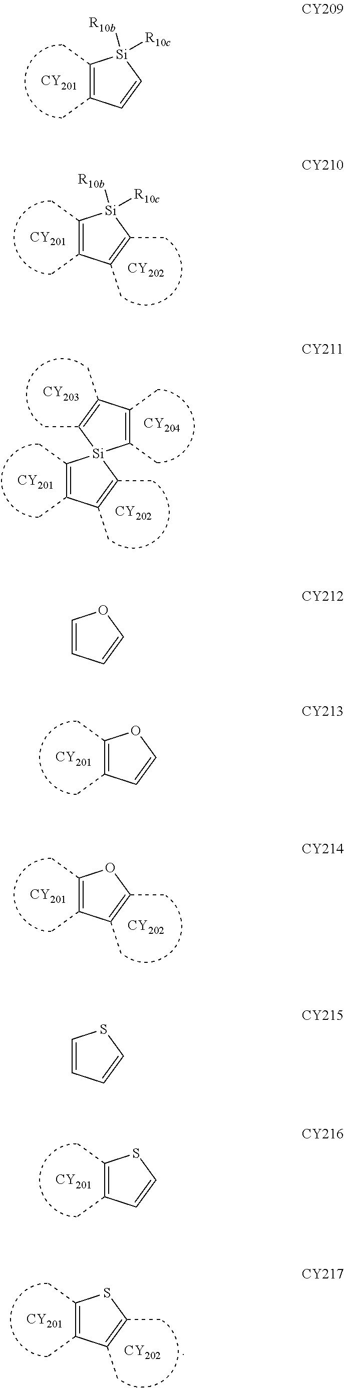

wherein, in Formulae CY₂₀₁ to CY217, R_{10b} and R_{10c} may each be as described in connection with R₁₀ₐ, ring CY₂₀₁ to ring CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In one or more embodiments, ring CY₂₀₁ to ring CY₂₀₄ in Formulae CY201 to CY217 may each independently be a benzene group, a naphthalene group, a phenanthrene group, or an anthracene group.

In one or more embodiments, each of Formulae 201 and 202 may include at least one of (e.g., selected from among) groups represented by Formulae CY201 to CY203.

In one or more embodiments, Formula 201 may include at least one of (e.g., selected from among) the groups represented by Formulae CY201 to CY203 and at least one of (e.g., selected from among) groups represented by Formulae CY204 to CY217.

In one or more embodiments, in Formula 201, xa1 may be 1, R₂₀₁ may be a group represented by one of (e.g., selected from among) Formulae CY201 to CY203, xa2 may be 0, and R₂₀₂ may be a group represented by one of (e.g., selected from among) Formulae CY204 to CY207.

In one or more embodiments, each of Formulae 201 and 202 may not include (e.g., may exclude) groups represented by Formulae CY201 to CY203.

In one or more embodiments, Formulae 201 and 202 may each not include groups represented by Formulae CY201 to CY203 and may include at least one selected from among groups represented by Formulae CY204 to CY217.

In one or more embodiments, each of Formulae 201 and 202 may not include (e.g., may exclude) groups represented by Formulae CY201 to CY217.

In one or more embodiments, the hole transport region may include at least one of (e.g., one or more) selected from among Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzene sulfonic acid (PANI/DBSA), poly(3,4-ethylene dioxythiophene)/poly(4-styrene sulfonate) (PEDOT/PSS), polyaniline/camphor sulfonic acid (PANI/CSA), polyaniline/poly(4-styrene sulfonate) (PANI/PSS), or any combination thereof:

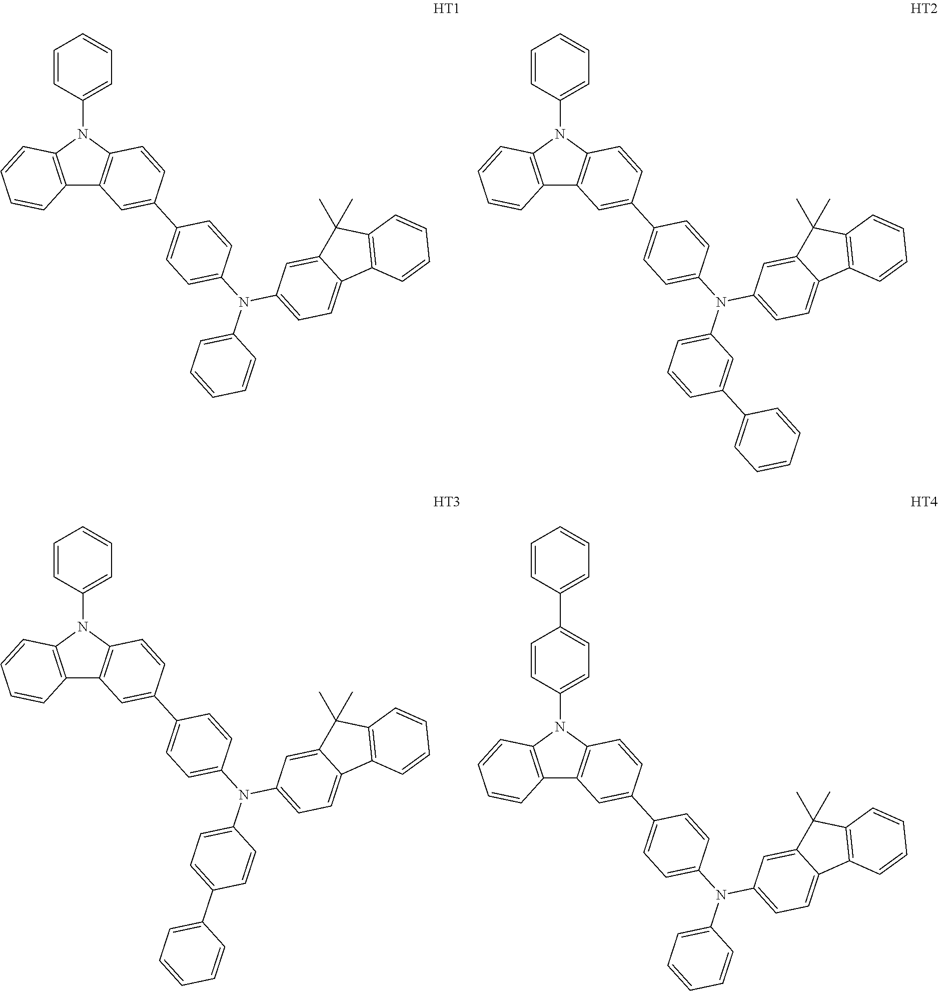 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

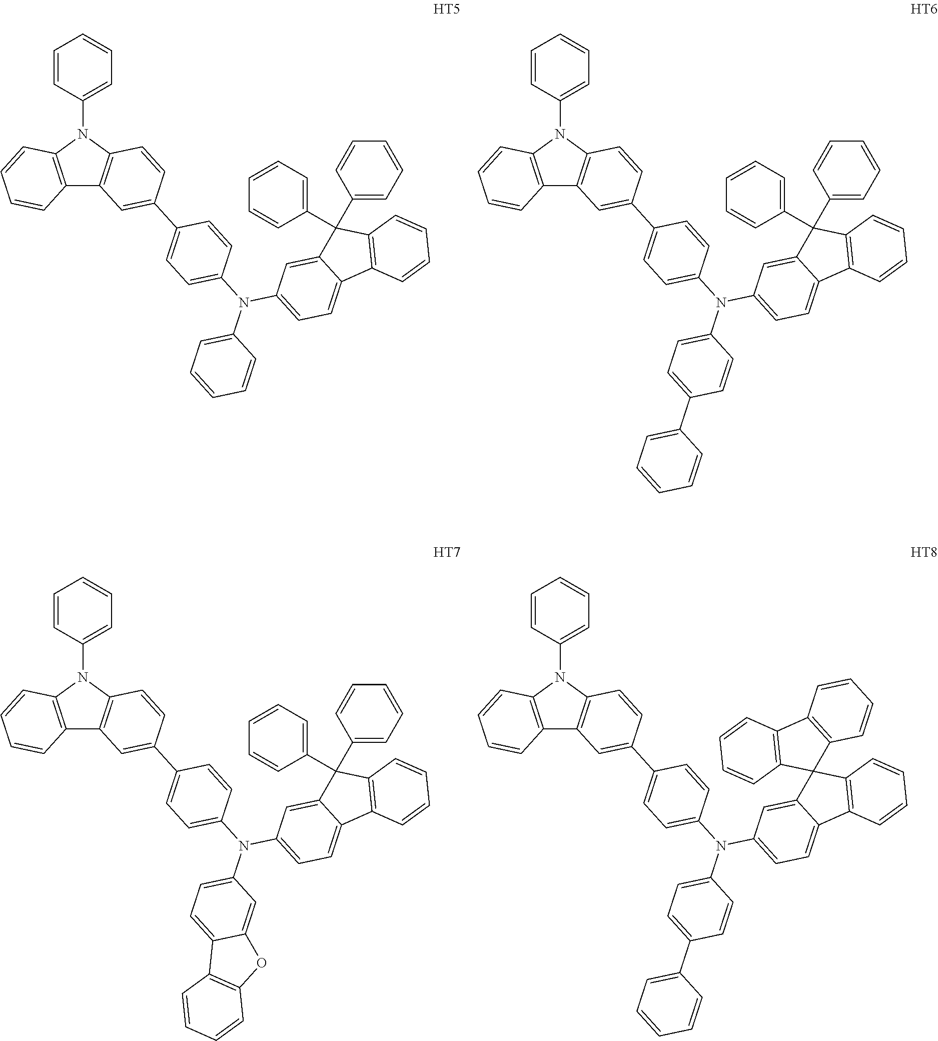 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

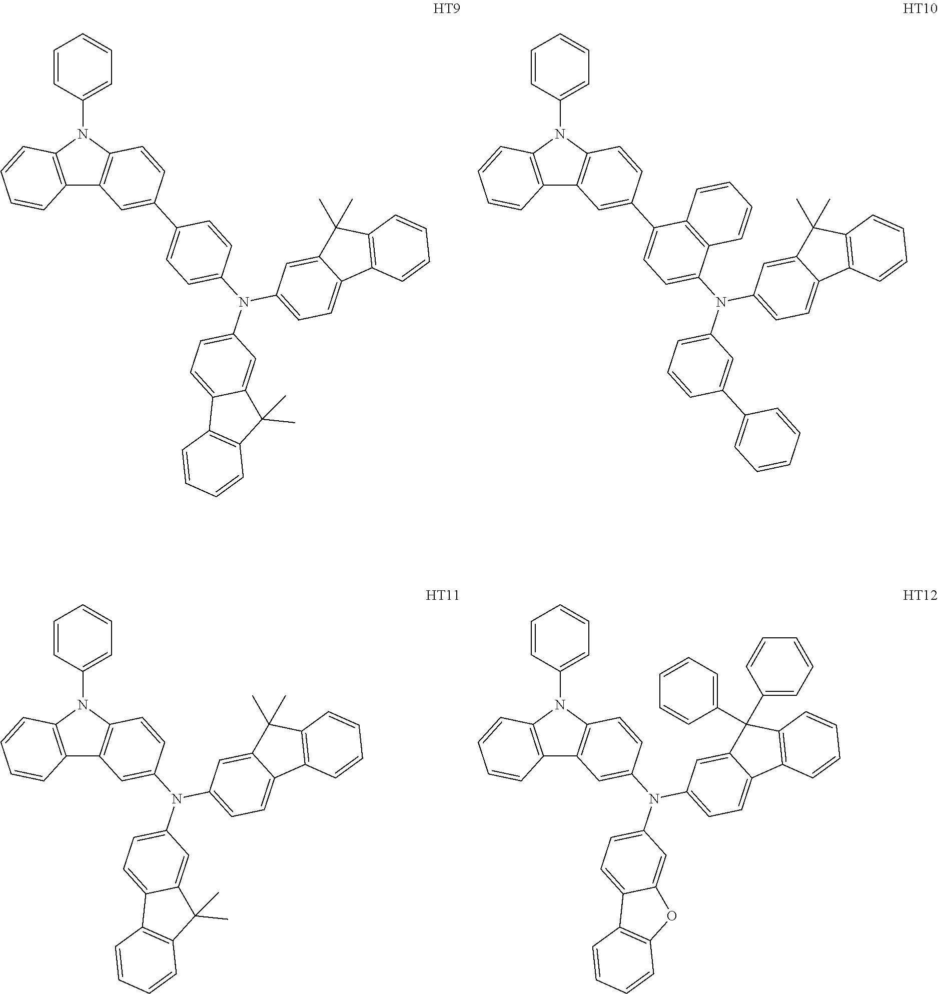 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

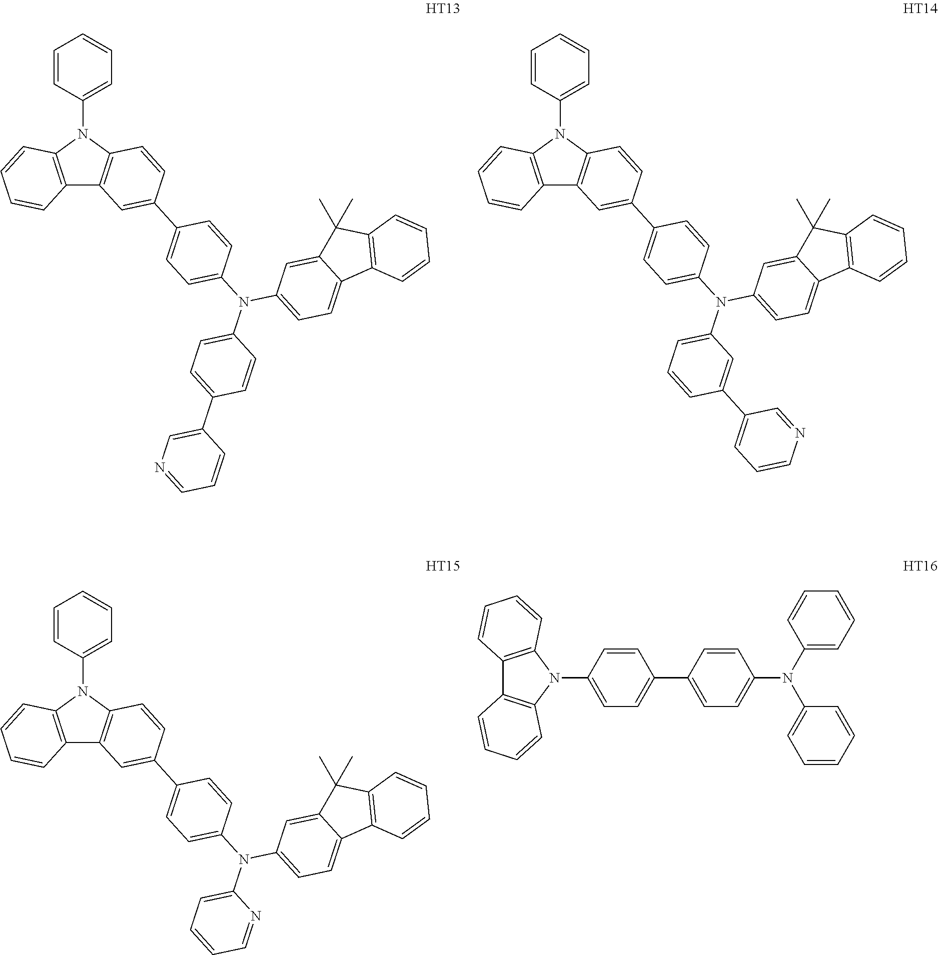 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

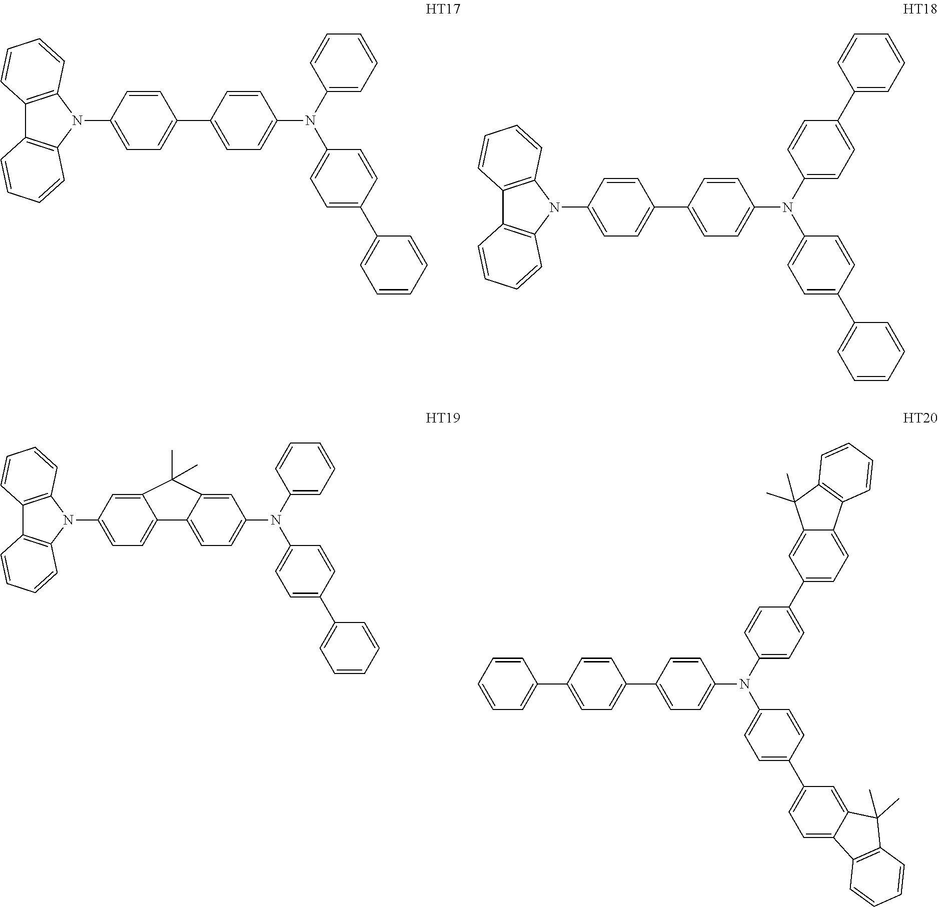 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

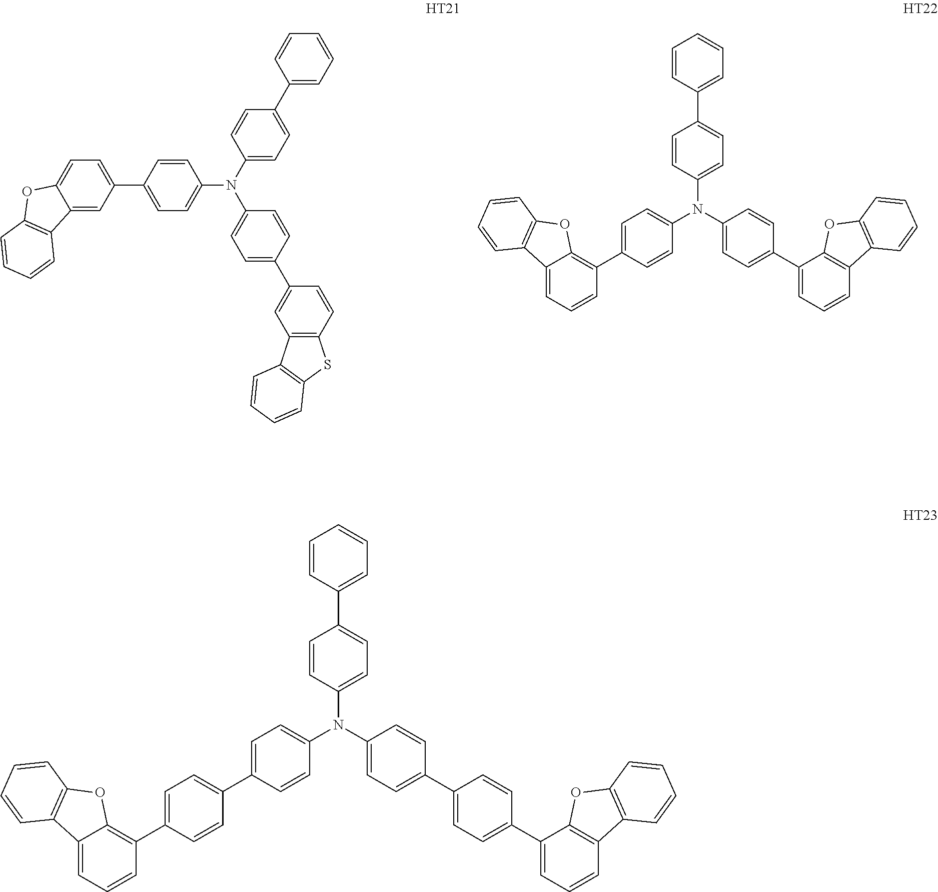 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

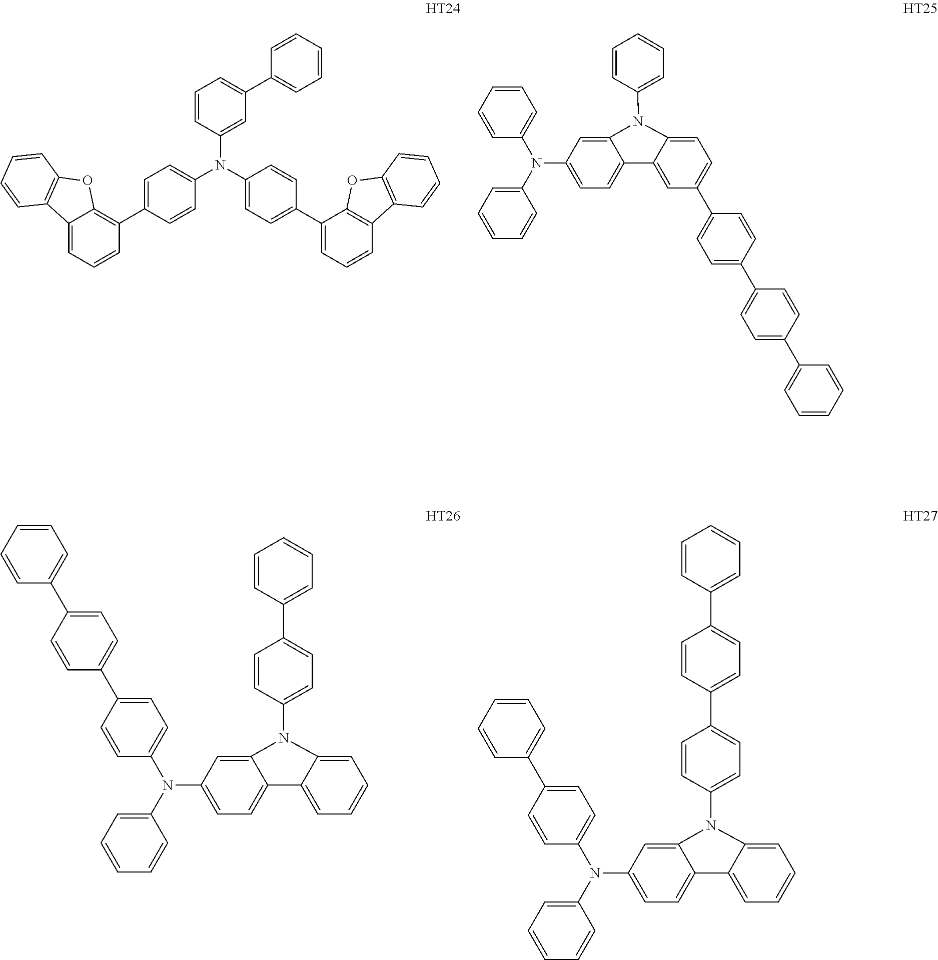 [SMILES: c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

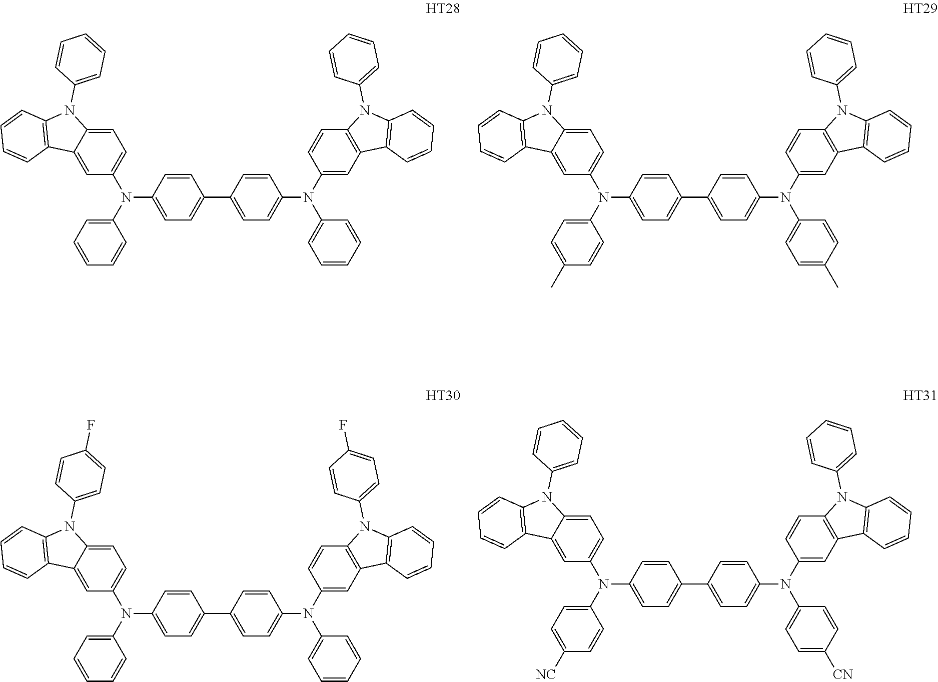 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1 ; N#Cc1ccc(N(c2ccc(-c3ccc(N(c4ccc(C#N)cc4)c4ccc5c(c4)c4ccccc4n5-c4ccccc4)cc3)cc2)c2ccc3c(c2)c2ccccc2n3-c2ccccc2)cc1]

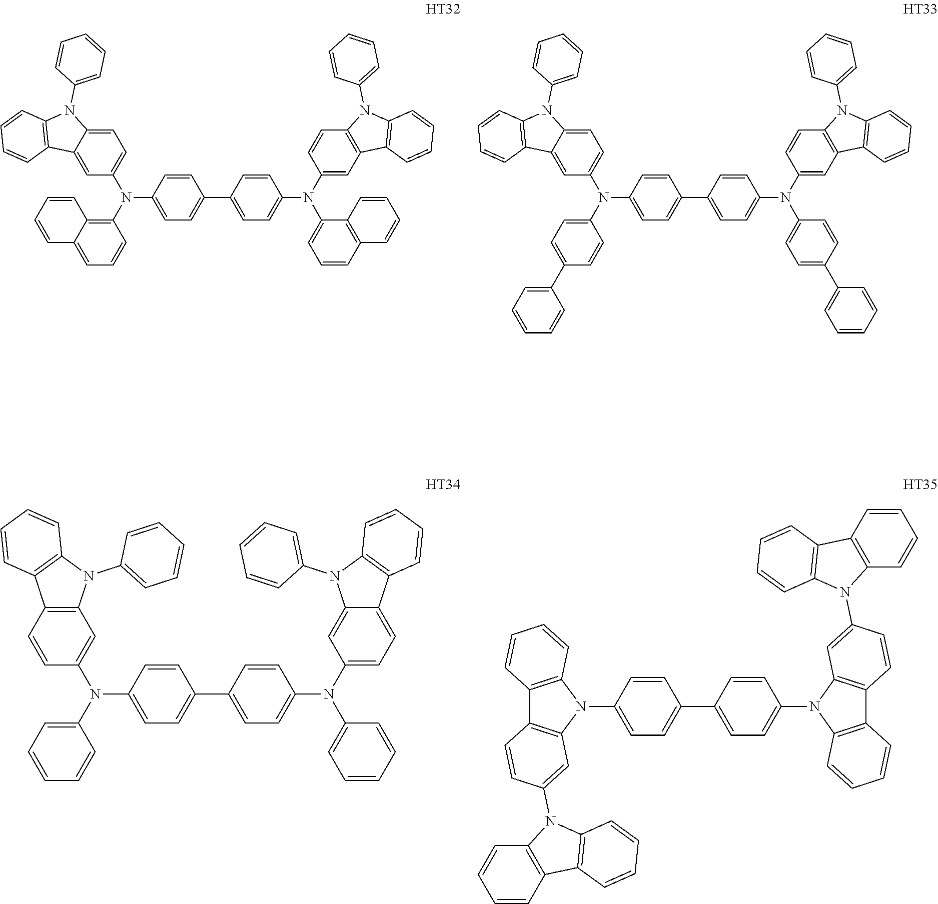 [SMILES: c1ccc(-n2c3ccccc3c3cc(N(c4ccc(-c5ccc(N(c6ccc7c(c6)c6ccccc6n7-c6ccccc6)c6cccc7ccccc67)cc5)cc4)c4cccc5ccccc45)ccc32)cc1 ; c1ccc(-c2ccc(N(c3ccc(-c4ccc(N(c5ccc(-c6ccccc6)cc5)c5ccc6c(c5)c5ccccc5n6-c5ccccc5)cc4)cc3)c3ccc4c(c3)c3ccccc3n4-c3ccccc3)cc2)cc1 ; c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1 ; c1ccc2c(c1)c1ccccc1n2-c1ccc2c3ccccc3n(-c3ccc(-c4ccc(-n5c6ccccc6c6ccc(-n7c8ccccc8c8ccccc87)cc65)cc4)cc3)c2c1]

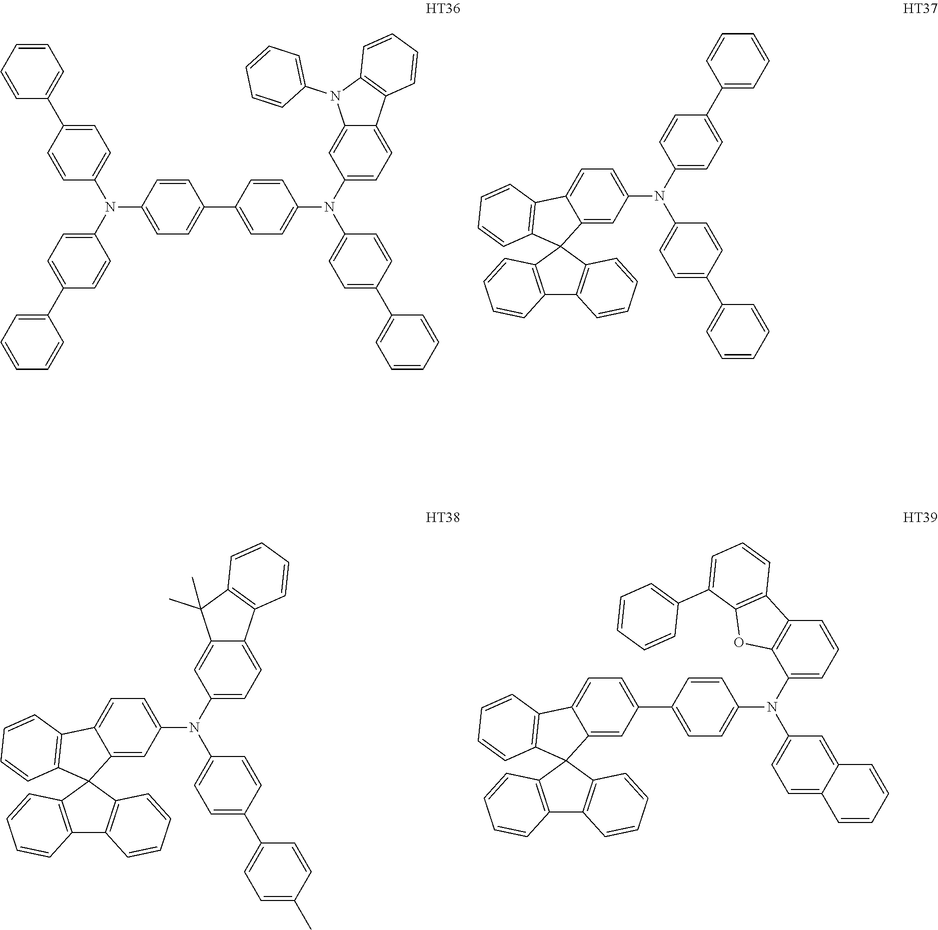 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

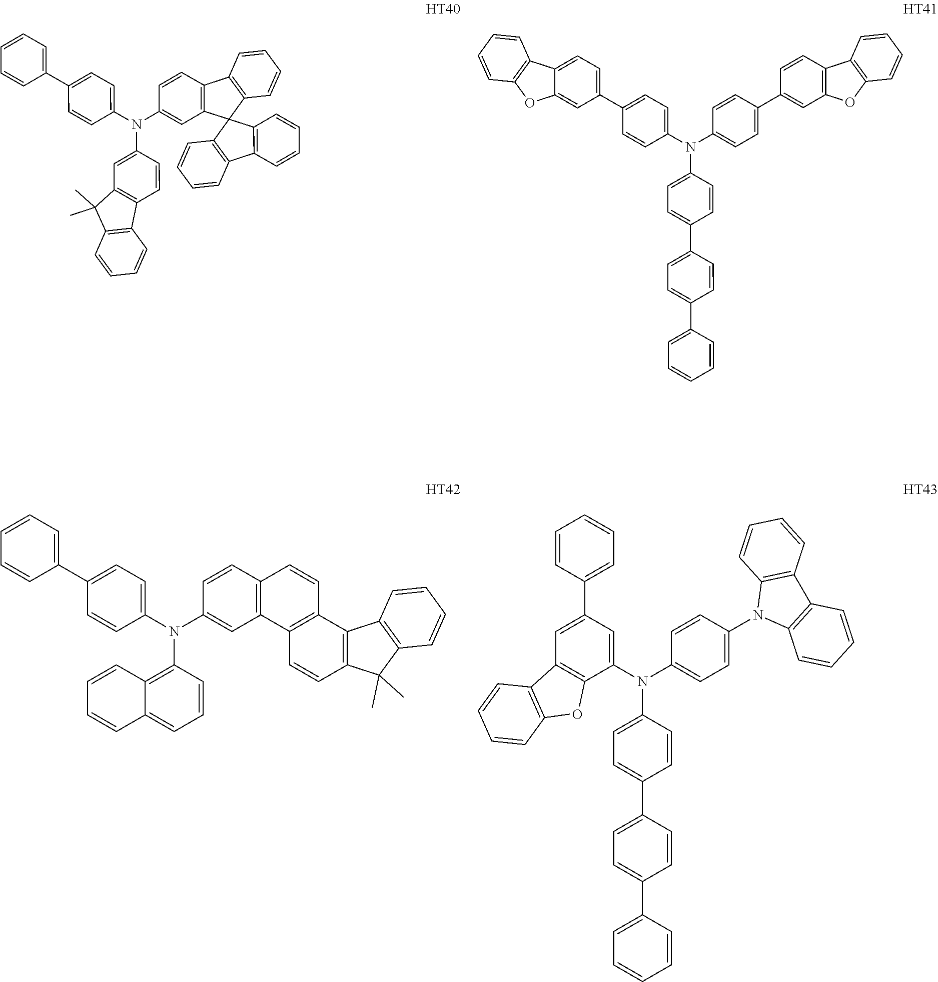 [SMILES: CC1(C)c2ccccc2-c2ccc(N(c3ccc(-c4ccccc4)cc3)c3ccc4c(c3)C3(c5ccccc5-c5ccccc53)c3ccccc3-4)cc21 ; c1ccc(-c2ccc(-c3ccc(N(c4ccc(-c5ccc6c(c5)oc5ccccc56)cc4)c4ccc(-c5ccc6c(c5)oc5ccccc56)cc4)cc3)cc2)cc1 ; CC1(C)c2ccccc2-c2c1ccc1c2ccc2ccc(N(c3ccc(-c4ccccc4)cc3)c3cccc4ccccc34)cc21 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

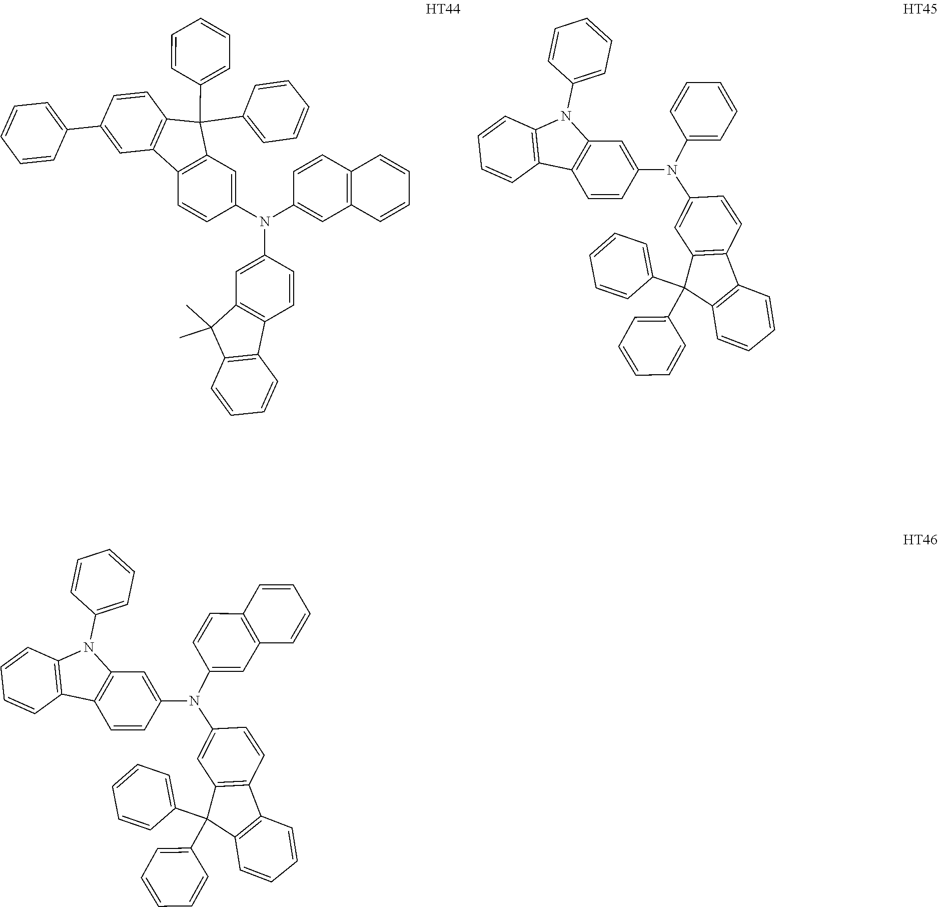 [SMILES: CC1(C)c2ccccc2-c2ccc(N(c3ccc4c(c3)C(c3ccccc3)(c3ccccc3)c3ccc(-c5ccccc5)cc3-4)c3ccc4ccccc4c3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; c1ccc(-n2c3ccccc3c3ccc(N(c4ccc5c(c4)C(c4ccccc4)(c4ccccc4)c4ccccc4-5)c4ccc5ccccc5c4)cc32)cc1]

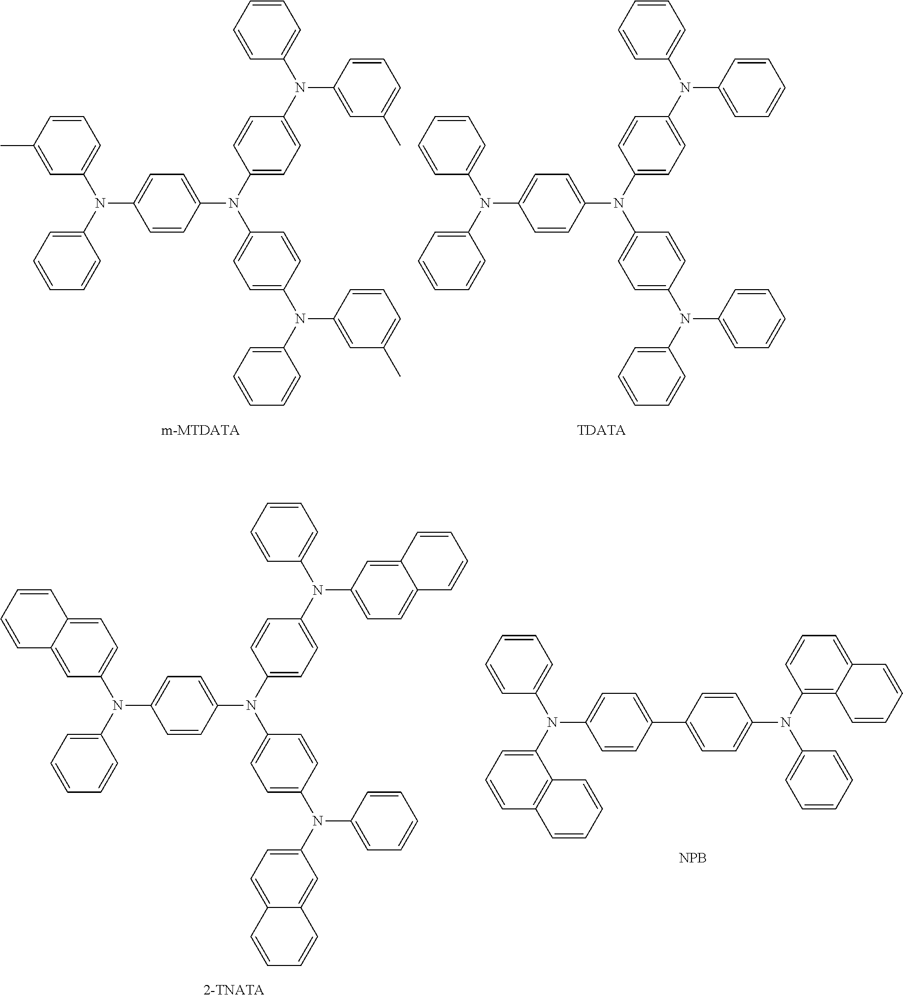

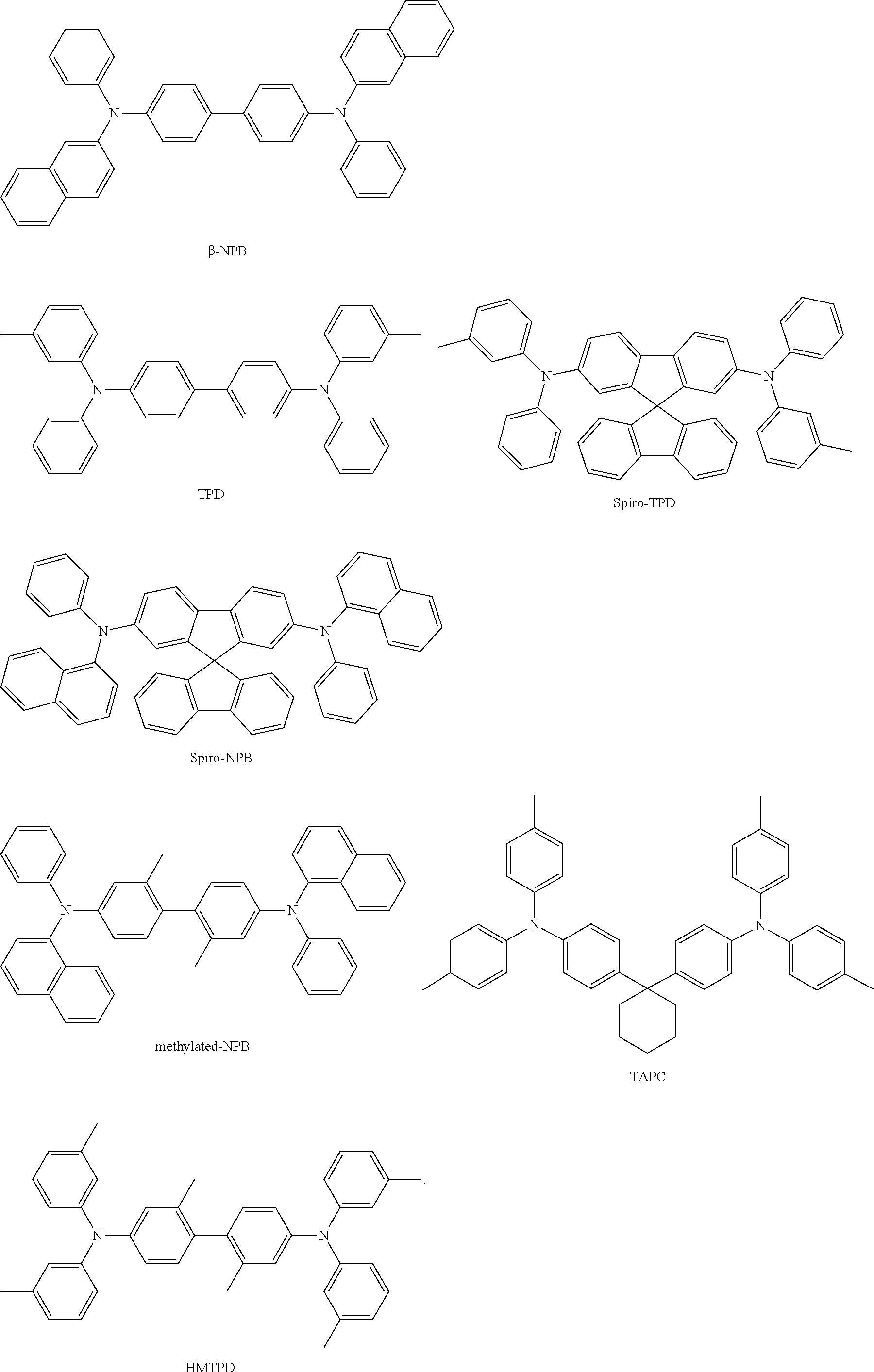

The thickness of the hole transport region 120 may be about 50 Å to about 10,000 Å, for example, about 100 Å to about 4,000 Å. When the hole transport region 120 includes a hole injection layer, a hole transport layer, or any combination thereof, the thickness of the hole injection layer may be about 100 Å to about 9,000 Å, for example, about 100 Å to about 1,000 Å, and the thickness of the hole transport layer may be about 50 Å to about 2,000 Å, for example, about 100 Å to about 1,500 Å. When the thicknesses of the hole transport region, the hole injection layer, and the hole transport layer are within the ranges described herein, satisfactory hole transporting characteristics may be obtained without a substantial increase in driving voltage.

The emission auxiliary layer may serve to increase light-emission efficiency by compensating for an optical resonance distance according to the wavelength of light emitted by the emission layer. The electron blocking layer may serve to prevent or reduce electron leakage from the emission layer to the hole transport region. Materials that may be included in the hole transport region may be included in the emission auxiliary layer and the electron blocking layer.

p-Dopant

The hole transport region 120 may further include, in addition to the aforementioned materials, a charge-generation material for the improvement of conductive properties. The charge-generation material may be uniformly (e.g., substantially uniformly) or non-uniformly (e.g., substantially non-uniformly) dispersed in the hole transport region (for example, in the form of a single layer including (e.g., consisting of) a charge-generation material).

The charge-generation material may be, for example, a p-dopant.

For example, the LUMO energy level of the p-dopant may be about −3.5 eV or less.

In one or more embodiments, the p-dopant may include a quinone derivative, a cyano group-containing compound, a compound including an element EL1 and an element EL2, or any combination thereof.

Examples of the quinone derivative may include TCNQ, F4-TCNQ, and/or the like.

Examples of the cyano group-containing compound may include HAT-CN, a compound represented by Formula 221, and/or the like.

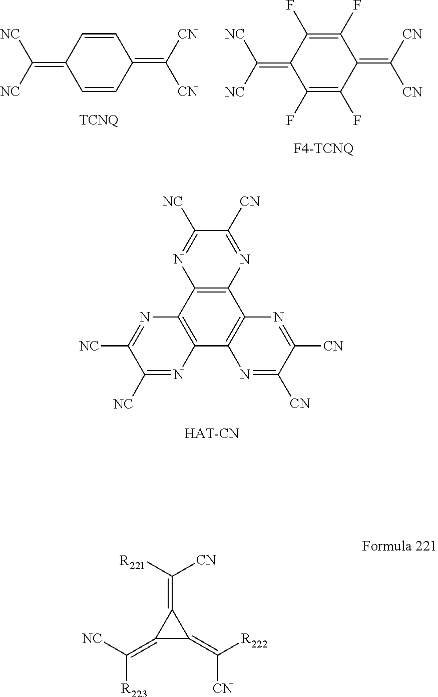

In Formula 221,
R₂₂₁ to R₂₂₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ, and
at least one of (e.g., selected from among) R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound including the element EL1 and the element EL2, the element EL1 may be a metal, a metalloid, and/or a (e.g., any suitable) combination thereof, and the element EL2 may be a non-metal, a metalloid, and/or a (e.g., any suitable) combination thereof.

Examples of the metal may include an alkali metal (for example, lithium (Li), sodium (Na), potassium (K), rubidium (Rb), cesium (Cs), and/or the like); an alkaline earth metal (for example, beryllium (Be), magnesium (Mg), calcium (Ca), strontium (Sr), barium (Ba), and/or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); a post-transition metal (for example, zinc (Zn), indium (In), tin (Sn), and/or the like); and a lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), and/or the like).

Examples of the metalloid may include silicon (Si), antimony (Sb), tellurium (Te), and/or the like.

Examples of the non-metal may include oxygen (O) and halogen (for example, F, Cl, Br, I, and/or the like).

Examples of the compound including the element EL1 and the element EL2 may include a metal oxide, a metal halide (for example, a metal fluoride, a metal chloride, a metal bromide, a metal iodide, and/or the like), a metalloid halide (for example, a metalloid fluoride, a metalloid chloride, a metalloid bromide, a metalloid iodide, and/or the like), a metal telluride, or any combination thereof.

Examples of the metal oxide may include a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (for example, MoO, Mo₂O₃, MoO₂, MoO₃, Mo₂O₅, and/or the like), a rhenium oxide (for example, ReO₃, and/or the like), and/or the like.

Examples of the metal halide may include an alkali metal halide, an alkaline earth metal halide, a transition metal halide, a post-transition metal halide, and a lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, Mg₁₂, CaI₂, SrI₂, and BaI₂.

Examples of the transition metal halide may include a titanium halide (for example, TiF₄, TiC₄, TiBr₄, TiI₄, and/or the like), a zirconium halide (for example, ZrF₄, ZrCl₄, ZrBr₄, ZrI₄, and/or the like), a hafnium halide (for example, HfF₄, HfCl₄, HfBr₄, Hfl₄, and/or the like), a vanadium halide (for example, VF₃, VCI₃, VBr₃, VI₃, and/or the like), a niobium halide (for example, NbF₃, NbCl₃, NbBr₃, NbI₃, and/or the like), a tantalum halide (for example, TaF₃, TaCl₃, TaBr₃, TaI₃, and/or the like), a chromium halide (for example, CrF₃, CrCl₃, CrBr₃, CrI₃, and/or the like), a molybdenum halide (for example, MoF₃, MoClK, MoBr₃, MoI₃, and/or the like), a tungsten halide (for example, WF₃, WCl₃, WBr₃, WI₃, and/or the like), a manganese halide (for example, MnF₂, MnCl₂, MnBr₂, MnI₂, and/or the like), a technetium halide (for example, TcF₂, TcCl₂, TcBr₂, TcI₂, and/or the like), a rhenium halide (for example, ReF₂, ReCl₂, ReBr₂, ReI₂, and/or the like), an Iron(II) halide (for example, FeF₂, FeCl₂, FeBr₂, FeI₂, and/or the like), a ruthenium halide (for example, RuF₂, RuCl₂, RuBr₂, RuI₂, and/or the like), an osmium halide (for example, OsF₂, OsCl₂, OsBr₂, OsI₂, and/or the like), a cobalt halide (for example, CoF₂, COCl₂, CoBr₂, CoI₂, and/or the like), a rhodium halide (for example, RhF₂, RhCl₂, RhBr₂, RhI₂, and/or the like), an iridium halide (for example, IrF₂, IrCl₂, IrBr₂, IrI₂, and/or the like), a nickel halide (for example, NiF₂, NiCl₂, NiBr₂, NiI₂, and/or the like), a palladium halide (for example, PdF₂, PdCl₂, PdBr₂, PdI₂, and/or the like), a platinum halide (for example, PtF₂, PtCl₂, PtBr₂, PtI₂, and/or the like), a Copper(I) halide (for example, CuF, CuCl, CuBr, CuI, and/or the like), a silver halide (for example, AgF, AgCl, AgBr, AgI, and/or the like), and a gold halide (for example, AuF, AuCl, AuBr, AuI, and/or the like).

Examples of the post-transition metal halide may include a zinc halide (for example, ZnF₂, ZnCl₂, ZnBr₂, ZnI₂, and/or the like), an indium halide (for example, Ink₃, and/or the like), a tin halide (for example, SnI₂, and/or the like), and/or the like.

Examples of the lanthanide metal halide may include YbF, YbF₂, YbF₃, SmF₃, YbCl, YbCl₂, YbCl₃, SmCl₃, YbBr, YbBr₂, YbBr₃, SmBr₃, YbI, YbI₂, YbI₃, SmI₃, and/or the like.

Examples of the metalloid halide may include an antimony halide (for example, SbCl₅, and/or the like).

Examples of the metal telluride may include an alkali metal telluride (for example, Li₂Te, Na₂Te, K₂Te, Rb₂Te, Cs₂Te, and/or the like), an alkaline earth metal telluride (for example, BeTe, MgTe, CaTe, SrTe, BaTe, and/or the like), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), a post-transition metal telluride (for example, ZnTe, and/or the like), and a lanthanide metal telluride (for example, LaTe, CeTe, PrTe, NdTe, PmTe, EuTe, GdTe, TbTe, DyTe, HoTe, ErTe, TmTe, YbTe, LuTe, and/or the like).

### Emission Layer 130

When the light-emitting device 10 is a full-color light-emitting device, the emission layer 130 may be patterned into a red emission layer, a green emission layer, and/or a blue emission layer, according to a sub-pixel. In one or more embodiments, the emission layer 130 may have a stacked structure of two or more layers of a red emission layer, a green emission layer, and a blue emission layer, in which the two or more layers contact each other or are separated from each other, to emit white light. In one or more embodiments, the emission layer 130 may include two or more materials of a red light-emitting material, a green light-emitting material, and a blue light-emitting material, in which the two or more materials are mixed with each other in a single layer, to emit white light.

The emission layer 130 may include a host and a dopant. The dopant may include a phosphorescent dopant, a fluorescent dopant, or any combination thereof.

The amount of the dopant in the emission layer 130 may be about 0.01 parts by weight to about 15 parts by weight, based on 100 parts by weight of the host.

In one or more embodiments, the emission layer 130 may include a quantum dot.

The emission layer 130 may include a delayed fluorescence material. The delayed fluorescence material may act as a host or a dopant in the emission layer.

The thickness of the emission layer 130 may be about 100 Å to about 1,000 Å, for example, about 200 Å to about 600 Å. When the thickness of the emission layer is within the ranges described herein, excellent or suitable (e.g., desired) luminescence characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21} Formula 301
wherein, in Formula 301,
Ar₃₀₁ and L₃₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each as described in connection with Q₁.

In one or more embodiments, if (e.g., when) xb11 in Formula 301 is 2 or more, two or more of Ar₃₀₁ may be linked to each other via a single bond.

In one or more embodiments, the host may include a compound represented by Formula 301-1, a compound represented by Formula 301-2, or any combination thereof:

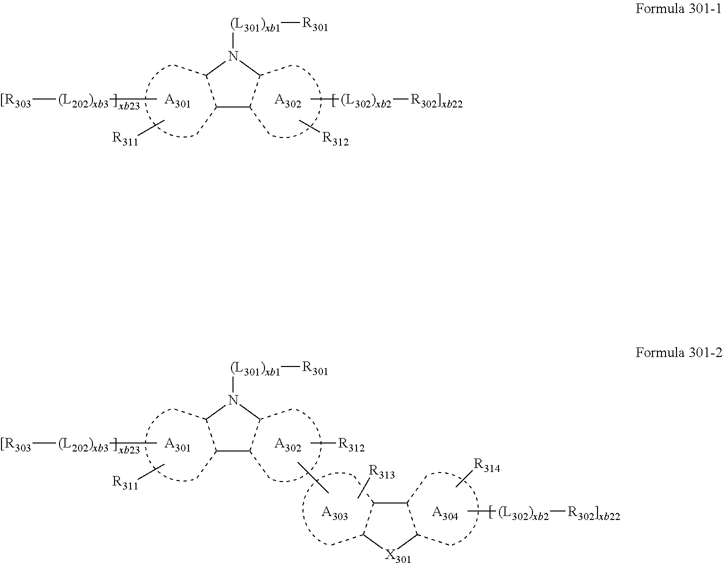

wherein, in Formula 301-1 and 301-2,
ring A₃₀₁ to ring A₃₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are each as described in the present specification,
L₃₀₂ to L₃₀₄ may each independently be as described in connection with L₃₀₁,
xb2 to xb4 may each independently be as described in connection with xb1, and
R₃₀₂ to R₃₀₅ and R₃₁₁ to R₃₁₄ are each as described in connection with R₃₀₁.

In one or more embodiments, the host may include an alkaline earth metal complex, a post-transition metal complex, or any combination thereof. In one or more embodiments, the host may include a Be complex (for example, Compound H55), an Mg complex, a Zn complex, or any combination thereof.

In one or more embodiments, the host may include: at least one of (e.g., one or more selected from among) Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(carbazol-9-yl)benzene (mCP); 1,3,5-tri(carbazol-9-yl)benzene (TCP); or any combination thereof:

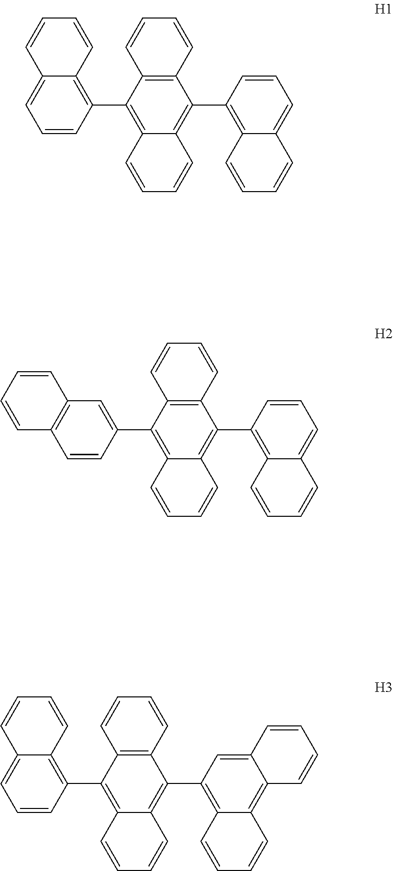

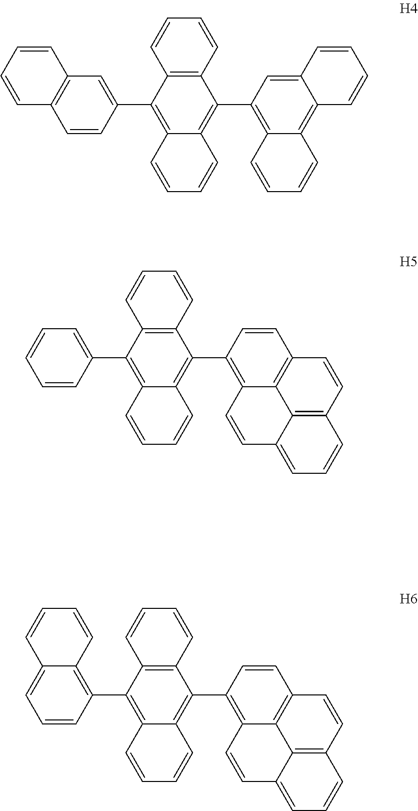

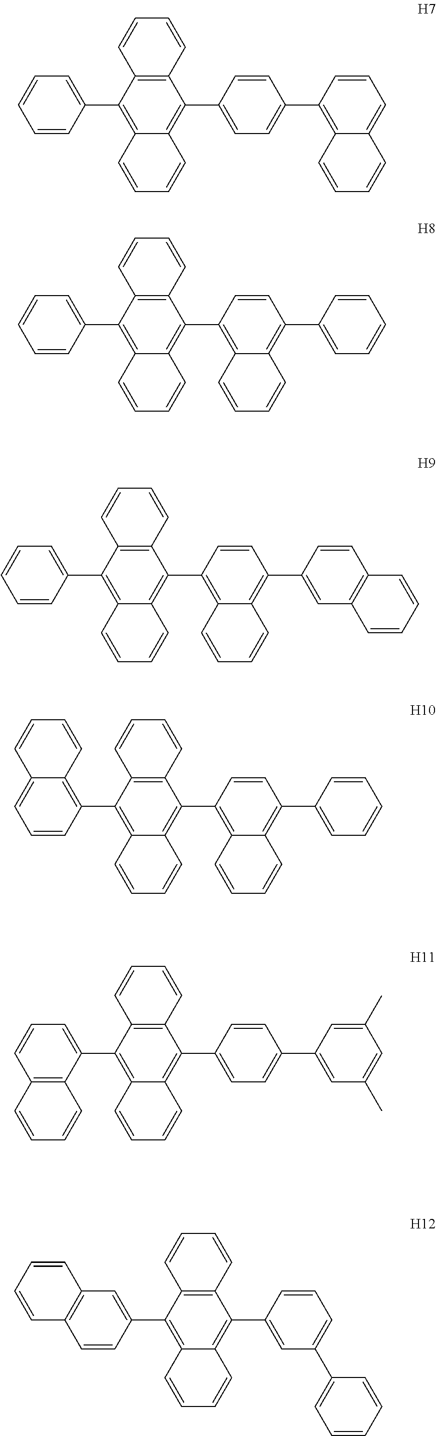

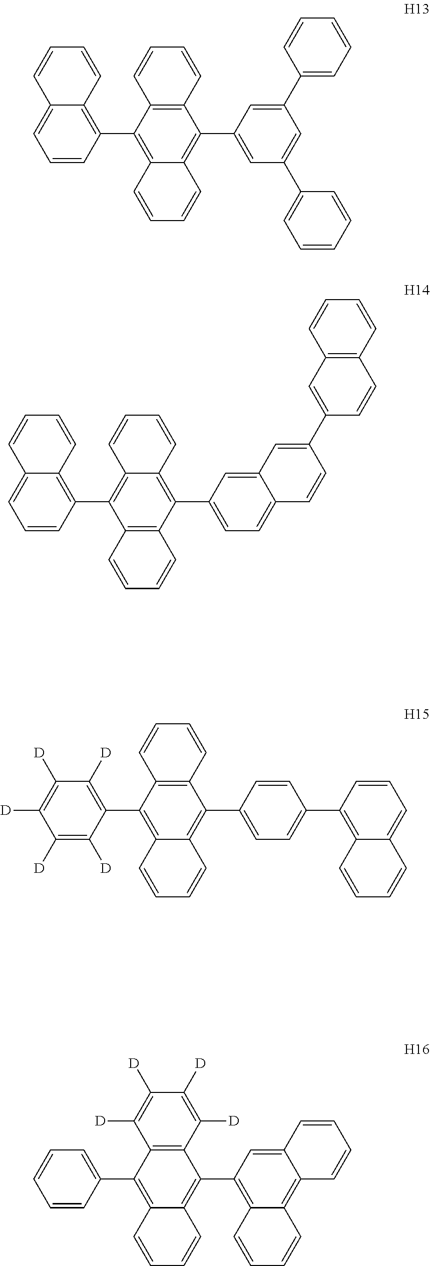

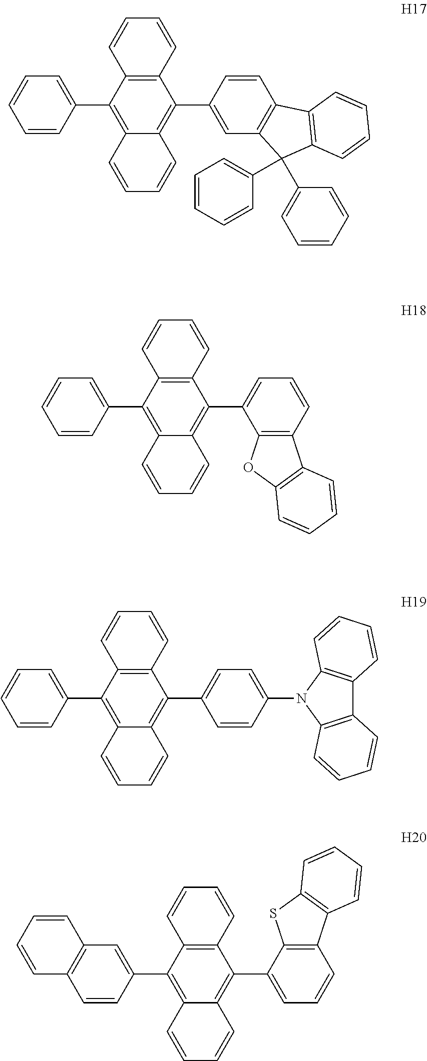

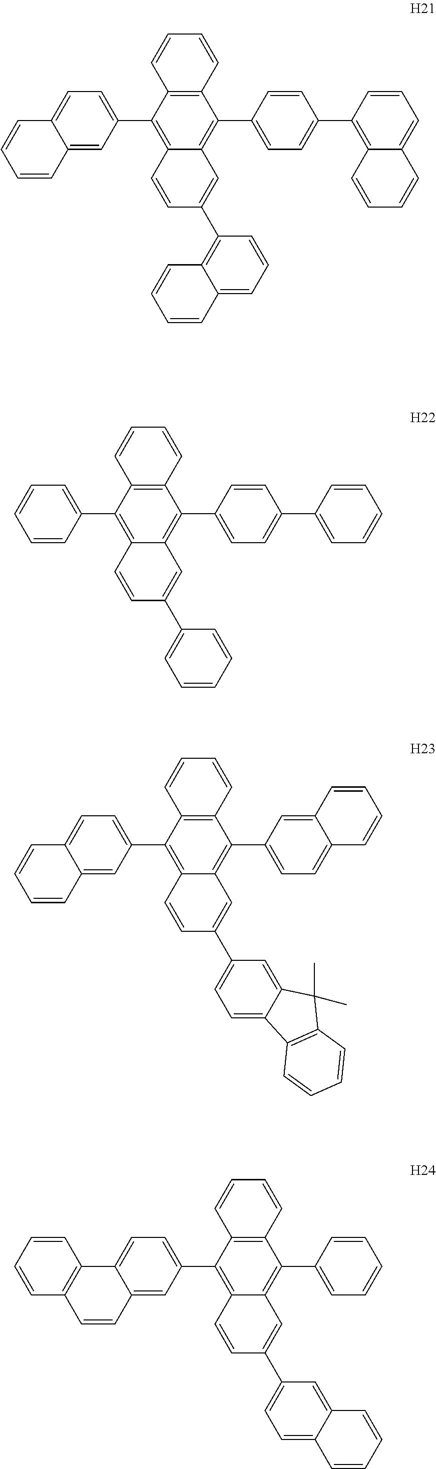

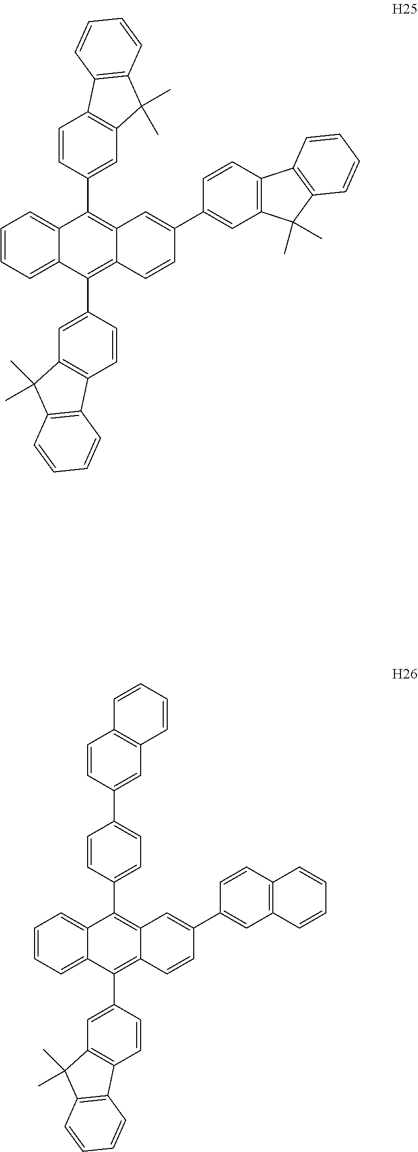

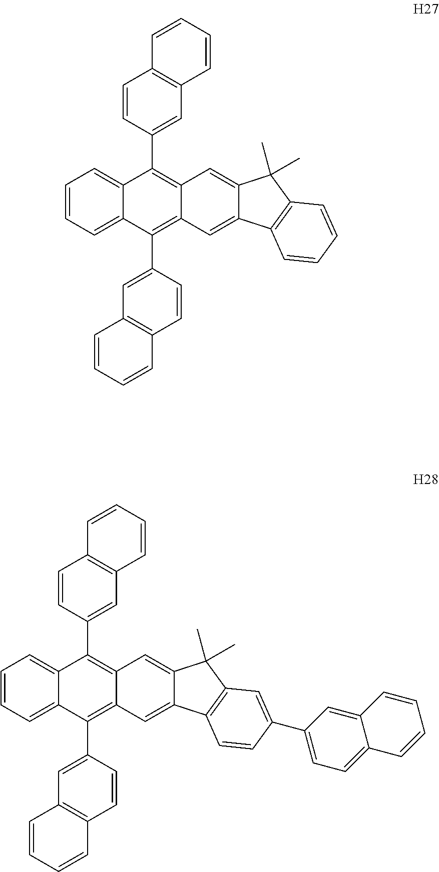

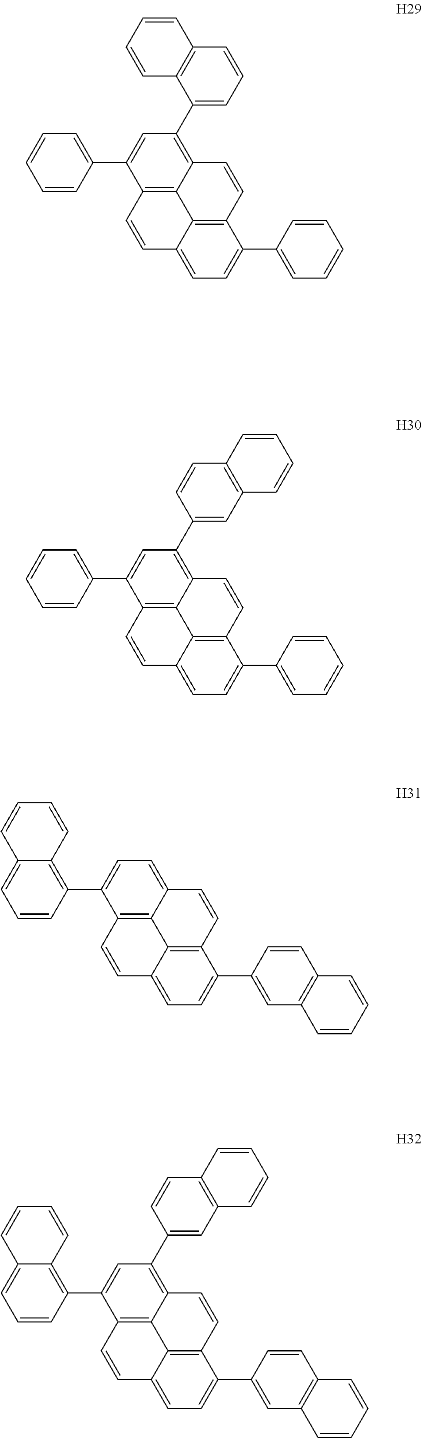

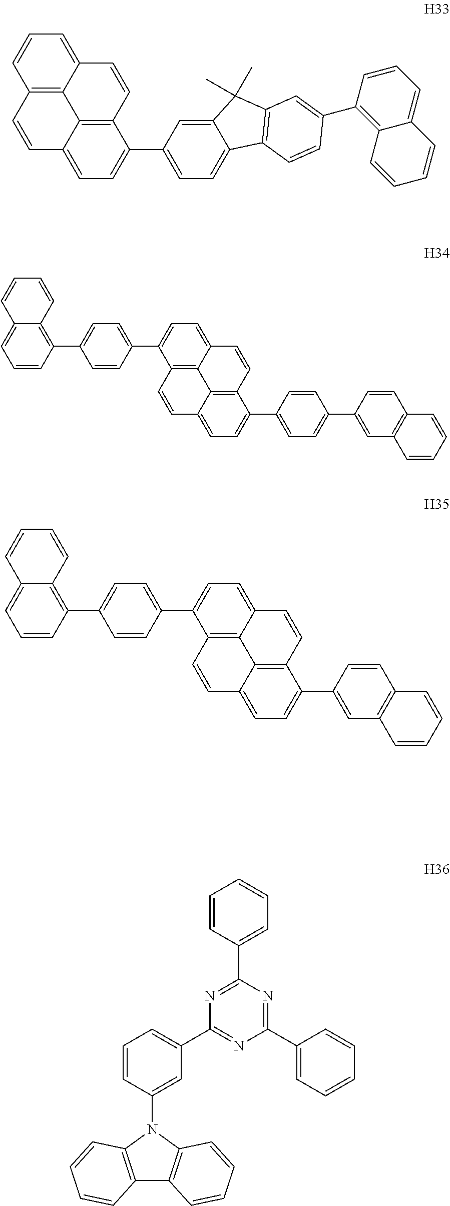

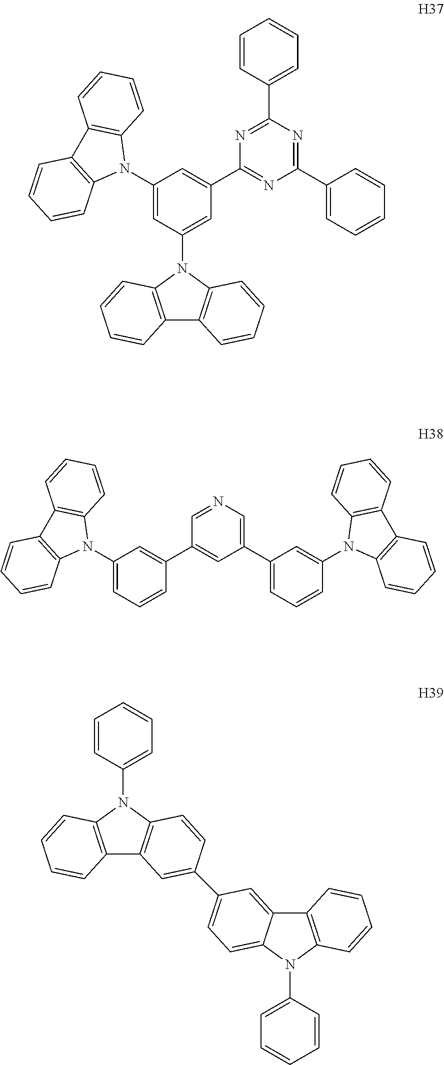 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1]

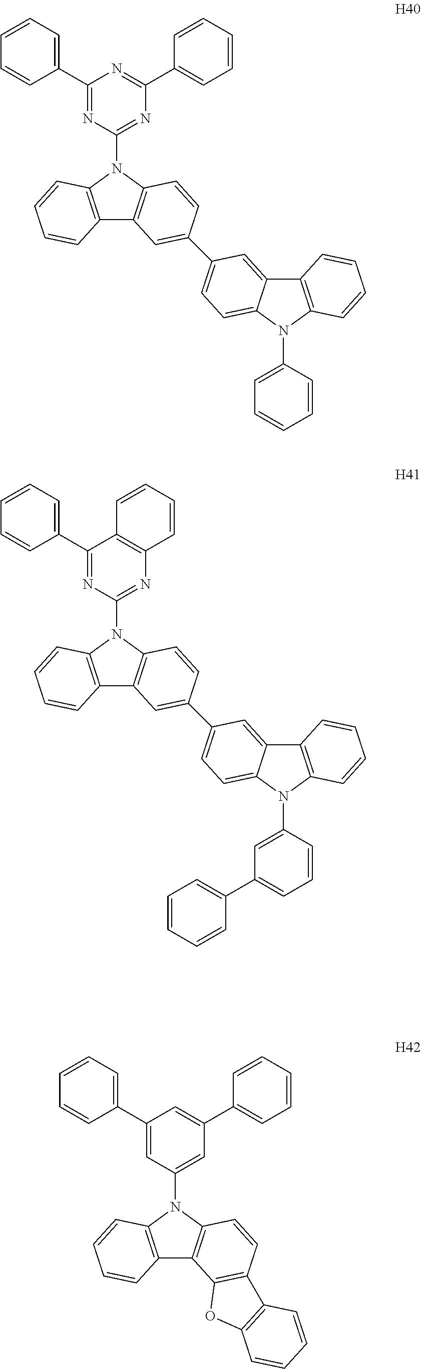 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

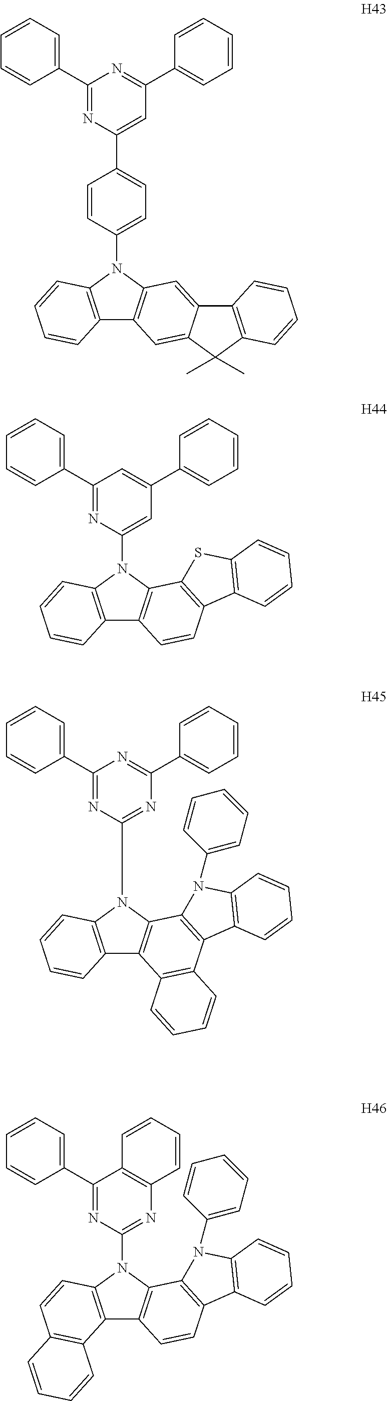 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

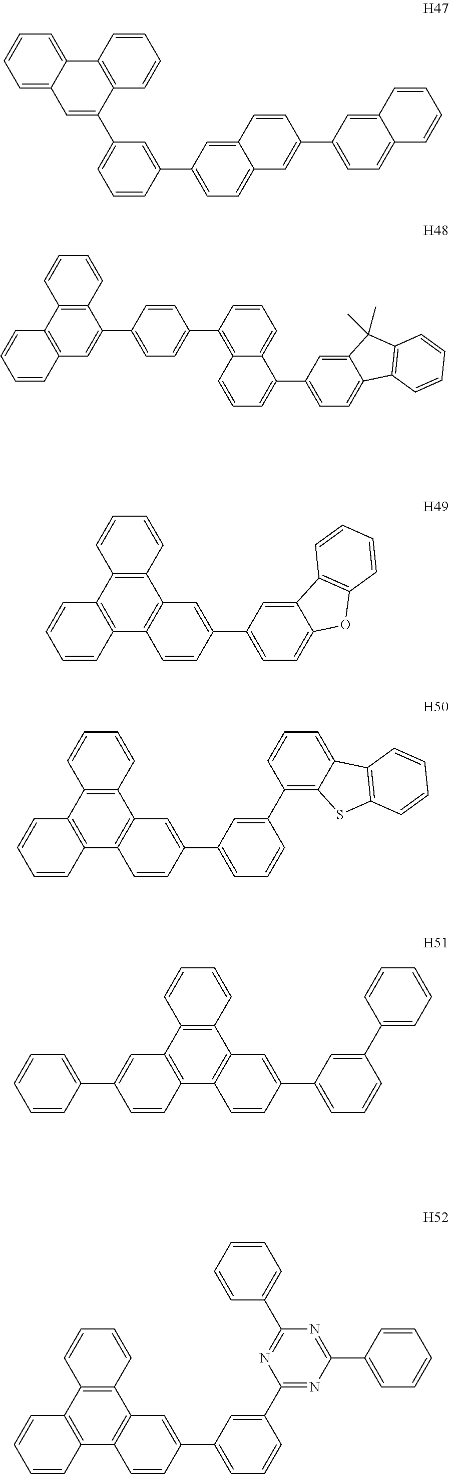

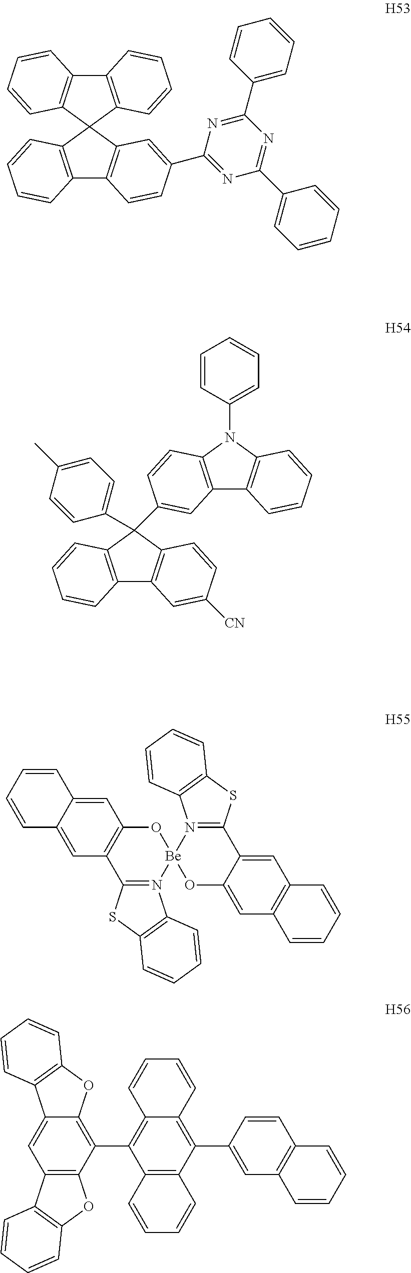

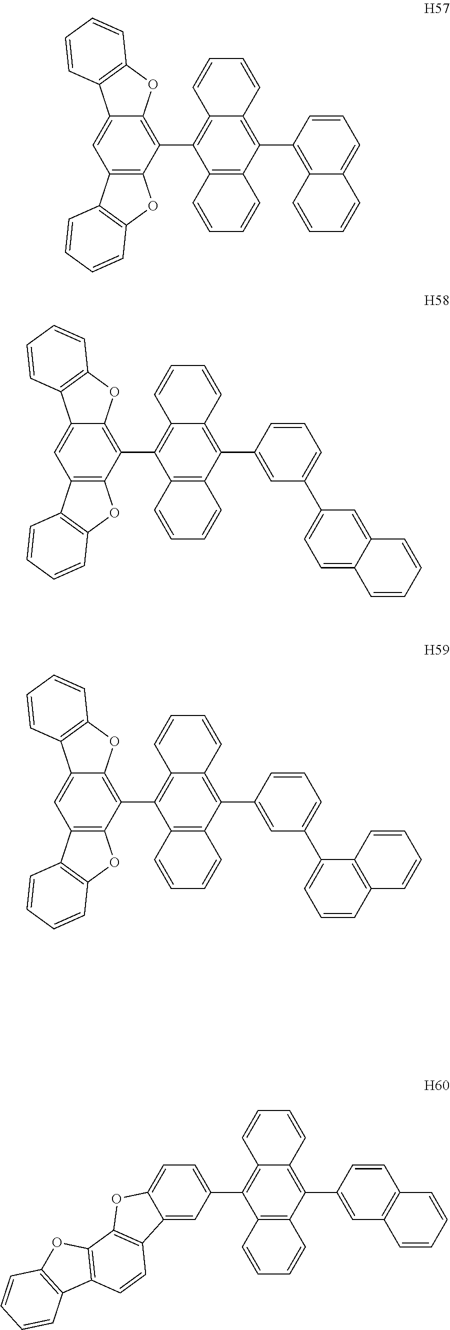 [SMILES: c1ccc2c(-c3c4ccccc4c(-c4c5oc6ccccc6c5cc5c4oc4ccccc45)c4ccccc34)cccc2c1 ; c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1]

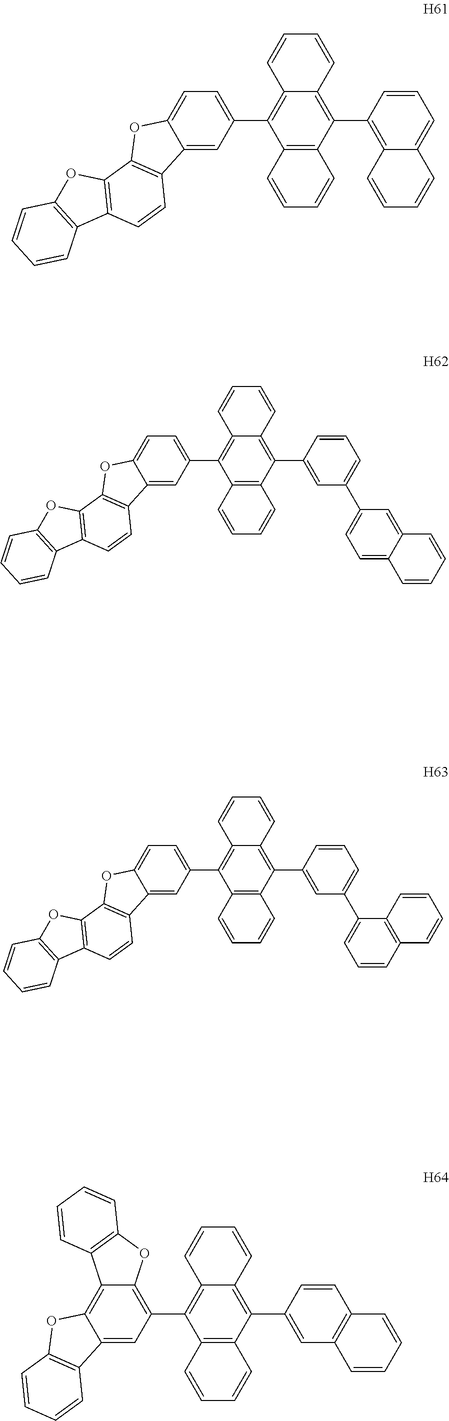 [SMILES: c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1 ; c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1]

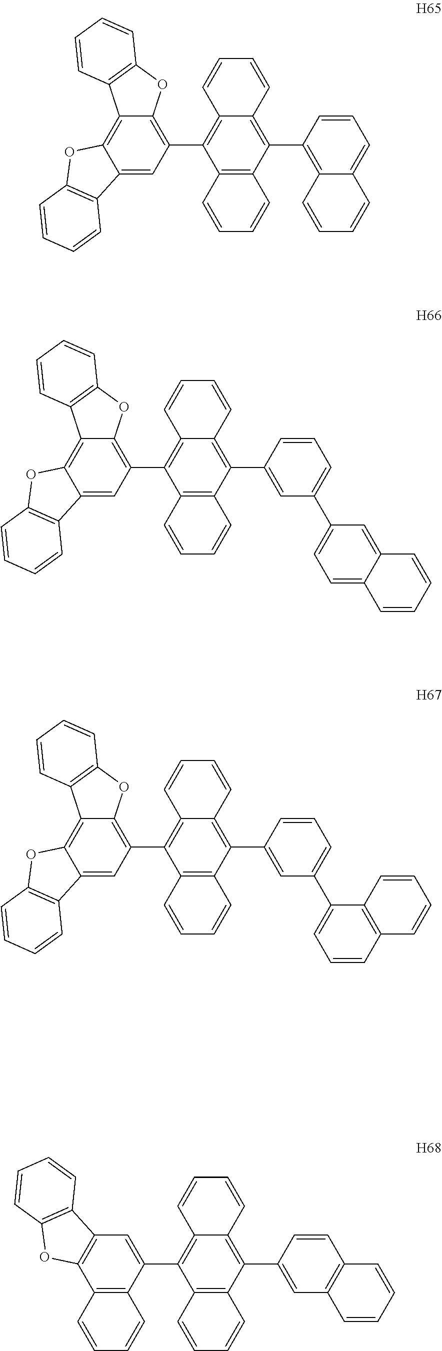 [SMILES: c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1 ; c1ccc2cc(-c3c4ccccc4c(-c4cc5c6ccccc6oc5c5ccccc45)c4ccccc34)ccc2c1]

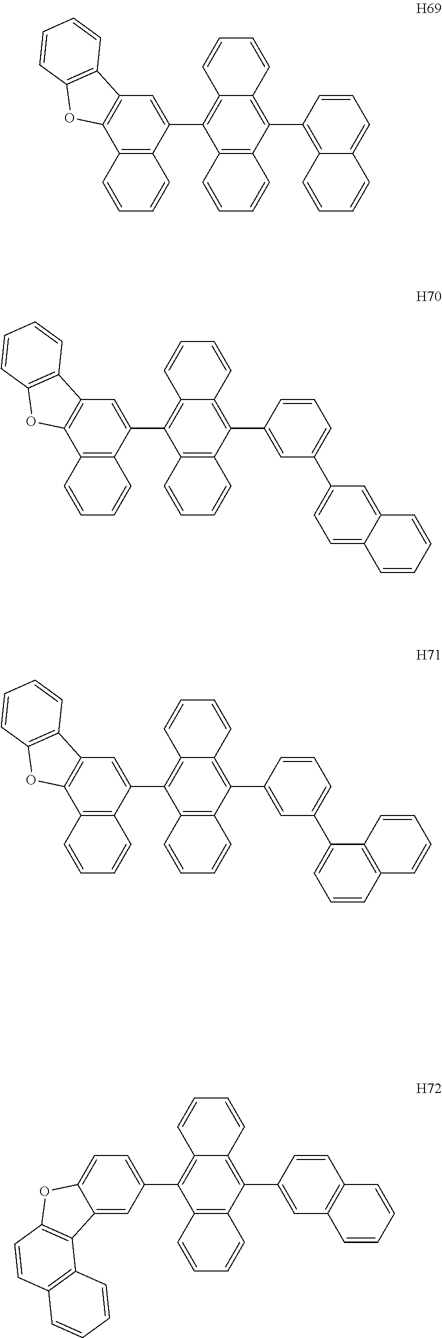 [SMILES: c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

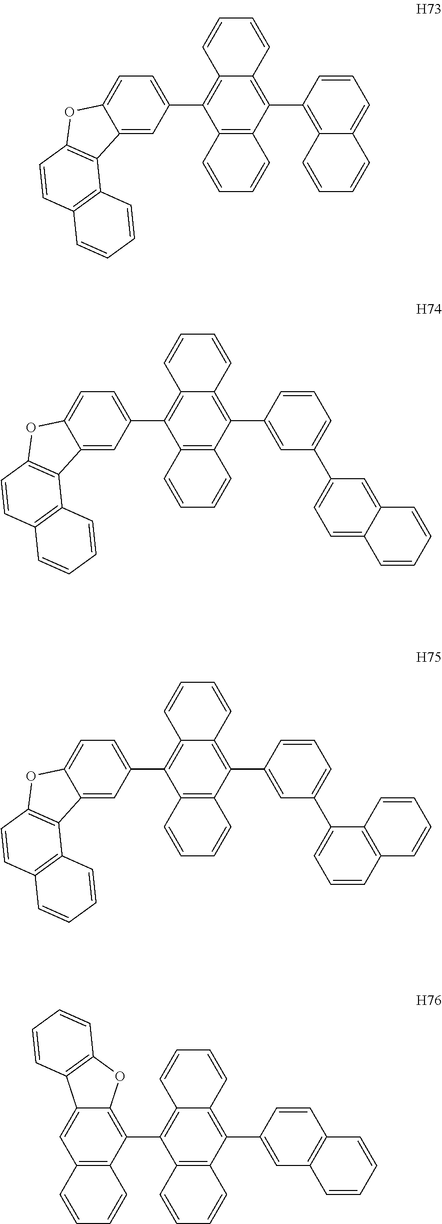 [SMILES: c1ccc2c(-c3c4ccccc4c(-c4ccc5oc6ccc7ccccc7c6c5c4)c4ccccc34)cccc2c1 ; c1cc(-c2ccc3ccccc3c2)cc(-c2c3ccccc3c(-c3ccc4oc5ccc6ccccc6c5c4c3)c3ccccc23)c1 ; c1cc(-c2cccc3ccccc23)cc(-c2c3ccccc3c(-c3ccc4oc5ccc6ccccc6c5c4c3)c3ccccc23)c1 ; c1ccc2cc(-c3c4ccccc4c(-c4c5ccccc5cc5c4oc4ccccc45)c4ccccc34)ccc2c1]

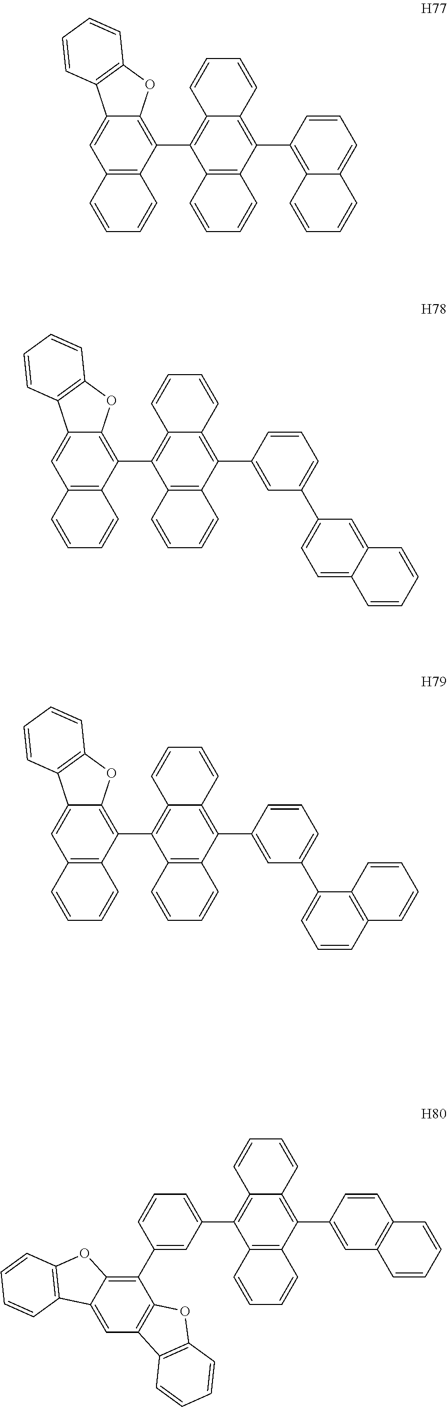 [SMILES: c1ccc2c(-c3c4ccccc4c(-c4c5ccccc5cc5c4oc4ccccc45)c4ccccc34)cccc2c1 ; c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1]

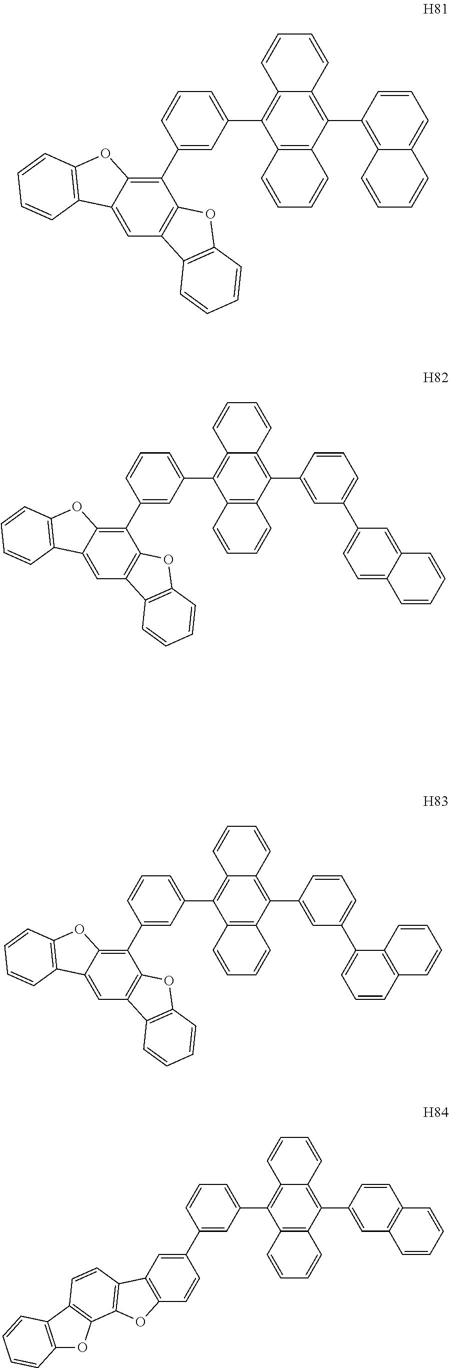 [SMILES: c1cc(-c2c3ccccc3c(-c3cccc4ccccc34)c3ccccc23)cc(-c2c3oc4ccccc4c3cc3c2oc2ccccc23)c1 ; c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1]

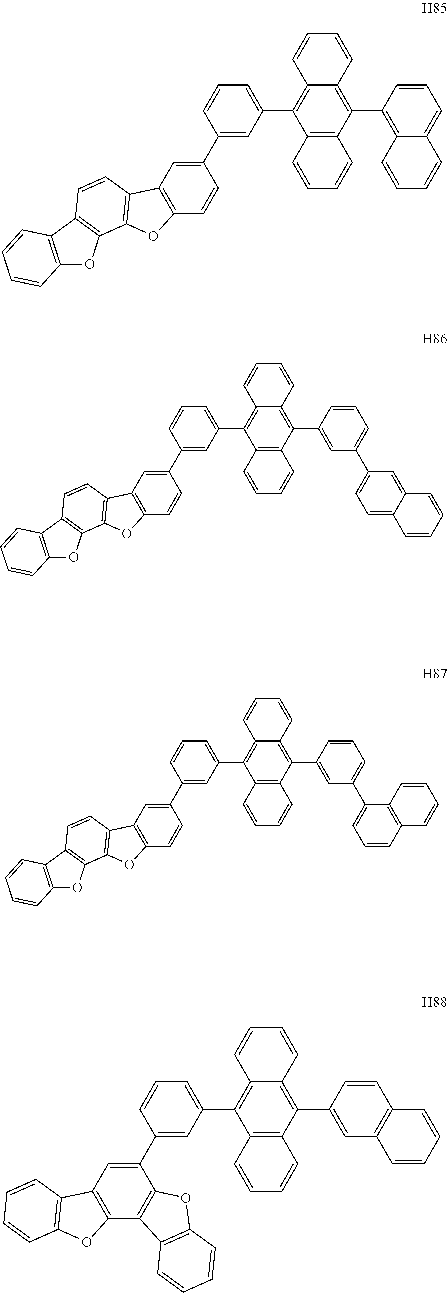 [SMILES: c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1 ; c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1]

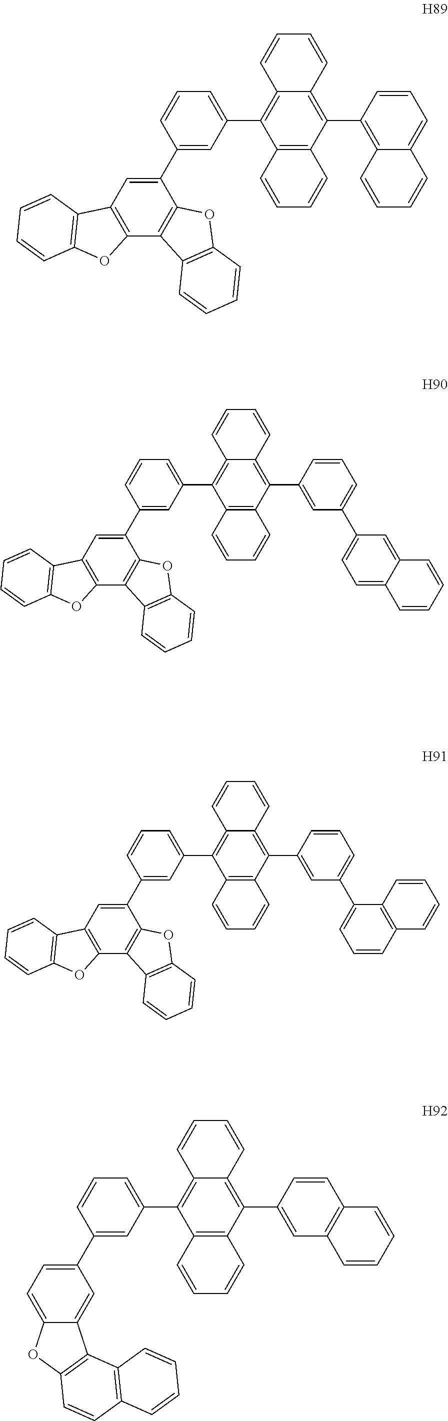 [SMILES: c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1 ; c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1]

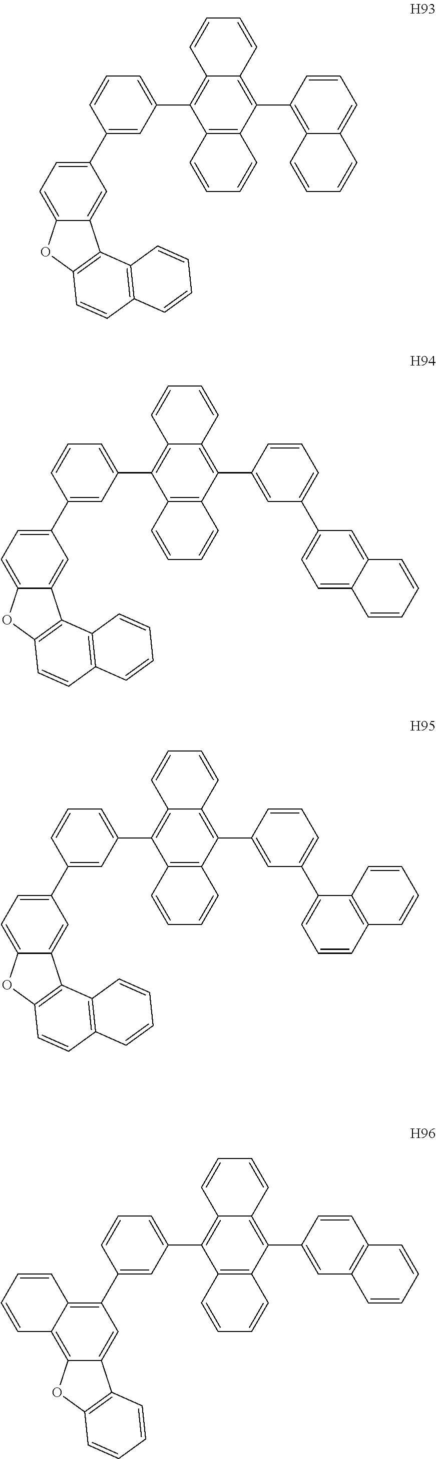 [SMILES: c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3ccccc23)c1]

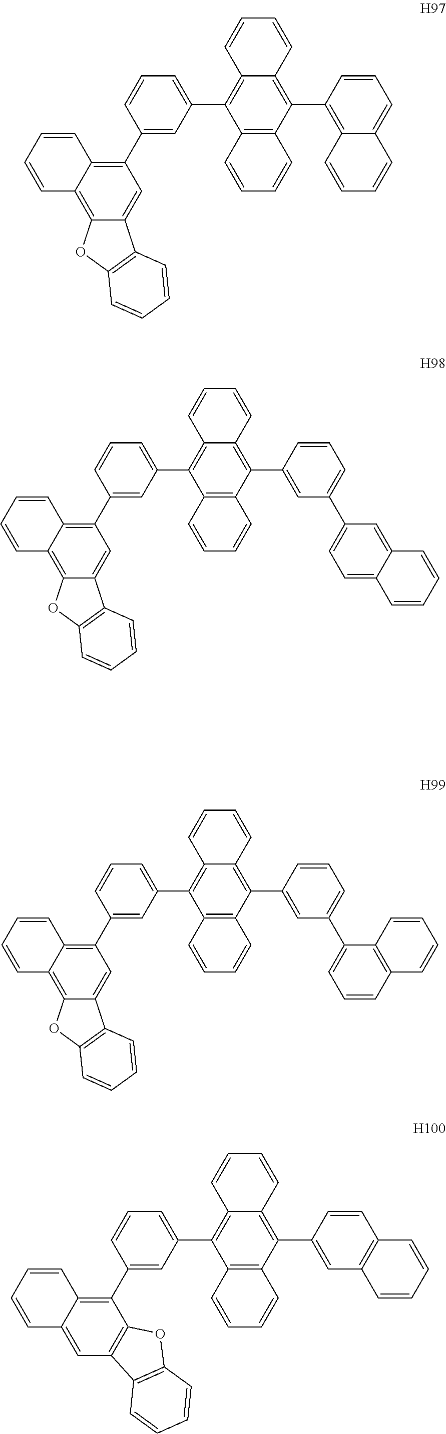 [SMILES: c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1 ; c1cc(-c2c3ccccc3c(-c3ccc4ccccc4c3)c3ccccc23)cc(-c2c3ccccc3cc3c2oc2ccccc23)c1]

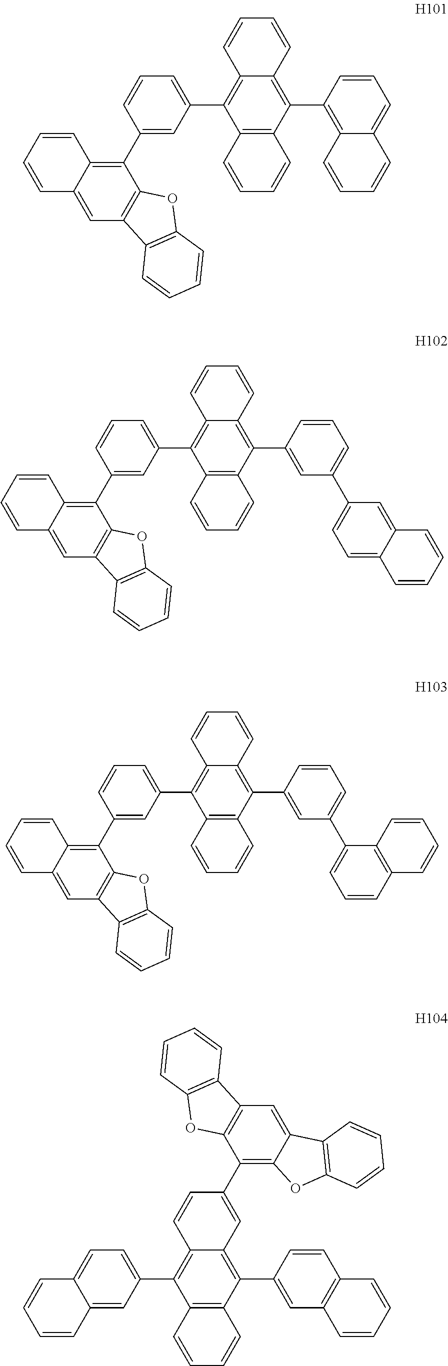 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

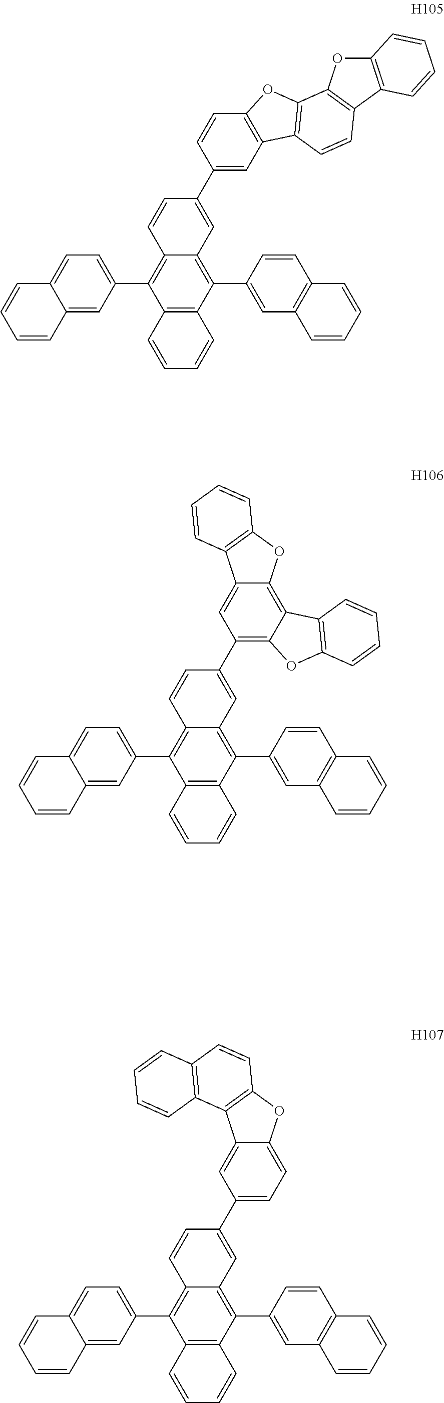 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1]

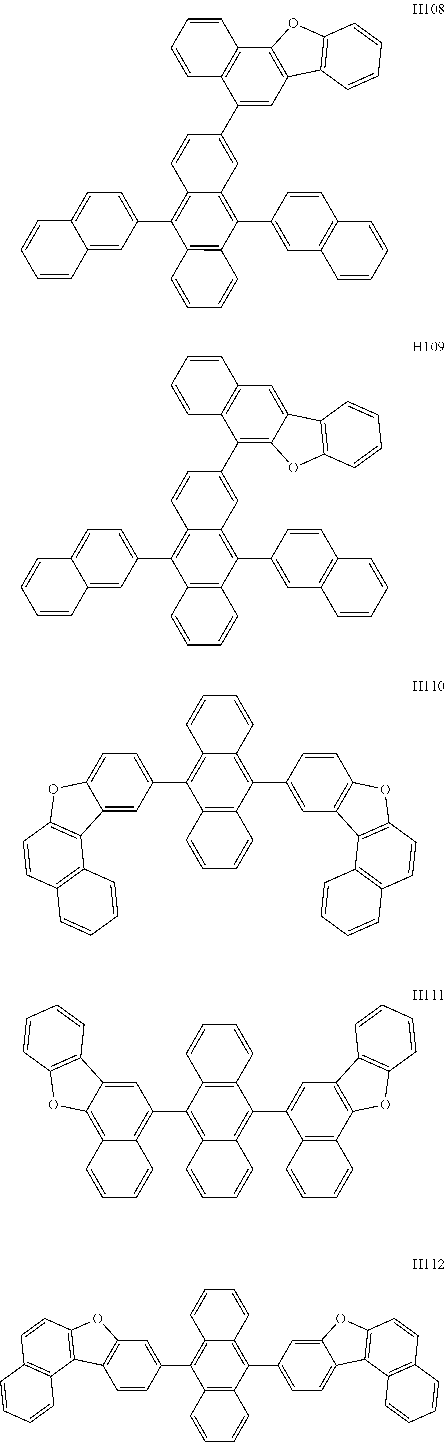 [SMILES: c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12 ; c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12]

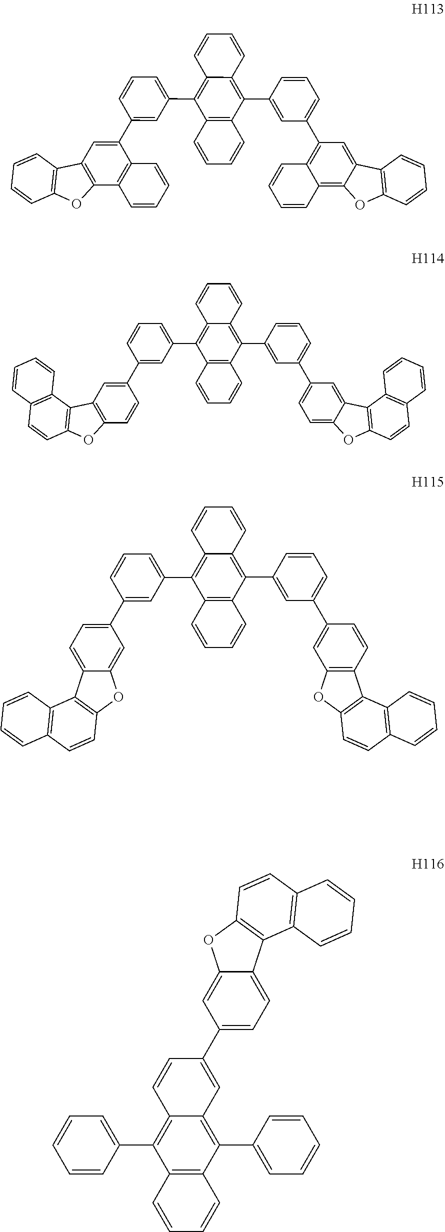 [SMILES: c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1]

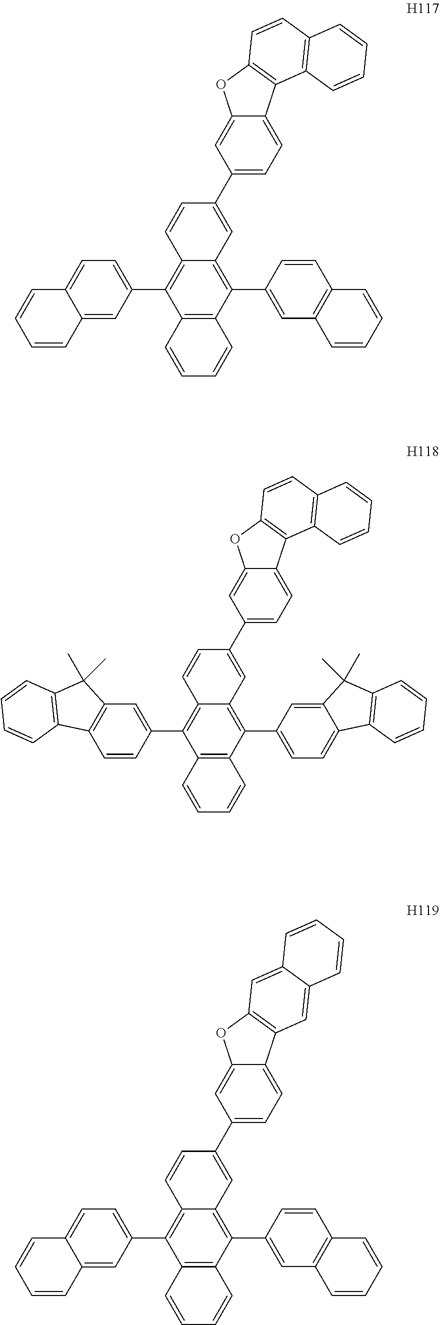 [SMILES: c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21 ; c1ccc2cc(-c3c4ccccc4c(-c4ccc5ccccc5c4)c4cc(-c5ccc6c(c5)oc5cc7ccccc7cc56)ccc34)ccc2c1]

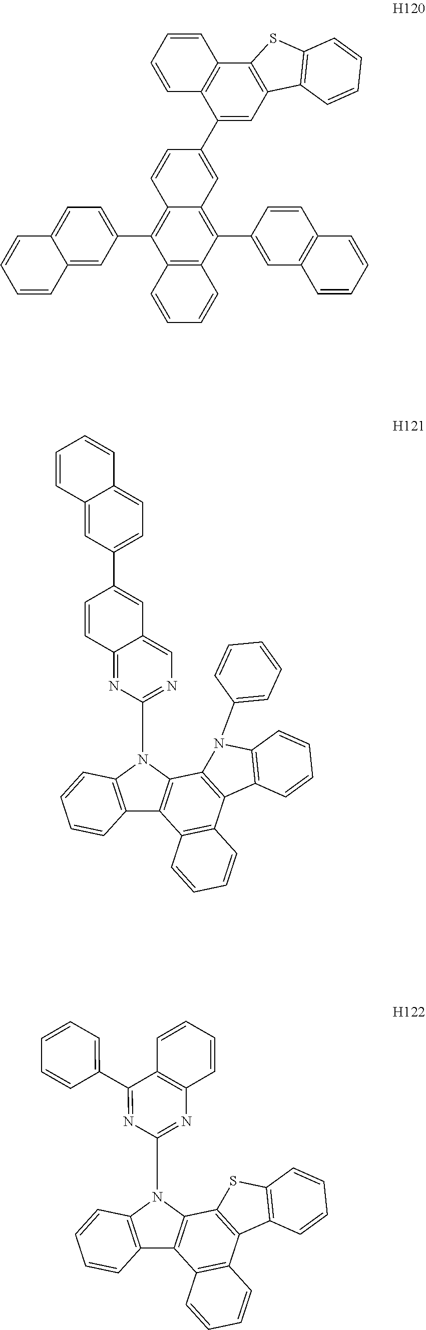 [SMILES: c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1 ; c1ccc(-c2nc(-n3c4ccccc4c4c5ccccc5c5c6ccccc6sc5c43)nc3ccccc23)cc1]

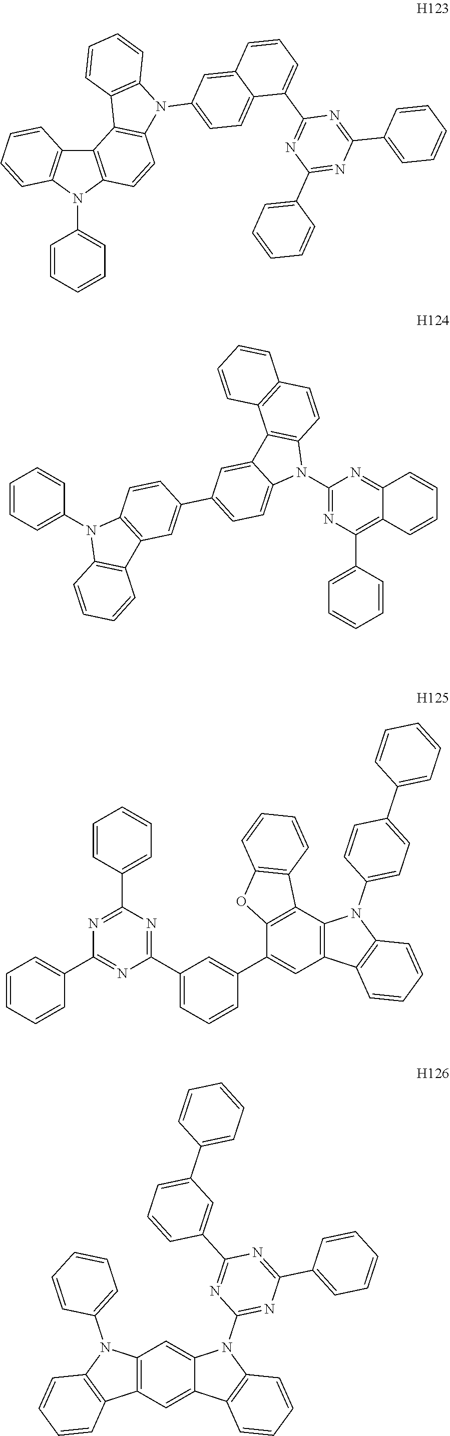 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1]

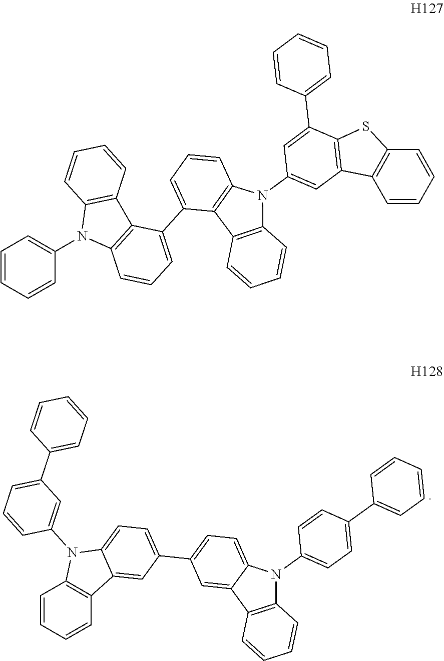 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In one or more embodiments, the phosphorescent dopant may include an organometallic compound represented by Formula 401:

M(L₄₀₁)_{xc1}(L₄₀₂)_{xc2} Formula 401

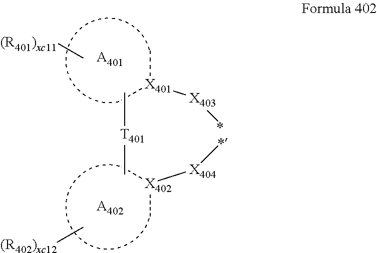

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, if (e.g., when) xc1 is 2 or more, two or more of L₄₀₁ may be substantially identical to or different from each other,
L₄₀₂ may be an organic ligand, and xc2 may be 0, 1, 2, 3, or 4, wherein, if (e.g., when) xc2 is 2 or more, two or more of L₄₀₂ may be substantially identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *=C═*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃) (Q₄₁₄),
Q₄₁₁ to Q₄₁₄ are each as described in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group that is unsubstituted or substituted with at least one R₁₀ₐ, a C₁-C₂₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ are each as described in connection with Q₁,
xc11 and xc12 may each independently be an integer from 0 to 10, and
* and *′ in Formula 402 each indicate a binding site to M in Formula 401.

For example, in Formula 402, i) X₄₀₁ may be nitrogen and X₄₀₂ may be carbon, or ii) each of X₄₀₁ and X₄₀₂ may be nitrogen.

In one or more embodiments, if (e.g., when) xc1 in Formula 401 is 2 or more, two ring A₄₀₁ among two or more of L₄₀₁ may be optionally linked together via T₄₀₂, which is a linking group, and two ring A₄₀₂ may be optionally linked together via T₄₀₃, which is a linking group (see Compounds PD1 to PD4 and PD7). T₄₀₂ and T₄₀₃ are each as described in connection with T₄₀₁.

In Formula 401, L₄₀₂ may be an organic ligand. For example, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, and/or the like), or any combination thereof.

The phosphorescent dopant may include, for example, at least one of (e.g., one or more selected from among) Compounds PD1 to PD39, or any combination thereof:

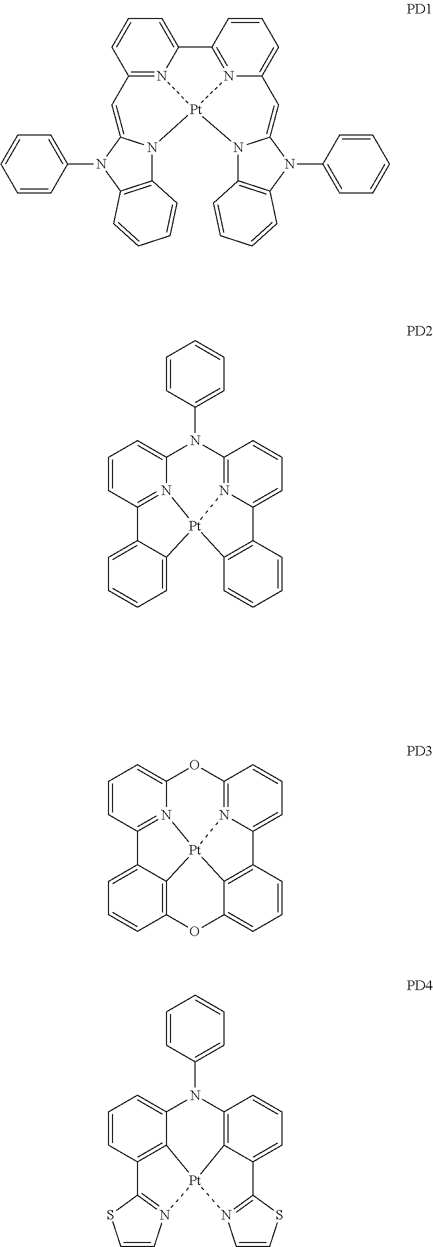

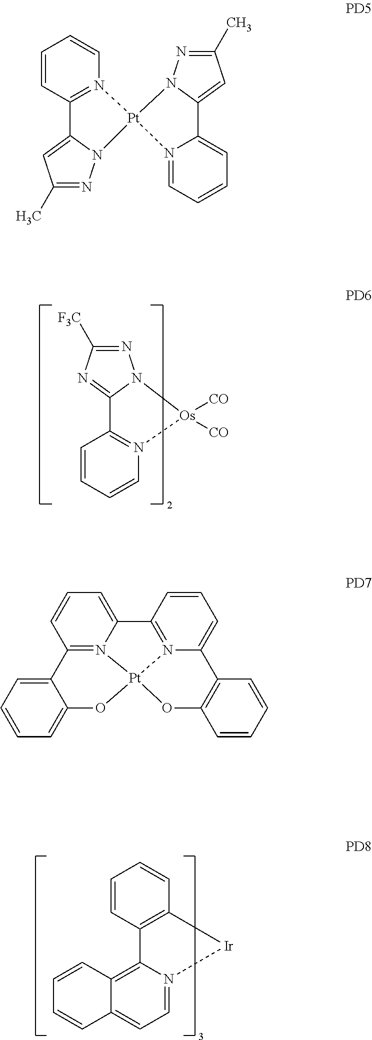

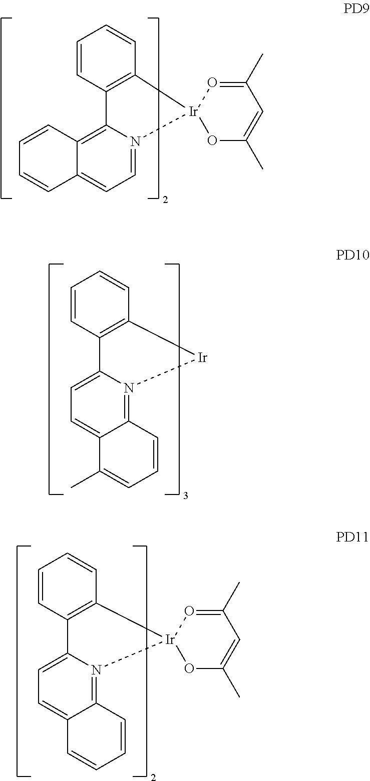

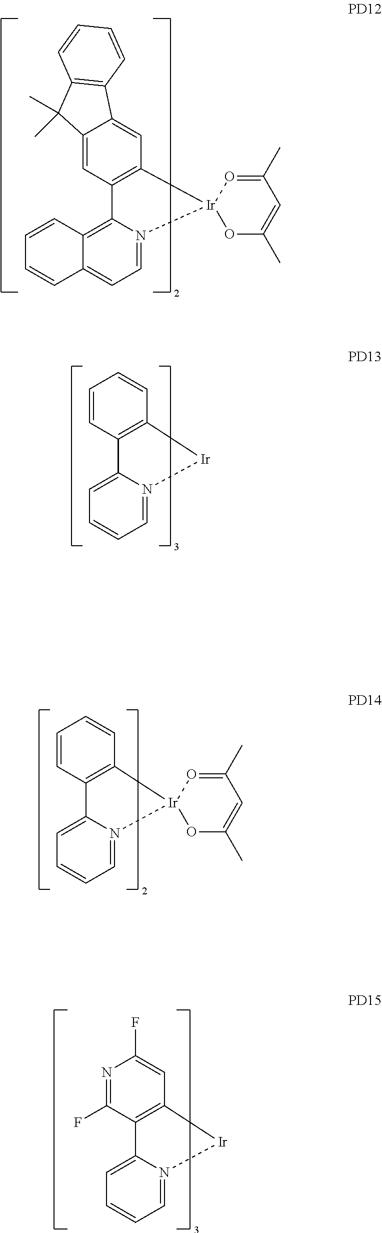

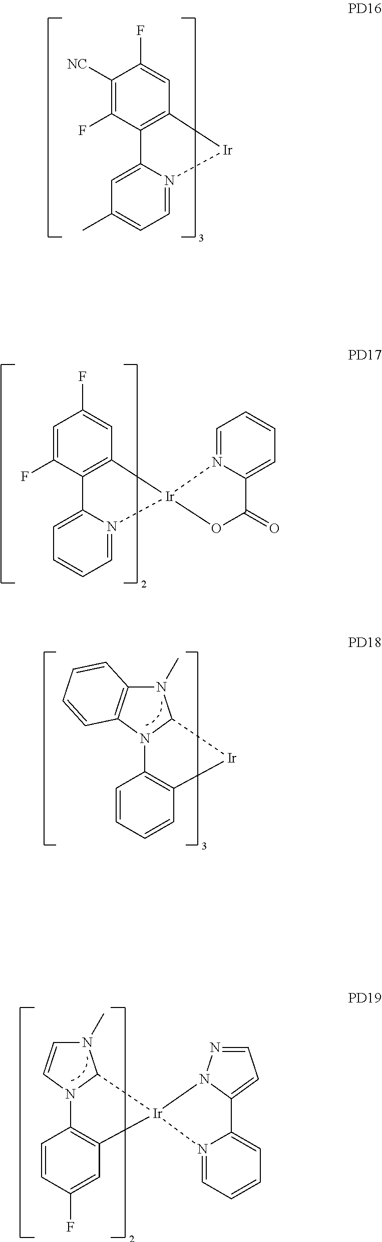

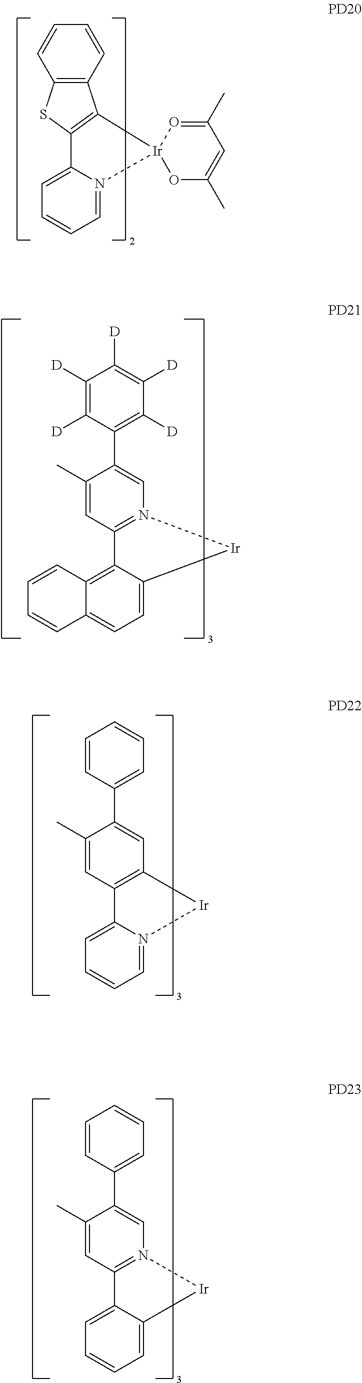

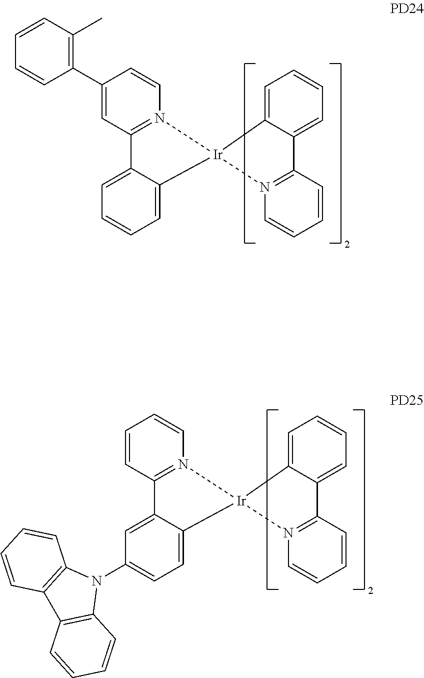

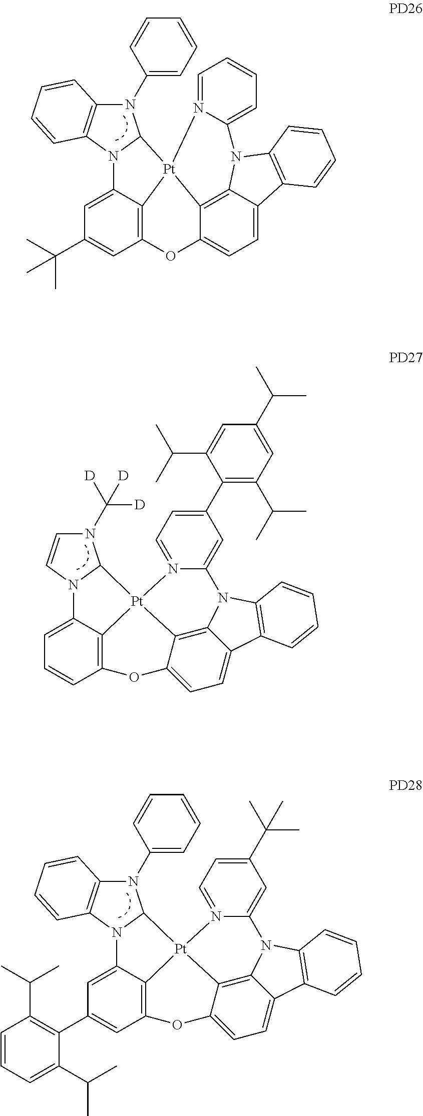

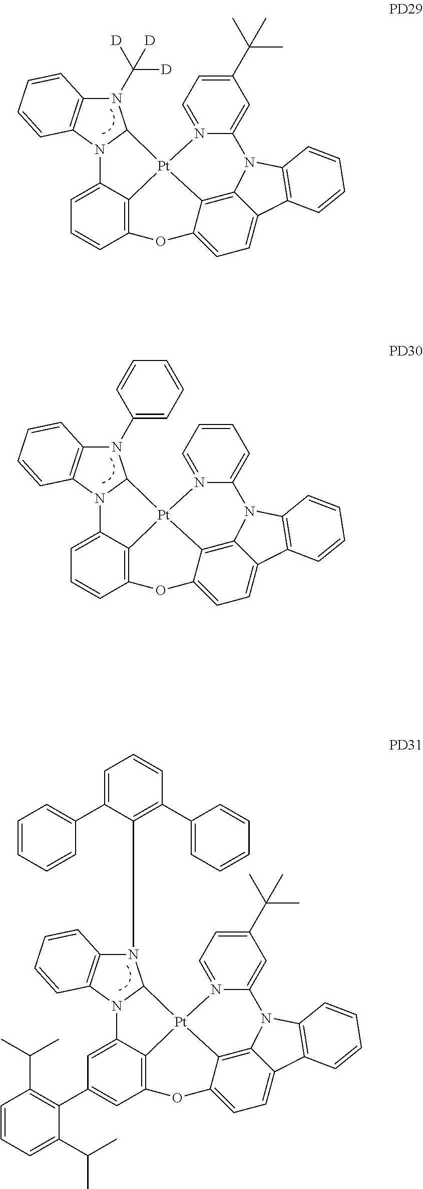

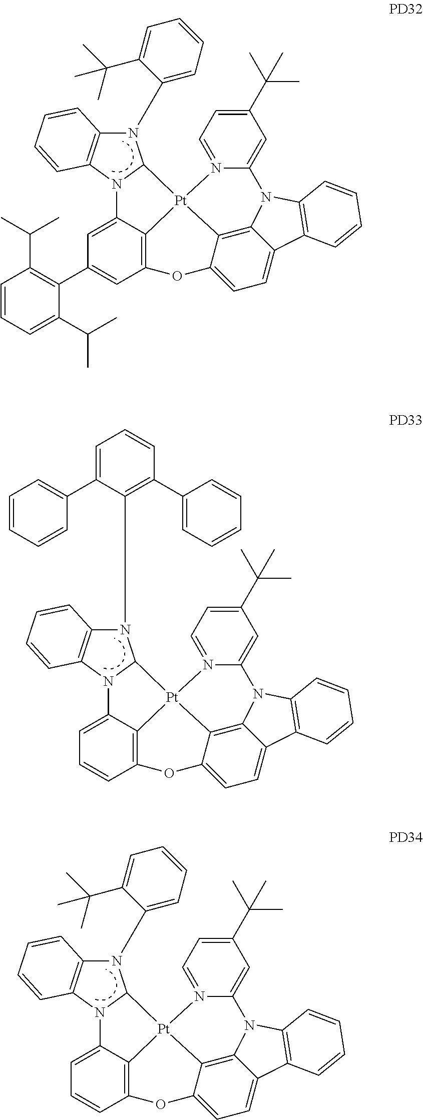

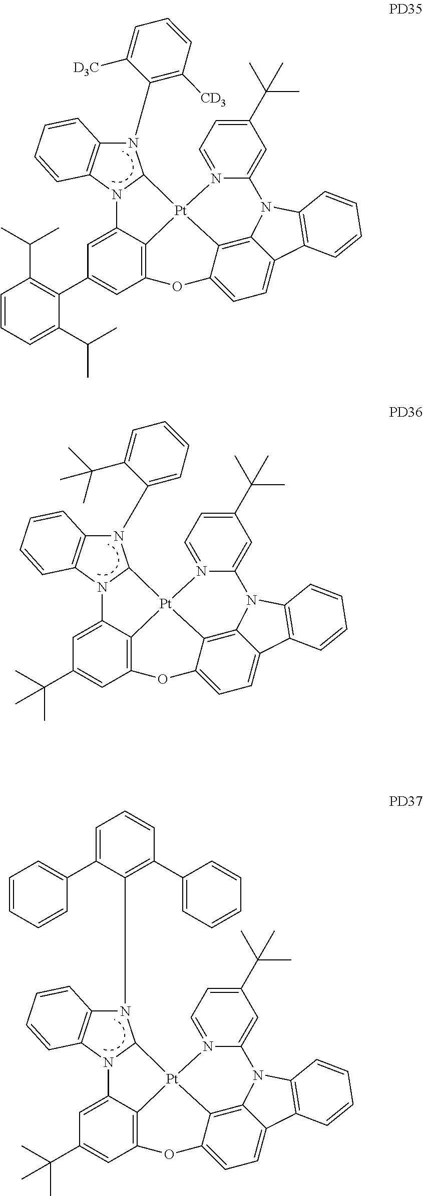

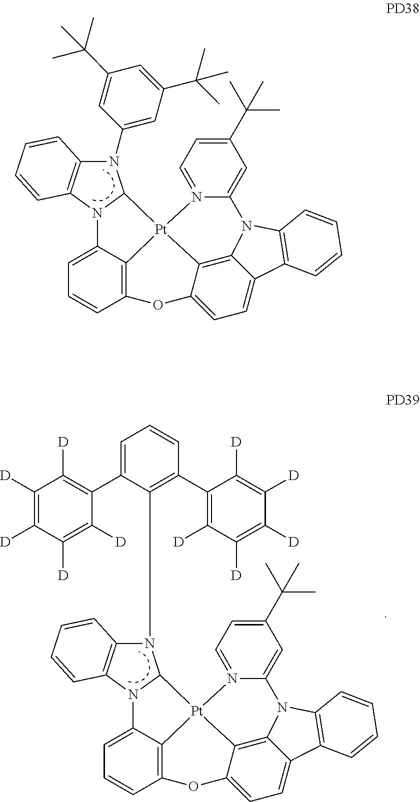

### Fluorescent Dopant

The fluorescent dopant may include an amine group-containing compound, a styryl group-containing compound, or any combination thereof.

For example, the fluorescent dopant may include a compound represented by Formula 501:

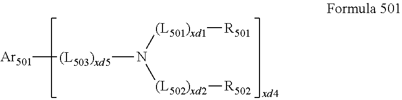

wherein, in Formula 501,
Ar₅₀₁, L₅₀₁ to L₅₀₃, R₅₀₁, and R₅₀₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, and/or the like) in which three or more monocyclic groups are condensed together.

In one or more embodiments, xd4 in Formula 501 may be 2.

In one or more embodiments, the fluorescent dopant may include: at least one of (e.g., one or more selected from among) Compounds FD1 to FD37; DPVBi; DPAVBi; or any combination thereof:

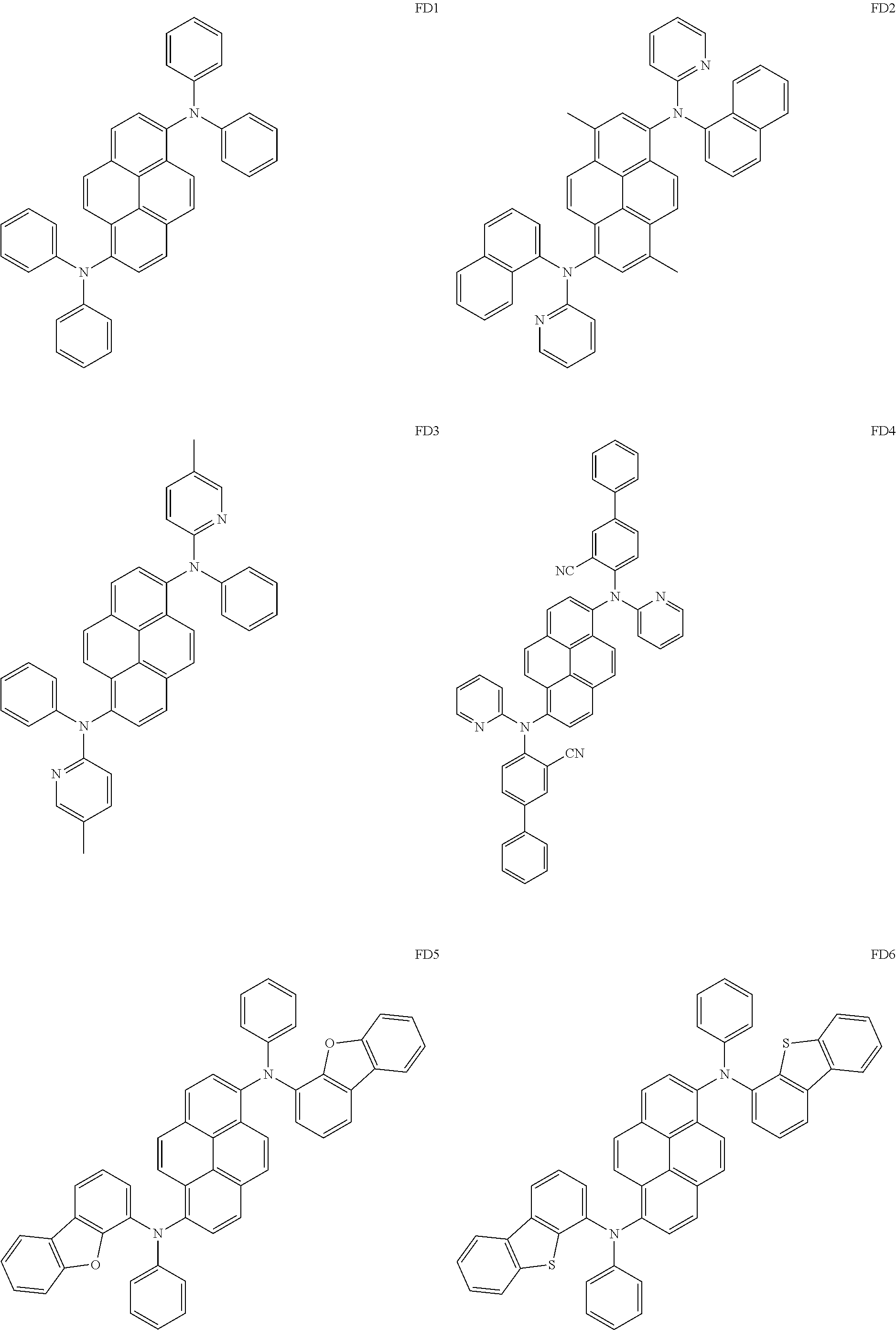

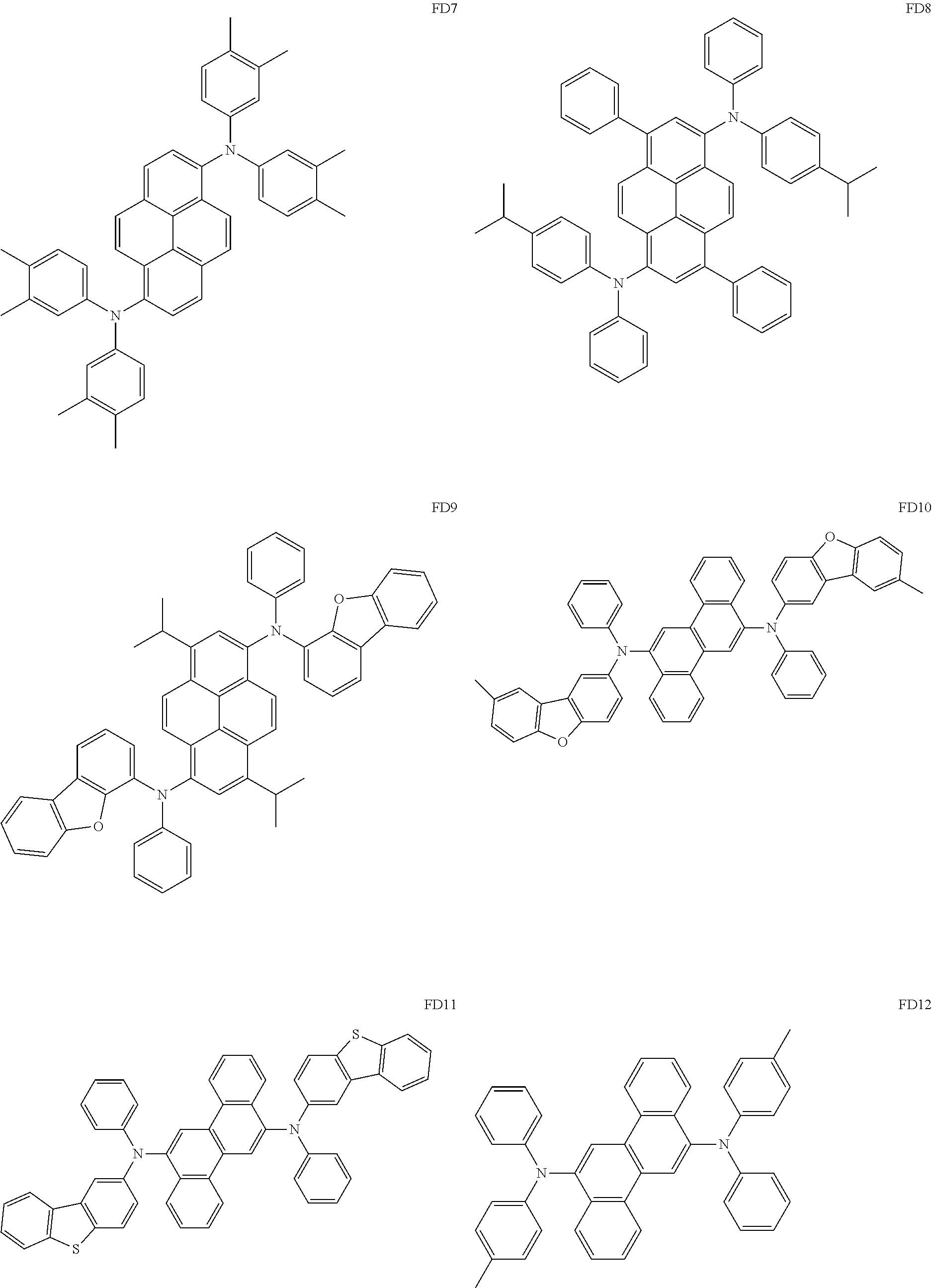

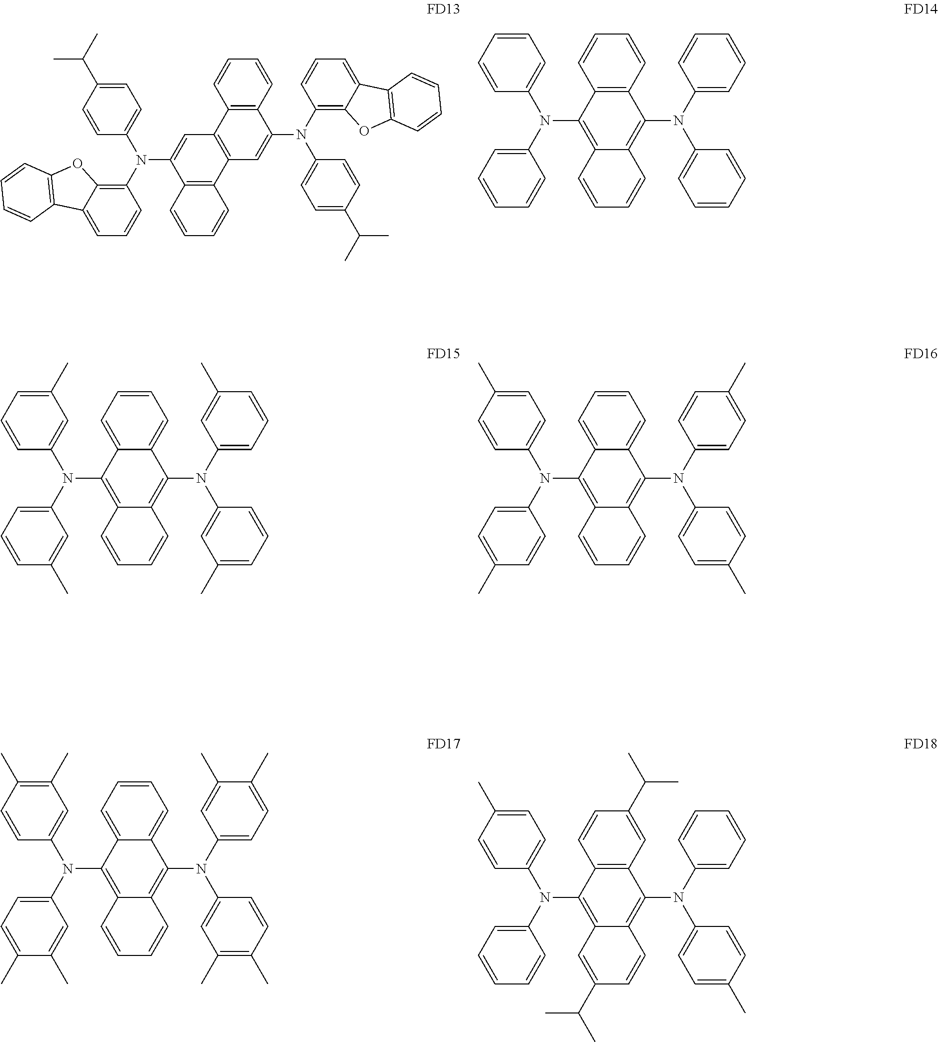

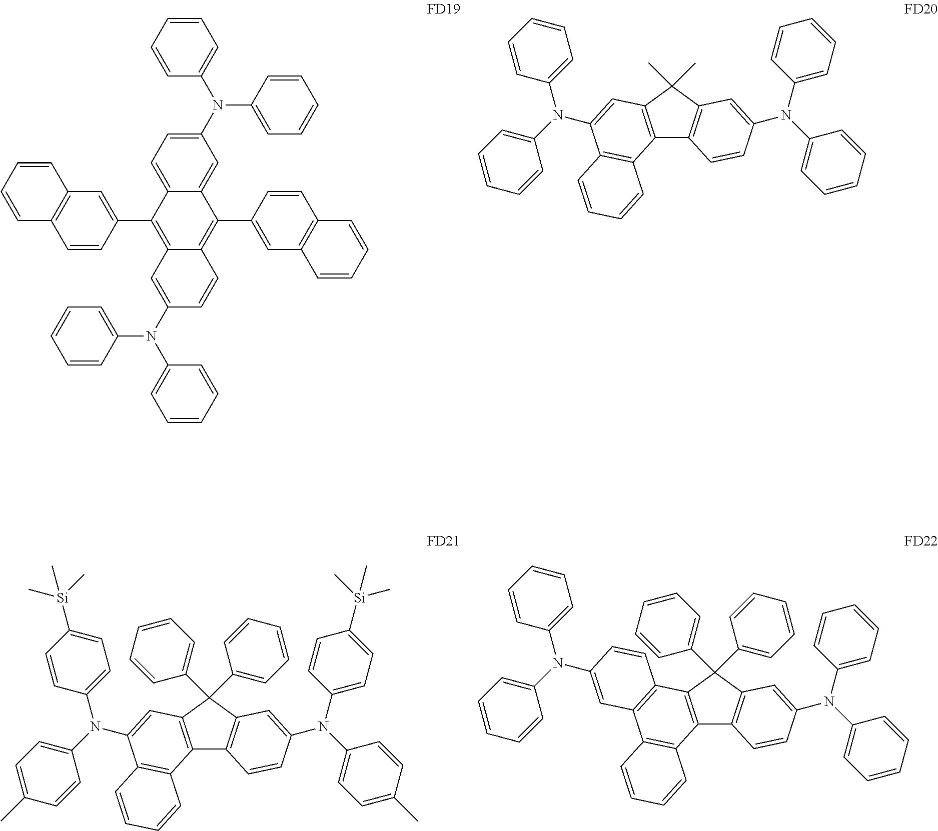

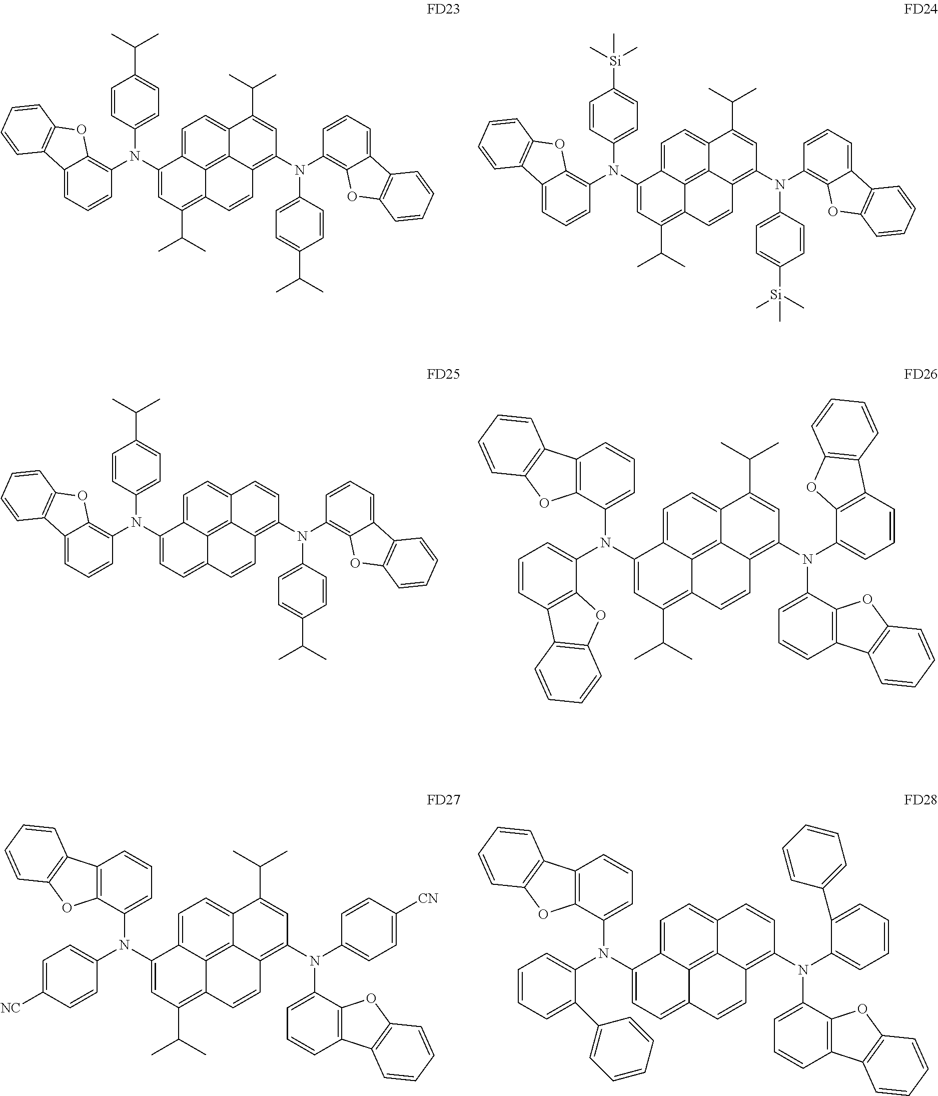

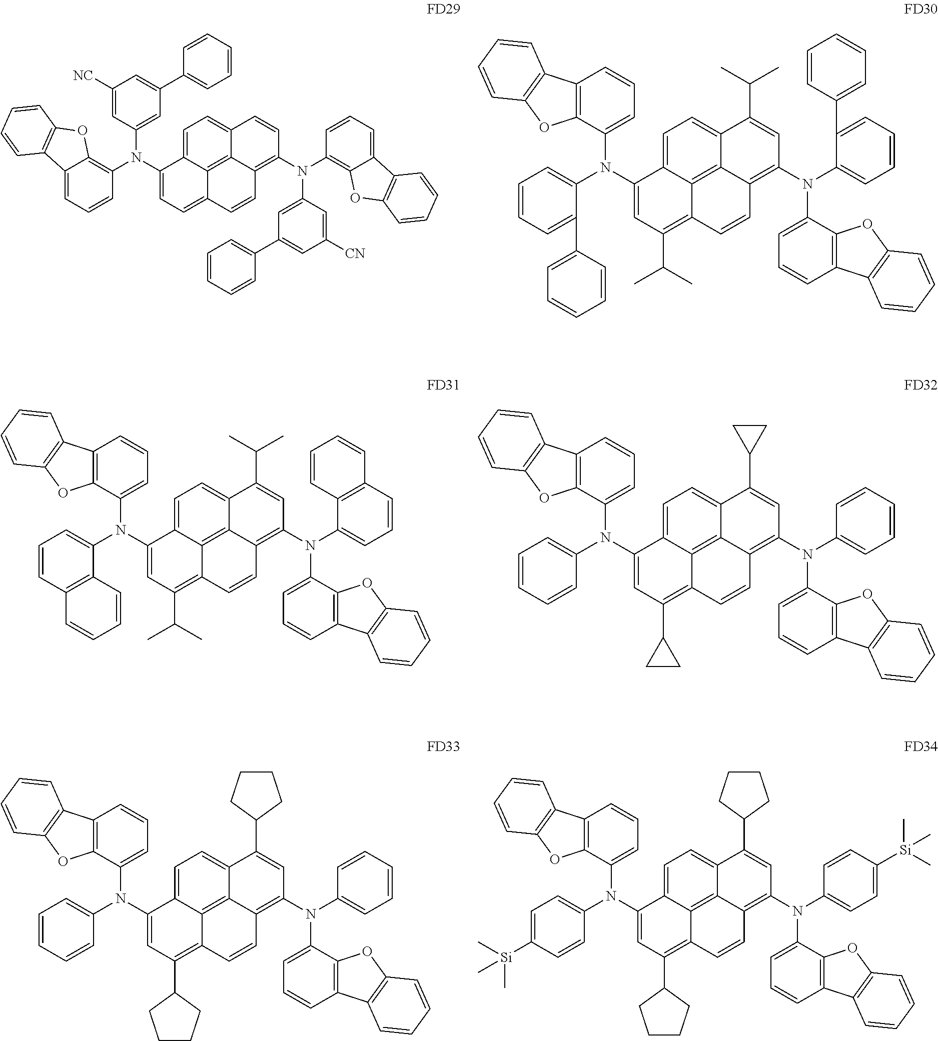

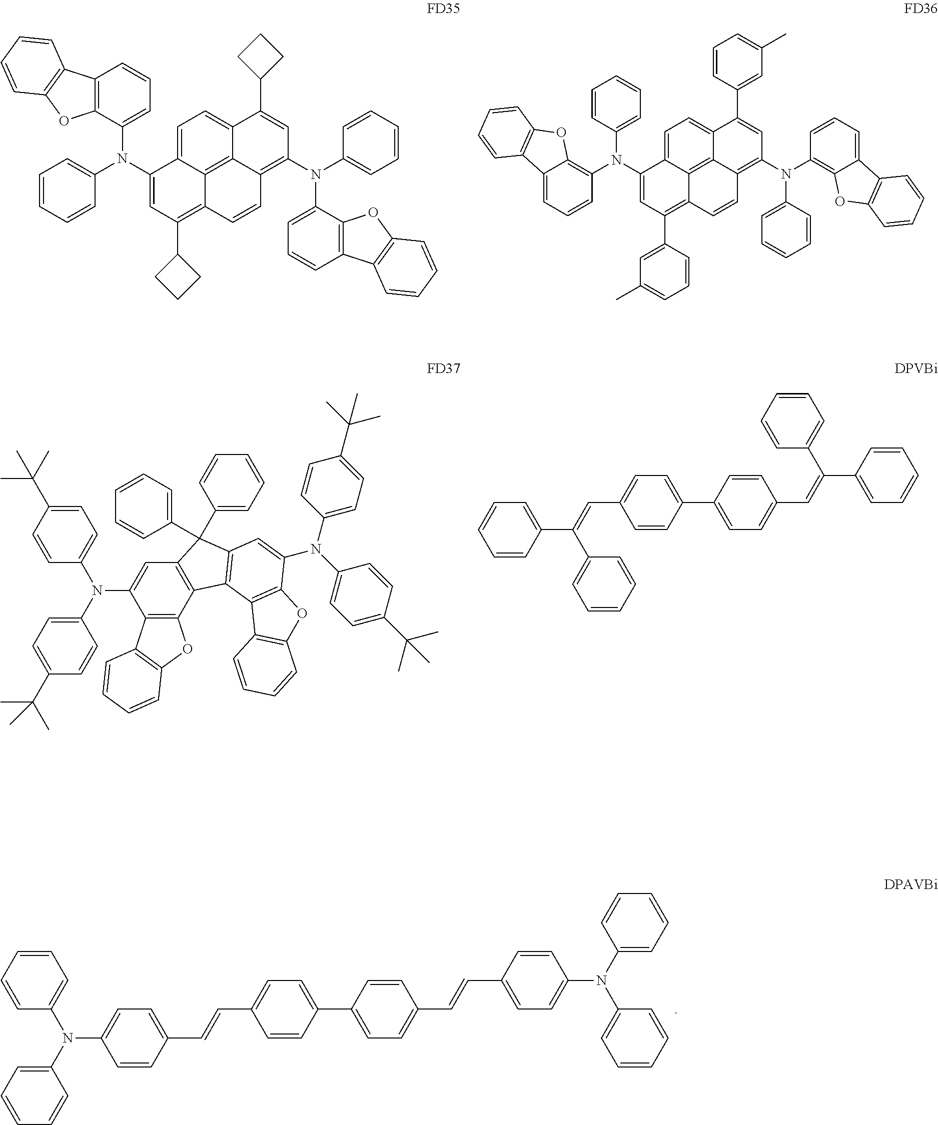

### Delayed Fluorescence Material

The emission layer may include a delayed fluorescence material.

Herein, the delayed fluorescence material may be selected from among compounds capable of emitting delayed fluorescence based on a delayed fluorescence emission mechanism.

The delayed fluorescence material included in the emission layer may act as a host or a dopant depending on the type or kind of other materials included in the emission layer.

In one or more embodiments, a difference between the triplet energy level (eV) of the delayed fluorescence material and the singlet energy level (eV) of the delayed fluorescence material may be about 0 eV to about 0.5 eV. When the difference between the triplet energy level (eV) of the delayed fluorescence material and the singlet energy level (eV) of the delayed fluorescence material is within the herein range, up-conversion from the triplet state to the singlet state of the delayed fluorescence materials may effectively occur, and thus, the light-emitting device 10 may have improved luminescence efficiency.

In one or more embodiments, the delayed fluorescence material may include: i) a material including at least one electron donor (for example, a π electron-rich C₃-C₆₀ cyclic group such as a carbazole group) and at least one electron acceptor (for example, a sulfoxide group, a cyano group, a π electron-deficient nitrogen-containing C₁-C₆₀ cyclic group, and/or the like), ii) a material including a C₈-C₆₀ polycyclic group including at least two cyclic groups that are condensed with each other while sharing boron (B).

Examples of the delayed fluorescence material may include at least one of (e.g., one or more selected from among) Compounds DF1 to DF14:

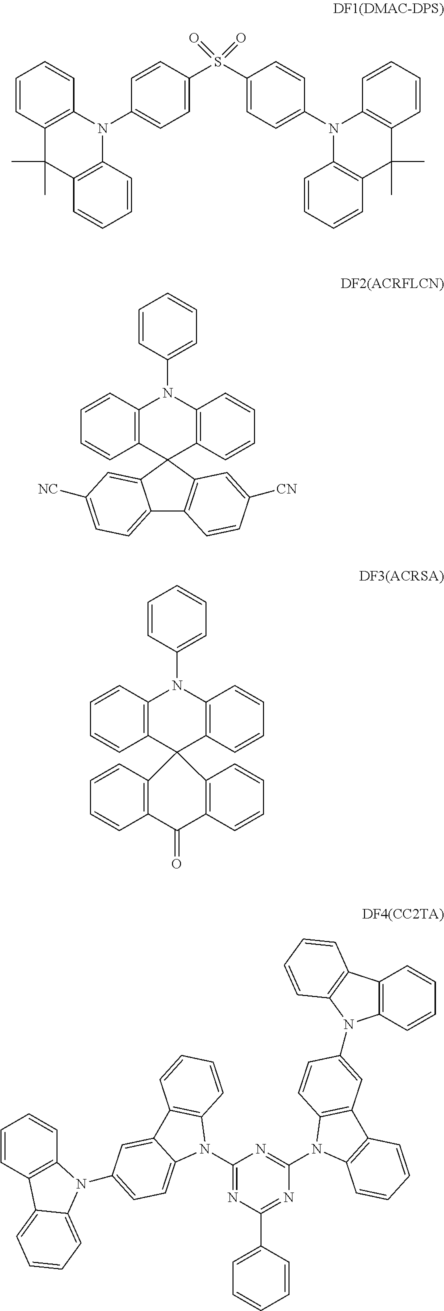

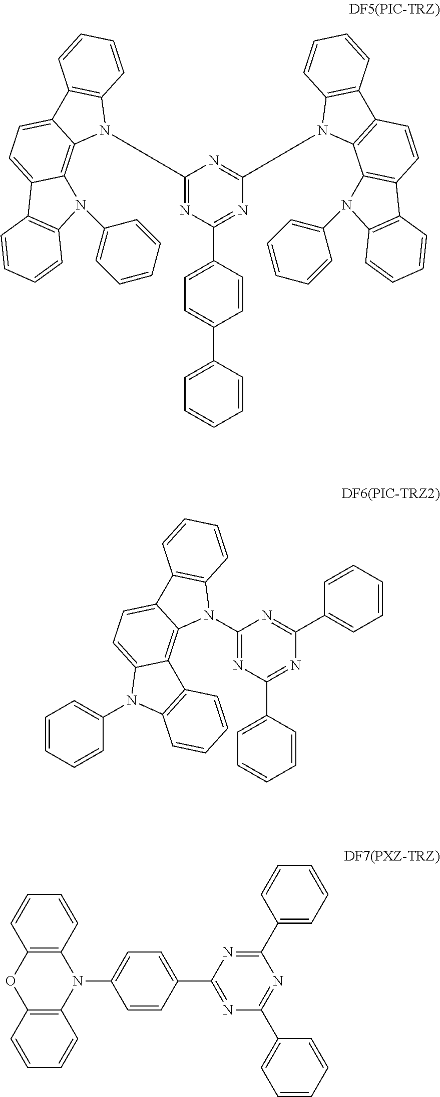 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

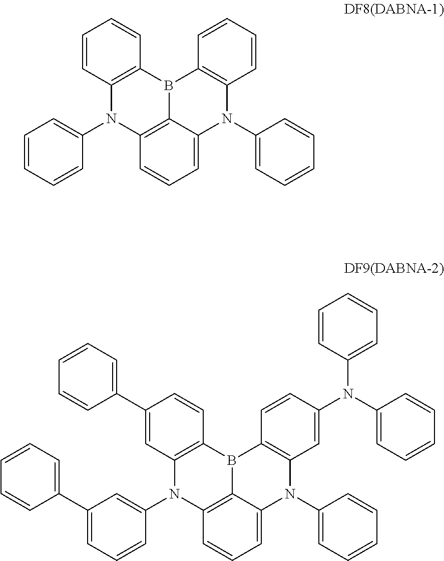 [SMILES: c1ccc(N2c3ccccc3B3c4ccccc4N(c4ccccc4)c4cccc2c43)cc1 ; c1ccc(-c2cccc(N3c4cc(-c5ccccc5)ccc4B4c5ccc(N(c6ccccc6)c6ccccc6)cc5N(c5ccccc5)c5cccc3c54)c2)cc1]

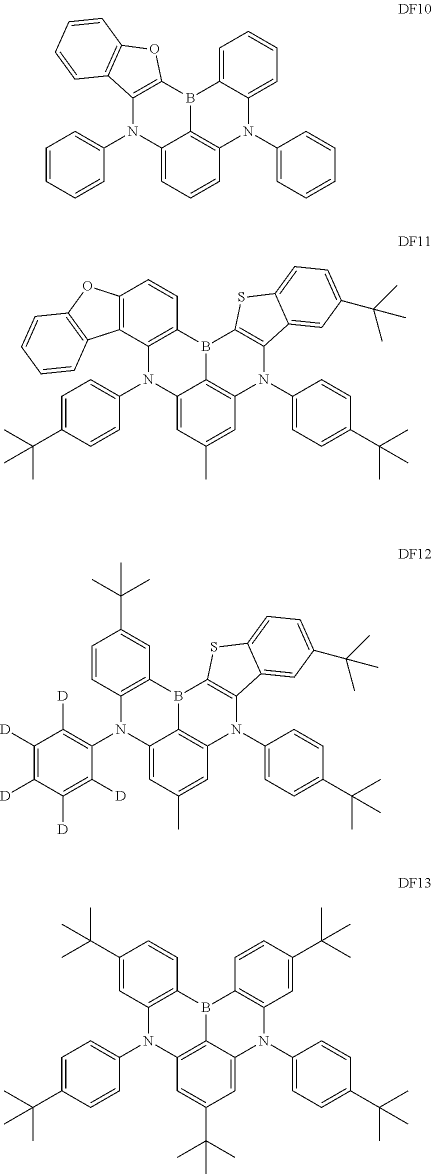

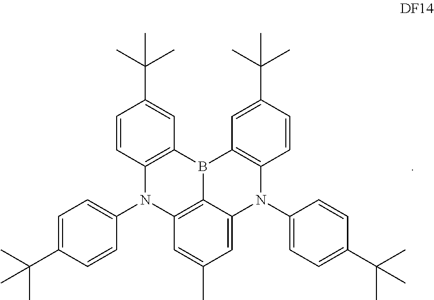 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer 130 may include a quantum dot.

The term “quantum dot” as used herein refers to a crystal of a semiconductor compound. Quantum dots may be to emit light of one or more suitable emission wavelengths according to the size of the crystal. Quantum dots may also emit light of one or more suitable emission wavelengths by adjusting the ratio of elements constituting the quantum dots.

The diameter of the quantum dots may be, for example, about 1 nanometer (nm) to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing quantum dot particle crystals. When the crystal grows, the organic solvent naturally acts as a dispersant coordinated on the surface of the quantum dot crystal and controls the growth of the crystal so that the growth of quantum dot particles may be controlled or selected through a process which costs lower, and is easier than vapor deposition methods, such as metal organic chemical vapor deposition (MOCVD) or molecular beam epitaxy (MBE).

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, MgS, and/or the like; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and/or the like; a quaternary compound, such as CdZnSeS, CdZnSeTe, CdZnSTe, CdHgSeS, CdHgSeTe, CdHgSTe, HgZnSeS, HgZnSeTe, HgZnSTe, and/or the like; or any combination thereof.

Examples of the Group III-V semiconductor compound may include: a binary compound, such as GaN, GaP, GaAs, GaSb, AlN, AlP, AlAs, AlSb, InN, InP, InAs, InSb, and/or the like; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, InPSb, and/or the like; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and/or the like; or any combination thereof. In one or more embodiments, the Group Ill-V semiconductor compound may further include a Group II element. Examples of the Group III-V semiconductor compound further including the Group II element may include InZnP, InGaZnP, InAlZnP, and/or the like.

Examples of the Group III-VI semiconductor compound may include: a binary compound, such as GaS, Ga₂S₃, GaSe, Ga₂Se₃, GaTe, InS, InSe, In₂S₃, In₂Se₃, InTe, and/or the like; a ternary compound, such as InGaS₃, InGaSe₃, and/or the like; or any combination thereof.

Examples of the Group I-III-VI semiconductor compound may include: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, and/or the like; a quaternary compound, such as AgInGaS₂, AgInGaSe₂, and/or the like; or any combination thereof.

Examples of the Group IV-VI semiconductor compound may include: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, SnPbTe, and/or the like; a quaternary compound, such as SnPbSSe, SnPbSeTe, SnPbSTe, and/or the like; or any combination thereof.

Examples of the Group IV element or compound may include: a single element, such as Si, Ge, and/or the like; a binary compound, such as SiC, SiGe, and/or the like; or any combination thereof.

Each element included in a multi-element compound, such as the binary compound, the ternary compound, and the quaternary compound, may be present at a substantially uniform concentration or substantially non-uniform concentration in a particle. For example, the preceding formulae refer to types (kinds) of elements included in the compound, wherein the element ratios in the compound may vary. For example, AgInGaS₂ may refer to AgInₓGa₁₋ₓS₂ (wherein x is a real number between 0 and 1).

In one or more embodiments, the quantum dot may have a single structure in which the concentration of each element in the quantum dot is substantially uniform, or may have a core-shell dual structure. In one or more embodiments, a material included in the core and a material included in the shell may be different from each other.

The shell of the quantum dot may act as a protective layer which prevents chemical denaturation of the core to maintain semiconductor characteristics, and/or as a charging layer which imparts electrophoretic characteristics to the quantum dot. The shell may be a single layer or a multilayer. The interface between the core and the shell may have a concentration gradient in which the concentration of an element existing in the shell decreases toward the center of the core.

Examples of the shell of the quantum dot may be an oxide of a metal or non-metal, a semiconductor compound, and/or a (e.g., any suitable) combination thereof. Examples of the oxide of a metal or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, NiO, and/or the like; a ternary compound, such as MgAl₂O₄, CoFe₂O₄, NiFe₂O₄, CoMn₂O₄, and/or the like; or any combination thereof. Examples of the semiconductor compound may include: a Group Ill-VI semiconductor compound; a Group II-VI semiconductor compound; a Group Ill-V semiconductor compound; a Group 1-Ill-VI semiconductor compound; a Group IV-VI semiconductor compound; or any combination thereof, as described herein. For example, the semiconductor compound may include CdS, CdSe, CdTe, ZnS, ZnSe, ZnTe, ZnSeS, ZnTeS, GaAs, GaP, GaS, GaSe, AgGaS, AgGaS₂, GaSb, HgS, HgSe, HgTe, InAs, InP, InGaP, InSb, AlAs, AlP, AlSb, or any combination thereof.

Each element included in a multi-element compound, such as the binary compound and the ternary compound, may be present at a substantially uniform concentration or substantially non-uniform concentration in a particle. For example, the preceding formulae refer to types (kinds) of elements included in the compound, wherein the element ratios in the compound may vary.

The quantum dot may have a full width at half maximum (FWHM) of the emission wavelength spectrum of less than or equal to about 45 nm, less than or equal to about 40 nm, or for example, less than or equal to about 30 nm. When the FWHM of the quantum dot is within these ranges, the quantum dot may have improved color purity or improved color reproducibility. In some embodiments, because light emitted through the quantum dot is emitted in all directions, the wide viewing angle may be improved.

In some embodiments, the quantum dot may be in the form of spherical, pyramidal, multi-arm, or cubic nanoparticles, nanotubes, nanowires, nanofibers, or nanoplate particles.

Because the energy band gap may be controlled or selected by adjusting the size of the quantum dots or the ratio of elements in the quantum dot compound, light of one or more suitable wavelengths may be obtained from the quantum dot-containing emission layer. Therefore, by using the aforementioned quantum dots (using quantum dots of different sizes or having different element ratios in the quantum dot compound), a light-emitting device emitting light of one or more suitable wavelengths may be implemented. For example, the control of the size of the quantum dots or the ratio of elements in the quantum dot compound may be selected to emit red light, green light, and/or blue light. In some embodiments, the size of the quantum dots may be configured to emit white light by combination of light of one or more suitable colors.

### Electron Transport Region 140

The electron transport region 140 may have i) a single-layer structure including (e.g., consisting of) a single layer including a single material, ii) a single-layer structure including (e.g., consisting of) a single layer including a plurality of materials that are different from each other, or iii) a multilayer structure including (e.g., consisting of) a plurality of layers including a plurality of materials that are different from each other.

The electron transport region 140 may include a buffer layer, a hole blocking layer, an electron control layer, an electron transport layer, an electron injection layer, or any combination thereof.

In one or more embodiments, the electron transport region 140 may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein, for each structure, constituting layers are sequentially stacked from the emission layer 130.

The electron transport region 140 (for example, the buffer layer, the hole blocking layer, the electron control layer, or the electron transport layer in the electron transport region) may include a metal-free compound including at least one π electron-deficient nitrogen-containing C₁-C₆₀ cyclic group.

For example, the electron transport region 140 may include a compound represented by Formula 601.

[Ar₆₀₁]ₓₑ₁₁-[(L₆₀₁)ₓₑ₁-R₆₀₁]ₓₑ₂₁ Formula 601

In Formula 601,
Ar₆₀₁ and L₆₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ are each as described in connection with Q₁,
xe21 may be 1, 2, 3, 4, or 5, and
at least one of (e.g., one or more selected from among) Ar₆₀₁, L₆₀₁, and/or R₆₀₁ may each independently be a π electron-deficient nitrogen-containing C₁-C₆₀ cyclic group that is unsubstituted or substituted with at least one R₁₀ₐ.

In one or more embodiments, if (e.g., when) xe11 in Formula 601 is 2 or more, two or more of Ar₆₀₁ may be linked together via a single bond.

In one or more embodiments, Ar₆₀₁ in Formula 601 may be an anthracene group that is unsubstituted or substituted with at least one R₁₀ₐ.

In one or more embodiments, the electron transport region 140 may include a compound represented by Formula 601-1:

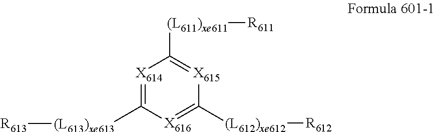

wherein, in Formula 601-1,
X₆₁₄ may be N or C(R₆₁₄), X₆₁₅ may be N or C(R₆₁₅), X₆₁₆ may be N or C(R₆₁₆), and at least one of (e.g., one or more selected from among) X₆₁₄ to X₆₁₆ may be N,
L₆₁₁ to L₆₁₃ are each as described in connection with L₆₀₁,
xe611 to xe613 are each as described in connection with xe1,
R₆₁₁ to R₆₁₃ are each as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In one or more embodiments, xe1 and xe611 to xe613 in Formulae 601 and 601-1 may each independently be 0, 1, or 2.

The electron transport region 140 may include at least one of (e.g., one or more selected from among) Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

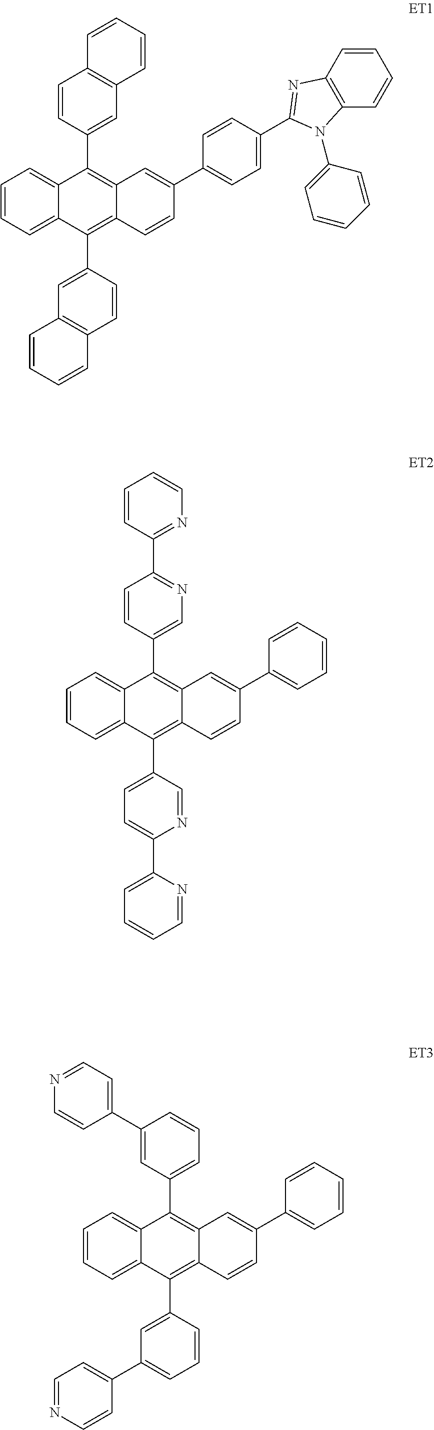 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

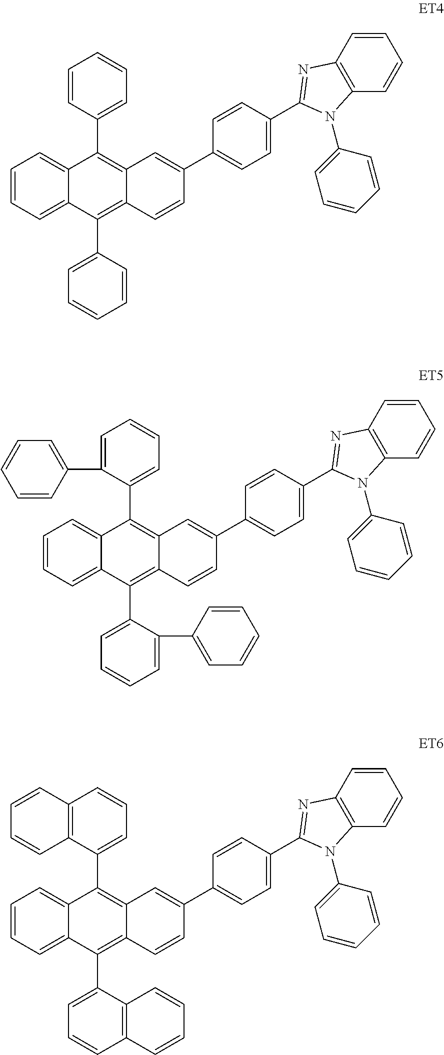 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

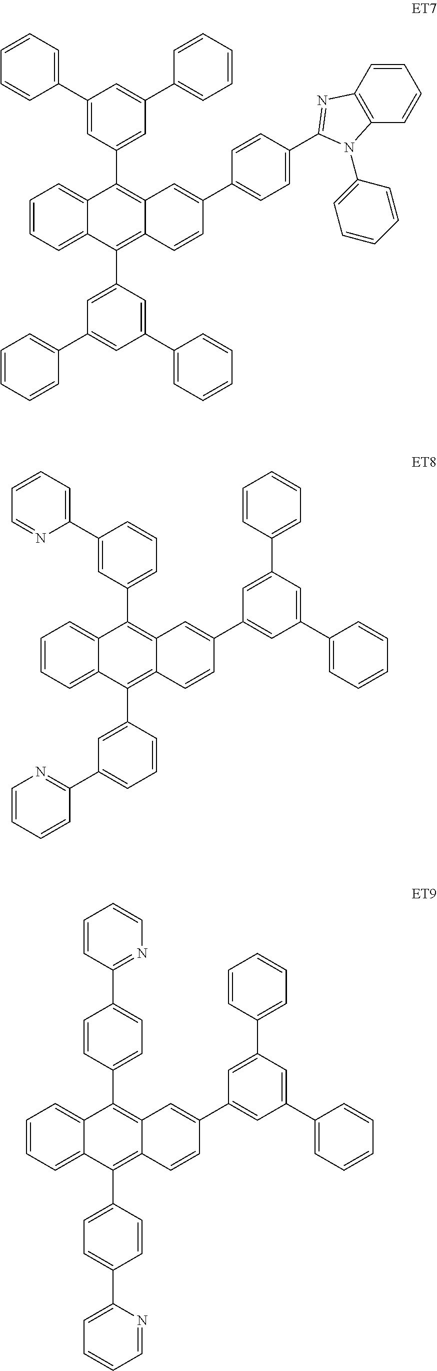 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

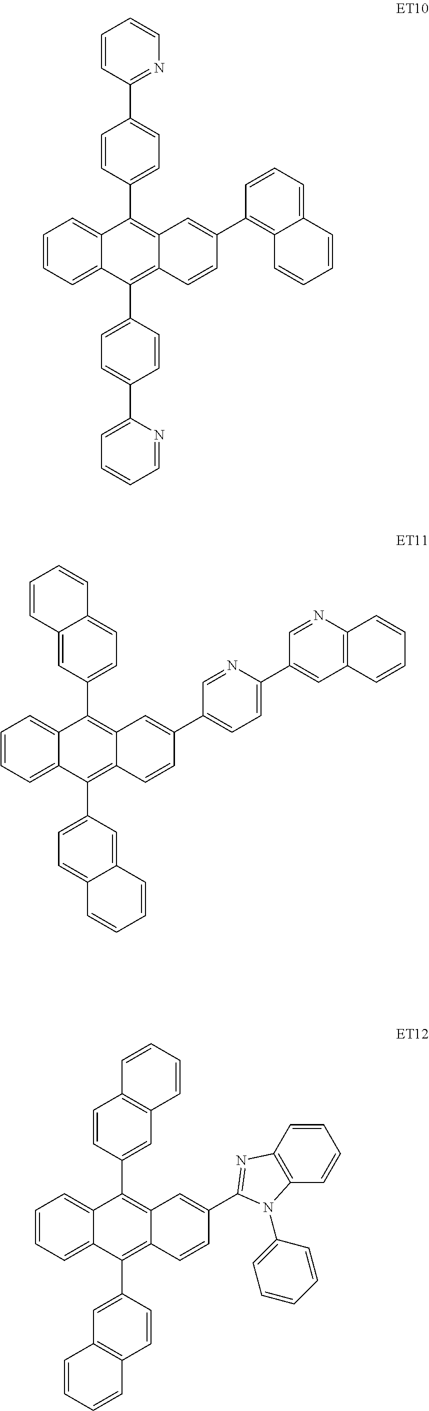 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

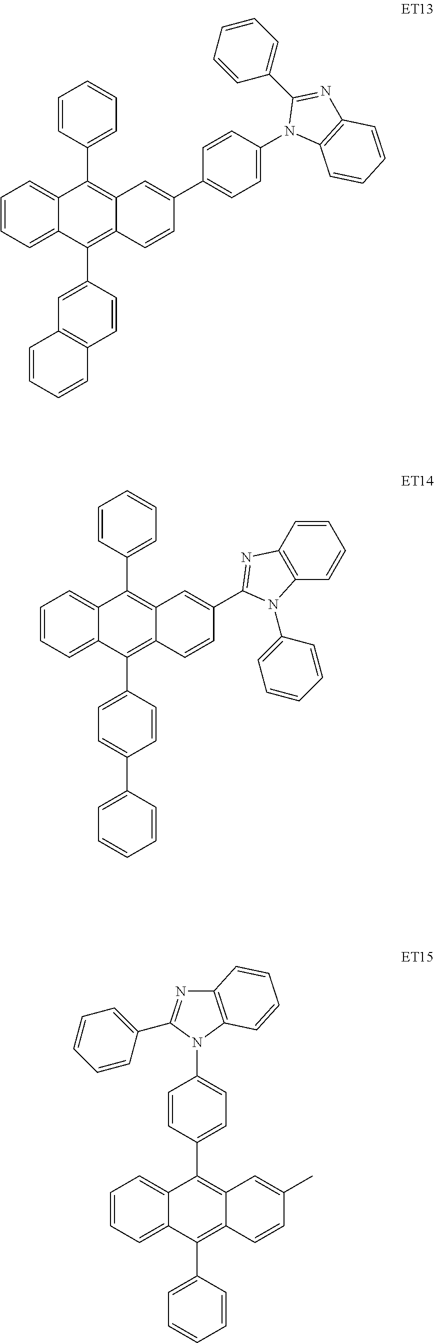 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

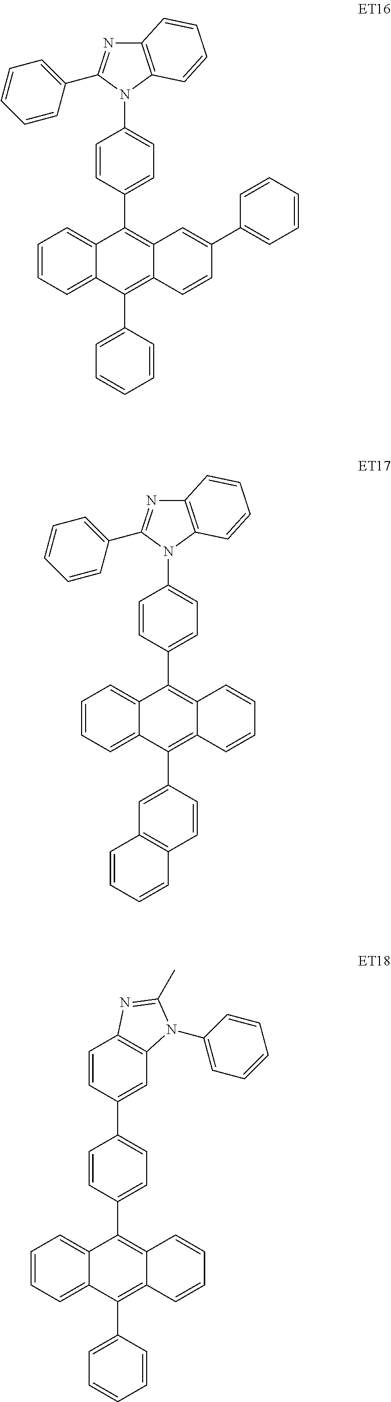 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

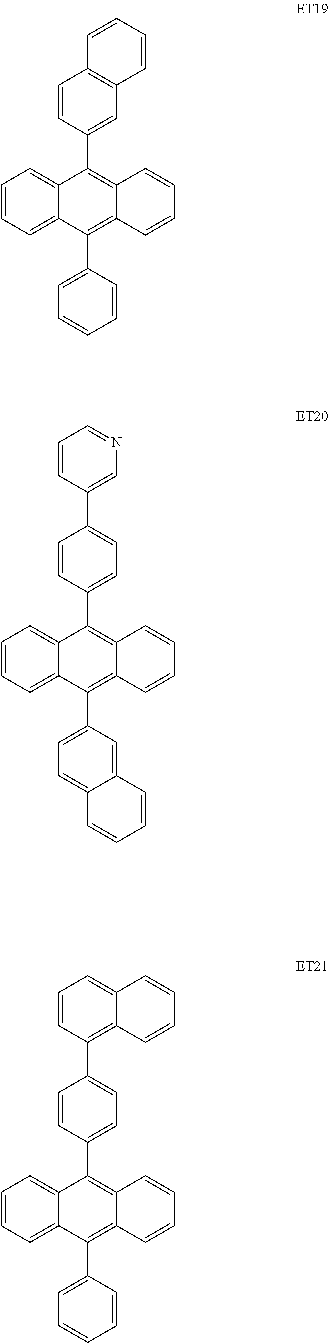

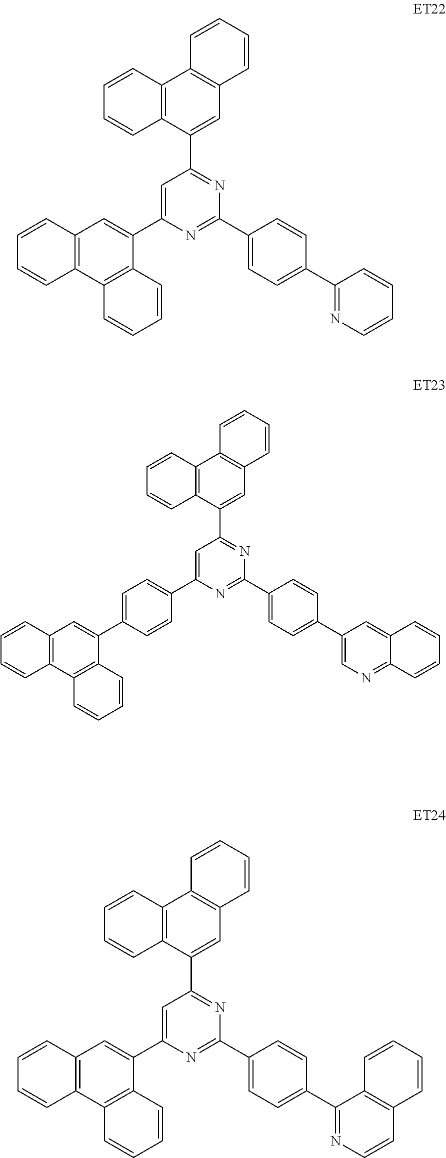 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

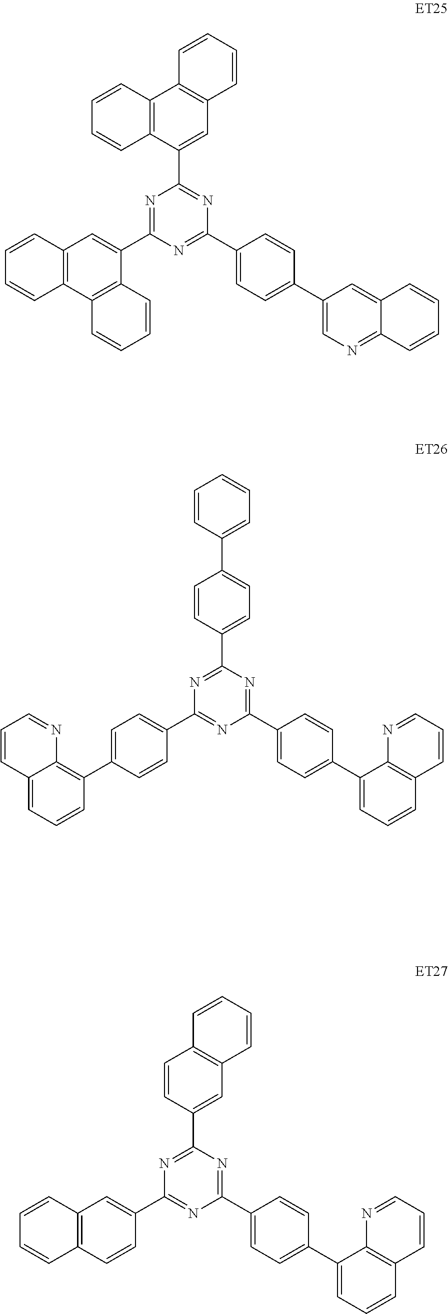 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

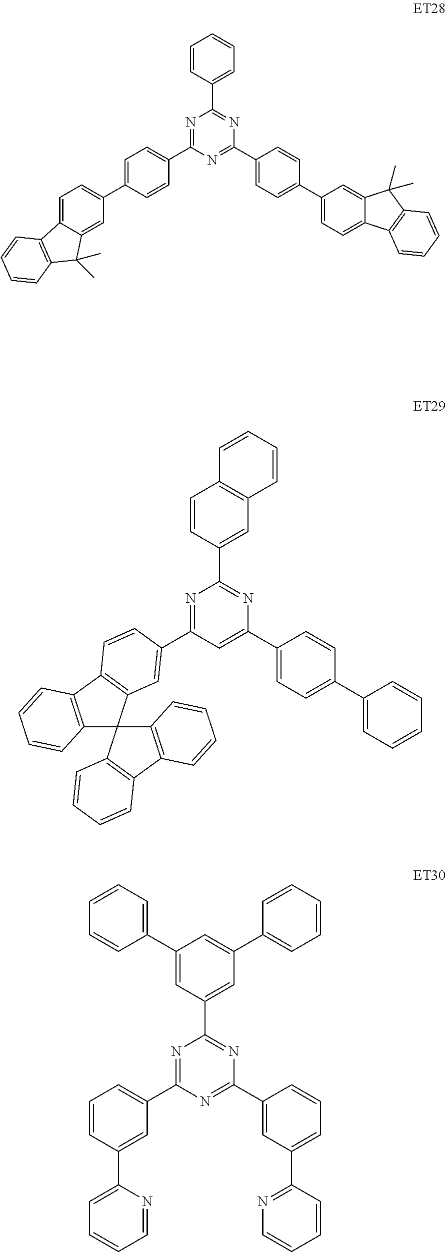 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

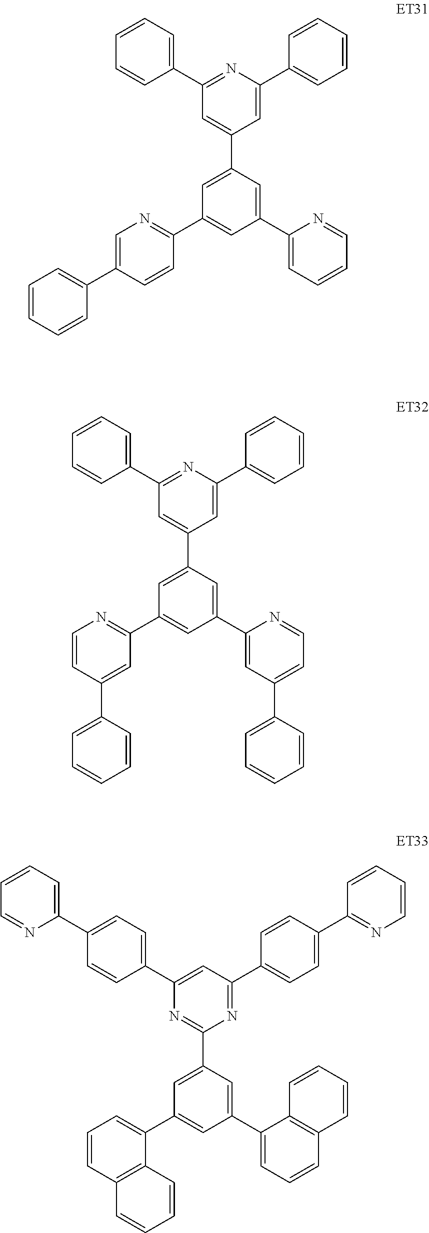 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

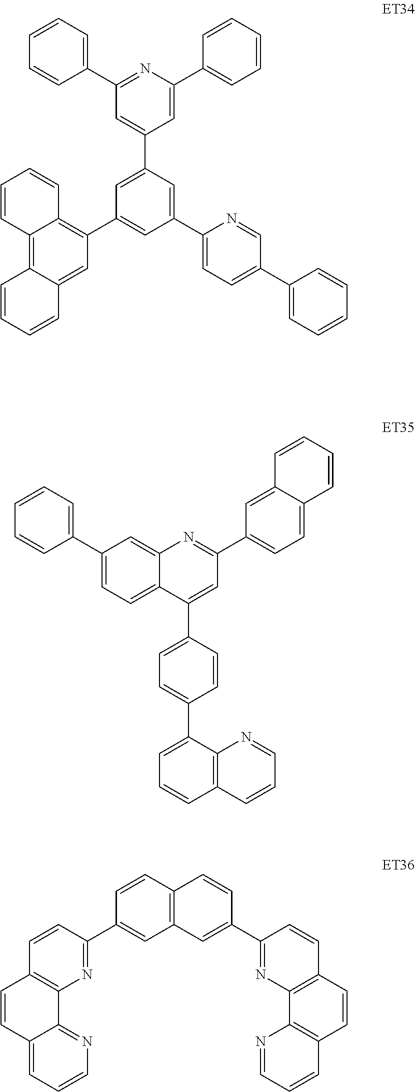 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

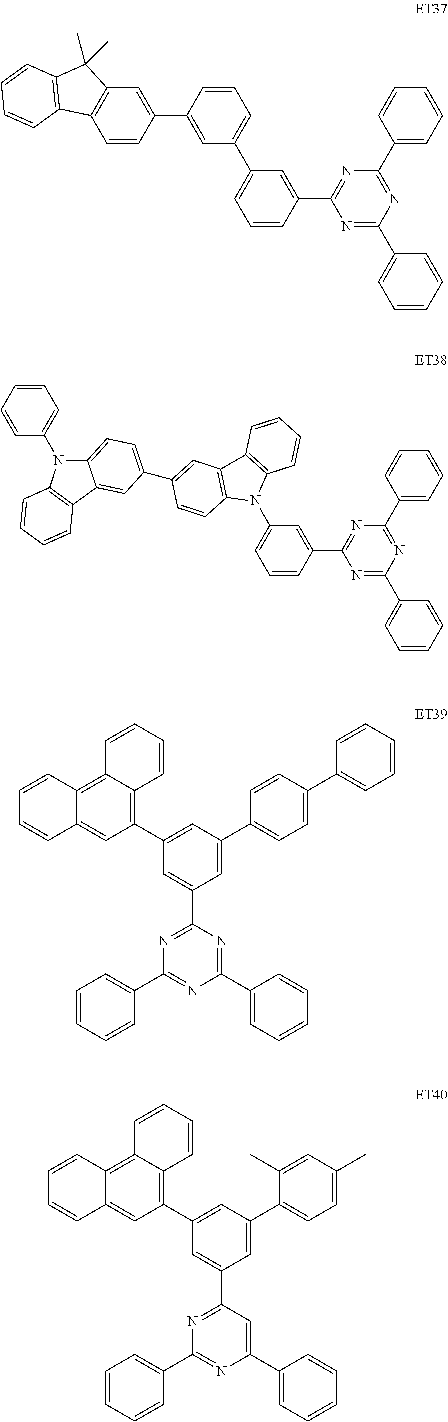 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

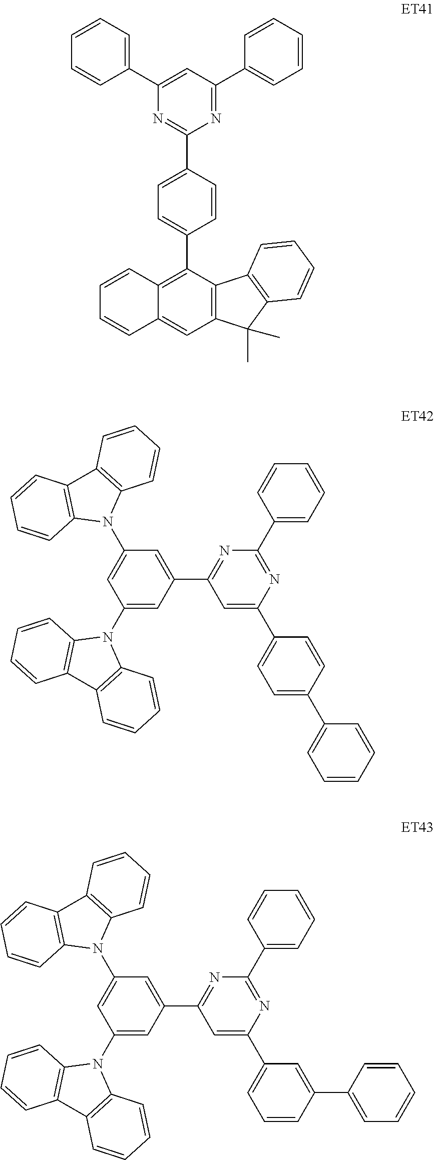 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

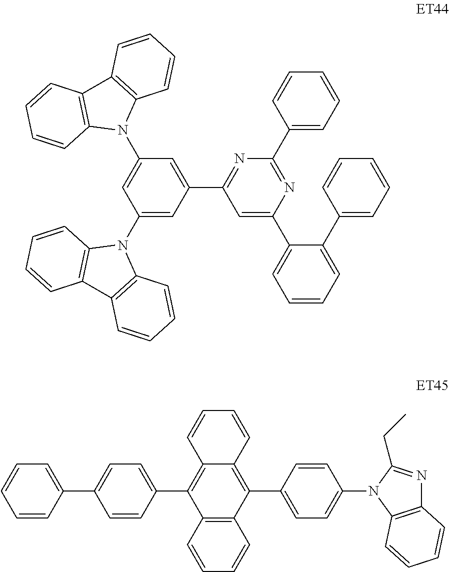 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

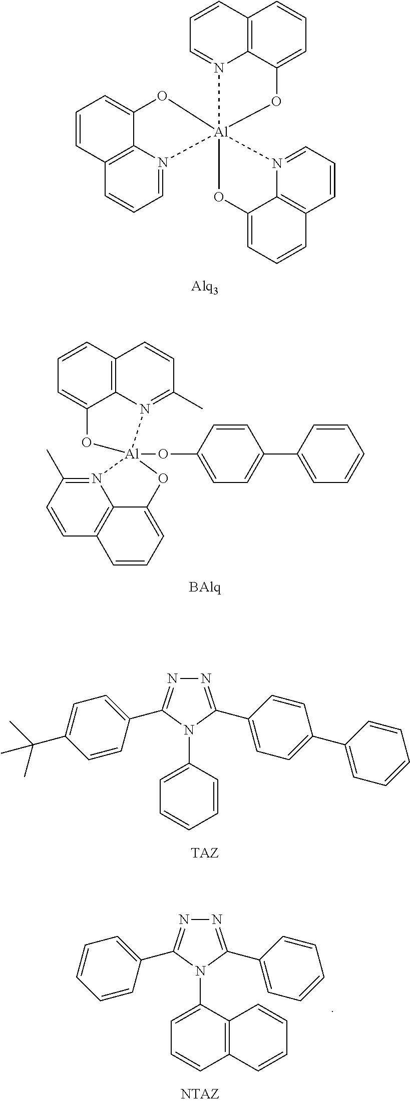

The thickness of the electron transport region 140 may be about 100 Å to about 5,000 Å, for example, about 160 Å to about 4,000 Å. When the electron transport region 140 includes a buffer layer, a hole blocking layer, an electron control layer, an electron transport layer, or any combination thereof, the thickness of the buffer layer, the hole blocking layer, or the electron control layer may each independently be about 20 Å to about 1,000 Å, for example, about 30 Å to about 300 Å, and the thickness of the electron transport layer may be about 100 Å to about 1,000 Å, for example, about 150 Å to about 500 Å. When the thickness of the buffer layer, the hole blocking layer, the electron control layer, the electron transport layer, and/or the electron transport region are within these ranges, satisfactory electron transporting characteristics may be obtained without a substantial increase in driving voltage.

The electron transport region 140 (for example, an electron transport layer in the electron transport region) may further include, in addition to the aforementioned materials, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In one or more embodiments, the metal-containing material may include a Li complex. The Li complex may include, for example, Compound ET-D1 (LiQ) and/or ET-D2:

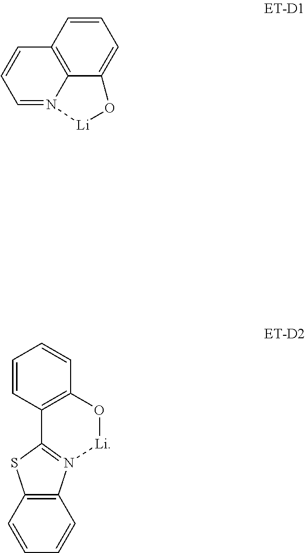

The electron transport region 140 may include an electron injection layer that facilitates injection of electrons from the second electrode 150. The electron injection layer may directly contact the second electrode 150.

The electron injection layer may have: i) a single-layered structure including (e.g., consisting of) a single layer including (e.g., consisting of) a single material, ii) a single-layered structure including (e.g., consisting of) a single layer including a plurality of materials that are different from each other, or iii) a multilayer structure including a plurality of layers including a plurality of materials that are different from each other.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, iodides, and/or the like), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaₓSr₁₋ₓO (x is a real number satisfying 0<x<1), or BaₓCa₁₋ₓO (x is a real number satisfying 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of (e.g., selected from among) ions of the alkali metal, the alkaline earth metal, and the rare earth metal and ii) a ligand bonded to the metal ion(s), (e.g., the selected metal ion(s)), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described herein. In one or more embodiments, the electron injection layer may further include an organic material (for example, a compound represented by Formula 601).

In one or more embodiments, the electron injection layer may include (e.g., consist of) i) an alkali metal-containing compound (for example, alkali metal halide), ii) a) an alkali metal-containing compound (for example, alkali metal halide); and b) an alkali metal, an alkaline earth metal, a rare earth metal, or any combination thereof. In one or more embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly (e.g., substantially uniformly) or non-uniformly (e.g., substantially non-uniformly) dispersed in a matrix including the organic material.

The thickness of the electron injection layer may be about 1 Å to about 100 Å, and, for example, about 3 Å to about 90 Å. When the thickness of the electron injection layer is within the range as described herein, satisfactory electron injection characteristics may be obtained without a substantial increase in driving voltage.

### Second Electrode 150

The second electrode 150 may be arranged above the electron transport region 140. The second electrode 150 may be a cathode, which is an electron injection electrode, and as a material for forming the second electrode 150, a metal, an alloy, an electrically conductive compound, or any combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layer structure or a multilayer structure including a plurality of layers.

### Capping Layer

The first capping layer may be arranged outside (and e.g., on) the first electrode 110, and/or the second capping layer may be arranged outside (and e.g., on) the second electrode 150. In one or more embodiments, the light-emitting device 10 may have a structure in which the first capping layer, the first electrode 110, the interlayer, and the second electrode 150 are sequentially stacked in the stated order, a structure in which the first electrode 110, the interlayer, the second electrode 150, and the second capping layer are sequentially stacked in the stated order, or a structure in which the first capping layer, the first electrode 110, the interlayer, the second electrode 150, and the second capping layer are sequentially stacked in the stated order.

Light generated in the emission layer 130 of the light-emitting device 10 may pass through the first electrode 110, which is a semi-transmissive electrode or a transmissive electrode, and through the first capping layer to the outside. Light generated in the emission layer 130 of the light-emitting device 10 may pass through the second electrode 150, which is a semi-transmissive electrode or a transmissive electrode, and through the second capping layer to the outside.

The first capping layer and the second capping layer may increase external emission efficiency according to the aspect of constructive interference. Accordingly, the light extraction efficiency of the light-emitting device 10 is increased, such that the luminescence efficiency of the light-emitting device 10 may be increased.

Each of the first capping layer and the second capping layer may include a material having a refractive index of about 1.2 or higher (at 460 nm).

The first capping layer and the second capping layer may each independently be an organic capping layer including an organic material, an inorganic capping layer including an inorganic material, or an organic-inorganic composite capping layer including an organic material and an inorganic material.

At least one of (e.g., selected from among) the first capping layer and/or the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In one or more embodiments, at least one of (e.g., selected from among) the first capping layer and/or the second capping layer may each independently include an amine group-containing compound.

In one or more embodiments, at least one of (e.g., selected from among) the first capping layer and/or the second capping layer may each independently include a compound represented by Formula 201, a compound represented by Formula 202, or any combination thereof.

In one or more embodiments, at least one of (e.g., selected from among) the first capping layer and/or the second capping layer may each independently include at least one of (e.g., one or more selected from among) Compounds HT28 to HT33, at least one of (e.g., one or more selected from among) Compounds CP1 to CP6, β-NPB, or any combination thereof:

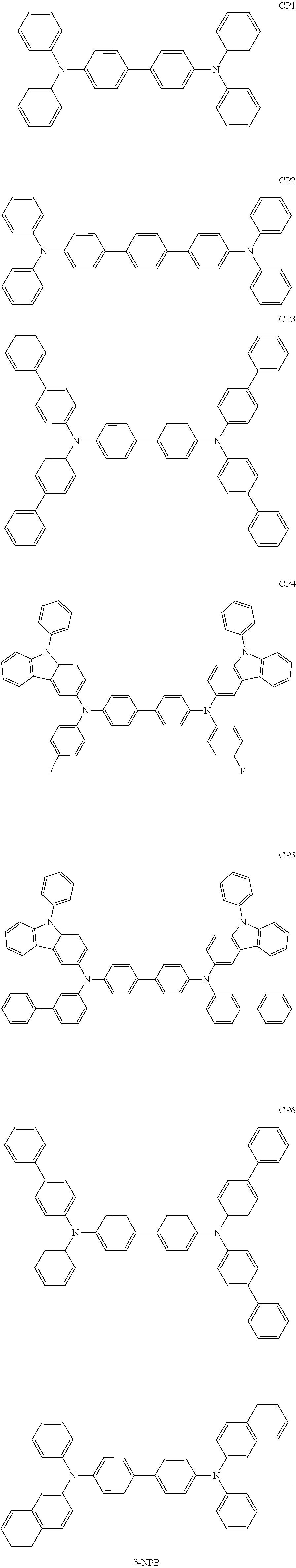

### Film

The electronic apparatus may further include a film. The film may be, for example, an optical member (or a light control component) (for example, a color filter, a color conversion member, a capping layer, a light extraction efficiency enhancement layer, a selective light absorbing layer, a polarizing layer, a quantum dot-containing layer, or like), a light-blocking member (for example, a light reflective layer, a light absorbing layer, and/or the like), a protective member (for example, an insulating layer, a dielectric layer, and/or the like).

### Electronic Apparatus

The light-emitting device 10 may be included in one or more suitable electronic apparatuses. In one or more embodiments, an electronic apparatus including the light-emitting device 10 may be a display apparatus or an authentication apparatus.

The electronic apparatus (for example, a display apparatus) may further include, in addition to the light-emitting device 10, i) a color filter, ii) a color conversion layer, or iii) a color filter and a color conversion layer. The color filter and/or the color conversion layer may be arranged in at least one direction in which light emitted from the light-emitting device 10 travels. For example, the light emitted from the light-emitting device 10 may be blue light or white light. A more detailed description of the light-emitting device 10 is provided herein. In one or more embodiments, the color conversion layer may include quantum dots. The quantum dots may be, for example, the quantum dots as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths. In one or more embodiments, the first color light may be red light, the second color light may be green light, and the third color light may be blue light. In one or more embodiments, the plurality of color filter areas (or the plurality of color conversion areas) may include quantum dots. For example, the first area may include red quantum dots, the second area may include green quantum dots, and the third area may not include (e.g., may exclude any) quantum dot. A detailed description of the quantum dots is provided herein. The first area, the second area, and/or the third area may each further include a scatterer.

In one or more embodiments, the light-emitting device 10 may be to emit first light, the first area may be to absorb the first light to emit first-1 color light, the second area may be to absorb the first light to emit second-1 color light, and the third area may be to absorb the first light to emit third-1 color light. In this case, the first-1 color light, the second-1 color light, and the third-1 color light may have different maximum emission wavelengths. For example, the first light may be blue light, the first-1 color light may be red light, the second-1 color light may be green light, and the third-1 color light may be blue light.

The electronic apparatus may further include a thin-film transistor, in addition to the light-emitting device 10. The thin-film transistor may include a source electrode, a drain electrode, and an activation layer, wherein any one of the source electrode or the drain electrode may be electrically connected to any one of the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

Various functional layers may be additionally arranged on the sealing portion, in addition to the color filter and/or the color conversion layer, according to the use of the electronic apparatus. Examples of the functional layers may include a touch screen layer and a polarizing layer. The touch screen layer may be a pressure-sensitive touch screen layer, a capacitive touch screen layer, or an infrared touch screen layer. The authentication apparatus may be, for example, a biometric authentication apparatus that authenticates an individual by using biometric information of a living body (for example, fingertips, pupils, and/or the like).

The authentication apparatus may further include, in addition to the light-emitting device as described herein, a biometric information collector.

The electronic apparatus may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Equipment

The light-emitting device 10 may be included in one or more suitable electronic equipment.

For example, electronic equipment including the light-emitting device 10 may be at least one of (e.g., one or more selected from among) a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a 3D display, a virtual or augmented reality display, a vehicle, a video wall including multiple displays tiled together, a theater or stadium screen, a phototherapy device, a sign, and/or a (e.g., any suitable) combination thereof.

Because the light-emitting device 10 has improved luminescence efficiency, improve lifespan, and/or the like, the electronic equipment including the light-emitting device 10 may have high luminance, high resolution, and low power consumption.

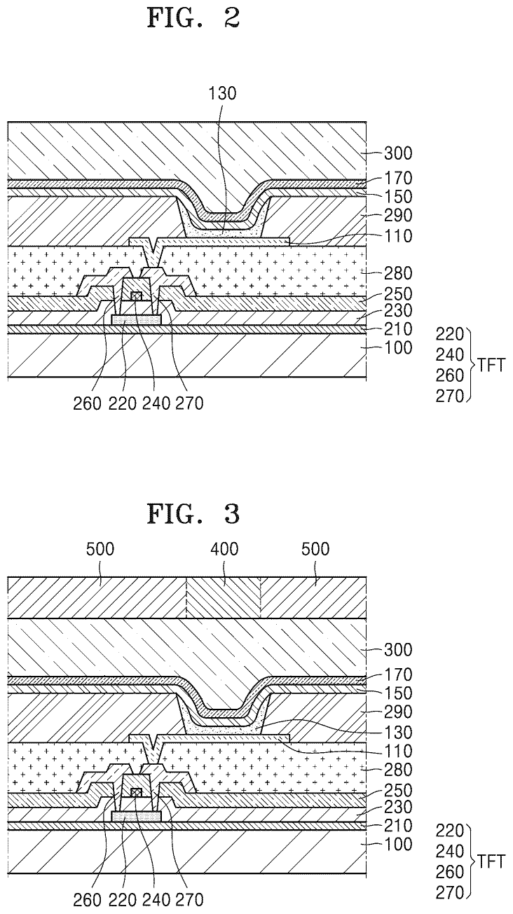

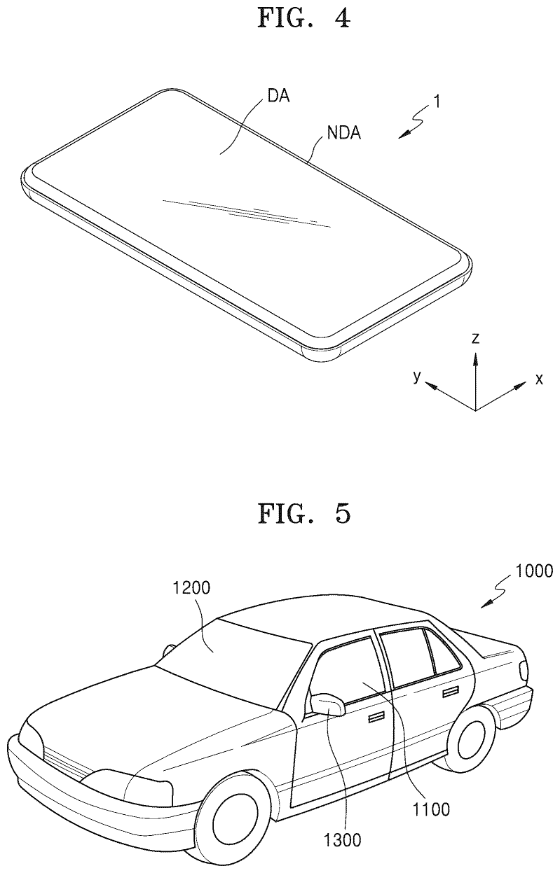

### Description of FIGS. 2 and 3

FIG. 2 is a cross-sectional view of an electronic apparatus according to one or more embodiments.

The electronic apparatus in FIG. 2 may include a substrate 100, a thin-film transistor (TFT), a light-emitting device, and an encapsulation portion 300.

The substrate 100 may be a flexible substrate, a glass substrate, or a metal substrate. A buffer layer 210 may be arranged on the substrate 100. The buffer layer 210 may prevent or reduce penetration of impurities through the substrate 100 and may provide a flat surface on the substrate 100.

The TFT may be arranged on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating the activation layer 220 from the gate electrode 240 may be arranged above the activation layer 220, and the gate electrode 240 may be arranged above the gate insulating film 230.

An interlayer insulating film 250 may be arranged above the gate electrode 240. The interlayer insulating film 250 may be located between the gate electrode 240 and the source electrode 260 to insulate the gate electrode 240 from the source electrode 260 and between the gate electrode 240 and the drain electrode 270 to insulate the gate electrode 240 from the drain electrode 270.

The source electrode 260 and the drain electrode 270 may be arranged on the interlayer insulating film 250. The interlayer insulating film 250 and the gate insulating film 230 may be formed to expose the source region and the drain region of the activation layer 220, and the source electrode 260 and the drain electrode 270 may be arranged in contact with the exposed portions of the source region and the drain region of the activation layer 220.

The TFT may be electrically connected to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device may include the first electrode 110, the interlayer, and the second electrode 150.

The first electrode 110 may be arranged on the passivation layer 280. The passivation layer 280 may be arranged to expose a portion of the drain electrode 270, not fully covering the drain electrode 270, and the first electrode 110 may be arranged to be connected to the exposed portion of the drain electrode 270.

A pixel-defining film 290 including an insulating material may be arranged on the first electrode 110. The pixel-defining film 290 may expose a certain region of the first electrode 110, and the interlayer may be formed in the exposed region of the first electrode 110. The pixel-defining film 290 may be a polyimide-based organic film or a polyacrylic organic film. In one or more embodiments, at least some layers of the interlayer may extend to the upper portion of the pixel-defining film 290 and may be arranged in the form of a common layer.

The second electrode 150 may be arranged on the interlayer, and a capping layer 170 may be further formed on the second electrode 150. The capping layer 170 may be formed to cover the second electrode 150.

The encapsulation portion 300 may be arranged on the capping layer 170. The encapsulation portion 300 may be arranged on a light-emitting device to protect the light-emitting device from moisture or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; and/or a (e.g., any suitable) combination of the inorganic film and the organic film.

FIG. 3 is a cross-sectional view of an electronic apparatus according to one or more embodiments.

The electronic apparatus in FIG. 3 is the same as the electronic apparatus in FIG. 2, except that a light-shielding pattern 500 and a functional region 400 are additionally arranged on the encapsulation portion 300. The functional region 400 may be i) a color filter area, ii) a color conversion area, or iii) a combination of the color filter area and the color conversion area. In one or more embodiments, the light-emitting device included in the electronic apparatus in FIG. 3 may be a tandem light-emitting device.

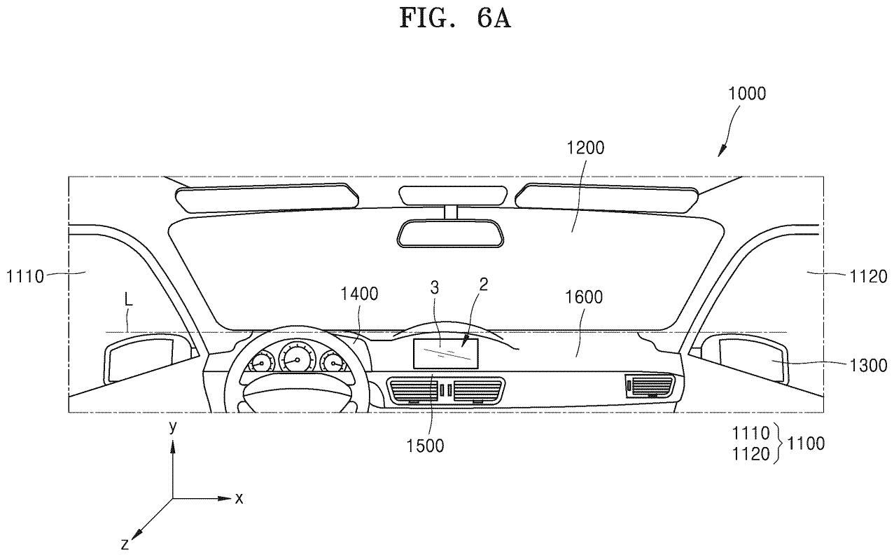

### Description of FIG. 4

FIG. 4 is a schematic perspective view of electronic equipment 1 including a light-emitting device, according to one or more embodiments. The electronic equipment 1 may be, as an apparatus that displays a moving image or a still image, a portable electronic device, such as a mobile phone, a smartphone, a tablet personal computer (PC), a mobile communication terminal, an electronic notebook, an electronic book, a portable multimedia player (PMP), a navigation, or a ultra-mobile PC (UMPC), as well as one or more suitable products, such as a television, a laptop, a monitor, a billboard, or an Internet of things (IOT). The electronic equipment 1 may be such a product as described herein or a part thereof. In some embodiments, the electronic equipment 1 may be a wearable device, such as a smart watch, a watch phone, a glasses-type or kind display, or a head mounted display (HMD), or a part of the wearable device. However, embodiments are not limited thereto. In one or more embodiments, the electronic equipment 1 may be a dashboard of a vehicle, a center information display (CID) arranged on a center fascia or dashboard of a vehicle, a room mirror display instead of a side-view mirror of a vehicle, an entertainment for the back seat of a vehicle, or a display arranged on the back of the front seat of a vehicle, a head up display (HUD) installed on the front of a vehicle or projected on a front window glass, or a computer generated hologram augmented reality head up display (CGH AR HUD). FIG. 4 illustrates a case in which the electronic equipment 1 is a smartphone for convenience of explanation.

The electronic equipment 1 may include a display area DA and a non-display area NDA outside the display area DA. The electronic equipment 1 may implement an image through an array of a plurality of pixels that are two-dimensionally arranged in the display area DA.

The non-display area NDA is an area that does not display an image, and may entirely be around (e.g., surround) the display area DA. On the non-display area NDA, a driver for providing electrical signals or power to display devices arranged on the display area DA may be arranged. On the non-display area NDA, a pad, which is an area to which an electronic element or a printed circuit board, may be electrically connected may be arranged.

In the electronic equipment 1, the length in an x-axis direction and the length in a y-axis direction may be different from each other. In one or more embodiments, as shown in FIG. 4, the length in the x-axis direction may be less than the length in the y-axis direction. In one or more embodiments, the length in the x-axis direction may be the same as the length in the y-axis direction. In one or more embodiments, the length in the x-axis direction may be greater than the length in the y-axis direction.

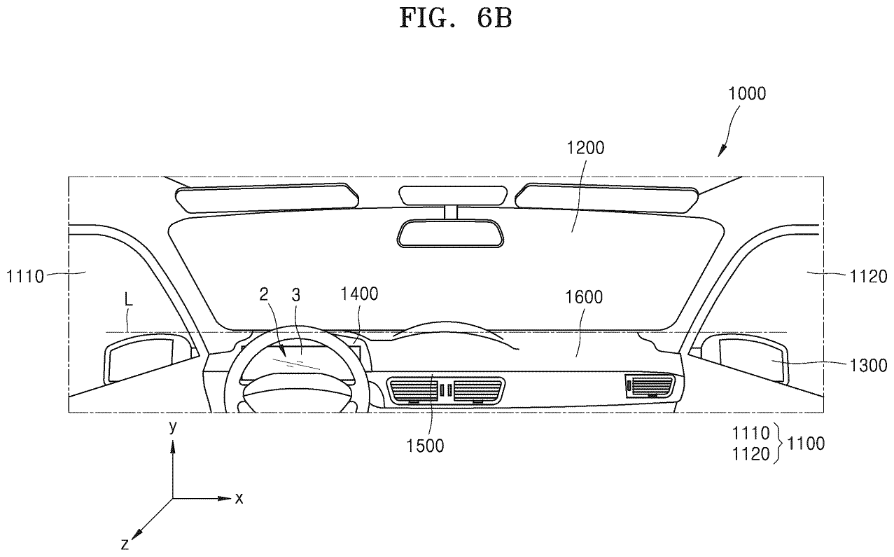

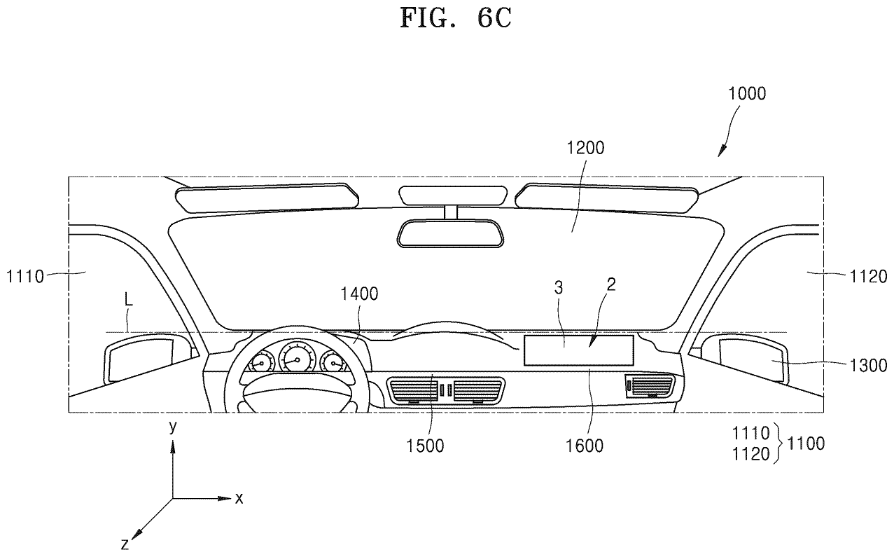

### Descriptions of FIGS. 5 and 6A to 6C

FIG. 5 is a schematic view of the exterior of a vehicle 1000 as electronic equipment including a light-emitting device, according to one or more embodiments. FIGS. 6A to 6C are schematic views each being of the interior of the vehicle 1000 according to one or more embodiments.

Referring to FIGS. 5 and 6A to 6C, the vehicle 1000 may refer to one or more suitable apparatuses for moving a subject to be transported, such as a human, an aspect, or an animal, from a departure point to a destination point. The vehicle 1000 may include a vehicle traveling on a road or track, a vessel moving over the sea or river, an airplane flying in the sky using the action of air, and/or the like.

The vehicle 1000 may travel on a road or a track. The vehicle 1000 may move in a certain direction according to rotation of at least one wheel. In one or more embodiments, the vehicle 1000 may include a three-wheeled or four-wheeled vehicle, a construction machine, a two-wheeled vehicle, a prime mover device, a bicycle, and a train running on a track.

The vehicle 1000 may include a vehicle body having an interior and an exterior, and a chassis in which mechanical apparatuses necessary for driving are installed as other parts except for the vehicle body. The exterior of the vehicle body may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a pillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced and/or apart (e.g., spaced apart or separated) from each other in an x direction or a −x direction. In one or more embodiments, the first side window glass 1110 and the second side window glass 1120 may be spaced and/or apart (e.g., spaced apart or separated) from each other in the x direction or the −x direction. For example, an imaginary straight line L connecting the side window glasses 1100 may extend in the x direction or the −x direction. In one or more embodiments, an imaginary straight line L connecting the first side window glass 1110 and the second side window glass 1120 to each other may extend in the x direction or the −x direction.

The front window glass 1200 may be installed in front of the vehicle 1000. The front window glass 1200 may be arranged between the side window glasses 1100 opposite to (e.g., facing) each other.

The side-view mirror 1300 may provide a rear view of the vehicle 1000. The side-view mirror 1300 may be installed on the exterior of the vehicle body. In one or more embodiments, a plurality of side-view mirrors 1300 may be provided. Any one of the plurality of side-view mirrors 1300 may be arranged outside the first side window glass 1110. The other one of the plurality of side-view mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, a tachograph, an automatic shift selector indicator lamp, a door open warning lamp, an engine oil warning lamp, and/or a low fuel warning light.

The center fascia 1500 may include a control panel on which a plurality of buttons for adjusting an audio device, an air conditioning device, and a heater of a seat are arranged. The center fascia 1500 may be arranged on one side of the cluster 1400.

The passenger seat dashboard 1600 may be spaced and/or apart (e.g., spaced apart or separated) from the cluster 1400 with the center fascia 1500 arranged therebetween. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 opposite to (e.g., facing) each other. The display apparatus 2 may be arranged on at least one selected from among the cluster 1400, the center fascia 1500, and the passenger seat dashboard 1600.

The display apparatus 2 may include an organic light-emitting display device, an inorganic electroluminescent (EL) display device, a quantum dot display device, and/or the like. Hereinafter, as the display apparatus 2 according to one or more embodiments, an organic light-emitting display apparatus including the light-emitting device according to the disclosure will be described as an example, but one or more suitable types (kinds) of display devices as described herein may be used in embodiments of the disclosure.

Referring to FIG. 6A, the display apparatus 2 may be arranged on the center fascia 1500. In one or more embodiments, the display apparatus 2 may display navigation information. In one or more embodiments, the display apparatus 2 may display audio, video, or information regarding vehicle settings.

Referring to FIG. 6B, the display apparatus 2 may be arranged on the cluster 1400. In this case, the cluster 1400 may display driving information and/or the like through the display apparatus 2. For example, the cluster 1400 may be implemented digitally. The digital cluster 1400 may display vehicle information and driving information as images. In one or more embodiments, a needle and a gauge of a tachometer and one or more suitable warning light icons may be displayed by a digital signal.

Referring to FIG. 6C, the display apparatus 2 may be arranged on the passenger seat dashboard 1600. The display apparatus 2 may be embedded in the passenger seat dashboard 1600 or arranged on the passenger seat dashboard 1600. In one or more embodiments, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information different from information displayed on the cluster 1400 and/or information displayed on the center fascia 1500.

### Manufacturing Method

The layers included in the hole transport region 120, the emission layer 130, and the layers included in the electron transport region 140 may be formed in a certain region by using one or more suitable methods such as vacuum deposition, spin coating, casting, a Langmuir-Blodgett (LB) method, ink-jet printing, laser-printing, and laser-induced thermal imaging (LITI).

When the layers included in the hole transport region 120, the emission layer 130, and the layers included in the electron transport region 140 are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as used herein refers to a cyclic group including (e.g., consisting of) carbon only as a ring-forming atom and having 3 to 60 carbon atoms.

The term “C₁-C₆₀ heterocyclic group” as used herein refers to a cyclic group that has 1 to 60 carbon atoms and further has, in addition to carbon, a heteroatom as a ring-forming atom.

The C₃-C₆₀ carbocyclic group and the C₁-C₆₀ heterocyclic group may each be a monocyclic group including (e.g., consisting of) one ring or a polycyclic group in which two or more rings are condensed with each other. In one or more embodiments, the number of ring-forming atoms of the C₁-C₆₀ heterocyclic group may be 3 to 61.

The “cyclic group” as used herein may include both (e.g., simultaneously) the C₃-C₆₀ carbocyclic group and the C₁-C₆₀ heterocyclic group.

The term “π electron-rich C₃-C₆₀ cyclic group” as used herein refers to a cyclic group that has 3 to 60 carbon atoms and does not include *—N=*′ as a ring-forming moiety.

The term “π electron-deficient nitrogen-containing C₁-C₆₀ cyclic group” as used herein refers to a heterocyclic group that has 1 to 60 carbon atoms and includes *—N=*′ as a ring-forming moiety.

In one or more embodiments,
the C₃-C₆₀ carbocyclic group may be i) Group T1 or ii) a condensed cyclic group in which two or more of Group T1 are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T2, ii) a condensed cyclic group in which two or more of Group T2 are condensed with each other, or iii) a condensed cyclic group in which at least one Group T2 and at least one Group T1 are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, a xanten group, and/or the like).

The π electron-rich C₃-C₆₀ cyclic group may be i) Group T1, ii) a condensed cyclic group in which two or more of Group T1 are condensed with each other, iii) Group T3, iv) a condensed cyclic group in which two or more of Group T3 are condensed with each other, or v) a condensed cyclic group in which at least one Group T3 and at least one Group T1 are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like).

The π electron-deficient nitrogen-containing C₁-C₆₀ cyclic group may be i) Group T4, ii) a condensed cyclic group in which two or more of Group T4 are condensed with each other, iii) a condensed cyclic group in which at least one Group T4 and at least one Group T1 are condensed with each other, iv) a condensed cyclic group in which at least one Group T4 and at least one Group T3 are condensed with each other, or v) a condensed cyclic group in which at least one Group T4, at least one Group T1, and at least one Group T3 are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like).

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group.

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

Group T3 may be a furan group, a thiophene group, a 1H-pyrrole group, a silole group, or a borole group.

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group,” “the C₃-C₆₀ carbocyclic group,” “the C₁-C₆₀ heterocyclic group,” “the π electron-rich C₃-C₆₀ cyclic group,” or “the π electron-deficient nitrogen-containing C₁-C₆₀ cyclic group,” as used herein, refer to a monovalent or polyvalent group (for example, a divalent group, a trivalent group, a tetravalent group, or the like) that is condensed with (e.g., combined together with) a cyclic group according to the structure of a formula for which the corresponding term is used.

In one or more embodiments, the “benzene group” may be a benzo group, a phenyl group, a phenylene group, and/or the like, which may be easily understood by those of ordinary skill in the art according to the structure of a formula including the “benzene group.”

Depending on context, a divalent group may refer or be a polyvalent group (e.g., trivalent, tetravalent, etc., and not just divalent) per, e.g., the structure of a formula in connection with which of the terms are utilized.

Examples of the monovalent C₃-C₆₀ carbocyclic group and monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as used herein refers to a linear or branched aliphatic hydrocarbon monovalent group that has 1 to 60 carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group.

The term “C₁-C₆₀ alkylene group” as used herein refers to a divalent group having the same structure as the C₁-C₆₀ alkyl group.

The term “C₂-C₆₀ alkenyl group” as used herein refers to a monovalent hydrocarbon group having at least one carbon-carbon double bond in the middle or at the terminus of the C₂-C₆₀ alkyl group, and examples thereof include an ethenyl group, a propenyl group, and a butenyl group.

The term “C₂-C₆₀ alkenylene group” as used herein refers to a divalent group having the same structure as the C₂-C₆₀ alkenyl group.

The term “C₂-C₆₀ alkynyl group” as used herein refers to a monovalent hydrocarbon group having at least one carbon-carbon triple bond in the middle or at the terminus of the C₂-C₆₀ alkyl group, and examples thereof include an ethynyl group and a propynyl group.

The term “C₂-C₆₀ alkynylene group” as used herein refers to a divalent group having the same structure as the C₂-C₆₀ alkynyl group.

The term “C₁-C₆₀ alkoxy group” as used herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as used herein refers to a monovalent saturated hydrocarbon cyclic group that has 3 to 10 carbon atoms, and examples thereof include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group.

The term “C₃-C₁₀ cycloalkylene group” as used herein refers to a divalent group having the same structure as the C₃-C₁₀ cycloalkyl group.

The term “C₁-C₁₀ heterocycloalkyl group” as used herein refers to a monovalent cyclic group that has 1 to 10 carbon atoms and further includes, in addition to the carbon atoms, at least one heteroatom as a ring-forming atom, and examples thereof include a 1,2,3,4-oxatriazolidinyl group, a tetrahydrofuranyl group, and a tetrahydrothiophenyl group.

The term “C₁-C₁₀ heterocycloalkylene group” as used herein refers to a divalent group having the same structure as the C₁-C₁₀ heterocycloalkyl group.

The term “C₃-C₁₀ cycloalkenyl group” as used herein refers to a monovalent cyclic group that has 3 to 10 carbon atoms, at least one carbon-carbon double bond in the ring thereof, and no aromaticity, and examples thereof include a cyclopentenyl group, a cyclohexenyl group, and a cycloheptenyl group.

The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent cyclic group that has 1 to 10 carbon atoms, further includes, in addition to the carbon atoms, at least one heteroatom as a ring-forming atom, and has at least one double bond in the ring thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group.

The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system of 6 to 60 carbon atoms.

The term “C₆-C₆₀ arylene group” as used herein refers to a divalent group having a carbocyclic aromatic system of 6 to 60 carbon atoms.

Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group.

When the C₆-C₆₀ aryl group and the C₆-C₆₀ arylene group each include two or more rings, the two or more rings may be condensed with each other.

The term “C₁-C₆₀ heteroaryl group” as used herein refers to a monovalent group having a heterocyclic aromatic system that has 1 to 60 carbon atoms and further includes, in addition to the carbon atoms, at least one heteroatom as a ring-forming atom.

The term “C₁-C₆₀ heteroarylene group” as used herein refers to a divalent group having a heterocyclic aromatic system that has 1 to 60 carbon atoms and further includes, in addition to the carbon atoms, at least one heteroatom as a ring-forming atom.

Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group.

When the C₁-C₆₀ heteroaryl group and the C₁-C₆₀ heteroarylene group each include two or more rings, the two or more rings may be condensed with each other.

The term “monovalent non-aromatic condensed polycyclic group” as used herein refers to a monovalent group having two or more rings condensed with each other, only carbon atoms (for example, eight to sixty carbon atoms) as ring-forming atoms, and no aromaticity in its molecular structure if (e.g., when) considered as a whole. Examples of the monovalent non-aromatic condensed polycyclic group include an indenyl group, a fluorenyl group, a spiro-bifluorenyl group, a benzofluorenyl group, an indenophenanthrenyl group, and an indeno anthracenyl group.

The term “divalent non-aromatic condensed polycyclic group” as used herein refers to a divalent group having the same structure as the monovalent non-aromatic condensed polycyclic group.

The term “monovalent non-aromatic condensed heteropolycyclic group” as used herein refers to a monovalent group that has two or more rings condensed with each other, further includes, in addition to carbon atoms (for example, one to sixty carbon atoms), at least one heteroatom as a ring-forming atom, and has no aromaticity in its molecular structure if (e.g., when) considered as a whole. Examples of the monovalent non-aromatic condensed heteropolycyclic group include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group.

The term “divalent non-aromatic condensed heteropolycyclic group” as used herein refers to a divalent group having the same structure as the monovalent non-aromatic condensed heteropolycyclic group.

The term “C₆-C₆₀ aryloxy group” as used herein indicates —OA₁₀₂ (wherein A₁₀₂ is the C₆-C₆₀ aryl group).

The term “C₆-C₆₀ arylthio group” as used herein indicates —SA₁₀₃ (wherein A₁₀₃ is the C₆-C₆₀ aryl group).

The term “C₇-C₆₀ arylalkyl group” as used herein refers to —A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as used herein refers to —A₁₀₆A₁₀₇ (wherein A₁₀₆ is a C₁-C₅₉ alkylene group, and A₁₀₇ is a C₁-C₅₉ heteroaryl group).

The term “R₁₀ₐ” as used herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂—C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ used herein may each independently be: hydrogen; deuterium; —F; —Cl; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as used herein refers to any atom other than a carbon atom. Examples of the heteroatom include O, S, N, P, Si, B, Ge, Se, or any combination thereof.

The term “transition metal” as used herein includes hafnium (Hf), tantalum (Ta), tungsten (W), rhenium (Re), osmium (Os), iridium (Ir), platinum (Pt), and gold (Au).

Throughout the present specification, “D” may represent deuterium, “Ph” may represent a phenyl group, “Me” may represent a methyl group, “Et” may represent an ethyl group, “tert-Bu,” “^{t}Bu,” or “Bu^{t}” may represent a tert-butyl group, and “OMe” may represent a methoxy group.

The term “biphenyl group” as used herein refers to “a phenyl group that is substituted with a phenyl group.” For example, the “biphenyl group” may be a substituted phenyl group having a C₆-C₆₀ aryl group as a substituent.

The term “terphenyl group” as used herein refers to “a phenyl group substituted with a biphenyl group.” The term “terphenyl group” as used herein may refer to i) a substituted phenyl group wherein the substituent is a C₆-C₆₀ aryl group substituted with a C₆-C₆₀ aryl group, and ii) a substituted phenyl group wherein two substituents are present, and each substituent is a C₆-C₆₀ aryl group.

* and *′ as used herein, unless defined otherwise, each refer to a binding site to a neighboring atom in a corresponding formula or moiety.

Herein, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, or 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light-emitting device, the electronic apparatus, the electronic equipment, and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light-emitting device and/or the electronic apparatus or equipment may be formed on one integrated circuit (IC) chip or on separate IC chips. Further, the various components of the light-emitting device and/or the electronic apparatus or equipment may be implemented on a flexible printed circuit film, a tape carrier package (TCP), a printed circuit board (PCB), or formed on one substrate. Further, the various components of the device, apparatus, and/or equipment may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Hereinafter, organic compounds according to one or more embodiments and light-emitting devices according to one or more embodiments are described in more detail with reference to the following synthesis examples and examples.

### EXAMPLES

### Synthesis Example 1 (Synthesis of Compound 5)

### Synthesis of Intermediate 5-a

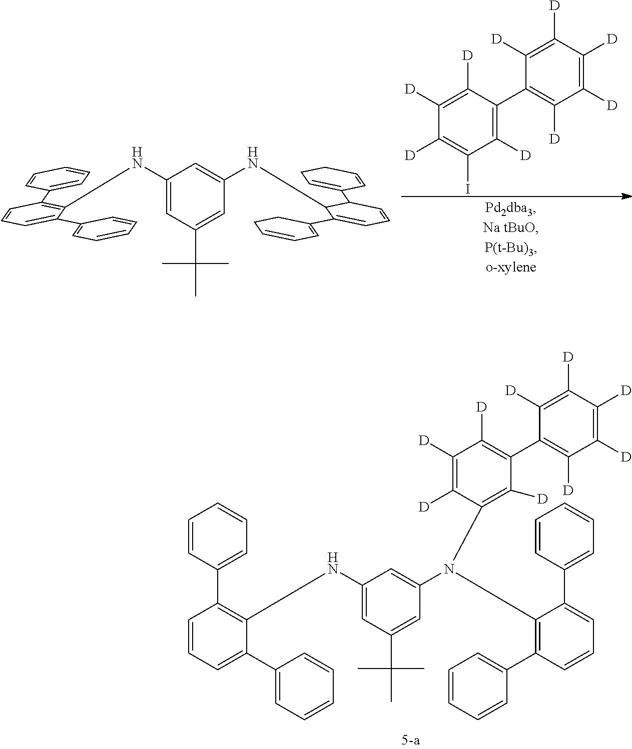 [SMILES: CC(C)(C)c1cc(Nc2c(-c3ccccc3)cccc2-c2ccccc2)cc(N[C@@H]2C(C3=CC=CCC3)=CC=CC2C2=CCCC=C2)c1 ; [2H]c1c([2H])c([2H])c(-c2c([2H])c([2H])c([2H])c(I)c2[2H])c([2H])c1[2H] ; [2H]c1c([2H])c([2H])c(-c2c([2H])c([2H])c([2H])c(N(c3cc(Nc4c(-c5ccccc5)cccc4-c4ccccc4)cc(C(C)(C)C)c3)c3c(-c4ccccc4)cccc3-c3ccccc3)c2[2H])c([2H])c1[2H]]

Under an argon atmosphere, N1,N3-di([1,1′:3′,1″-terphenyl]-2′-yl)-5-(tert-butyl)benzene-1,3-diamine (10 g, 16 mmol), 3-iodo-1,1′-biphenyl-2,2′,3′,4,4′,5,5′,6,6′-d9 (4.7 g, 16 mmol), Pd₂dba₃ (1.6 g, 1.9 mmol), tris-tert-butyl phosphine (P(t-Bu)₃, 1.6 mL, 3.8 mmol), and sodium tert-butoxide (Na tBuO, 11.5 g, 120 mmol) were placed in a 2 L flask and then dissolved in 300 mL of o-xylene, and then, the reaction solution was stirred at 140° C. for 2 hours. After cooling the mixture, water (1 L) and ethyl acetate (300 mL) were added for extraction to collect an organic layer, and then, the organic layer was dried with MgSO₄ and filtered. The solvent was removed from the filtrate under reduced pressure, and the obtained solid was purified and separated by column chromatography using silica gel by using CH₂Cl₂ and hexane as developing solvents, to obtain Intermediate 5-a (white solid, 8.1 g, 65%).

ESI-LCMS for Intermediate 5-a: [M]⁺: C₅₈H₃₉D₉N₂. 781.4416.

### Synthesis of Intermediate 5-b

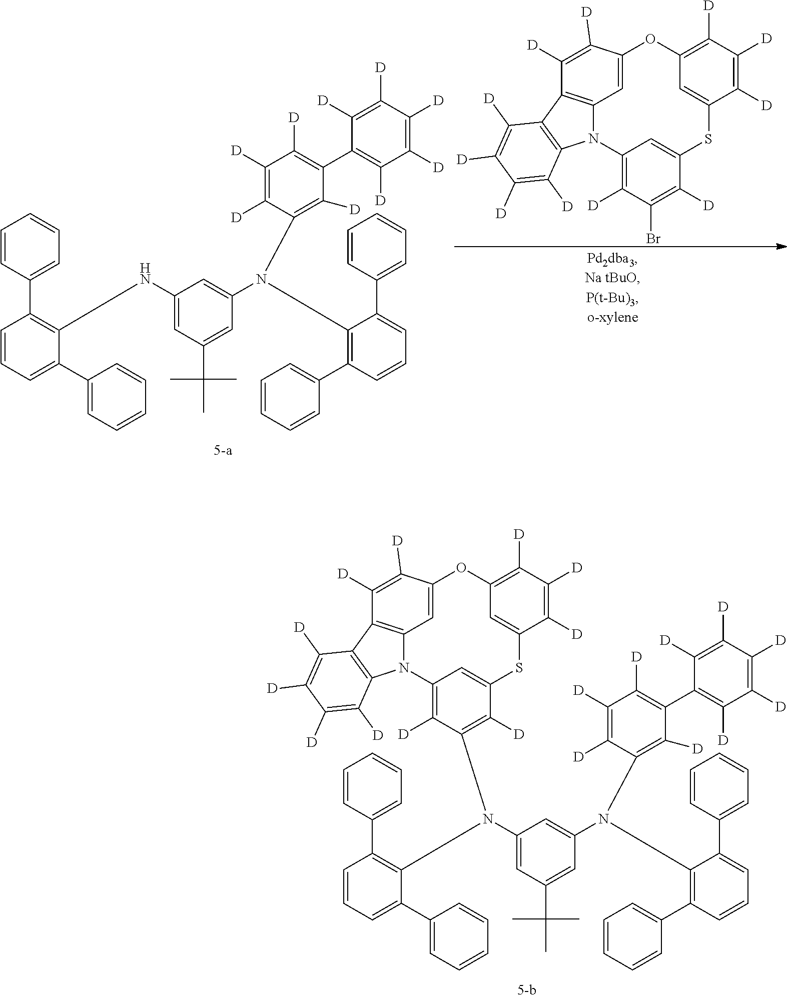

Under an argon atmosphere, Intermediate 5-a (8.1 g, 10 mmol), 25-bromo-19H-5-oxa-3-thia-1(9,2)-carbazola-2,4(1,3)-dibenzenacyclopentaphane-13,14,15,16,17,18,24,26,44,45,46-d11 (4.7 g, 10 mmol), Pd₂dba₃ (0.06 g, 0.12 mmol), tris-tert-butyl phosphine (0.06 mL, 0.24 mmol), and sodium tert-butoxide (1.4 g, 15 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 5-b (white solid, 8 g, 70%).

ESI-LCMS for Intermediate 5-b: [M]⁺: C₈₂H₄₁D₂₀N₃OS. 1155.5841

### Synthesis of Compound 5

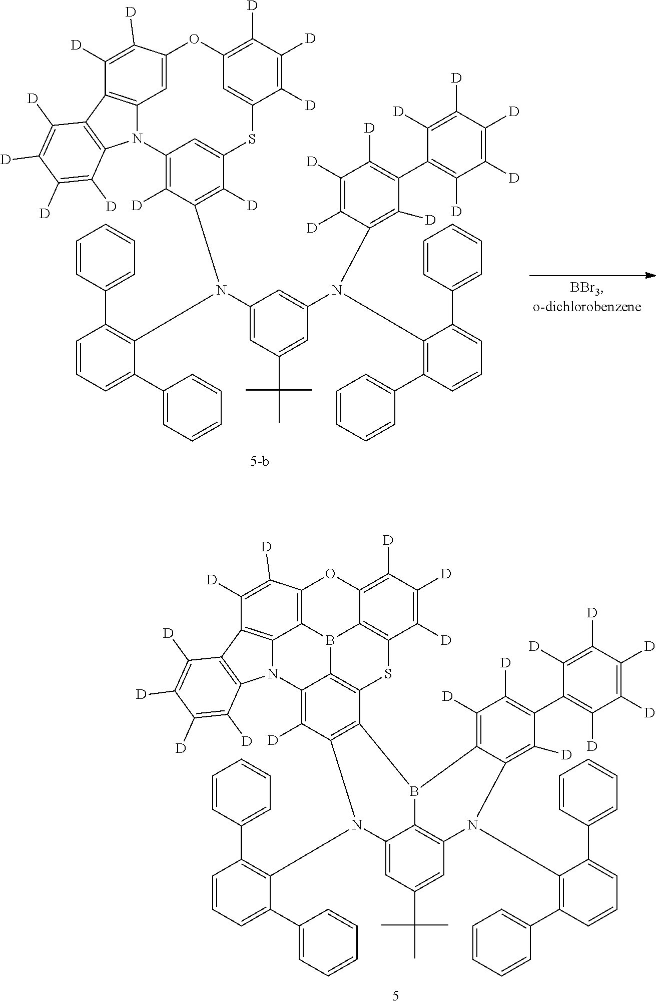

Under an argon atmosphere, Intermediate 5-b (8 g, 7 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 5 (yellow solid, 3.6 g, 45%).

ESI-LCMS for Compound 5: [M]⁺: C₈₂H₃₇D₁₈B₂N₃OS. 1169.5434

¹H-NMR for Compound 5 (CDCl₃): δ=8.22 (m, 4H), 7.43 (t, 2H), 7.32 (m, 12H), 7.12 (m, 8H), 7.01 (s, 2H), 1.32 (s, 27H).

### Synthesis Example 2 (Synthesis of Compound 7)

### Synthesis of Intermediate 7-b

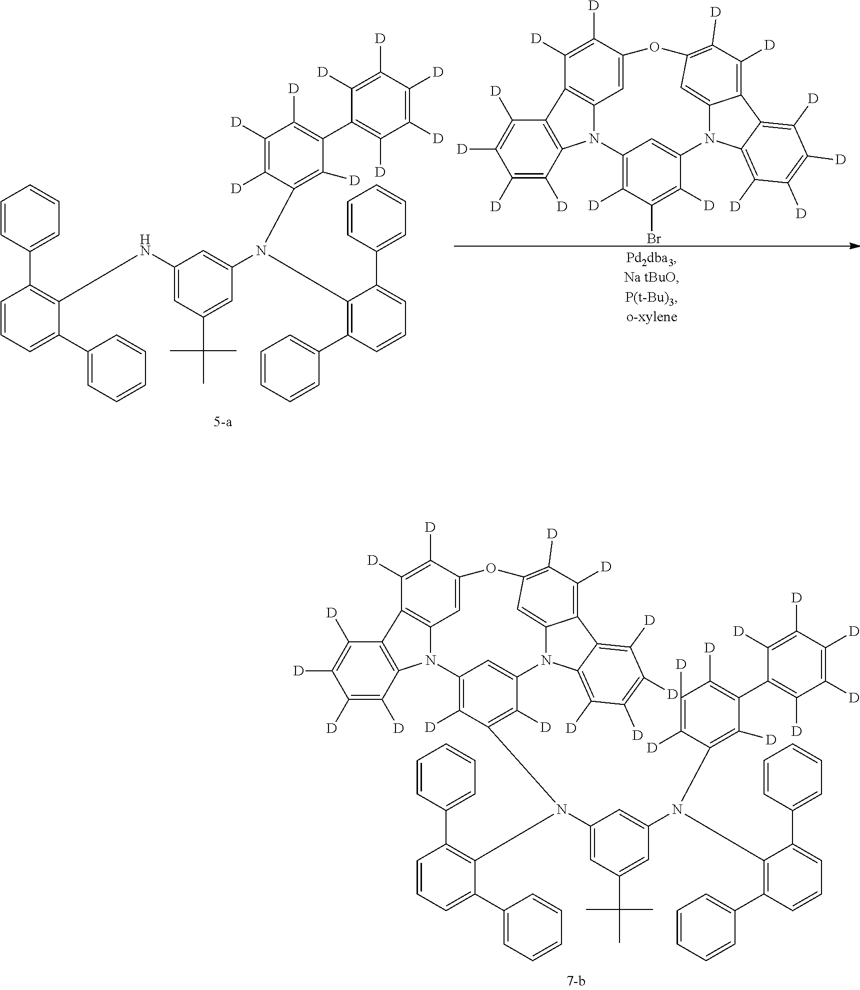

Under an argon atmosphere, Intermediate 5-a (8 g, 10 mmol), 8-bromo-16,18-(epiethane[1,2]diylidene)-20,22-etheno-6,10-(metheno)[1]oxa[5,11]diazacyclotetradecino[5,4-a:11,12-a′]diindole-1,2,3,4,7,9,12,13,14,15,23,24,25,26-d14 (5.2 g, 10 mmol), Pd₂dba₃ (0.06 g, 0.12 mmol), tris-tert-butyl phosphine (0.06 mL, 0.24 mmol), and sodium tert-butoxide (1.4 g, 15 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 7-b (white solid, 8.8 g, 72%).

ESI-LCMS for Intermediate 7-b: [M]⁺: C₈₈H₄₁D₂₃N₄O. 1215.6524

### Synthesis of Compound 7

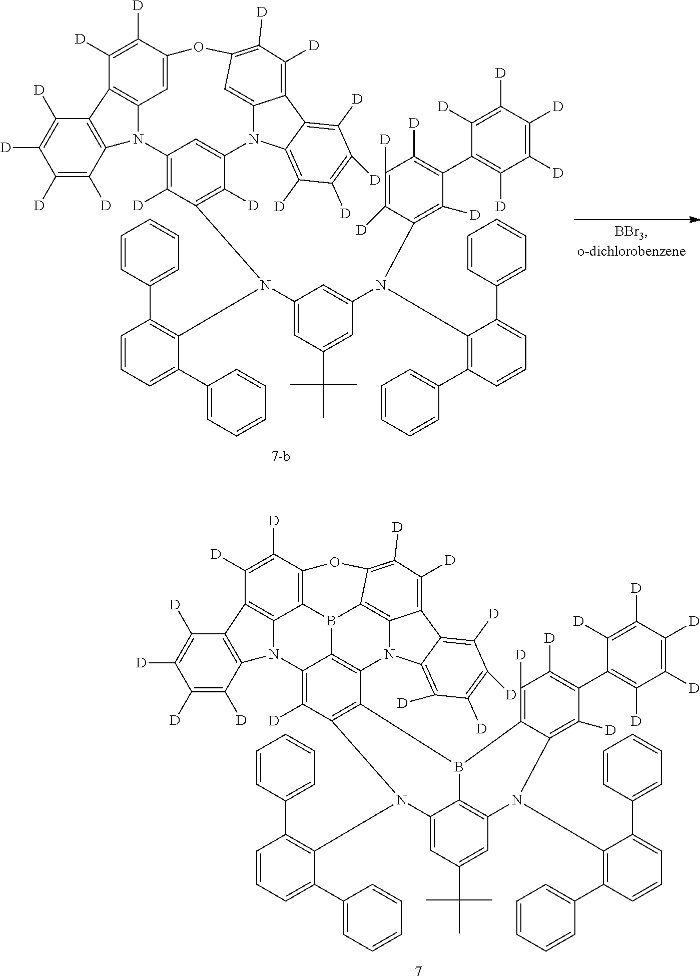

Under an argon atmosphere, Intermediate 7-b (8 g, 6.5 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 7 (yellow solid, 3 g, 37%).

ESI-LCMS for Compound 7: [M]⁺: C₈₈H₃₇D₂₁B₂N₄O. 1229.6107

¹H-NMR for Compound 7 (CDCl₃): δ=8.17 (m, 4H), 7.47 (t, 2H), 7.24 (m, 12H), 7.05 (m, 8H), 6.88 (s, 2H), 1.38 (s, 27H).

### Synthesis Example 3 (Synthesis of Compound 8)

### Synthesis of Intermediate 8-a

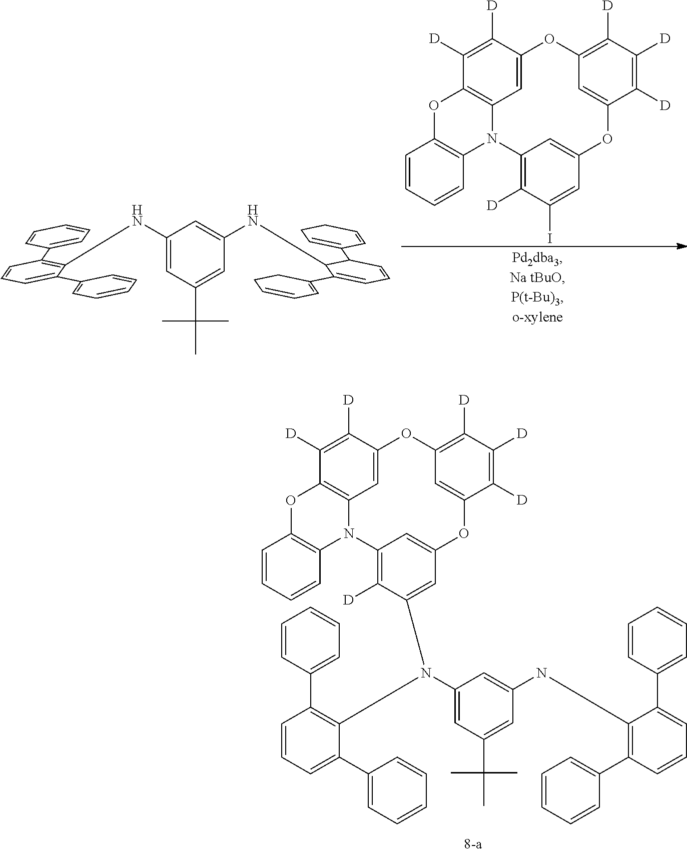 [SMILES: CC(C)(C)c1cc(Nc2c(-c3ccccc3)cccc2-c2ccccc2)cc(N[C@@H]2C(C3=CC=CCC3)=CC=CC2C2=CCCC=C2)c1 ; [2H]c1c(I)cc2cc1N1c3ccccc3Oc3c1cc(c([2H])c3[2H])Oc1cc(c([2H])c([2H])c1[2H])O2 ; [2H]c1c2cc(cc1N(c1cc(Nc3c(-c4ccccc4)cccc3-c3ccccc3)cc(C(C)(C)C)c1)c1c(-c3ccccc3)cccc1-c1ccccc1)Oc1cc(c([2H])c([2H])c1[2H])Oc1cc3c(c([2H])c1[2H])Oc1ccccc1N23]

Under an argon atmosphere, N1,N3-di([1,1′:3′,1″-terphenyl]-2′-yl)-5-(tert-butyl)benzene-1,3-diamine (10 g, 16 mmol), 25-iodo-110H-3,5-dioxa-1(10,2)-phenoxazina-2,4(1,3)-dibenzenacyclopentaphane-13,14,26,44,45,46-d6 (8 g, 16 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 200 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 8-a (white solid, 11 g, 71%).

ESI-LCMS for Intermediate 8-a: [M]⁺: C₇₀H₄₇D₆N₃O₃. 989.4532

### Synthesis of Intermediate 8-b

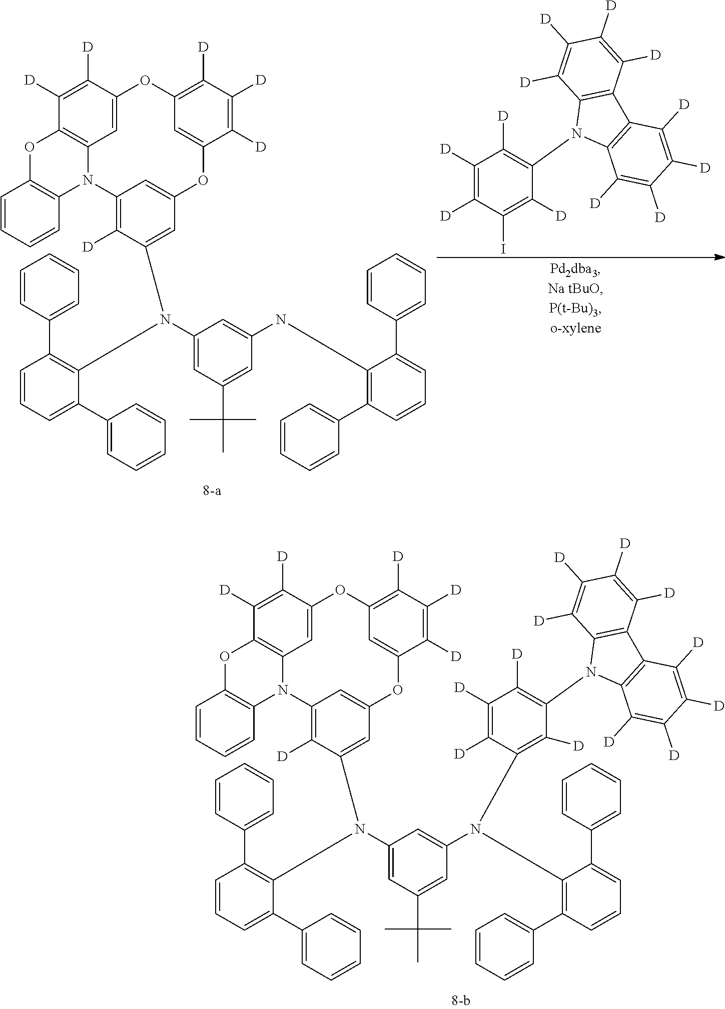

Under an argon atmosphere, Intermediate 8-a (10 g, 10 mmol), 9-(3-iodophenyl-2,4,5,6-d4)-9H-carbazole-1,2,3,4,5,6,7,8-d8 (3.9 g, 10 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 8-b (white solid, 8.2 g, 66%).

ESI-LCMS for Intermediate 8-b: [M]⁺: C₈₈H₄₆D₁₈N₄O₃. 1242.6138

### Synthesis of Compound 8

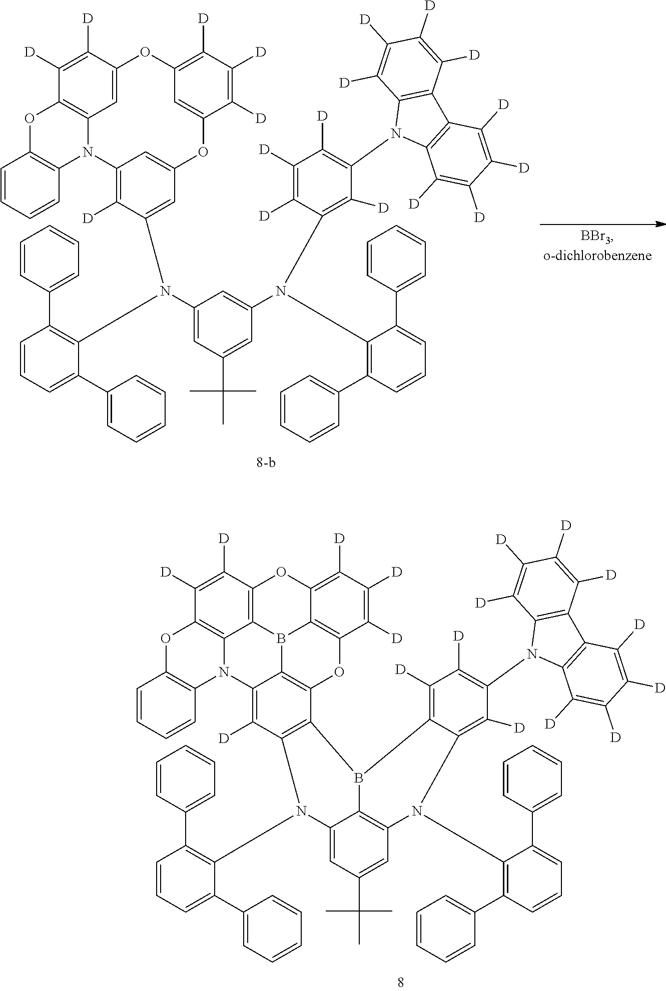

Under an argon atmosphere, Intermediate 8-b (8 g, 6.4 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 8 (yellow solid, 2.8 g, 35%).

ESI-LCMS for Compound 8: [M]⁺: C₈₈H₄₁D₁₇B2N₄O₃. 1257.5828

¹H-NMR for Compound 8 (CDCl₃): δ=7.47 (m, 2H), 7.42 (m, 4H), 7.30 (m, 12H), 7.08 (m, 8H), 7.01 (m, 4H), 6.94 (s, 2H), 1.31 (s, 9H).

### Synthesis Example 4 (Synthesis of Compound 13)

### Synthesis of Intermediate 13-a

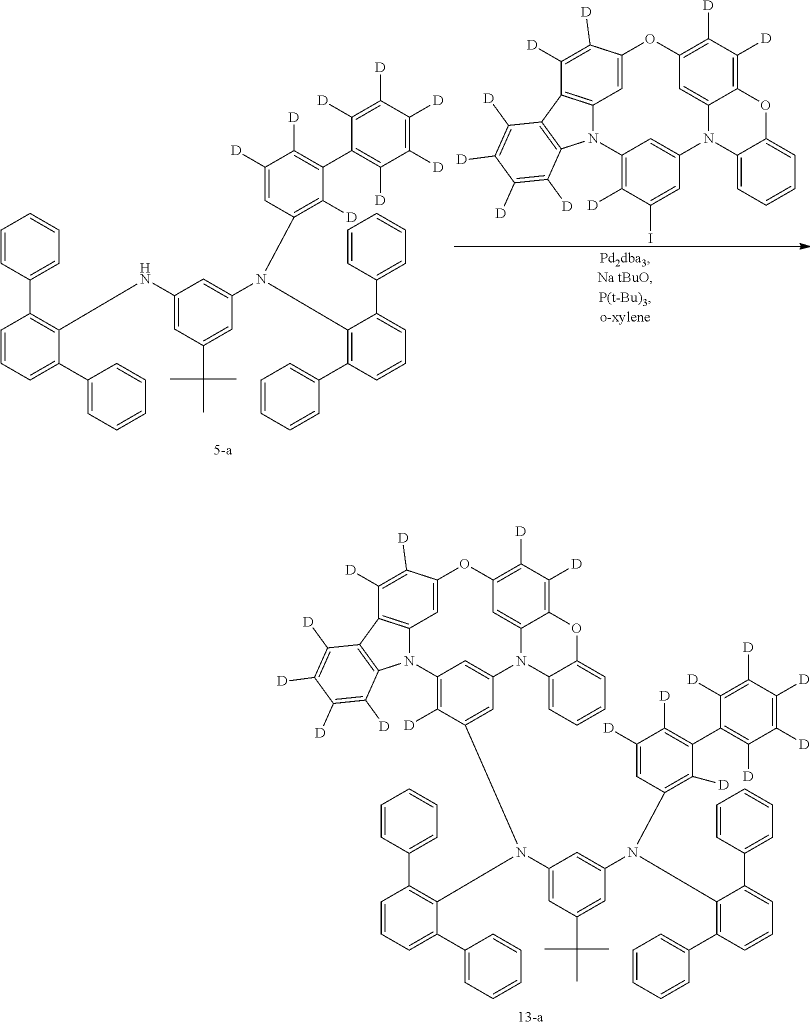

Under an argon atmosphere, Intermediate 5-a (10 g, 12.8 mmol), 8-iodo-16,18:20,22-diethieno-6,10-(metheno)[1]oxa[5,11]diazacyclotetradecino[5,4-a:11,12-a′]diindole-1,2,3,4,7,9,12,13,14,15,23,24,25,26-d14 (7.4 g, 12.8 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 13-a (white solid, 10 g, 64%).

ESI-LCMS for Intermediate 13-a: [M]⁺: C₈₈H₄₇D₁₇N₄O₂. 1225.6113

### Synthesis of Compound 13

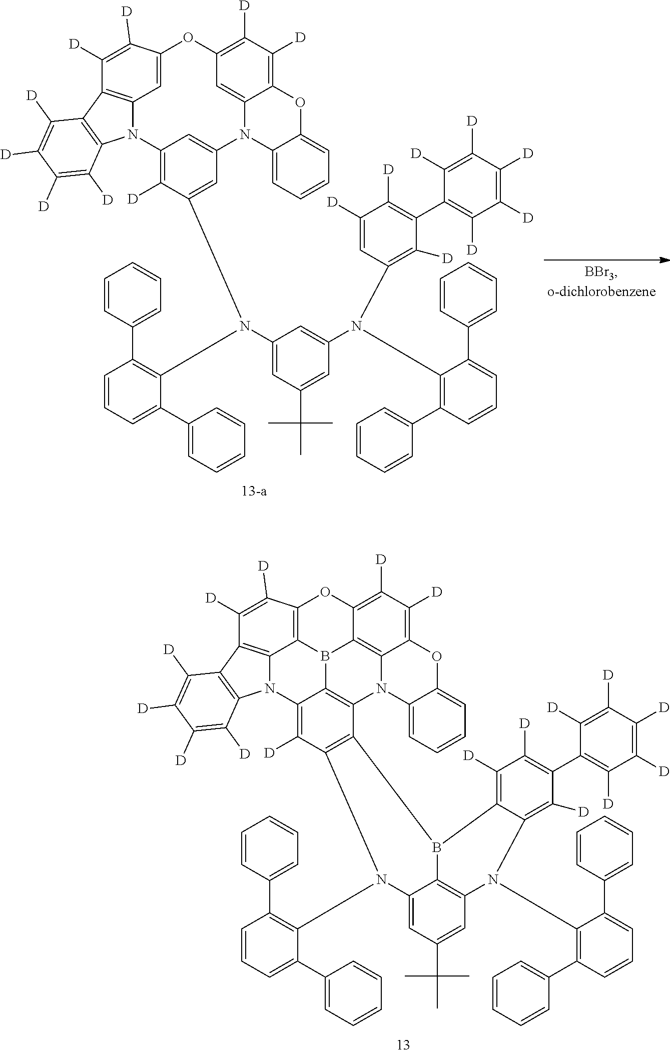

Under an argon atmosphere, Intermediate 13-a (10 g, 8.2 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 13 (yellow solid, 3.34 g, 33%).

ESI-LCMS for Compound 13: [M]⁺: C₈₈H₄₁D₁₇B2N₄O₂. 1241.5811

¹H-NMR for Compound 13 (CDCl₃): δ=7.45 (m, 2H), 7.40 (m, 4H), 7.27 (m, 12H), 7.06 (m, 8H), 7.00 (m, 4H), 6.98 (s, 2H), 1.28 (s, 9H).

### Synthesis Example 5 (Synthesis of Compound 14)

### Synthesis of Intermediate 14-a

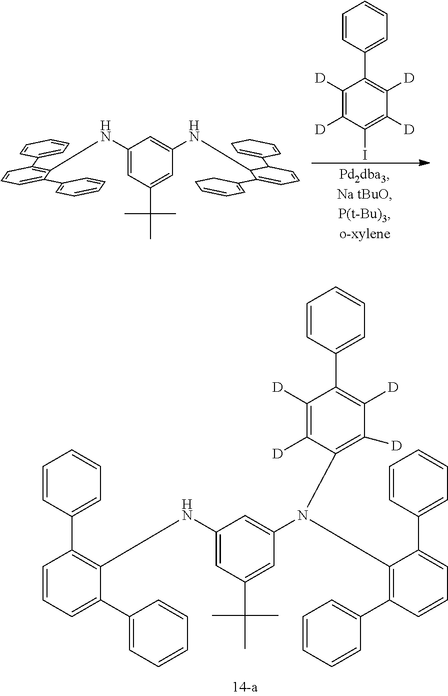 [SMILES: CC(C)(C)c1cc(Nc2c(-c3ccccc3)cccc2-c2ccccc2)cc(N[C@@H]2C(C3=CC=CCC3)=CC=CC2C2=CCCC=C2)c1 ; [2H]c1c([2H])c(-c2ccccc2)c([2H])c([2H])c1I ; [2H]c1c([2H])c(N(c2cc(Nc3c(-c4ccccc4)cccc3-c3ccccc3)cc(C(C)(C)C)c2)c2c(-c3ccccc3)cccc2-c2ccccc2)c([2H])c([2H])c1-c1ccccc1]

Under an argon atmosphere, N1,N3-di([1,1′:3′,1″-terphenyl]-2′-yl)-5-(tert-butyl)benzene-1,3-diamine (10 g, 16 mmol), 4-iodo-1,1′-biphenyl-2,3,5,6-d4 (4.5 g, 16 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 200 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 14-a (white solid, 8 g, 64%).

ESI-LCMS for Intermediate 14-a: [M]⁺: C₅₈H₄₄D₄N₂. 776.4123

### Synthesis of Intermediate 14-b

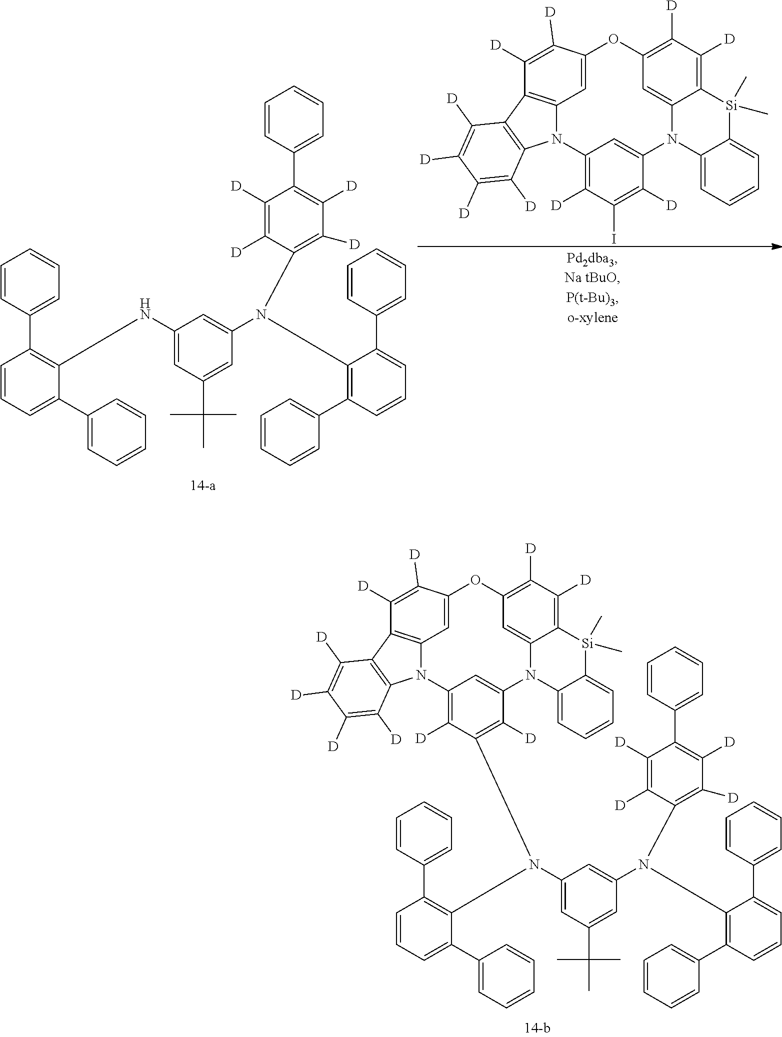 [SMILES: [2H]c1c([2H])c(N(c2cc(Nc3c(-c4ccccc4)cccc3-c3ccccc3)cc(C(C)(C)C)c2)c2c(-c3ccccc3)cccc2-c2ccccc2)c([2H])c([2H])c1-c1ccccc1 ; [2H]c1c2cc3c(c1[2H])[Si](C)(C)c1ccccc1N3c1cc(c([2H])c(I)c1[2H])-n1c3cc(c([2H])c([2H])c3c3c([2H])c([2H])c([2H])c([2H])c31)O2 ; [2H]c1c2cc3c(c1[2H])[Si](C)(C)c1ccccc1N3c1cc(c([2H])c(N(c3cc(N(c4c(-c5ccccc5)cccc4-c4ccccc4)c4c([2H])c([2H])c(-c5ccccc5)c([2H])c4[2H])cc(C(C)(C)C)c3)c3c(-c4ccccc4)cccc3-c3ccccc3)c1[2H])-n1c3cc(c([2H])c([2H])c3c3c([2H])c([2H])c([2H])c([2H])c31)O2]

Under an argon atmosphere, Intermediate 14-a (8 g, 10 mmol), 20-iodo-5,5-dimethyl-5H-6,8-(epiethane[1,2]diylidene)-10,12-etheno-18,22-(metheno)benzo[5′,6′][1,4]azasilino[1′,2′:11,12][1]oxa[5,11]diazacyclotetradecino[5,4-a]indole-7,11,13,14,15,16,19,21,25,26,27,28-d12 (6.4 g, 10 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 14-b (white solid, 8.2 g, 65%).

ESI-LCMS Intermediate 14-b: [M]⁺: C₉₀H₅₆D₁₄N₄OSi. 1264.6214

### Synthesis of Compound 14

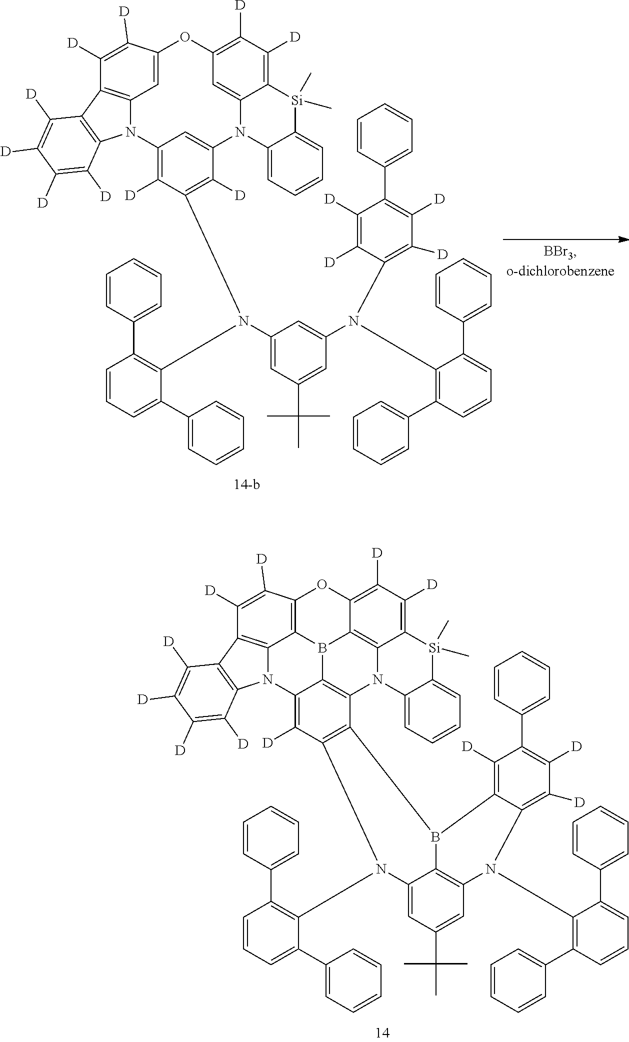 [SMILES: [2H]c1c2cc3c(c1[2H])[Si](C)(C)c1ccccc1N3c1cc(c([2H])c(N(c3cc(N(c4c(-c5ccccc5)cccc4-c4ccccc4)c4c([2H])c([2H])c(-c5ccccc5)c([2H])c4[2H])cc(C(C)(C)C)c3)c3c(-c4ccccc4)cccc3-c3ccccc3)c1[2H])-n1c3cc(c([2H])c([2H])c3c3c([2H])c([2H])c([2H])c([2H])c31)O2 ; [2H]c1c([2H])c2c3c4c1Oc1c([2H])c([2H])c5c6c([2H])c([2H])c([2H])c([2H])c6n6c5c1B4c1c(c4c(c([2H])c1-6)N(c1c(-c5ccccc5)cccc1-c1ccccc1)c1cc(C(C)(C)C)cc5c1B4c1c([2H])c(-c4ccccc4)c([2H])c([2H])c1N5c1c(-c4ccccc4)cccc1-c1ccccc1)N3c1ccccc1[Si]2(C)C]

Under an argon atmosphere, Intermediate 14-b (8 g, 6.3 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 14 (yellow solid, 2.34 g, 29%).

ESI-LCMS for Compound 14: [M]⁺: C₉₀H₅₂D₁₂B₂N₄OSi. 1278.4151

¹H-NMR for Compound 14 (CDCl₃): δ=7.43 (m, 2H), 7.39 (m, 4H), 7.29 (m, 17H), 7.10 (m, 8H), 7.01 (m, 4H), 6.95 (s, 2H), 1.33 (s, 9H).

### Synthesis Example 6 (Synthesis of Compound 15)

### Synthesis of Intermediate 15-a

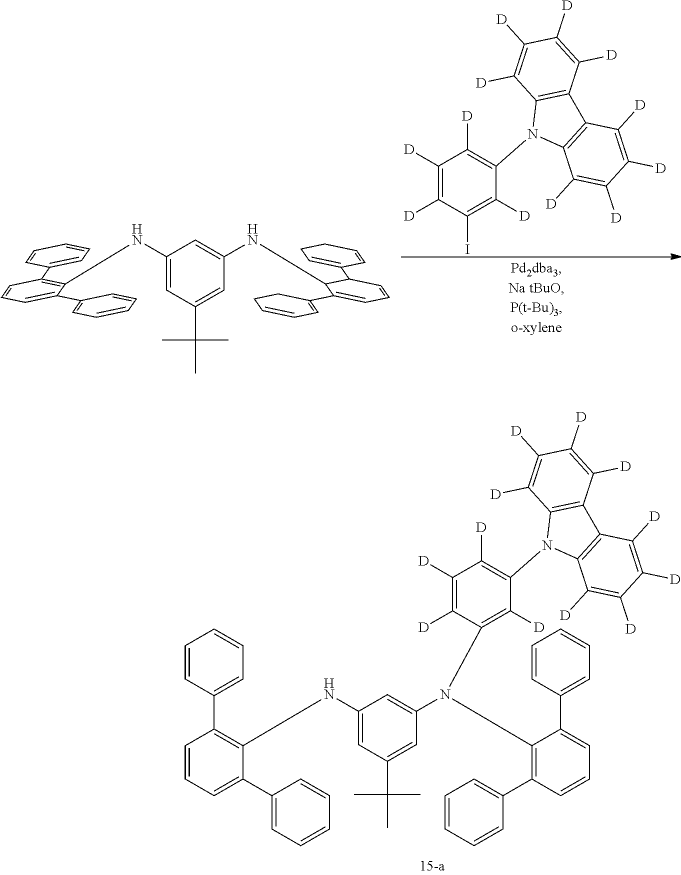 [SMILES: CC(C)(C)c1cc(Nc2c(-c3ccccc3)cccc2-c2ccccc2)cc(N[C@@H]2C(C3=CC=CCC3)=CC=CC2C2=CCCC=C2)c1 ; [2H]c1c([2H])c(I)c([2H])c(-n2c3c([2H])c([2H])c([2H])c([2H])c3c3c([2H])c([2H])c([2H])c([2H])c32)c1[2H] ; [2H]c1c([2H])c(N(c2cc(Nc3c(-c4ccccc4)cccc3-c3ccccc3)cc(C(C)(C)C)c2)c2c(-c3ccccc3)cccc2-c2ccccc2)c([2H])c(-n2c3c([2H])c([2H])c([2H])c([2H])c3c3c([2H])c([2H])c([2H])c([2H])c32)c1[2H]]

Under an argon atmosphere, N1,N3-di([1,1′:3′,1″-terphenyl]-2′-yl)-5-(tert-butyl)benzene-1,3-diamine (10 g, 16 mmol), 9-(3-iodophenyl-2,4,5,6-d4)-9H-carbazole-1,2,3,4,5,6,7,8-d8 (6.1 g, 16 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 200 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 15-a (white solid, 9.5 g, 68%).

ESI-LCMS for Intermediate 15-a: [M]⁺: C₆₄H₃₉D₁₂N₃. 874.2128

### Synthesis of Intermediate 15-b

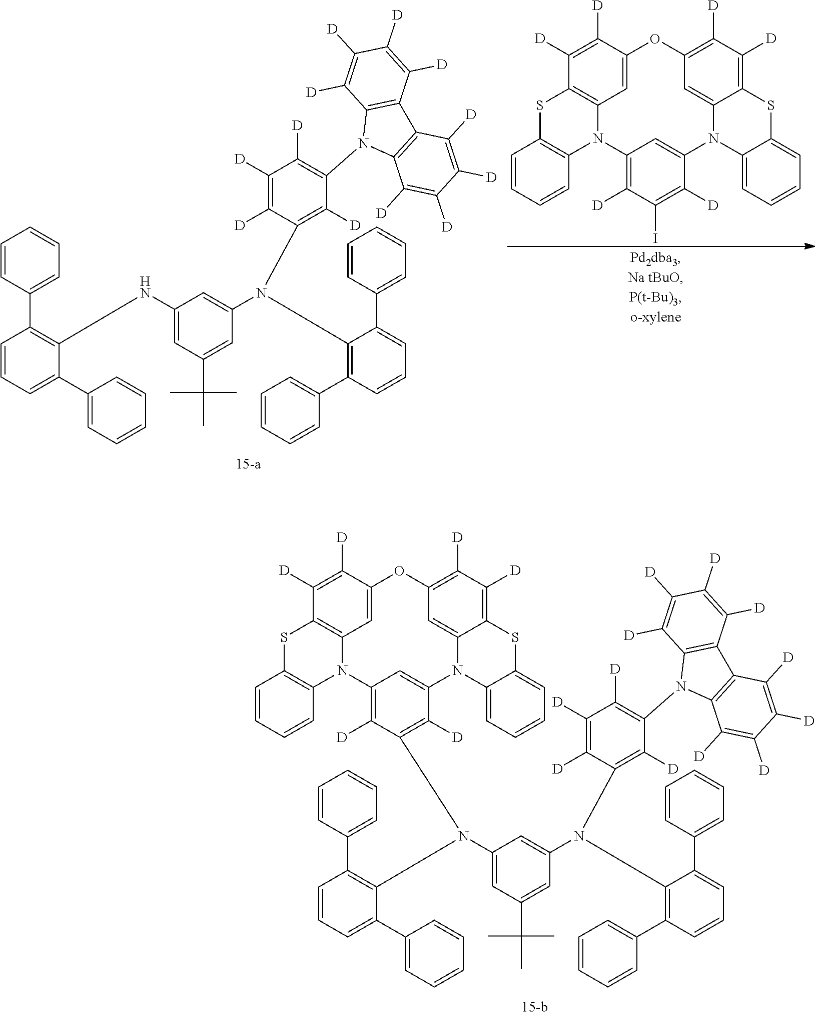

Under an argon atmosphere, Intermediate 15-a (9 g, 10 mmol), 21-iodo-6,8-(epiethane[1,2]diylidene)-10,12-etheno-19,23-(metheno)benzo[5,6][1,4]thiazino[3,4-d]benzo[5,6][1,4]thiazino[4,3-k][1]oxa[5,11]diazacyclotetradecine-20,22,26,27,28,29-d6 (6.4 g, 10 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 15-b (white solid, 8.2 g, 60%).

ESI-LCMS for Intermediate 15-b: [M]⁺: C₉₄H₄₉D₁₈N₅OS₂. 1363.5954

### Synthesis of Compound 15

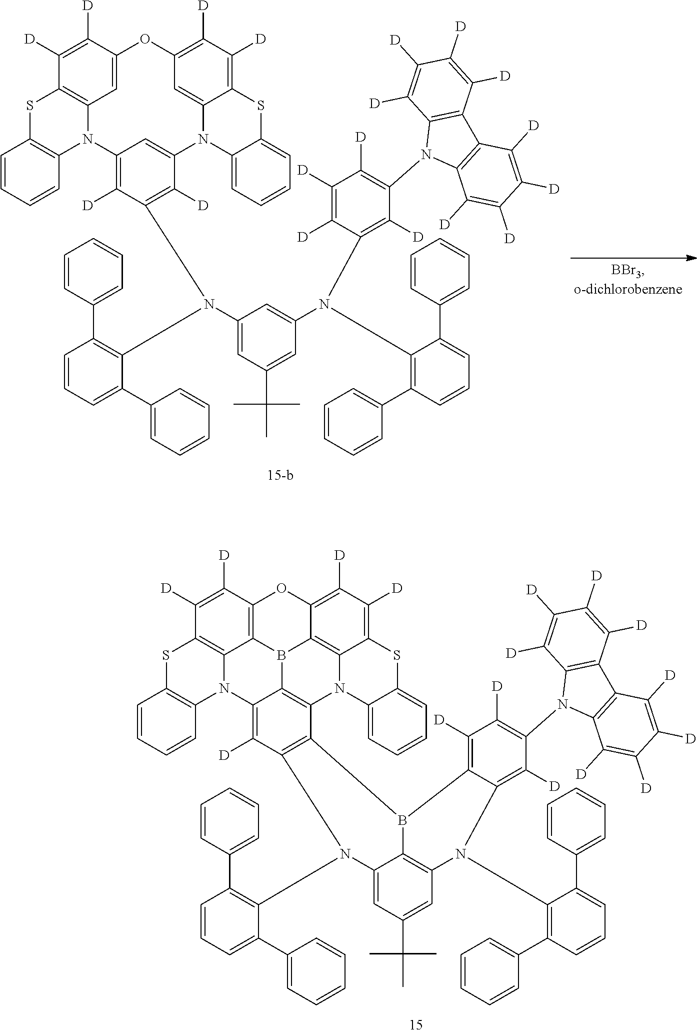

Under an argon atmosphere, Intermediate 15-b (8 g, 5.9 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 15 (yellow solid, 1.9 g, 23%).

ESI-LCMS for Compound 15: [M]⁺: C₉₄H₄₅D₁₆B2N₅OS₂. 1377.5514

¹H-NMR for Compound 15 (CDCl₃): δ=7.41 (m, 2H), 7.36 (m, 8H), 7.25 (m, 17H), 7.07 (m, 8H), 7.00 (m, 4H), 6.90 (s, 2H), 1.35 (s, 9H).

### Synthesis Example 7 (Synthesis of Compound 20)

### Synthesis of Intermediate 20-a

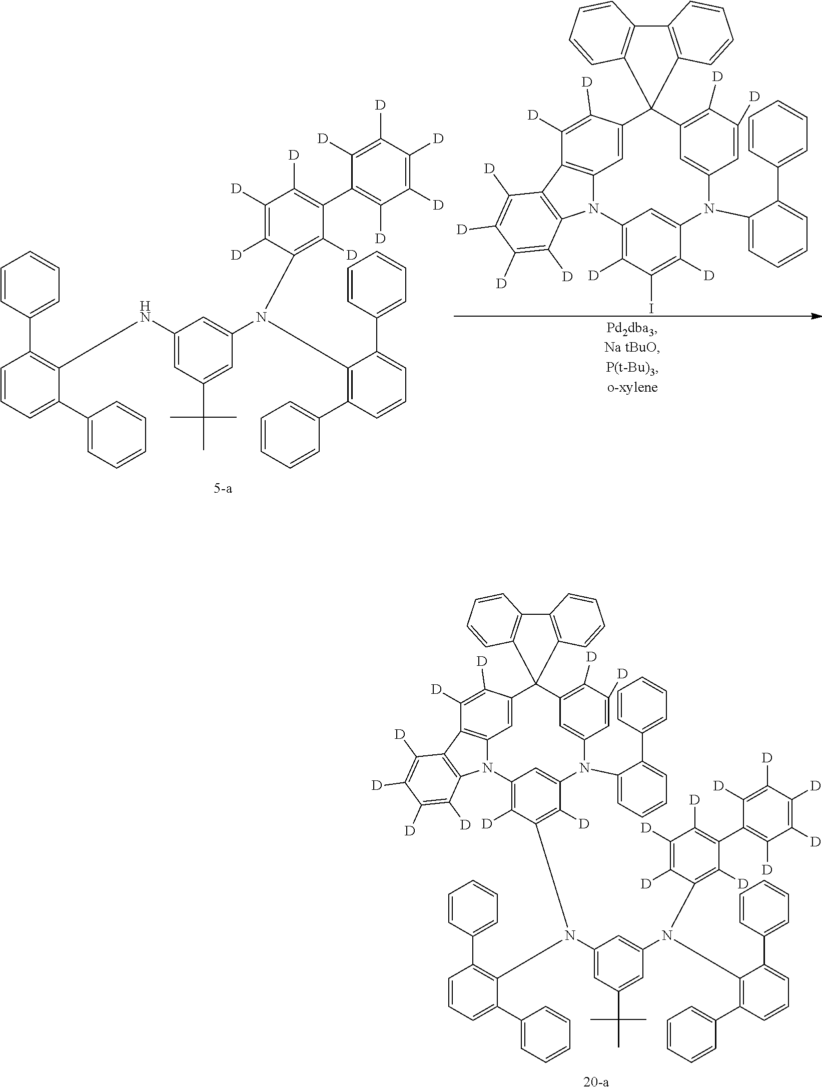

Under an argon atmosphere, Intermediate 5-a (10 g, 12.7 mmol), 3′-([1,1′-biphenyl]-2-yl)-5′-iodospiro[fluorene-9,5′-3-aza-1(9,2)-carbazola-2,4(1,3)-dibenzenacyclopentaphane]-3′,4′,4′,4′,5′,5′,6′,6′,7′,8′-d10 (10 g, 12.7 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 20-a (white solid, 11.8 g, 65%).

ESI-LCMS for Intermediate 20-a: [M]⁺: C₁₀₇H₅₉D₁₉N₄. 1437.7477

### Synthesis of Compound 20

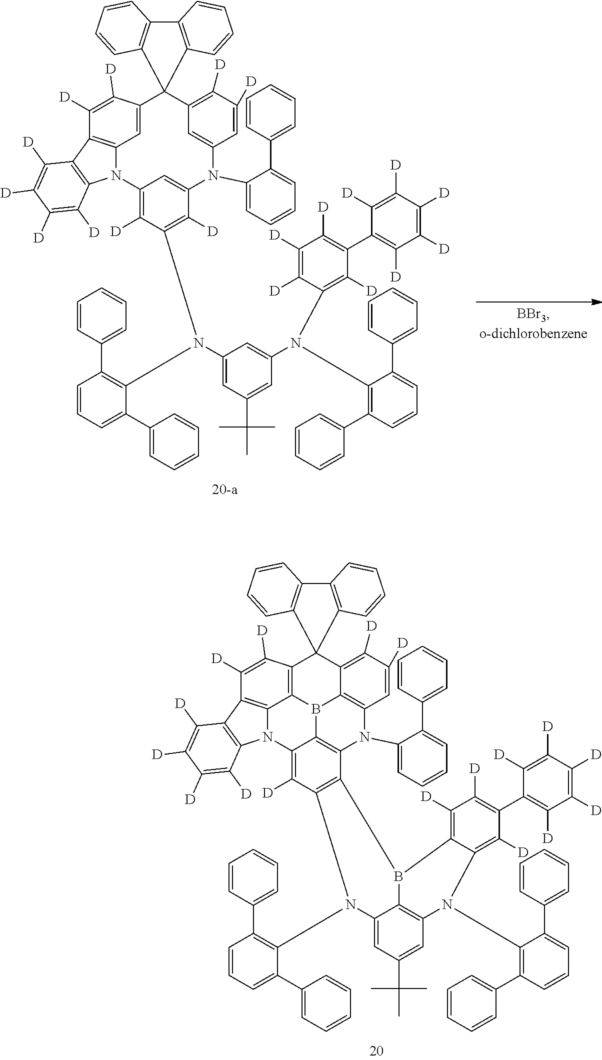

Under an argon atmosphere, Intermediate 20-a (10 g, 7 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 20 (yellow solid, 2.4 g, 24%).

ESI-LCMS for Compound 20: [M]⁺: C₁₀₇H₅₅D₁₇B₂N₄. 1451.7020

¹H-NMR for Compound 20 (CDCl₃): δ=8.10 (d, 1H), 7.90 (d, 2H), 7.56 (d, 2H), 7.45 (m, 5H), 7.41 (m, 6H), 7.35 (d, 2H), 7.29 (m, 12H), 7.17 (t, 2H), 7.05 (m, 10H), 7.01 (m, 1H), 6.95 (s, 2H), 6.90 (s, 1H), 1.36 (s, 9H).

### Synthesis Example 8 (Synthesis of Compound 37)

### Synthesis of Intermediate 37-a

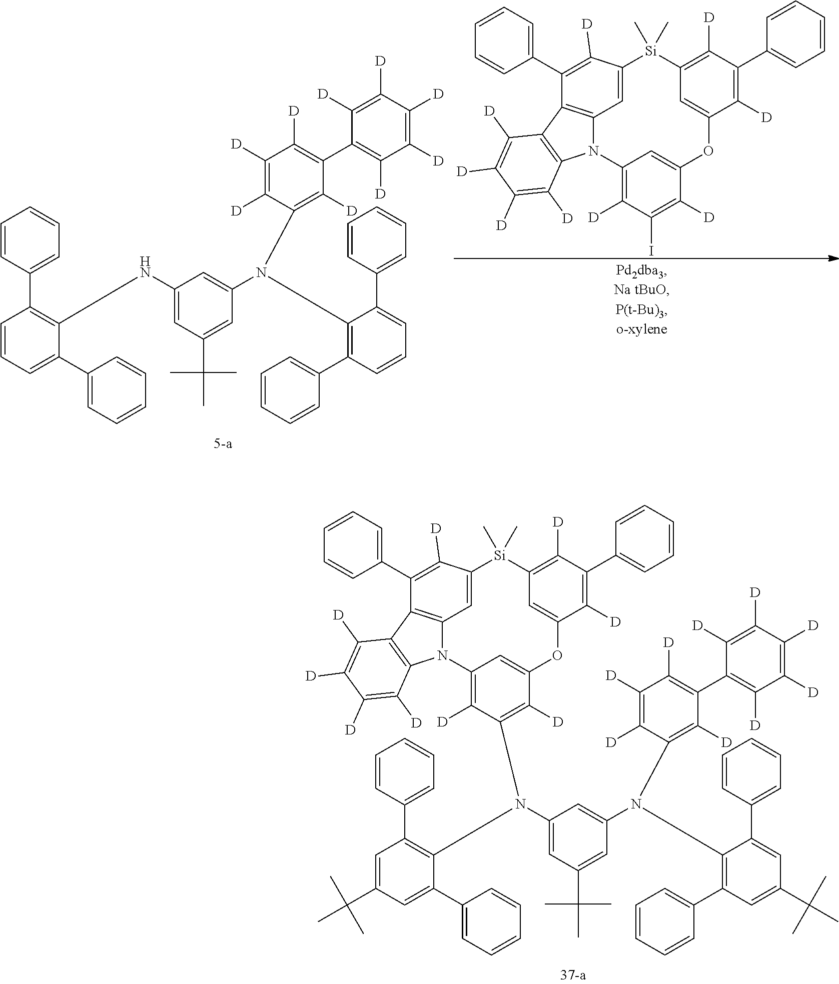

Under an argon atmosphere, Intermediate 5-a (10 g, 12.7 mmol), 25-iodo-5,5-dimethyl-14,45-diphenyl-19H-3-oxa-5-sila-1 (9,2)-carbazola-2,4(1,3)-dibenzenacyclopentaphane-13,15,16,17,18,24,26,44,46-d9 (6.8 g, 12.7 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 37-a (white solid, 11.5 g, 66%).

ESI-LCMS for Intermediate 37-a: [M]⁺: C₁₀₄H₇₃D₁₈N₃OSi. 1443.8062

### Synthesis of Compound 37

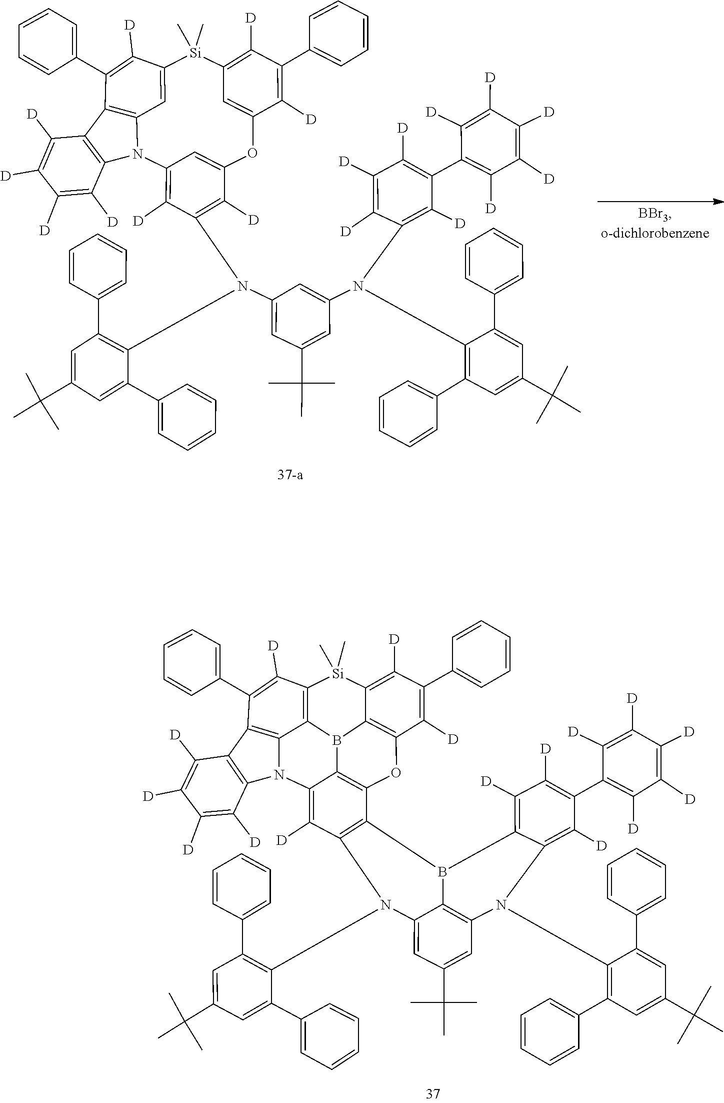

Under an argon atmosphere, Intermediate 37-a (10 g, 7 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 37 (yellow solid, 2.2 g, 22%).

ESI-LCMS for Compound 37: [M]⁺: C₁₀₄H₆₉D₁₆B₂N₃OSi. 1457.7781

¹H-NMR for Compound 37 (CDCl₃): δ=7.41 (s, 4H), 7.35 (m, 22H), 7.09 (m, 8H), 7.03 (s, 2H), 1.36 (s, 9H), 1.23 (s, 18H), 0.66 (s, 6H)

### Synthesis Example 9 (Synthesis of Compound 45)

### Synthesis of Intermediate 45-a

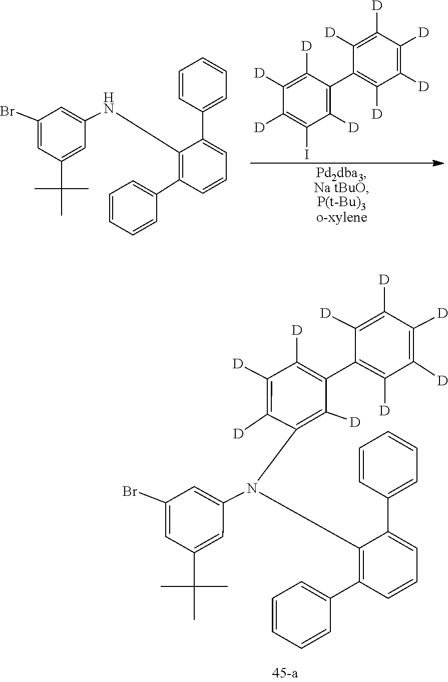

Under an argon atmosphere, N-(3-bromo-5-(tert-butyl)phenyl)-[1,1′:3′,1″-terphenyl]-2′-amine (10 g, 22 mmol), 3-iodo-1,1′-biphenyl-2,2′,3′,4,4′,5,5′,6,6′-d9 (6.3 g, 16 mmol), Pd₂dba₃ (1.6 g, 1.9 mmol), tris-tert-butyl phosphine (1.6 mL, 3.8 mmol), and sodium tert-butoxide (11.5 g, 120 mmol) were placed in a 2 L flask and then dissolved in 300 mL of o-xylene, and then, the reaction solution was stirred at 140° C. for 2 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 45-a (white solid, 10.4 g, 77%).

ESI-LCMS for Intermediate 45-a: [M]⁺: C₄₀H₂₅D₉BrN. 616.2421.

### Synthesis of Intermediate 45-b

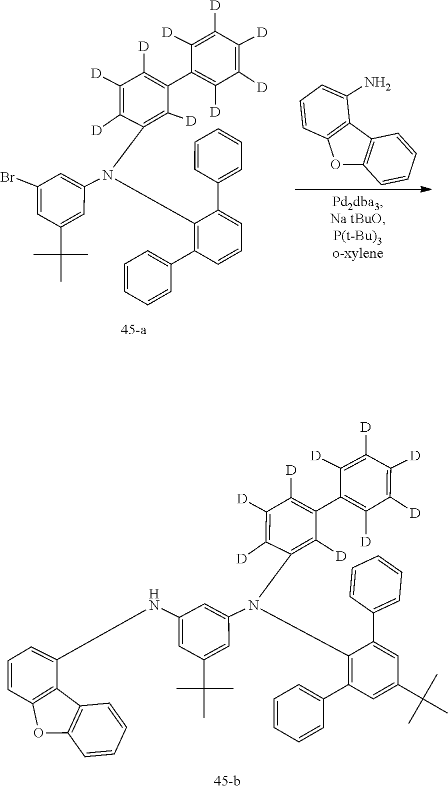 [SMILES: [2H]c1c([2H])c([2H])c(-c2c([2H])c([2H])c([2H])c(N(c3cc(Br)cc(C(C)(C)C)c3)c3c(-c4ccccc4)cccc3-c3ccccc3)c2[2H])c([2H])c1[2H] ; Nc1cccc2oc3ccccc3c12 ; [2H]c1c([2H])c([2H])c(-c2c([2H])c([2H])c([2H])c(N(c3cc(Nc4cccc5oc6ccccc6c45)cc(C(C)(C)C)c3)c3c(-c4ccccc4)cc(C(C)(C)C)cc3-c3ccccc3)c2[2H])c([2H])c1[2H]]

Under an argon atmosphere, Intermediate 45-a (10 g, 16 mmol), dibenzo[b,d]furan-1-amine (3 g, 16 mmol), Pd₂dba₃ (0.06 g, 0.12 mmol), tris-tert-butyl phosphine (0.06 mL, 0.24 mmol), and sodium tert-butoxide (1.4 g, 15 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 45-b (white solid, 8.3 g, 67%).

ESI-LCMS for Intermediate 45-b: [M]⁺: C₅₆H₄₁D₉N₂O. 775.4534

### Synthesis of Intermediate 45-c

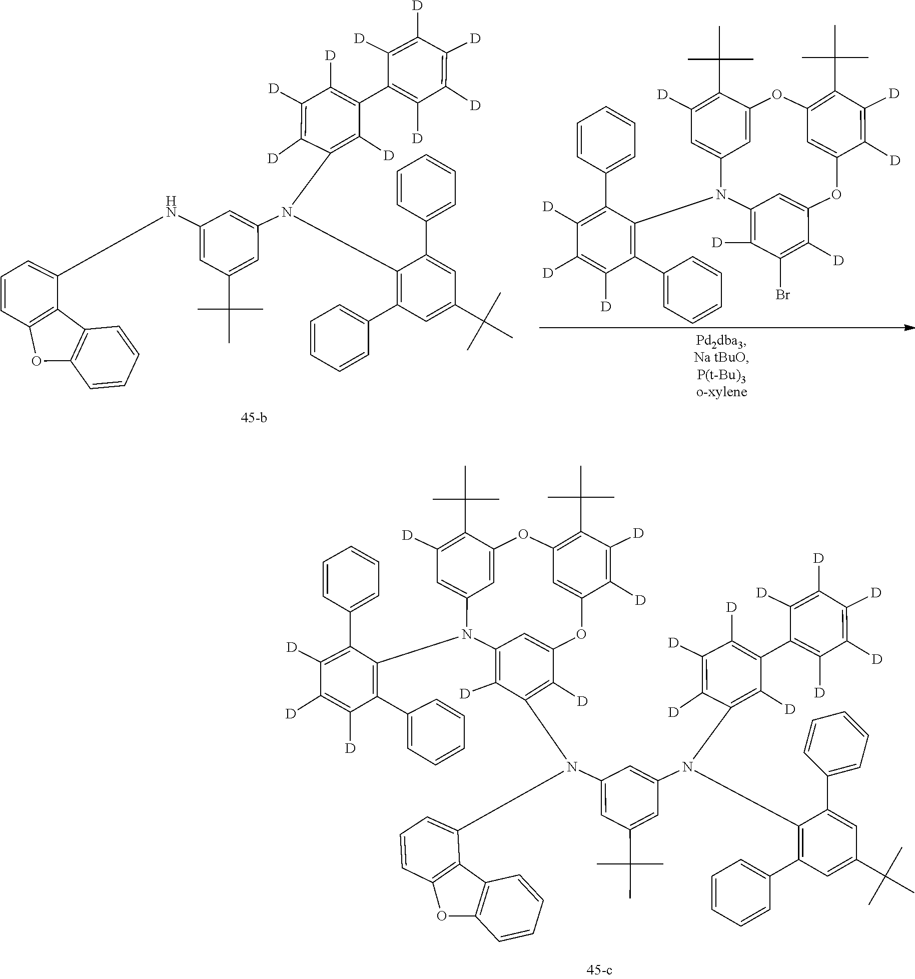

Under an argon atmosphere, Intermediate 45-b (8 g, 10 mmol), 6-([1,1′:3′,1″-terphenyl]-2′-yl-4′,5′,6′-d3)-15-bromo-34,56-di-tert-butyl-2,4-dioxa-6-aza-1,3,5(1,3)-tribenzenacyclohexaphane-14,16,35,36,55-d5 (7.3 g, 10 mmol), Pd₂dba₃ (0.06 g, 0.12 mmol), tris-tert-butyl phosphine (0.06 mL, 0.24 mmol), and sodium tert-butoxide (1.4 g, 15 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 45-c (white solid, 9 g, 65%).

ESI-LCMS for Intermediate 45-c: [M]⁺: C₁₀₀H₇₂D₁₇N₃O₃. 1396.8004

### Synthesis of Compound 45

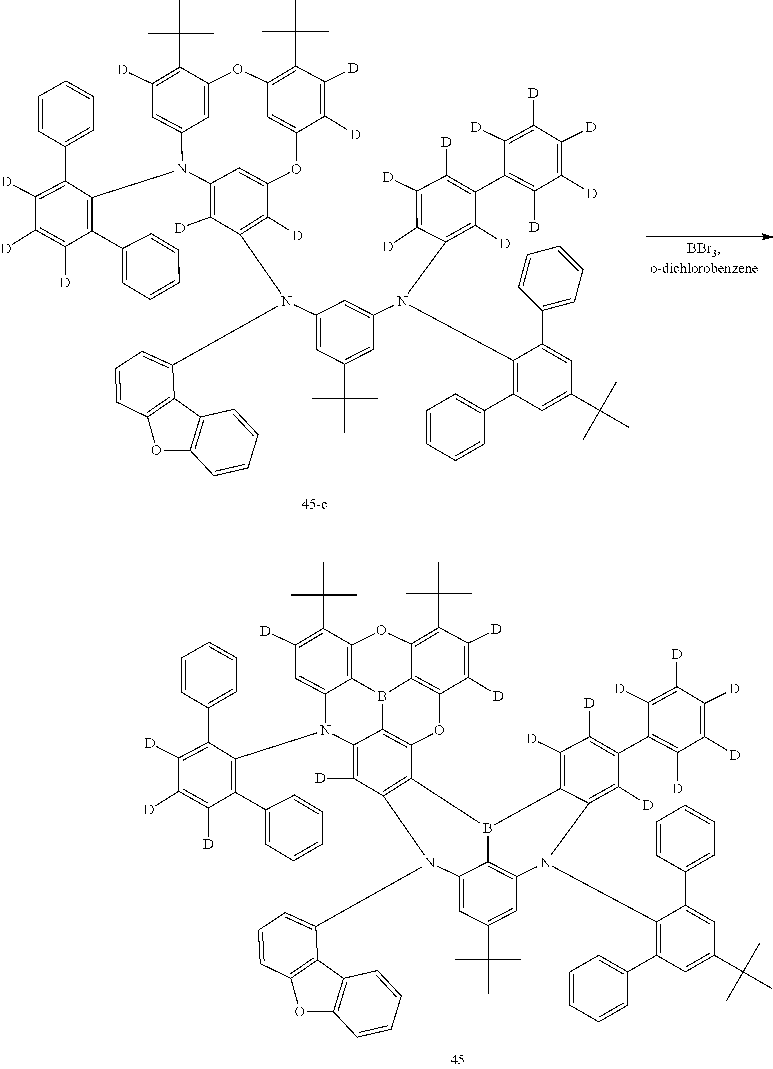

Under an argon atmosphere, Intermediate 45-c (9 g, 6.4 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 45 (yellow solid, 3.5 g, 39%).

ESI-LCMS for Compound 45: [M]⁺: C₁₀₀H₆₈D₁₅B₂N₃O₃. 1410.7667

¹H-NMR for Compound 45 (CDCl₃): δ=7.98 (d, 1H), 7.53 (s, 2H), 7.41 (m, 3H), 7.32 (m, 10H), 7.24 (m, 9H), 7.11 (m, 2H), 7.04 (m, 4H), 7.00 (s, 2H), 6.75 (s, 1H), 1.44 (s, 18H), 1.32 (s, 9H), 1.21 (s, 9H)

### Synthesis Example 10 (Synthesis of Compound 48)

### Synthesis of Intermediate 48-a

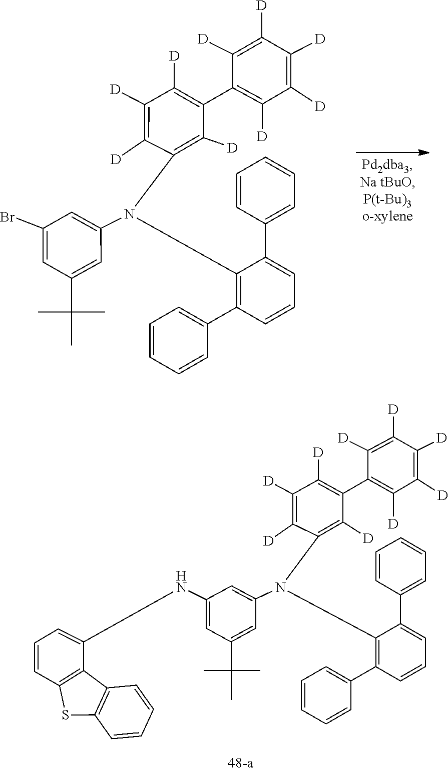 [SMILES: [2H]c1c([2H])c([2H])c(-c2c([2H])c([2H])c([2H])c(N(c3cc(Br)cc(C(C)(C)C)c3)c3c(-c4ccccc4)cccc3-c3ccccc3)c2[2H])c([2H])c1[2H] ; [2H]c1c([2H])c([2H])c(-c2c([2H])c([2H])c([2H])c(N(c3cc(Nc4cccc5sc6ccccc6c45)cc(C(C)(C)C)c3)c3c(-c4ccccc4)cccc3-c3ccccc3)c2[2H])c([2H])c1[2H]]

Under an argon atmosphere, N-([1,1′-biphenyl]-3-yl-d9)-N-(3-bromo-5-(tert-butyl)phenyl)-[1,1′:3′,1″-terphenyl]-2′-amine (10 g, 16 mmol), dibenzo[b,d]thiophen-1-amine (3.2 g, 12.7 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 48-a (white solid, 9 g, 77%).

ESI-LCMS for Intermediate 48-a: [M]⁺: C₅₂H₃₃D₉N₂S. 735.3668

### Synthesis of Intermediate 48-b

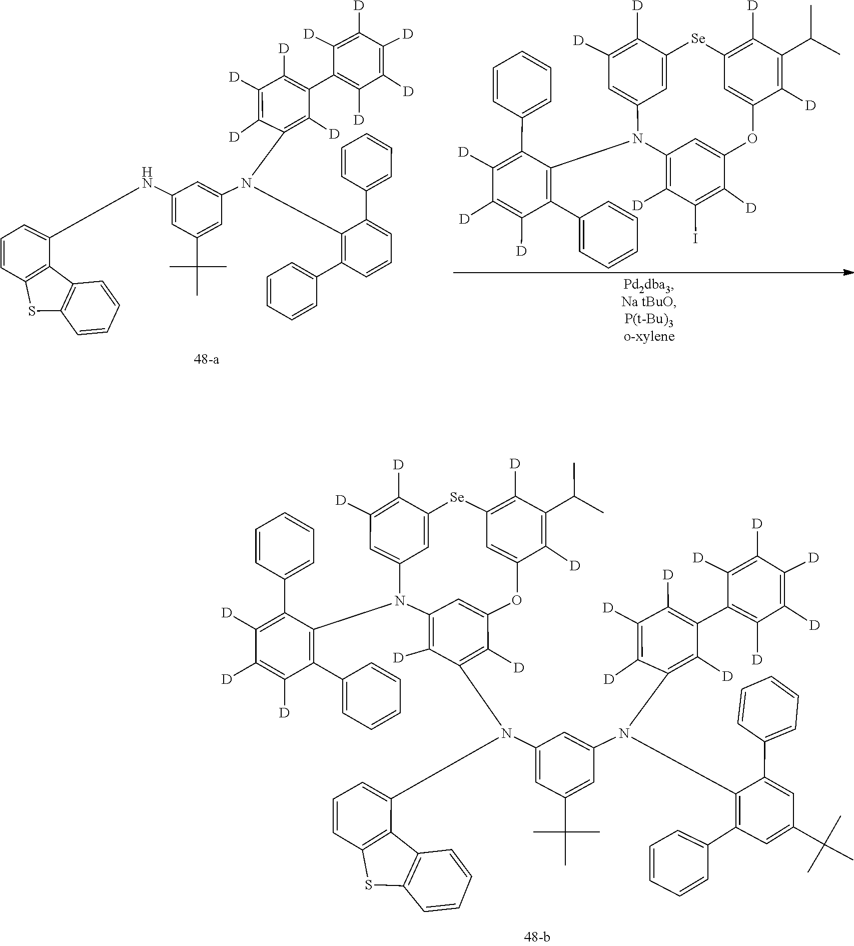

Under an argon atmosphere, Intermediate 48-a (10 g, 12.7 mmol), 4-([1,1′:3′,1″-terphenyl]-2′-yl-4′,5′,6′-d3)-35-iodo-15-isopropyl-2-oxa-6-selena-4-aza-1,3,5(1,3)-tribenzenacyclohexaphane-14,16,34,36,54,55-d6 (9.5 g, 12.7 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 48-b (white solid, 10.7 g, 60%).

ESI-LCMS for Intermediate 48-b: [M]⁺: C₉₅H₆₁D₁₈N₃OSSe. 1407.6212

### Synthesis of Compound 48

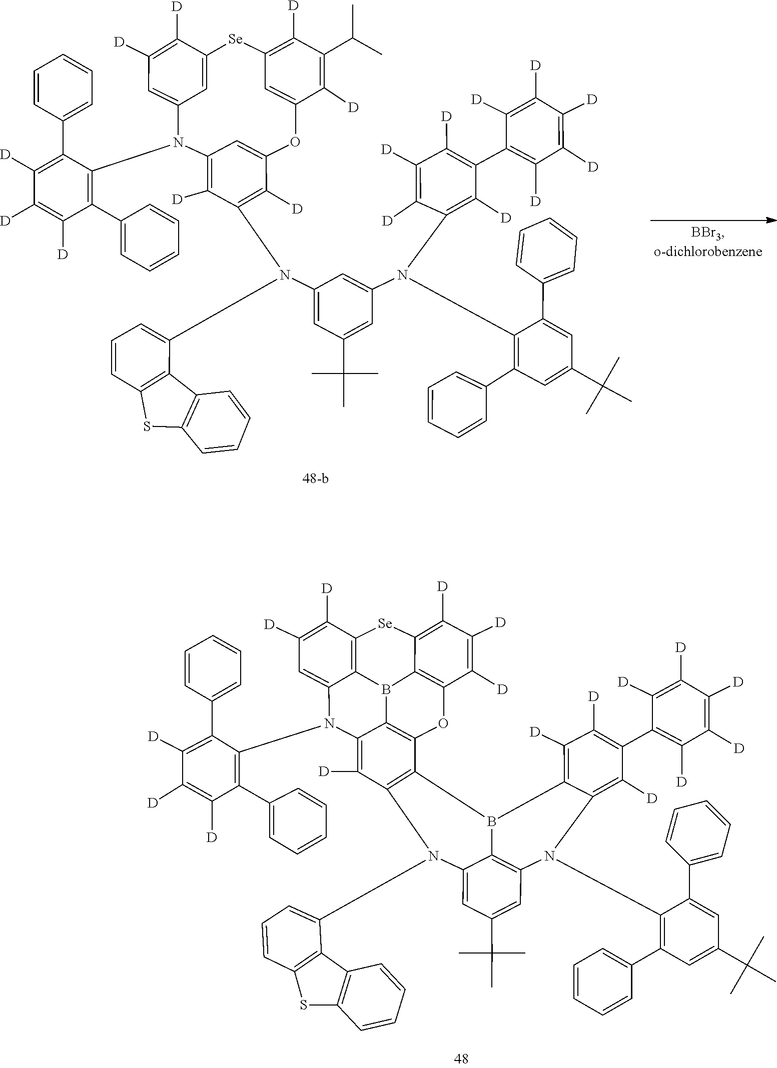

Under an argon atmosphere, Intermediate 48-b (109 g, 7.1 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 48 (yellow solid, 1.9 g, 19%).

ESI-LCMS for Compound 48: [M]⁺: C₉₅H₅₇D₁₆B₂N₃OSSe. 1421.5517

¹H-NMR for Compound 48 (CDCl₃): δ=8.45 (d, 1H), 7.93 (d, 1H), 7.59 (i, 5H), 7.41 (s, 2H), 7.36 (m, 16H), 7.21 (s, 1H), 7.06 (i, 8H), 7.00 (s, 2H), 2.88 (m, 1H), 1.33 (s, 9H), 1.21 (s, 18H), 1.12 (s, 6H)

### Synthesis Example 11 (Synthesis of Compound 53)

### Synthesis of Intermediate 53-a

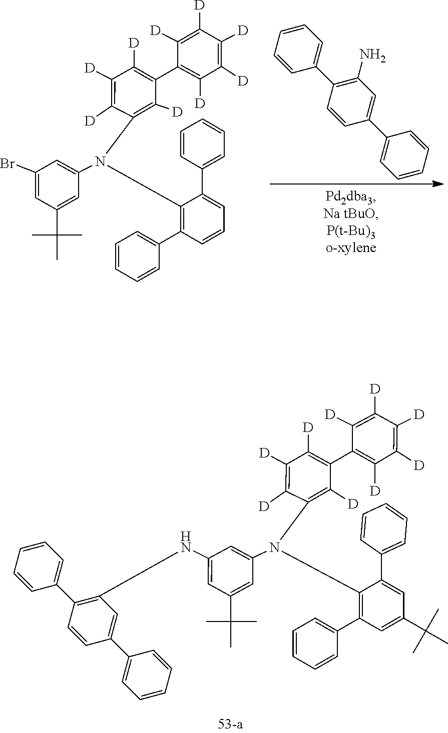 [SMILES: [2H]c1c([2H])c([2H])c(-c2c([2H])c([2H])c([2H])c(N(c3cc(Br)cc(C(C)(C)C)c3)c3c(-c4ccccc4)cccc3-c3ccccc3)c2[2H])c([2H])c1[2H] ; Nc1cc(-c2ccccc2)ccc1-c1ccccc1 ; [2H]c1c([2H])c([2H])c(-c2c([2H])c([2H])c([2H])c(N(c3cc(Nc4cc(-c5ccccc5)ccc4-c4ccccc4)cc(C(C)(C)C)c3)c3c(-c4ccccc4)cc(C(C)(C)C)cc3-c3ccccc3)c2[2H])c([2H])c1[2H]]

Under an argon atmosphere, N-([1,1′-biphenyl]-3-yl-d9)-N-(3-bromo-5-(tert-butyl)phenyl)-[1,1′:3′,1″-terphenyl]-2′-amine (10 g, 16 mmol), [1,1′:4′,1″-terphenyl]-2′-amine (3.9 g, 12.7 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 53-a (white solid, 9 g, 67%).

ESI-LCMS for Intermediate 53-a: [M]⁺: C₆₂H₄₇D₉N₂. 837.5001

### Synthesis of Intermediate 53-b

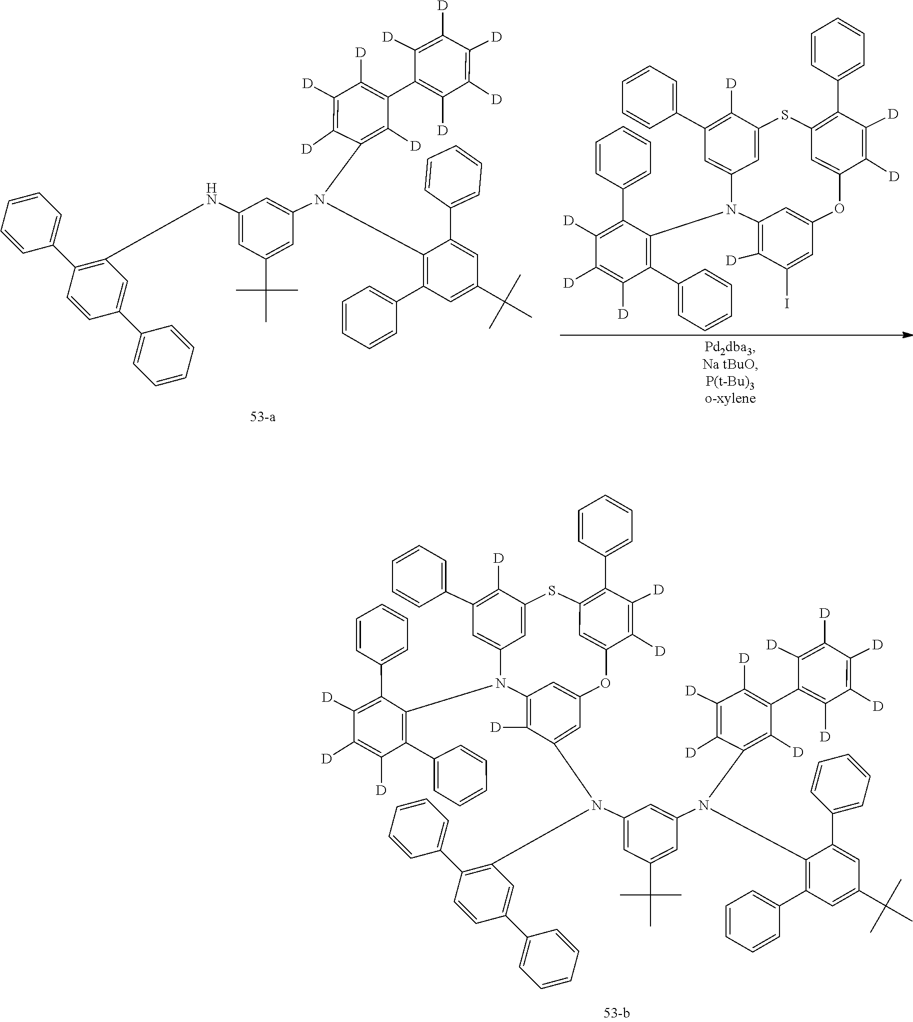

Under an argon atmosphere, Intermediate 53-a (9 g, 10.7 mmol), 4-([1,1′:3′,1″-terphenyl]-2″-yl-4′,5′,6′-d3)-35-iodo-14,55-diphenyl-2-oxa-6-thia-4-aza-1,3,5(1,3)-tribenzenacyclohexaphane-15,16,34,54-d4 (8.7 g, 10.7 mmol), Pd₂dba₃ (0.06 g, 0.12 mmol), tris-tert-butyl phosphine (0.06 mL, 0.24 mmol), and sodium tert-butoxide (4.3 g, 45 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 53-b (white solid, 10.2 g, 63%).

ESI-LCMS Intermediate 53-b: [M]⁺: C₁₁₀H₇₁D₁₆N₃OS. 1513.7618

### Synthesis of Compound 53

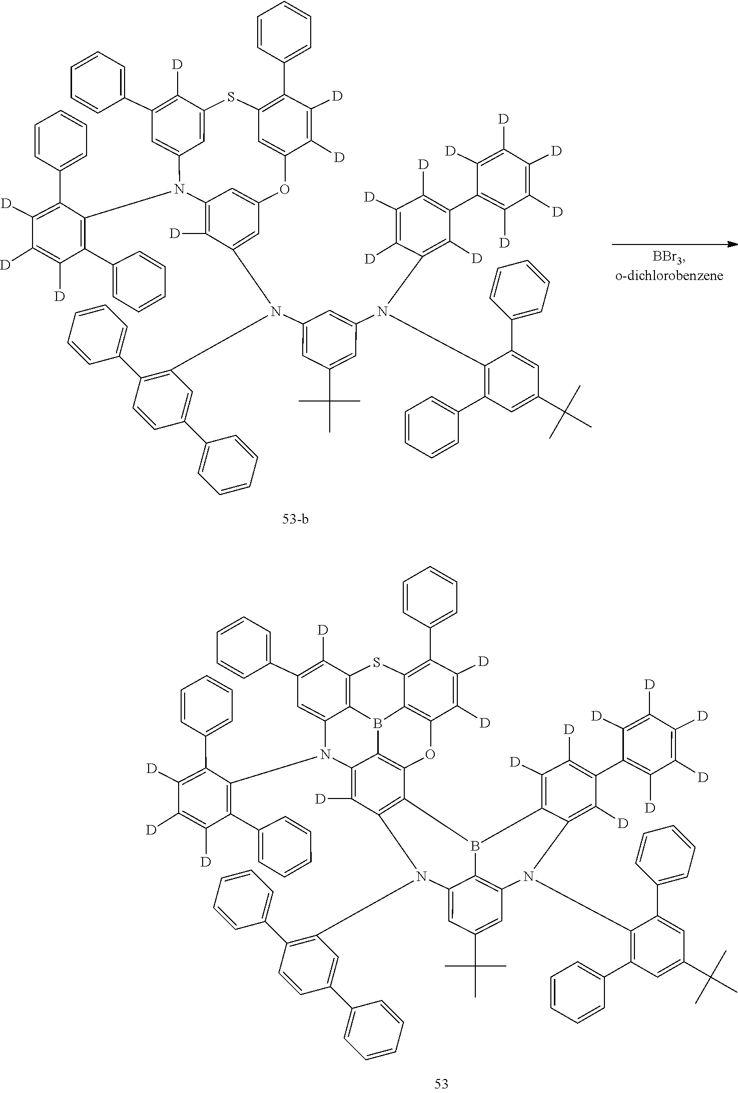

Under an argon atmosphere, Intermediate 53-b (10 g, 6.6 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 53 (yellow solid, 2.1 g, 21%).

ESI-LCMS for Compound 53: [M]⁺: C₁₁₀H₆₆D₁₅B₂NOS. 1528.7271

¹H-NMR for Compound 53 (CDCl₃): δ=7.92 (d, 1H), 7.75 (d, 2H), 7.51 (m, 8H), 7.41 (s, 2H), 7.36 (m, 25H), 7.08 (m, 6H), 7.01 (s, 2H), 6.91 (d, 1H), 1.32 (s, 9H), 1.24 (s, 9H)

### Synthesis Example 12 (Synthesis of Compound 74)

### Synthesis of Intermediate 74-a

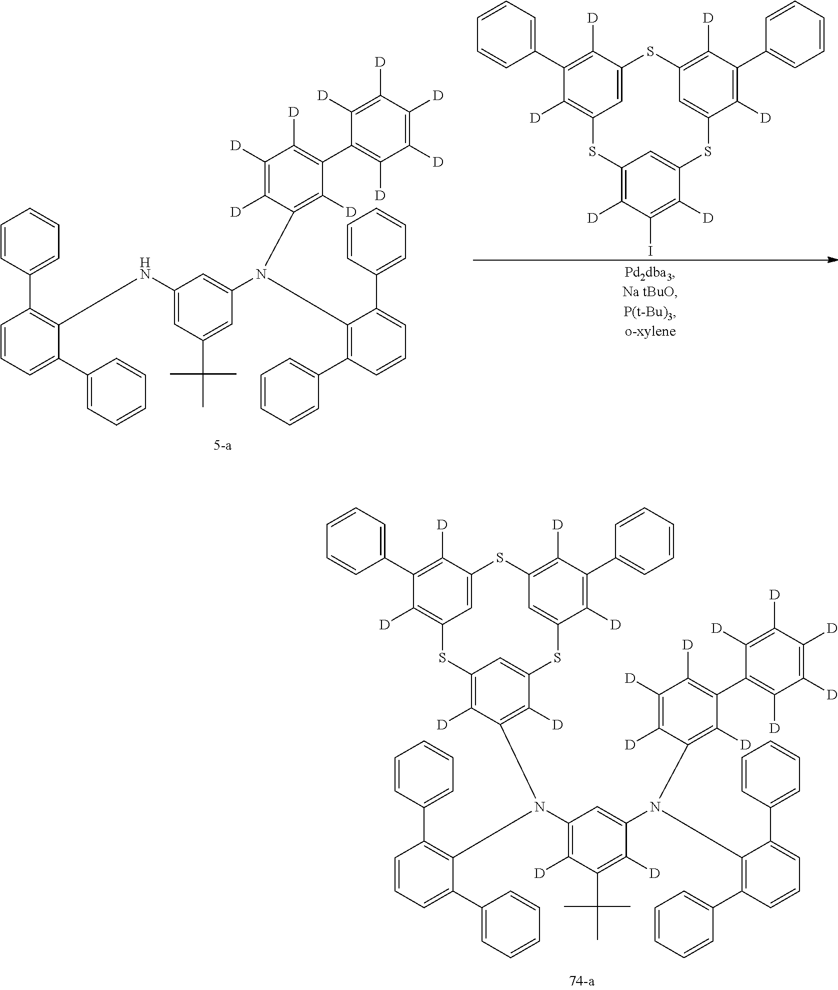 [SMILES: [2H]c1c([2H])c([2H])c(-c2c([2H])c([2H])c([2H])c(N(c3cc(Nc4c(-c5ccccc5)cccc4-c4ccccc4)cc(C(C)(C)C)c3)c3c(-c4ccccc4)cccc3-c3ccccc3)c2[2H])c([2H])c1[2H] ; [2H]c1c2cc(c([2H])c1I)Sc1cc(c([2H])c(-c3ccccc3)c1[2H])Sc1cc(c([2H])c(-c3ccccc3)c1[2H])S2 ; [2H]c1c([2H])c([2H])c(-c2c([2H])c([2H])c([2H])c(N(c3cc(N(c4c(-c5ccccc5)cccc4-c4ccccc4)c4c([2H])c5cc(c4[2H])Sc4cc(c([2H])c(-c6ccccc6)c4[2H])Sc4cc(c([2H])c(-c6ccccc6)c4[2H])S5)c([2H])c(C(C)(C)C)c3[2H])c3c(-c4ccccc4)cccc3-c3ccccc3)c2[2H])c([2H])c1[2H]]

Under an argon atmosphere, Intermediate 5-a (10 g, 13 mmol), 15-iodo-35,55-diphenyl-2,4,6-trithia-1,3,5(1,3)-tribenzenacyclohexaphane-14,16,34,36,54,56-d6 (7.9 g, 13 mmol), Pd₂dba₃ (0.06 g, 0.12 mmol), tris-tert-butyl phosphine (0.06 mL, 0.24 mmol), and sodium tert-butoxide (1.4 g, 15 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 74-a (white solid, 10 g, 61%).

ESI-LCMS for Intermediate 74-a: [M]⁺: C₈₈H₄₉D₁₇N₂S₃. 1263.5560

### Synthesis of Compound 74

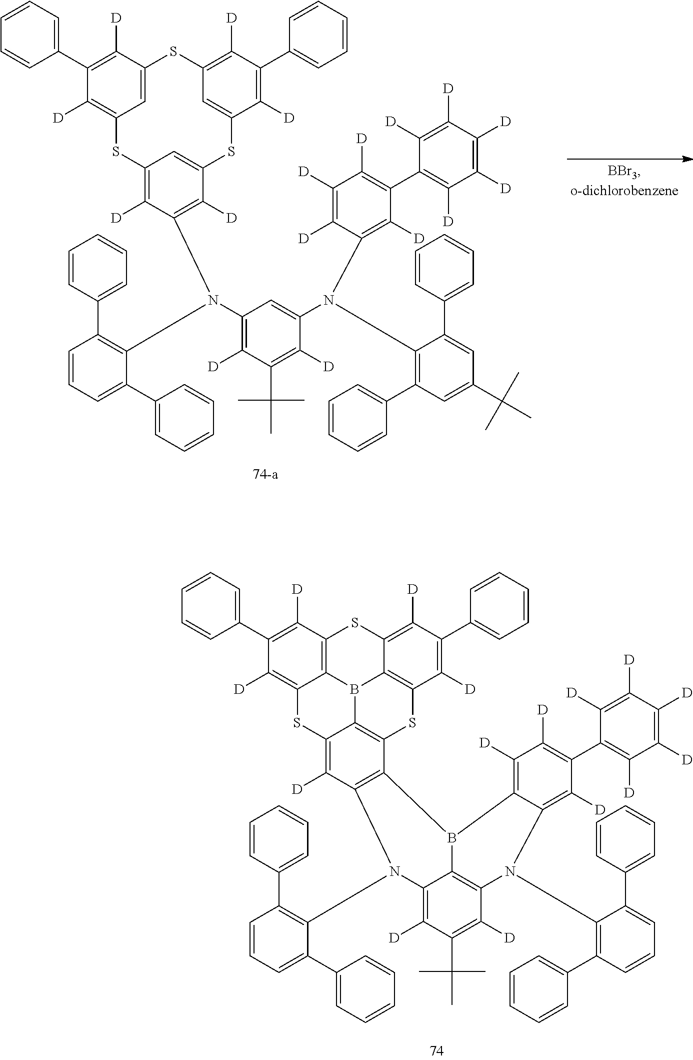

Under an argon atmosphere, Intermediate 74-a (10 g, 7.9 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 74 (yellow solid, 3.5 g, 32%).

ESI-LCMS for Compound 74: [M]⁺: C₈₈H₄₅D₁₅B₂N₂S₃. 1277.4937

¹H-NMR for Compound 74 (CDCl₃): δ=8.20 (d, 4H), 7.51 (m, 10H), 7.43 (m, 12H), 7.31 (m, 2H), 7.07 (m, 8H), 1.27 (s, 9H)

### Synthesis Example 13 (Synthesis of Compound 80)

### Synthesis of Intermediate 80-a

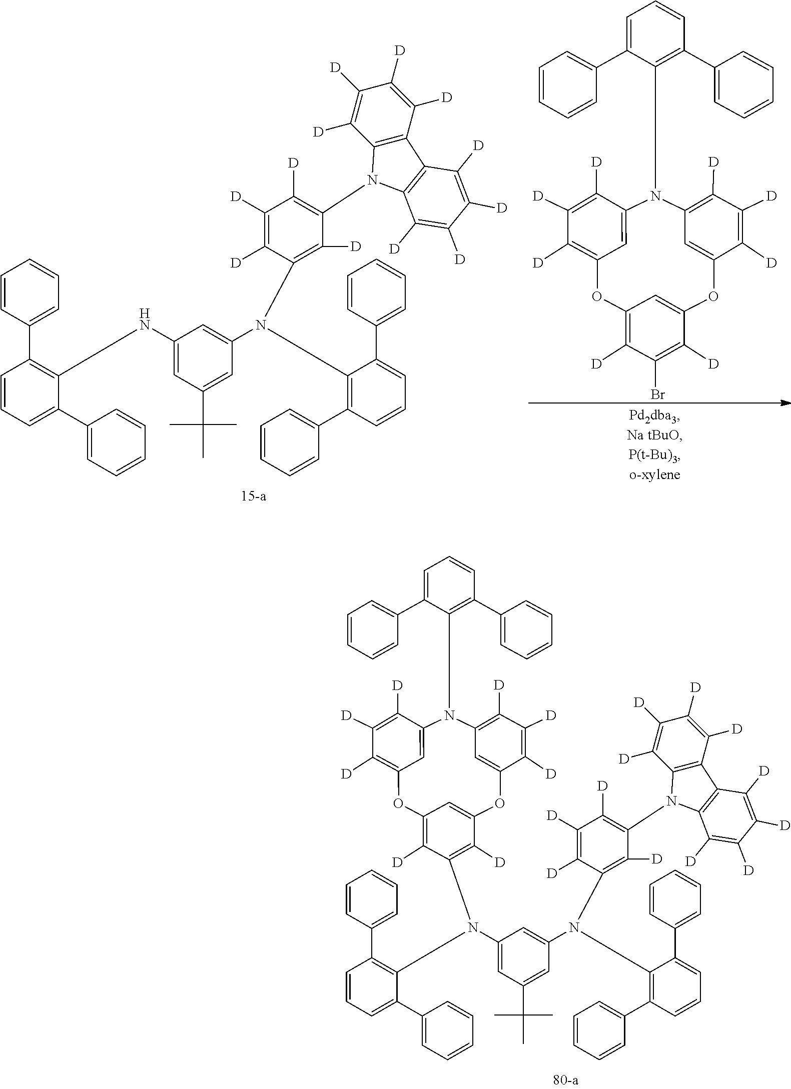

Under an argon atmosphere, Intermediate 15-a (10 g, 11 mmol), 6-([1,1′:3′,1″-terphenyl]-2″-yl)-35-bromo-2,4-dioxa-6-aza-1,3,5(1,3)-tribenzenacyclohexaphane-14,15,16,34,36,54,55,56-d8 (6.8 g, 11 mmol), Pd₂dba₃ (0.06 g, 0.12 mmol), tris-tert-butyl phosphine (0.06 mL, 0.24 mmol), and sodium tert-butoxide (1.4 g, 15 mmol) were placed in a 1 L flask and then dissolved in 100 mL of xylene, and then, the reaction solution was stirred at 140° C. for 12 hours. After cooling the mixture, water (1 L) and ethyl acetate (300 mL) were added for extraction to collect an organic layer, and then, the organic layer was dried with MgSO₄ and filtered. The solvent was removed from filtrate under reduced pressure, and the obtained solid was purified and separated by column chromatography using silica gel by using CH₂Cl₂ and hexane as developing solvents, to obtain Intermediate 80-a (white solid, 11 g, 73%).

ESI-LCMS for Intermediate 80-a: [M]⁺: C₁₀₀H₅₄D₂₀N₄O₂. 1382.7727

### Synthesis of Compound 80

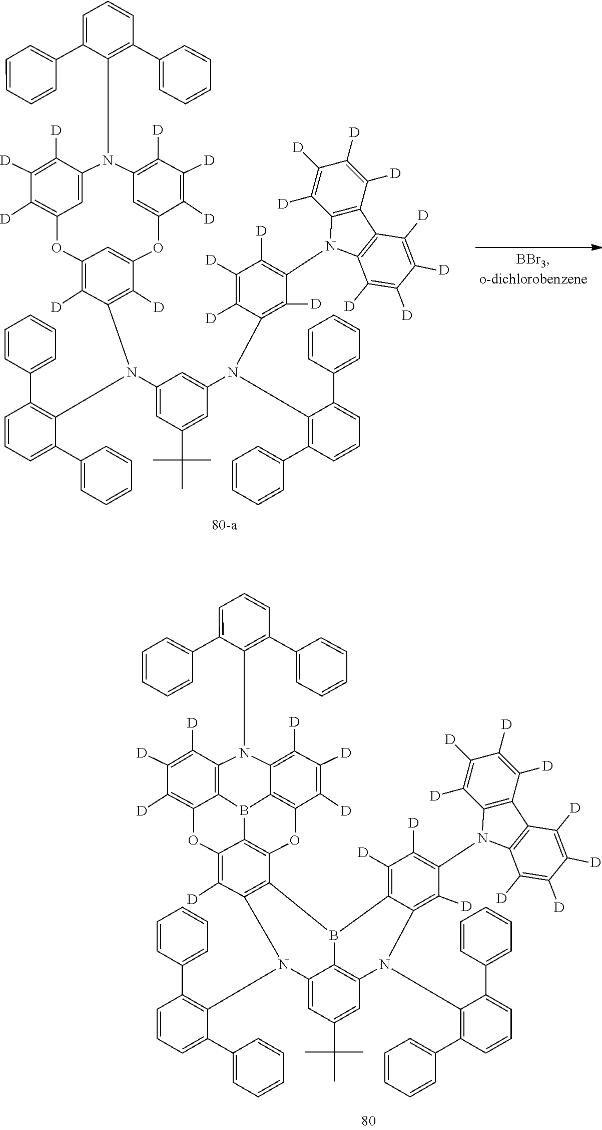

Under an argon atmosphere, Intermediate 80-a (11 g, 8 mmol) was placed in a 1 L flask and then dissolved in 200 mL of o-dichlorobenzene, and then, BBr₃ (2 equiv.) was added. The reaction solution was stirred at 140° C. for 12 hours. After cooling the mixture, triethylamine was added to terminate the reaction and the solvent was removed under reduced pressure, and the obtained solid was purified and separated by column chromatography using silica gel by using CH₂Cl₂ and hexane as developing agents, to obtain Compound 80 (yellow solid, 3 g, 27%).

ESI-LCMS for Compound 80: [M]⁺: C₁₀₀H₅₀D₁₈B₂N₄O₂. 1396.6768

¹H-NMR for Compound 80 (CDCl₃): δ=8.23 (d, 4H), 8.19 (d, 2H), 7.56 (m, 2H), 7.38 (m, 1H), 7.44 (m, 12H), 7.36 (m, 6H), 7.22 (m, 8H), 7.14 (m, 4H), 7.00 (m, 2H), 1.29 (s, 9H) Evaluation Example 1

The highest occupied molecular orbital (HOMO) energy level, absorption wavelength (λ_{abs}) in solution, emission wavelength (λₑₘᵢ) in solution, molar extinction coefficient (s), photoluminescence quantum yield (PLQY), and delayed fluorescence lifespan (TD) of each of Compounds 5, 7, 8, 13, 14, 15, 20, 37, 45, 48, 53, 74, and 80 and Comparative Compounds CE1 to CE4, which were synthesized in the preceding synthesis examples, were measured, and results thereof are shown in Table 1.

The HOMO energy level was measured by using the Smart Manager software of the SP2 electrochemical workstation from ZIVE LAB.

The absorption wavelength and molar extinction coefficient in solution was measured by using the Labsolution UV-Vis software in such a state that the UV-1800 UV/Visible Scanning Spectrophotometer from SHIMADZU was equipped with a deuterium/tungsten-halogen light source and a silicon photodiode.

The emission wavelength in solution was measured by using the FluorEssence software in such a state that a xenon light source and a monochromator were mounted on the HORIBA fluoromax+ spectrometer.

PLQY was measured by using PLQY measurement software in which a xenon light source, a monochromator, a photonic multi-channel analyzer, and an integrating sphere were mounted on a Quantaurus-QY Absolute PL quantum yield spectrometer from Hamamatsu.

The delayed fluorescence lifespan was measured at 300 K by using a fluorescence lifespan measurement device (C11367-01) from Hamamatsu.

TABLE 1

| | HOMO | λ_{abs} | λₑₘᵢ | PLQY | ε | _{T}D |
|---|---|---|---|---|---|---|
| Compound | (eV) | (nm) | (nm) | (%) | (cm⁻¹M⁻¹) | (μs) |
| Compound 5 | −5.50 | 449 | 459 | 98 | 90,000 | 7 |
| Compound 7 | −5.53 | 451 | 460 | 99 | 99,000 | 10 |
| Compound 8 | −5.45 | 445 | 453 | 99 | 95,000 | 12 |
| Compound 13 | −5.43 | 457 | 464 | 97 | 100,500 | 9 |
| Compound 14 | −5.46 | 454 | 462 | 99 | 89,000 | 11 |
| Compound 15 | −5.47 | 452 | 462 | 99 | 94,000 | 5 |
| Compound 20 | −5.45 | 450 | 460 | 99 | 110,000 | 6 |
| Compound 37 | −5.43 | 445 | 455 | 99 | 100,000 | 5 |
| Compound 45 | −5.40 | 446 | 457 | 97 | 87,000 | 9 |
| Compound 48 | −5.42 | 443 | 453 | 98 | 99,000 | 6 |
| Compound 53 | −5.45 | 445 | 455 | 97 | 100,000 | 7 |
| Compound 74 | −5.42 | 447 | 457 | 98 | 91,000 | 6 |
| Compound 80 | −5.49 | 451 | 458 | 99 | 105,000 | 2 |
| Comparative | −5.05 | 456 | 466 | 84 | 85,000 | 15 |
| Compound CE1 | | | | | | |
| Comparative | −5.11 | 449 | 463 | 69 | 43,000 | 21 |
| Compound CE2 | | | | | | |
| Comparative | −5.07 | 438 | 450 | 74 | 36,000 | 37 |
| Compound CE3 | | | | | | |
| Comparative | −5.03 | 429 | 448 | 65 | 47,000 | 89 |
| Compound CE4 | | | | | | |

### Example Compounds

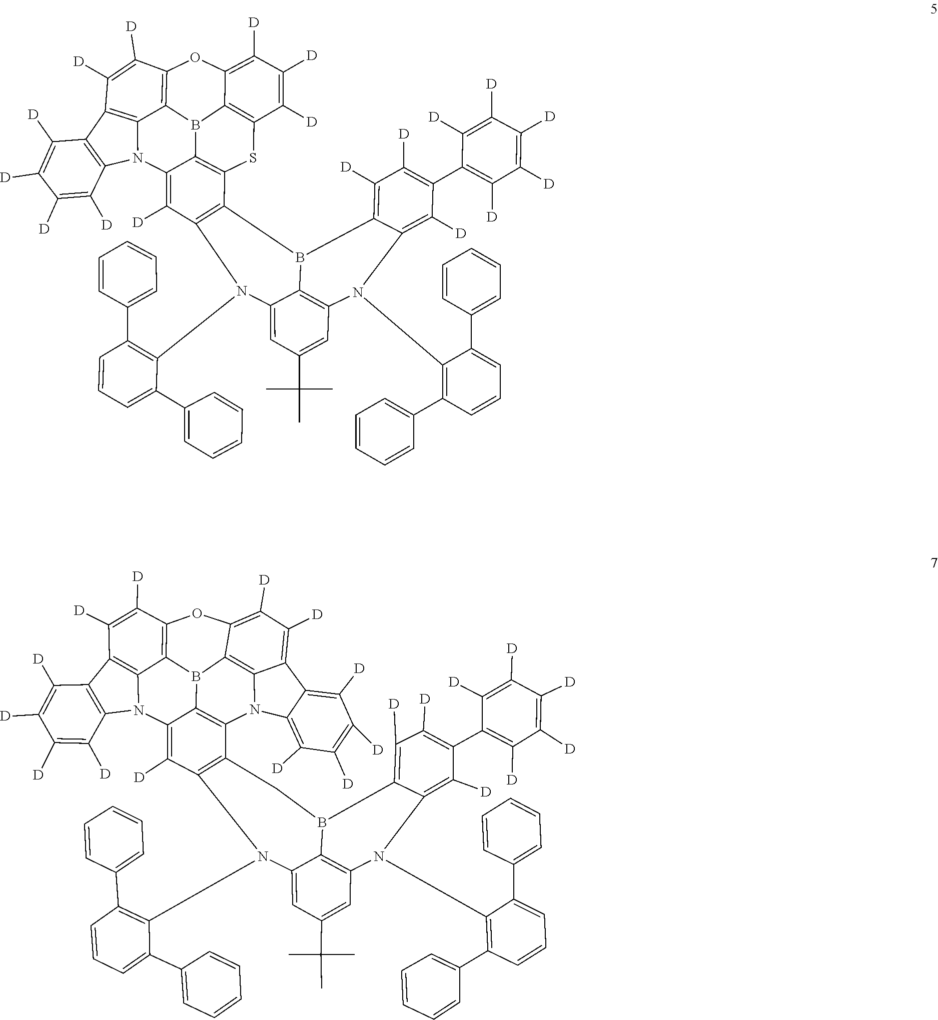

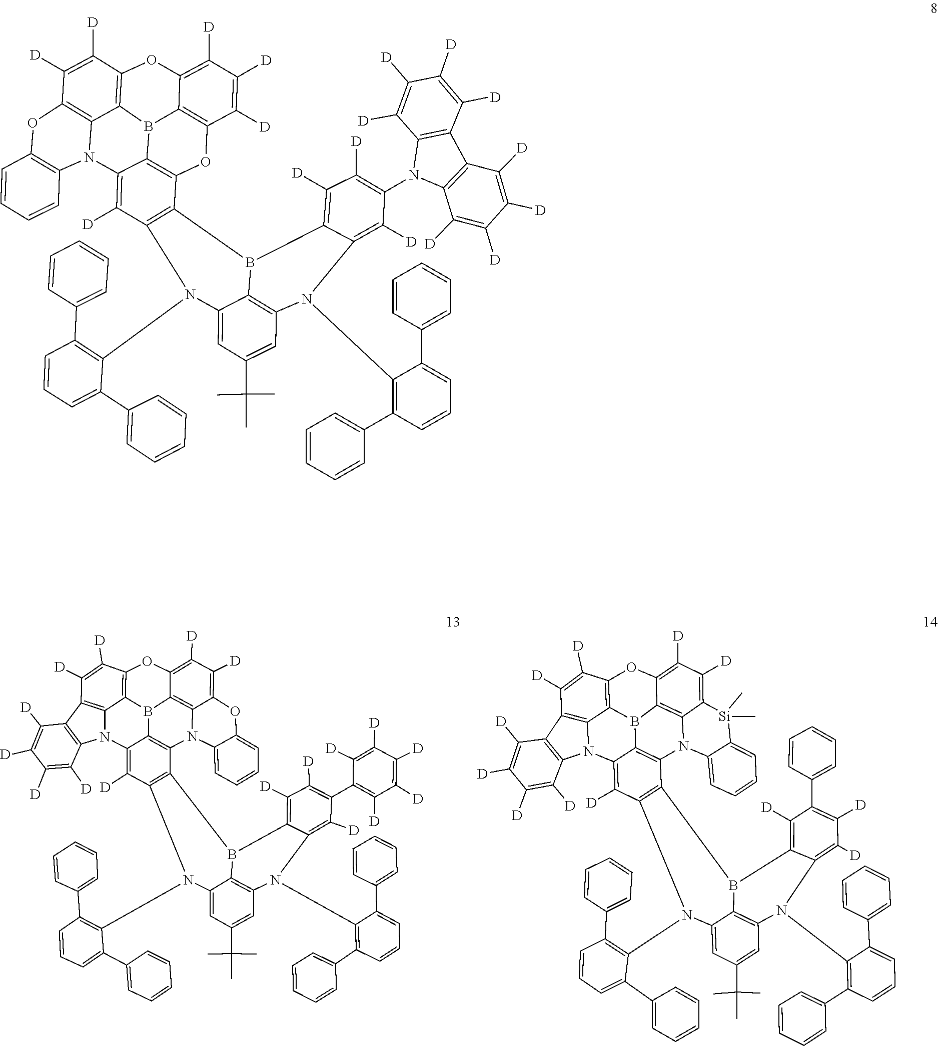

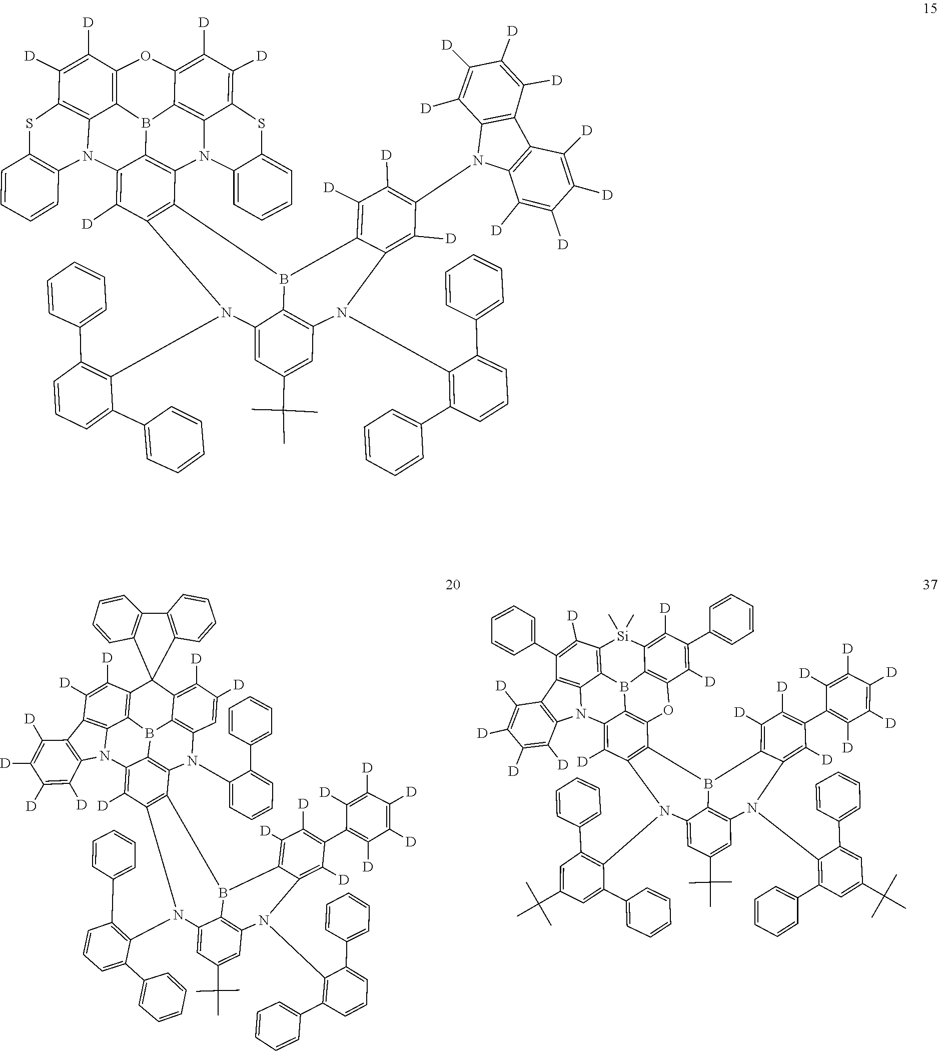

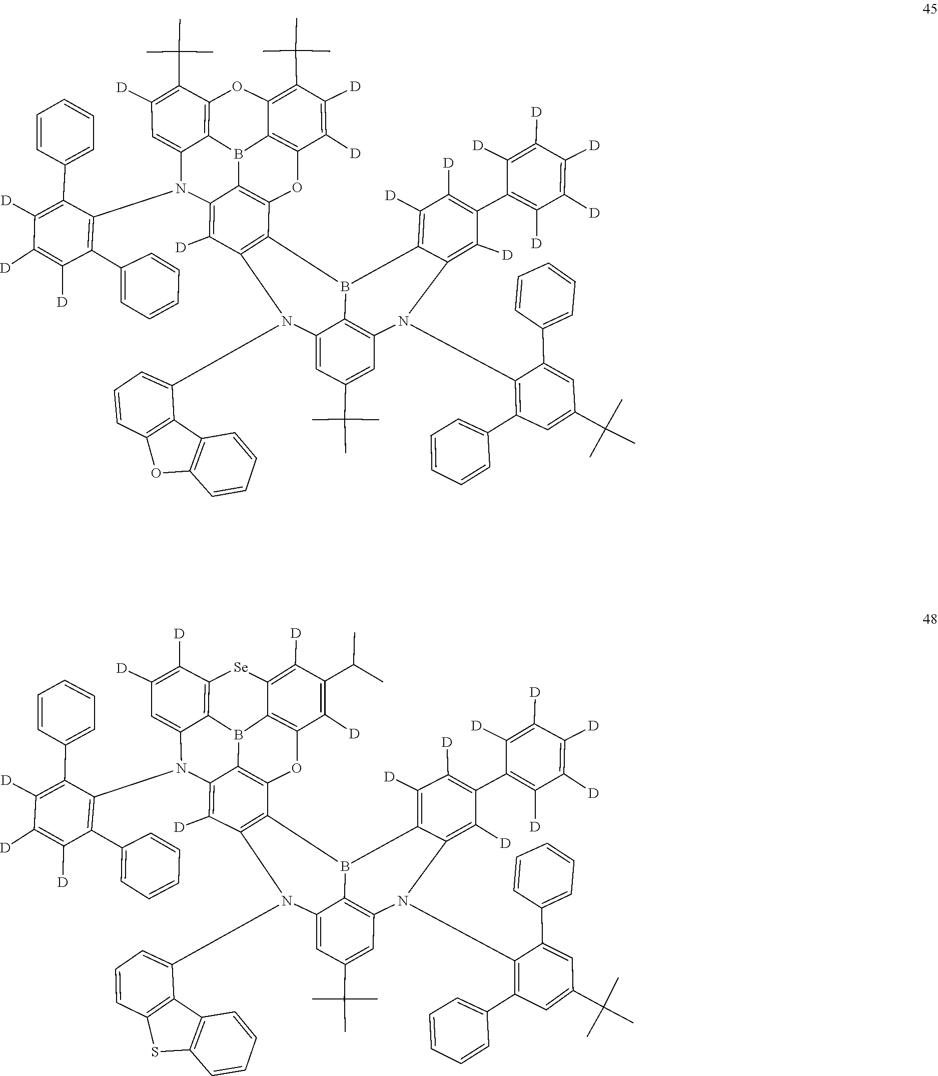

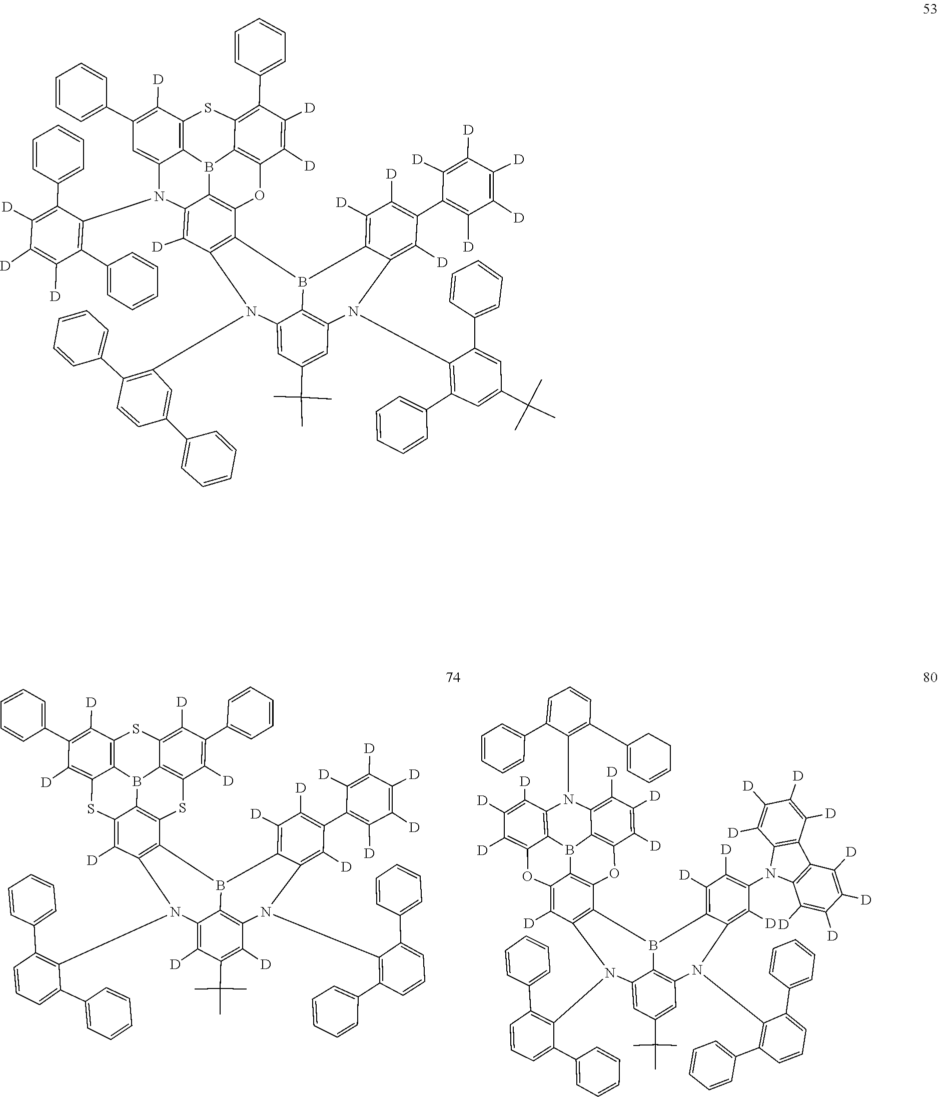

### Comparative Compounds

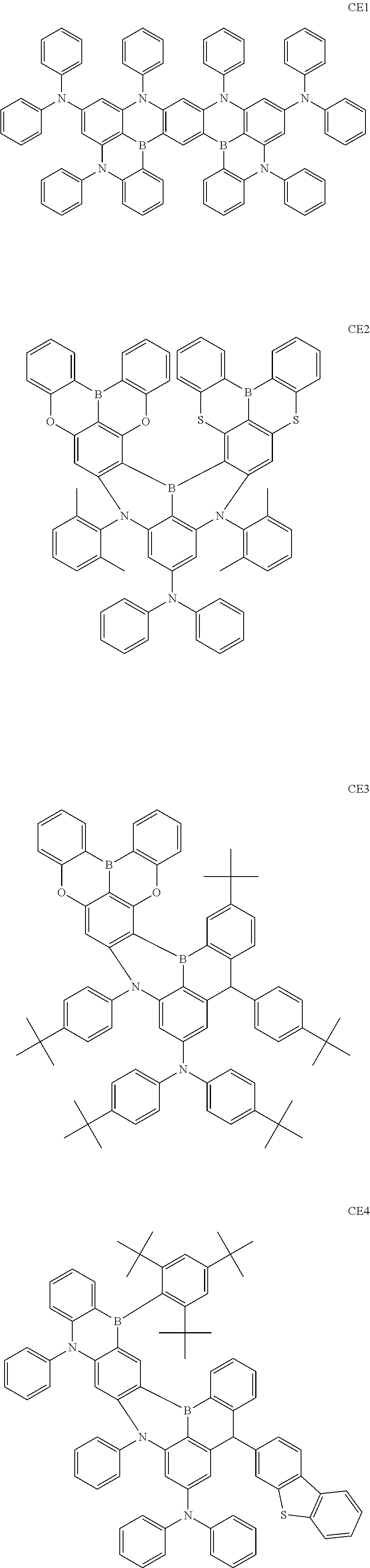

From Table 1, it was confirmed that each of Compounds 5, 7, 8, 13, 14, 15, 20, 37, 45, 48, 53, 74 and 80, which are represented by Formula 1, had a higher PLQY, a higher molar extinction coefficient, and lower delayed fluorescence lifespan than each of Comparative Compounds CE1 to CE4 that are not represented by Formula 1. Therefore, the compound represented by Formula 1 has high luminescence efficiency and emits prompt delayed fluorescence, and thus, is suitable for use as a thermally activated delayed fluorescence (TADF) material.

### Example 1

As an anode, a glass substrate (product of Corning Inc.) with a 15 ohm per square centimeter (0/cm²) (1,200 angstrom (A)) indium tin oxide (ITO) electrode formed thereon was cut to a size of 50 millimeter (mm)×50 mm×0.7 mm, sonicated by using isopropyl alcohol and pure water each for 5 minutes, washed by irradiation of ultraviolet rays and exposure of ozone thereto for 30 minutes, and then mounted on a vacuum deposition apparatus.

NPD was vacuum-deposited on the anode to form a hole injection layer having a thickness of 300 Å. HT6 was vacuum-deposited on the hole injection layer to form a hole transport layer having a thickness of 200 Å. CzSi was vacuum-deposited on the hole transport layer to form an electron blocking layer having a thickness of 100 Å.

A host mixture in which ETH2 (second compound) and HT-1 (third compound) were mixed at a ratio of 1:1, PS-1 (fourth compound, sensitizer), and Compound 5 (organic Compound) were vacuum-co-deposited on the electron blocking layer at a weight ratio of 85:14:1 to form an emission layer having a thickness of 200 Å.

TSPO1 was vacuum-deposited on the emission layer to form a hole blocking layer having a thickness of 200 Å. TPBi was vacuum-deposited on the hole blocking layer to form an electron transport layer having a thickness of 300 Å. LiF was vacuum-deposited on the electron transport layer to form an electron injection layer having a thickness of 10 Å, and then, Al was vacuum-deposited thereon to form a second electrode having a thickness of 3,000 Å, thereby forming an LiF/Al electrode. P4 was vacuum-deposited on the second electrode to form a capping layer having a thickness of 700 Å, thereby completing manufacture of a light-emitting device.

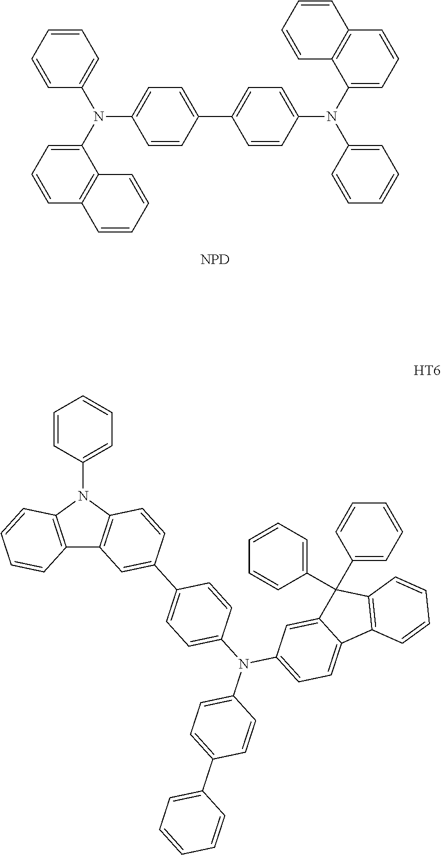 [SMILES: c1ccc(N(c2ccc(-c3ccc(N(c4ccccc4)c4cccc5ccccc45)cc3)cc2)c2cccc3ccccc23)cc1 ; "NPD" ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

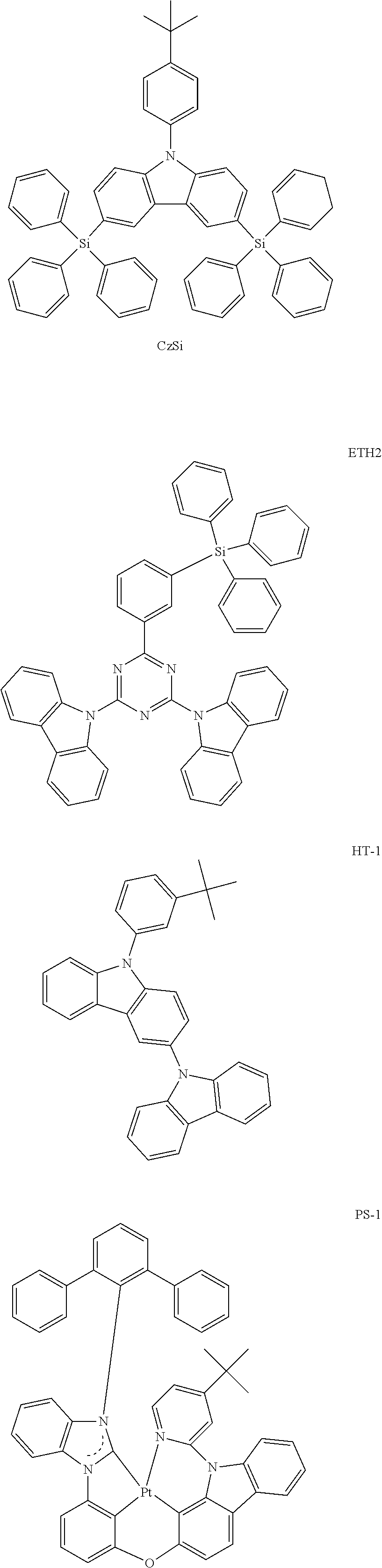

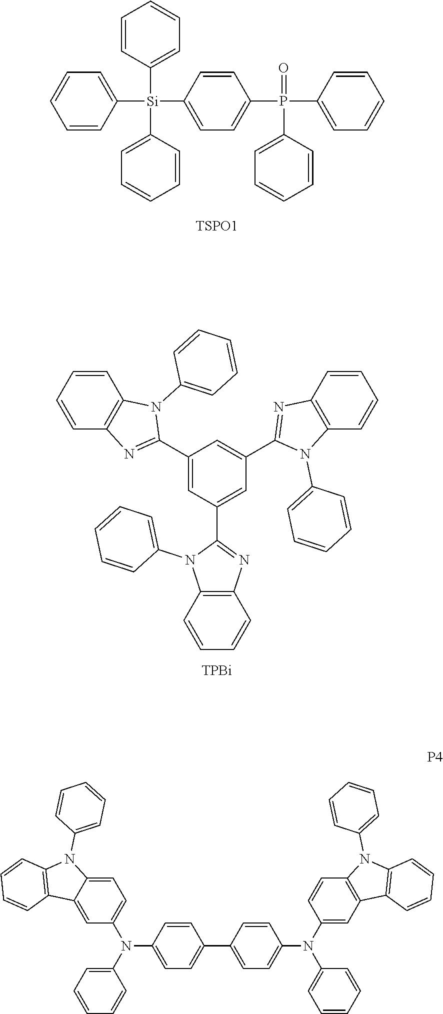

### Examples 2 to 13 and Comparative Examples 1 to 4

Light-emitting devices were manufactured in substantially the same manner as in Example 1, except that the compounds shown in Table 2 were used instead of Compound 5 in forming the emission layer.

### Evaluation Example 2

The driving voltage, luminescence efficiency, emission wavelength, lifespan, and color purity (CIEy coordinate) of each of the light-emitting devices manufactured in Examples 1 to 13 and Comparative Examples 1 to 4 were each measured at a current density of 10 milliampere per square centimeter (mA/cm²) by using the V7000 OLED IVL Test System (Polaronix), and results thereof are shown in Table 2. Lifespan (T₉₅) in Table 2 is a measure of the time (Hr) taken until the luminance reaches 95% relative to the initial luminance, and is expressed as a relative value based on the lifespan of Comparative Example 1.

TABLE 2

| | | | Lumine- | Emis- | | |
|---|---|---|---|---|---|---|
| | | | scence | sion | | |
| | | Driving | effici- | wave- | Life- | |
| | Organic | voltage | ency | length | span | |
| No. | compound | (V) | (cd/A/y) | (nm) | (T95) | CIEy |
| Example 1 | Compound 5 | 3.8 | 550 | 461 | 9.8 | 0.052 |
| Example 2 | Compound 7 | 3.7 | 565 | 462 | 11.7 | 0.053 |
| Example 3 | Compound 8 | 3.6 | 550 | 455 | 5.9 | 0.044 |
| Example 4 | Compound 13 | 3.5 | 570 | 466 | 7.2 | 0.059 |
| Example 5 | Compound 14 | 3.3 | 550 | 463 | 8.8 | 0.057 |
| Example 6 | Compound 15 | 3.8 | 580 | 463 | 6.5 | 0.055 |
| Example 7 | Compound 20 | 3.7 | 595 | 461 | 5.5 | 0.050 |
| Example 8 | Compound 37 | 3.6 | 560 | 456 | 9.4 | 0.045 |
| Example 9 | Compound 45 | 3.7 | 540 | 460 | 10.4 | 0.050 |
| Example 10 | Compound 48 | 3.5 | 520 | 455 | 7.2 | 0.041 |
| Example 11 | Compound 53 | 3.4 | 580 | 456 | 6.9 | 0.043 |
| Example 12 | Compound 74 | 3.8 | 560 | 460 | 8.7 | 0.051 |
| Example 13 | Compound 80 | 3.7 | 550 | 460 | 12.5 | 0.050 |
| Comparative | Comparative | 4.4 | 350 | 470 | 1 | 0.067 |
| Example 1 | Compound | | | | | |
| | CE1 | | | | | |
| Comparative | Comparative | 4.0 | 450 | 465 | 0.1 | 0.057 |
| Example 2 | Compound | | | | | |
| | CE2 | | | | | |
| Comparative | Comparative | 4.1 | 320 | 453 | 0.2 | 0.043 |
| Example 3 | Compound | | | | | |
| | CE3 | | | | | |
| Comparative | Comparative | 4.5 | 270 | 451 | 0.08 | 0.039 |
| Example 4 | Compound | | | | | |
| | CE4 | | | | | |

From Table 2, it was confirmed that Examples 1 to 13 respectively employing Compounds 5, 7, 8, 13, 14, 15, 20, 37, 45, 48, 53, 74 and 80, which are represented by Formula 1 and have high PLOY values, high molar extinction coefficients, and low delayed fluorescence lifespan characteristics, each had a higher luminescence efficiency and a longer lifespan than Comparative Examples 1 to 4 respectively employing Comparative Compounds CE1 to CE4 that are not represented by Formula 1.

Without being bound by any particular theory, it is believed that the organic compound represented by Formula 1 undergoes enhancement of a multiple-resonance (MR) effect and reinforcement of structural rigidity, leading to induction of a distortion effect. As a result, an local excited state (LE state) and a charge transfer state (CT state) are mixed, and thus, an orbital distribution between a lowest excited singlet state (S1) and a lowest excited triplet state (T1) may be changed. Therefore, spin-orbit coupling is strengthened, and thus, an inhibitory aspect according to the EI-Sayed rule may be offset, and reverse intersystem crossing (RISC) may be induced. For example, the organic compound may concurrently (e.g., simultaneously) have a high photoluminescence quantum yield (PLQY), a high molar extinction coefficient, and low delayed fluorescence lifespan, and the luminescence efficiency and lifespan of a light-emitting device including the organic compound may be improved.

It should be understood that embodiments described herein should be considered in a descriptive sense only and not for purposes of limitation. Descriptions of features or aspects within each embodiment should typically be considered as available for other similar features or aspects in one or more embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode opposite to the first electrode; and
an interlayer between the first electrode and the second electrode and comprising an emission layer,
wherein the interlayer comprises an organic compound represented by Formula 1: 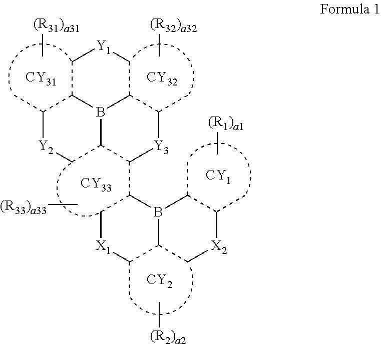
wherein, in Formula 1,
X₁ is selected from among O, S, Se, C(Ar₁₁)(Ar₁₂), Si(Ar₁₁)(Ar₁₂), and N(Ar₁₁),
X₂ is selected from among O, S, Se, C(Ar₂₁)(Ar₂₂), Si(Ar₂₁)(Ar₂₂), and N(Ar₂₁),
Y₁ is selected from among O, S, Se, C(Z₁₁)(Z₁₂), Si(Z₁₁)(Z₁₂), and N(Z₁₁),
Y₂ is selected from among O, S, Se, C(Z₂₁)(Z₂₂), Si(Z₂₁)(Z₂₂), and N(Z₂₁),
Y₃ is selected from among O, S, Se, C(Z₃₁)(Z₃₂), Si(Z₃₁)(Z₃₂), and N(Z₃₁),
Z₁₁ and Z₁₂ are bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
Z₂₁ and Z₂₂ are bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
Z₃₁ and Z₃₂ are bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
ring CY₁, ring CY₂, and ring CY₃₁ to ring CY₃₃ are each independently a C₃-C₆₀ carbocyclic group or a C₁-C₆₀ heterocyclic group,
Ar₁₁, Ar₁₂, Ar₂₁, and Ar₂₂ are each independently a C₃-C₆₀ carbocyclic group that is unsubstituted or substituted with at least one R₁₀ₐ or a C₁-C₆₀ heterocyclic group that is unsubstituted or substituted with at least one R₁₀ₐ,
Z₁₁, Z₁₂, Z₂₁, Z₂₂, Z₃₁, Z₃₂, R₁, R₂, and R₃₁ to R₃₃ are each independently hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a2, and a31 to a33 are each independently an integer from 0 to 20,
at least one selected from among Z₁₁ and Z₁₂ is bonded to ring CY₃₁ or ring CY₃₂ via a single bond, *—O—*′, *—S—*′, *—Se—*′, *—C(T₁₁)(T₁₂)-*′, or *—Si(T₁₁)(T₁₂)-*′ or Z₁₁ and Z₁₂ are not bonded to ring CY₃₁ and ring CY₃₂,
at least one selected from among Z₂₁ and Z₂₂ is bonded to ring CY₃₁ or ring CY₃₃ via a single bond, *—O—*′, *—S—*′, *—Se—*, *—C(T₂₁)(T₂₂)-*′, or *—Si(T₂₁)(T₂₂)-*′ or Z₂₁ and Z₂₂ are not bonded to ring CY₃₁ and ring CY₃₃,
at least one selected from among Z₃₁ and Z₃₂ is bonded to ring CY₃₂ or ring CY₃₃ via a single bond, *—O—*′, *—S—*′, *—Se—*′, *—C(T₃₁)(T₃₂)-*′ or *—Si(T₃₁)(T₃₂)-*′ or Z₃₁ and Z₃₂ are not bonded to ring CY₃₂ and ring CY₃₃,
T₁₁, T₁₂, T₂₁, T₂₂, T₃₁, and T₃₂ are each independently hydrogen, deuterium, —F, a cyano group, a C₁-C₆₀ alkyl group, a C₁-C₆₀ alkoxy group, a C₆-C₆₀ aryl group, or a C₁-C₆₀ heteroaryl group,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂—C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

2. The light-emitting device of claim 1, further comprising a second compound comprising at least one π electron-deficient nitrogen-containing C₁-C₆₀ heterocyclic group, a third compound comprising a group represented by Formula 3, a fourth compound comprising a transition metal, or any combination thereof,
wherein the organic compound, the second compound, the third compound, and the fourth compound are different from each other: 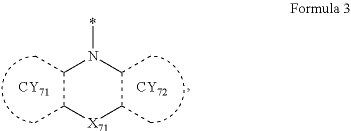
in Formula 3,
ring CY₇₁ and ring CY₇₂ are each independently a π electron-rich C₃-C₆₀ cyclic group or a pyridine group,
X₇₁ is a single bond, or a linking group comprising O, S, N, B, C, Si, or any combination thereof, and
* indicates a binding site to an atom of the third compound excluding an atom in Formula 3.

3. The light-emitting device of claim 2, wherein the second compound comprises a pyridine group, a pyrimidine group, a pyrazine group, a pyridazine group, a triazine group, or any combination thereof, and
the fourth compound is a compound comprising platinum and a monodentate ligand, a bidentate ligand, a tridentate ligand, a tetradentate ligand, or any combination thereof.

4. The light-emitting device of claim 2, wherein the emission layer comprises:
i) the organic compound; and
ii) the second compound, the third compound, the fourth compound, or any combination thereof, and
the emission layer is configured to emit blue light.

5. An electronic apparatus comprising: the light-emitting device of claim 1; and
a thin-film transistor electrically connected to the light-emitting device.

6. An electronic equipment comprising the light-emitting device of claim 1, wherein the electronic equipment is at least one selected from among a flat panel display, a curved display, a computer monitor, a medical monitor, a television, an advertisement board, an indoor or outdoor lighting and/or signaling light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a 3D display, a virtual or augmented reality display, a vehicle, a video wall comprising multiple displays tiled together, a theater or stadium screen, a phototherapy device, a sign, and combinations thereof.

7. An organic compound represented by Formula 1: 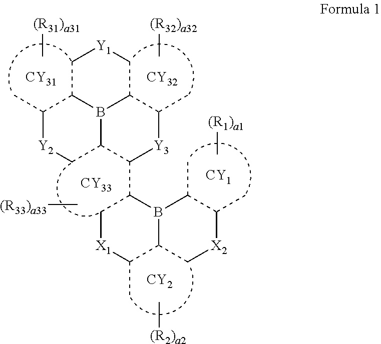
wherein, in Formula 1,
X₁ is selected from among O, S, Se, C(Ar₁₁)(Ar₁₂), Si(Ar₁₁)(Ar₁₂), and N(Ar₁₁),
X₂ is selected from among O, S, Se, C(Ar₂₁)(Ar₂₂), Si(Ar₂₁)(Ar₂₂), and N(Ar₂₁),
Y₁ is selected from among O, S, Se, C(Z₁₁)(Z₁₂), Si(Z₁₁)(Z₁₂), and N(Z₁₁),
Y₂ is selected from among O, S, Se, C(Z₂₁)(Z₂₂), Si(Z₂₁)(Z₂₂), and N(Z₂₁),
Y₃ is selected from among O, S, Se, C(Z₃₁)(Z₃₂), Si(Z₃₁)(Z₃₂), and N(Z₃₁),
Z₁₁ and Z₁₂ are bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
Z₂₁ and Z₂₂ are bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
Z₃₁ and Z₃₂ are bonded to each other to form a C₃-C₆₀ carbocyclic group that is unsubstituted or substituted with at least one R₁₀ₐ or a C₁-C₆₀ heterocyclic group that is unsubstituted or substituted with at least one R₁₀ₐ, or are not bonded,
ring CY₁, ring CY₂, and ring CY₃₁ to ring CY₃₃ are each independently a C₃-C₆₀ carbocyclic group or a C₁-C₆₀ heterocyclic group,
Ar₁₁, Ar₁₂, Ar₂₁, and Ar₂₂ are each independently a C₃-C₆₀ carbocyclic group that is unsubstituted or substituted with at least one R₁₀ₐ or a C₁-C₆₀ heterocyclic group that is unsubstituted or substituted with at least one R₁₀ₐ,
Z₁₁, Z₁₂, Z₂₁, Z₂₂, Z₃₁, Z₃₂, R₁, R₂, and R₃₁ to R₃₃ are each independently hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
a1, a2, and a31 to a33 are each independently an integer from 0 to 20,
at least one selected from among Z₁₁ and Z₁₂ is bonded to ring CY₃₁ or ring CY₃₂ via a single bond, *—O—*′, *—S—*′, *—Se—*, *—C(T₁₁)(T₁₂)-*′, or *—Si(T₁₁)(T₁₂)-*′ or Z₁₁ and Z₁₂ are not bonded to ring CY₃₁ and ring CY₃₂,
at least one selected from among Z₂₁ and Z₂₂ is bonded to ring CY₃₁ or ring CY₃₃ via a single bond, *—O—*′, *—S—*′, *—Se—*, *—C(T₂₁)(T₂₂)-*′, or *—Si(T₂₁)(T₂₂)-*′ or Z₂₁ and Z₂₂ are not bonded to ring CY₃₁ and ring CY₃₃,
at least one selected from among Z₃₁ and Z₃₂ is bonded to ring CY₃₂ or ring CY₃₃ via a single bond, *—O—*′, *—S—*′, *—Se—*′, *—C(T₃₁)(T₃₂)-*′ or *—Si(T₃₁)(T₃₂)-*′ or Z₃₁ and Z₃₂ are not bonded to ring CY₃₂ and ring CY₃₃,
T₁₁, T₁₂, T₂₁, T₂₂, T₃₁, and T₃₂ are each independently hydrogen, deuterium, —F, a cyano group, a C₁-C₆₀ alkyl group, a C₁-C₆₀ alkoxy group, a C₆-C₆₀ aryl group, or a C₁-C₆₀ heteroaryl group,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂—C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, or a nitro group; or
a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

8. The organic compound of claim 7, wherein i) X₁ is N(Ar₁₁); ii) X₂ is N(Ar₂₁); or iii) X₁ is N(Ar₁₁) and X₂ is N(Ar₂₁).

9. The organic compound of claim 7, wherein Ar₁₁ and Ar₂₁ are each independently a biphenyl group that is unsubstituted or substituted with at least one R₁₀ₐ, a terphenyl group that is unsubstituted or substituted with at least one R₁₀ₐ, a fluorenyl group that is unsubstituted or substituted with at least one R₁₀ₐ, a dibenzofuranyl group that is unsubstituted or substituted with at least one R₁₀ₐ, or a dibenzothiophenyl group that is unsubstituted or substituted with at least one R₁₀ₐ.

10. The organic compound of claim 7, wherein Ar₁₁ and Ar₂₁ are each independently a group represented by any one selected from among Formulae AR1 to AR4: 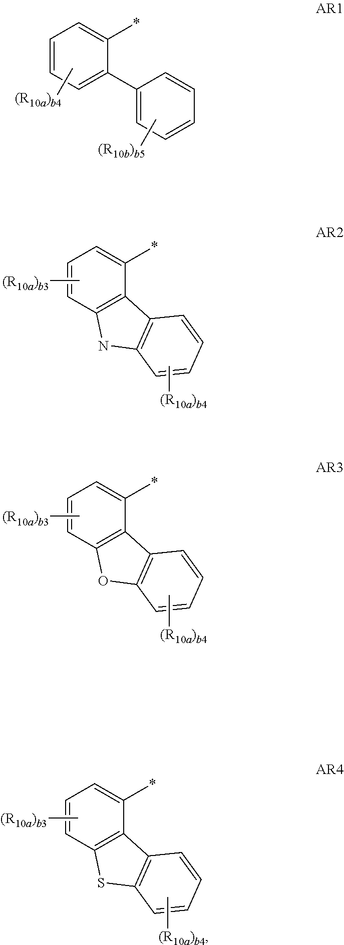
in Formulae AR1 to AR4,
b3 is an integer from 0 to 3,
b4 is an integer from 0 to 4,
b5 is an integer from 0 to 5,
R_{10b} is deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), or —P(═O)(Q₂₁)(Q₂₂), wherein Q₂₁, Q₂₂, and Q₂₃ are each independently defined as in Formula 1, and
* indicates a binding site to a neighboring atom.

11. The organic compound of claim 7, wherein
i)
Y₂ is selected from among O, S, Se, and N(Z₂₁), and
Z₂₁ is bonded to ring CY₃₁ via a single bond, *—O—*′, *—S—*′, *—Se—*′, *—C(T₂₁)(T₂₂)-*′, or *—Si(T₂₁)(T₂₂)-*′ or is not bonded to ring CY₃₁;
ii)
Y₃ is selected from among O, S, Se, and N(Z₃₁), and
Z₃₁ is bonded to ring CY₃₂ via a single bond, *—O—*′, *—S—*′, *—Se—*′, *—C(T₃₁)(T₃₂)-*′, or *—Si(T₃₁)(T₃₂)-*′ or is not bonded to ring CY₃₂; or
iii)
Y₂ is selected from among O, S, Se, and N(Z₂₁),
Y₃ is selected from among O, S, Se, and N(Z₃₁),
Z₂₁ is bonded to ring CY₃₁ via a single bond, *—O—*′, *—S—*′, *—Se—*′, *—C(T₂₁)(T₂₂)-*′, or *—Si(T₂₁)(T₂₂)-*′ or is not bonded to ring CY₃₁, and
Z₃₁ is optionally bonded to ring CY₃₂ via a single bond, *—O—*′, *—S—*′, *—Se—*′, *—C(T₃₁)(T₃₂)-*′, or *—Si(T₃₁)(T₃₂)-*′ or is not bonded to ring CY₃₂.

12. The organic compound of claim 7, wherein Z₂₁ and Z₃₁ are each independently a C₃-C₁ cycloalkyl group that is unsubstituted or substituted with at least one R₁₀ₐ, a C₁-C₁₀ heterocycloalkyl group that is unsubstituted or substituted with at least one R₁₀ₐ, a C₃-C₁₀ cycloalkenyl group that is unsubstituted or substituted with at least one R₁₀ₐ, a C₁-C₁₀ heterocycloalkenyl group that is unsubstituted or substituted with at least one R₁₀ₐ, a C₆-C₆₀ aryl group that is unsubstituted or substituted with at least one R₁₀ₐ, a C₁-C₆₀ heteroaryl group that is unsubstituted or substituted with at least one R₁₀ₐ, a monovalent non-aromatic condensed polycyclic group that is unsubstituted or substituted with at least one R₁₀ₐ, or a monovalent non-aromatic condensed heteropolycyclic group that is unsubstituted or substituted with at least one R₁₀ₐ.

13. The organic compound of claim 7, wherein ring CY1, ring CY2, and ring CY₃₁ to ring CY₃₃ are each independently a 6-membered ring.

14. The organic compound of claim 7, wherein ring CY₁, ring CY₂, and ring CY₃₁ to ring CY₃₃ are each independently a benzene group, a naphthalene group, a pyridine group, a pyrimidine group, a triazine group, a quinoline group, or an isoquinoline group.

15. The organic compound of claim 7, wherein a group represented by 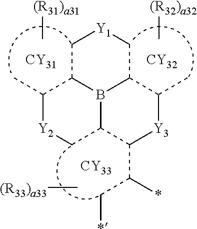
in Formula 1 is a group represented by any one selected from among Formulae FT1 to FT4: 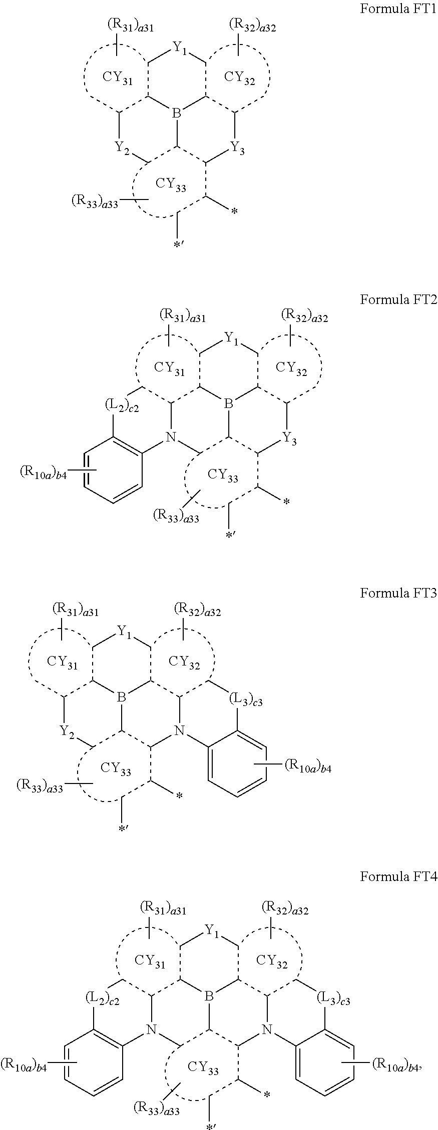
in Formulae FT1 to FT4,
ring CY₃₁, ring CY₃₂, ring CY₃₃, R₃₁, R₃₂, R₃₃, Y₁, a31, a32, and a33 are each independently defined as in Formula 1,
Y₂ and Y₃ are each independently selected from among O, S, and Se,
b4 is an integer from 0 to 4,
L₂ is O, S, Se, C(T₂₁)(T₂₂), or Si(T₂₁)(T₂₂),
L₃ is O, S, Se, C(T₃₁)(T₃₂), or Si(T₃₁)(T₃₂),
c2 is 0 or 1, wherein, when c2 is 0, (L₂)_{c2} is a single bond,
c3 is 0 or 1, wherein, when c3 is 0, (L₃)_{c3} is a single bond,
* indicates a binding site to B in Formula 1, and
*′ indicates a binding site to X₁ in Formula 1.

16. The organic compound of claim 15, wherein at least one selected from among Y₁ to Y₃ in Formula FT1 is O or S,
at least one selected from among Y₁, L₂, and Y₃ in Formula FT2 is O or S,
at least one selected from among Y₁, Y₂, and L₃ in Formula FT3 is O or S, and
at least one selected from among Y₁, L₂, and L₃ in Formula FT4 is O or S.

17. The organic compound of claim 7, wherein the organic compound comprises at least one deuterium.

18. The organic compound of claim 7, wherein R₁, R₂, and R₃₁ to R₃₃ are each independently selected from among:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, and a phenyl group;
a C₁-C₆₀ alkyl group, a phenyl group, a naphthyl group, an anthracenyl group, a phenanthrenyl group, a phenalenyl group, a triphenylenyl group, a pyrenyl group, a chrysenyl group, a pyrrolyl group, a furanyl group, a thiophenyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzoimidazolyl group, a benzofuranyl group, a dibenzofuranyl group, a benzothiophenyl group, a dibenzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a carbazolyl group, a benzocarbazolyl group, a fluorenyl group, a benzoisothiazolyl group, a benzoxazolyl group, a benzoisoxazolyl group, a phenoxazinyl group, an acridinyl group, and a xanthenyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a phenyl group, or any combination thereof; and
—Si(Q₁)(Q₂)(Q₃), and —N(Q₁)(Q₂).

19. The organic compound of claim 7, wherein at least one selected from among R₃₁ to R₃₃ is deuterium.

20. The organic compound of claim 7, wherein the organic compound is represented by any one selected from among Compounds 1 to 80: 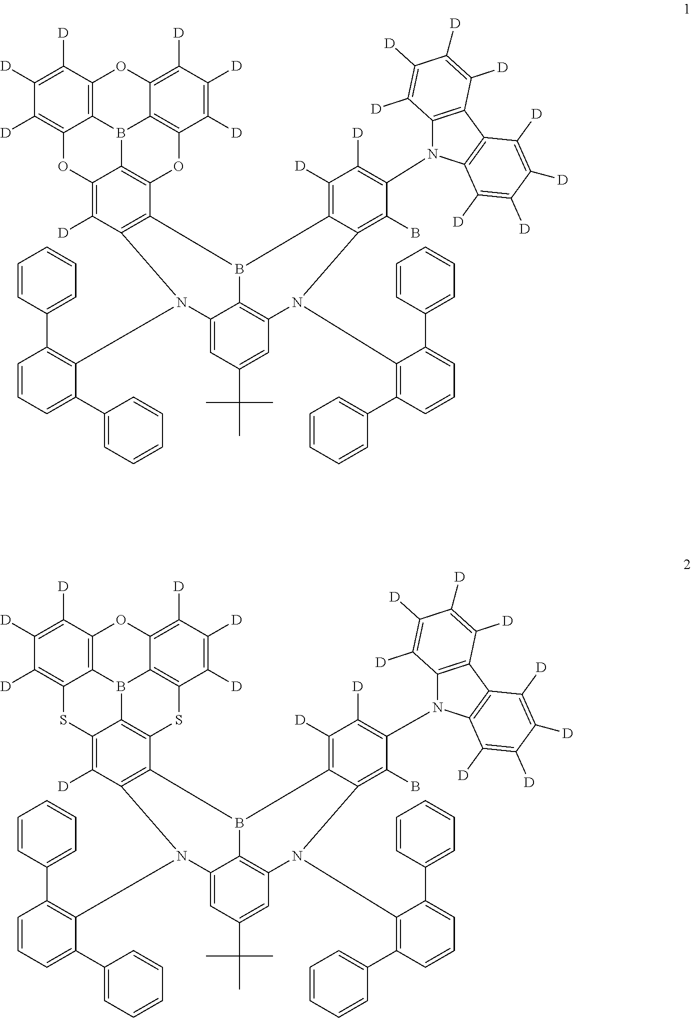 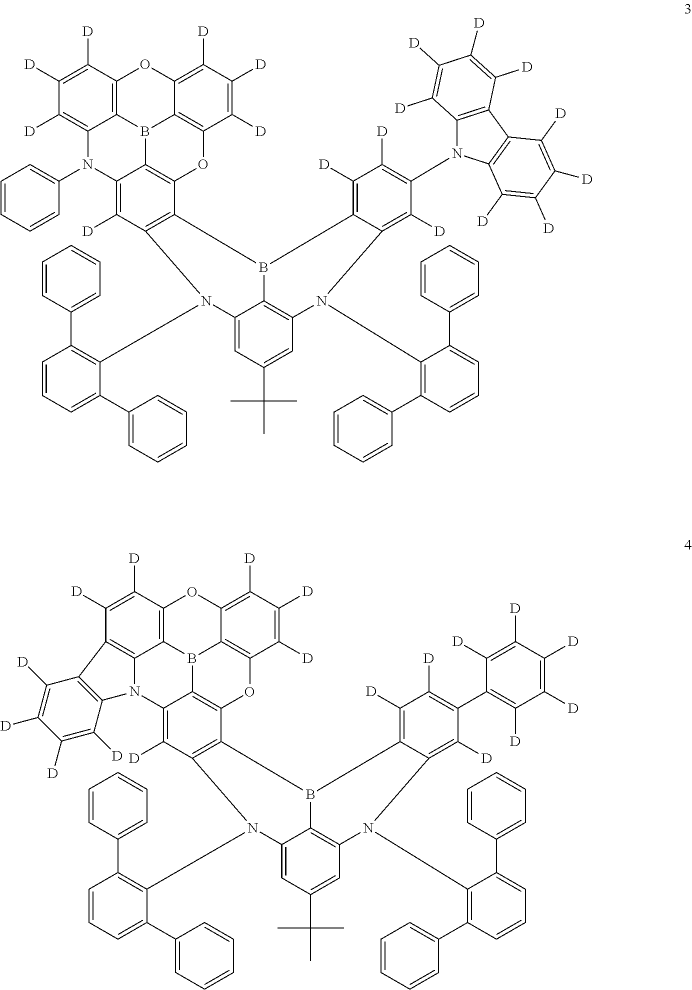 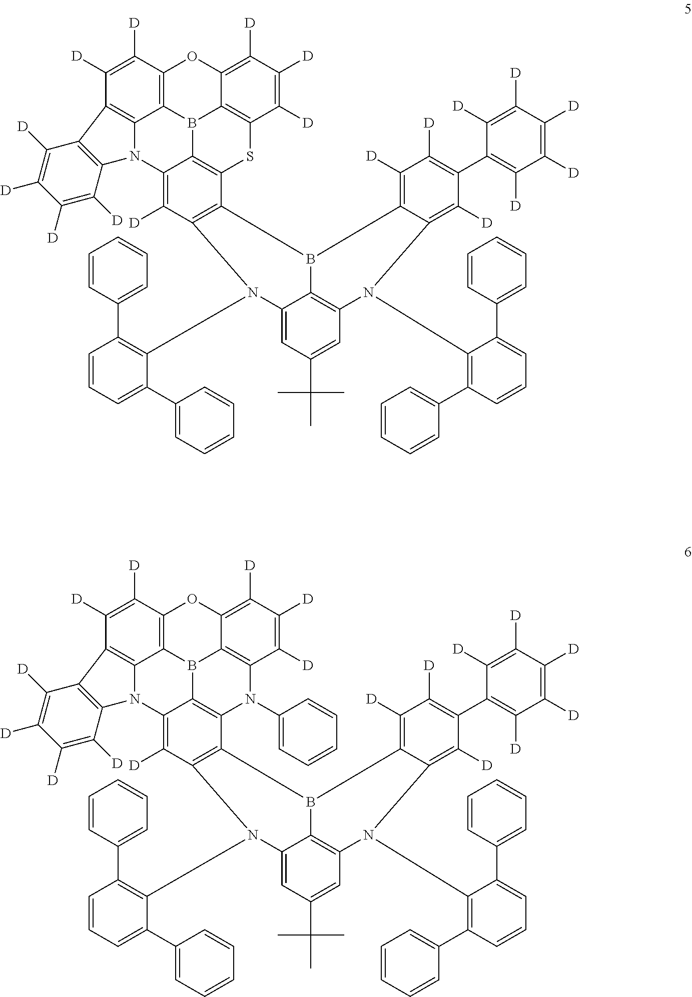 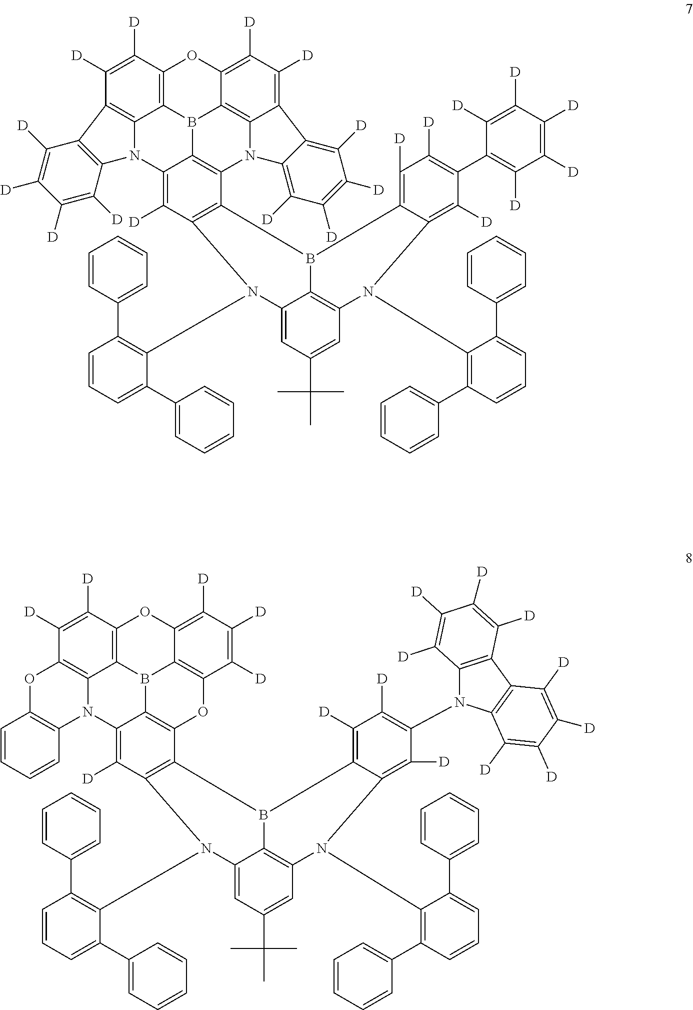 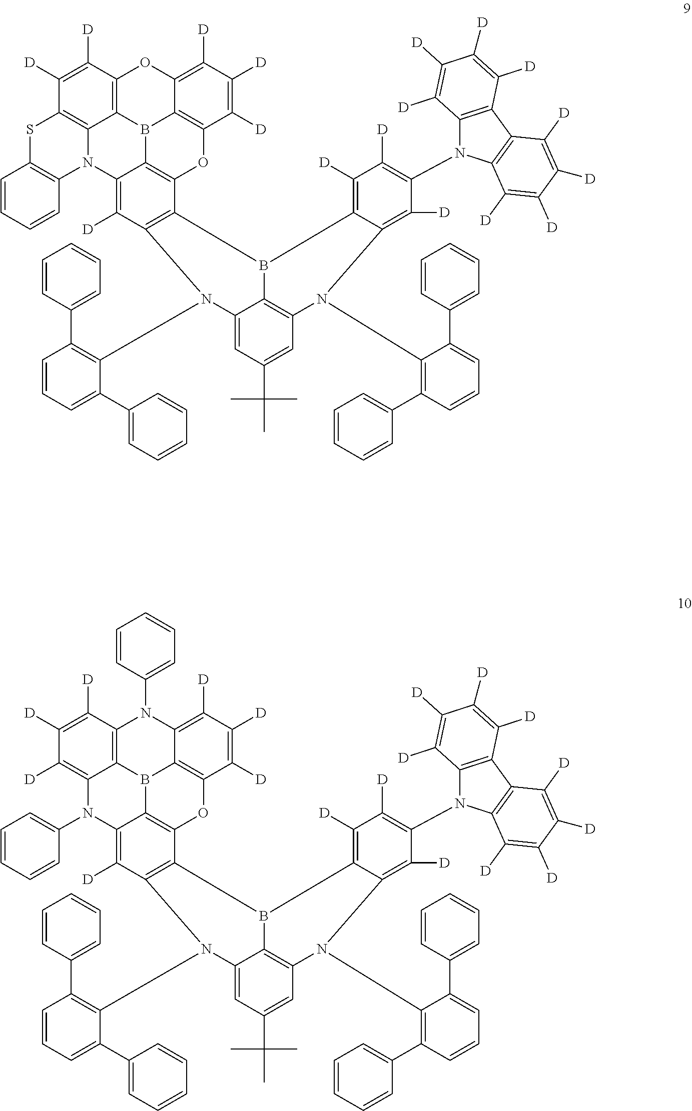 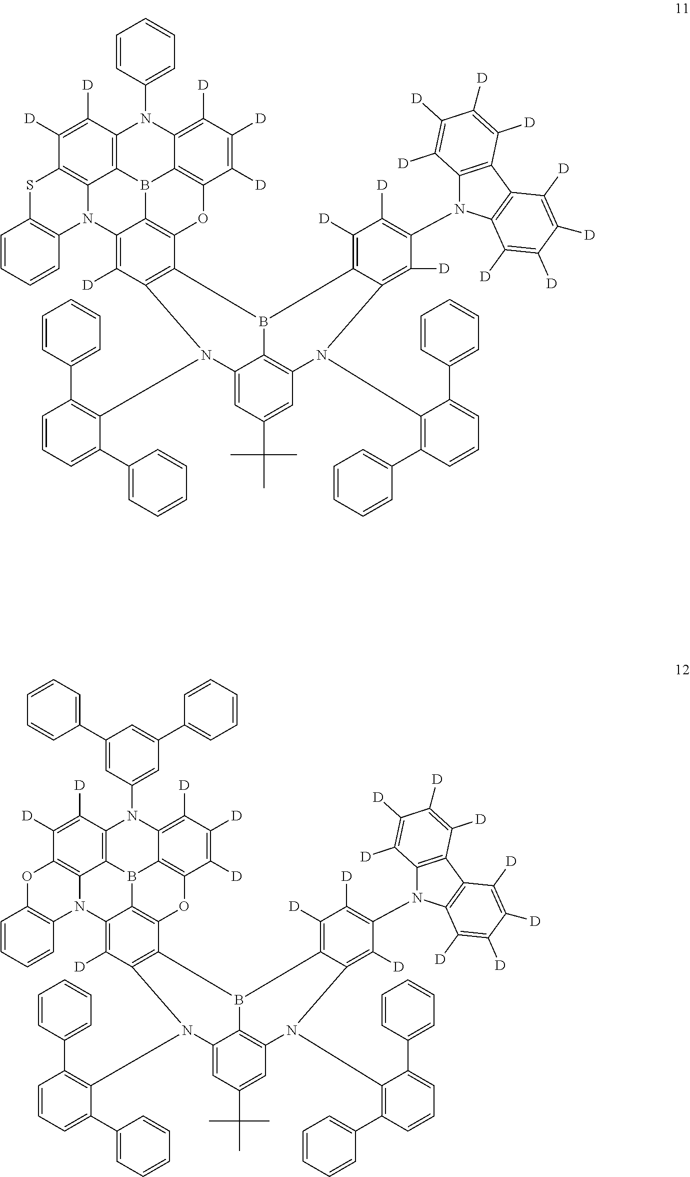 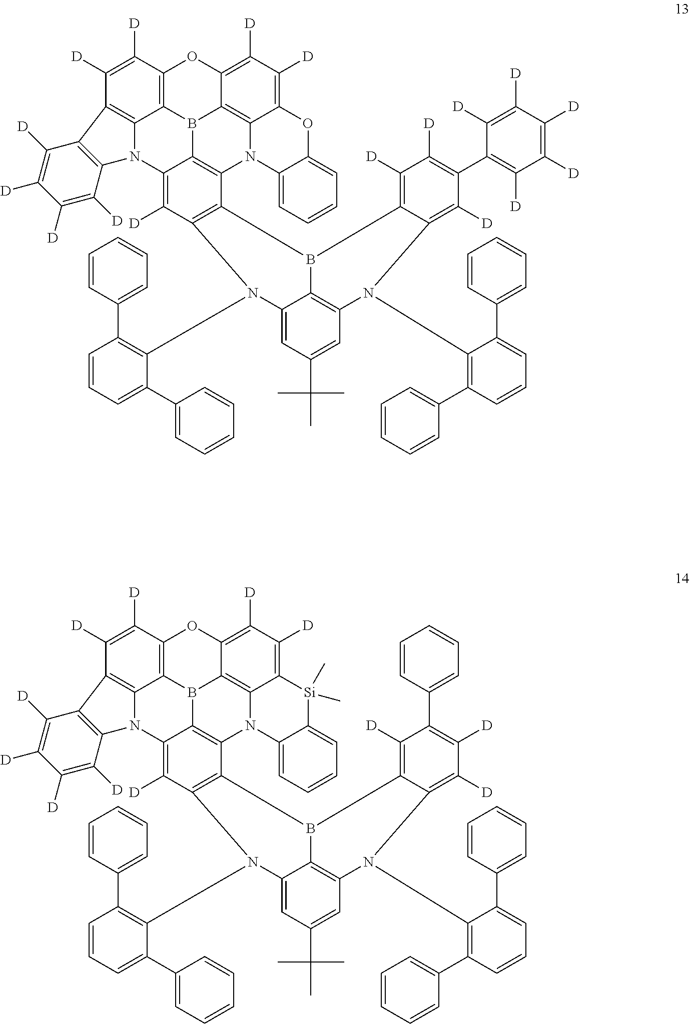 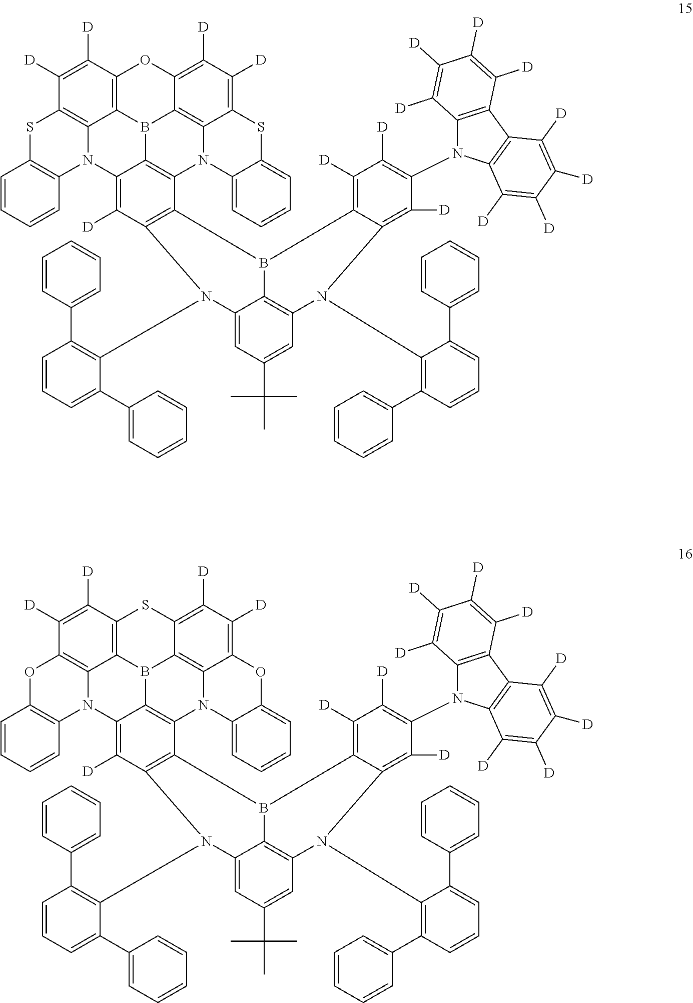 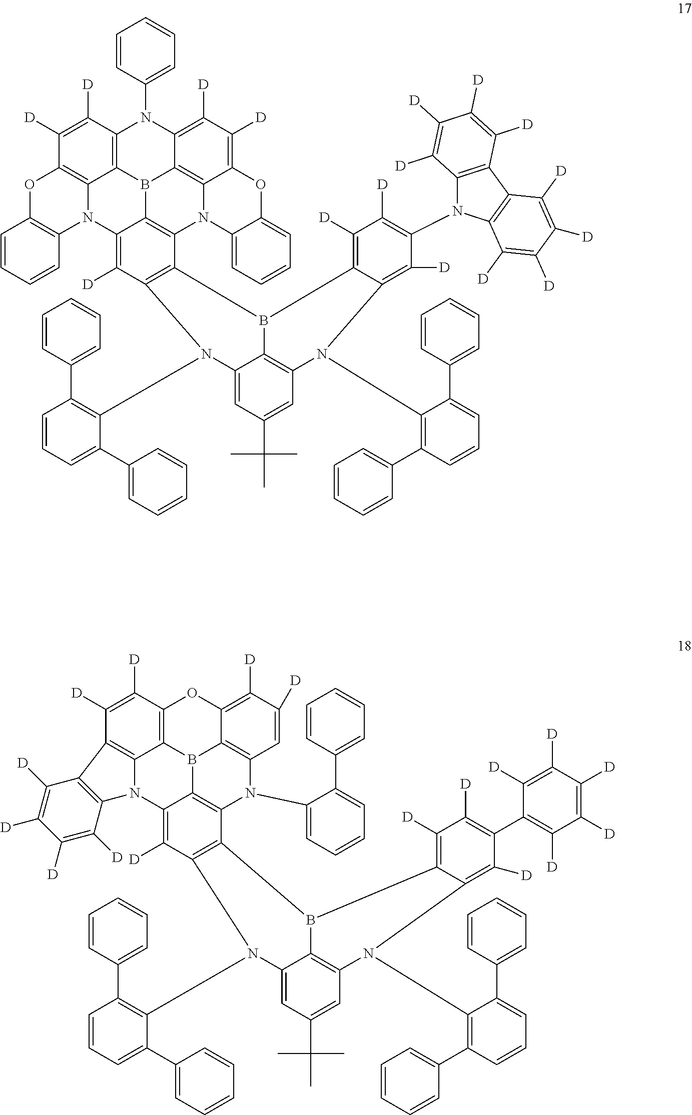 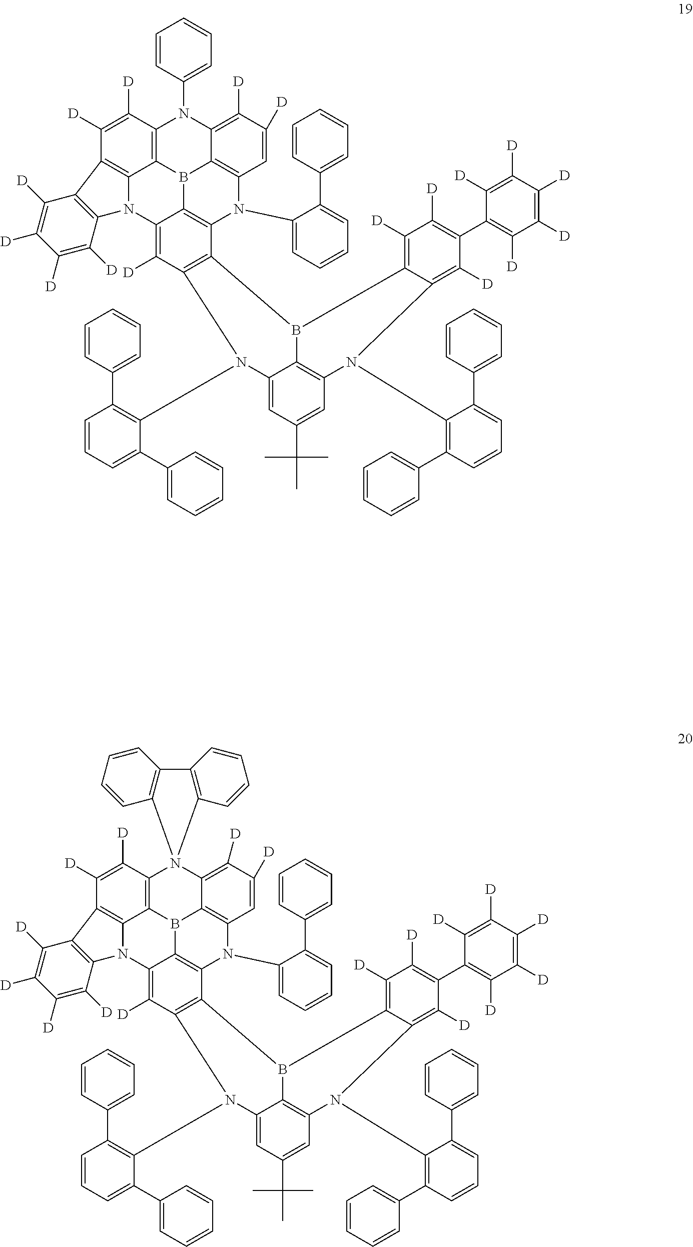 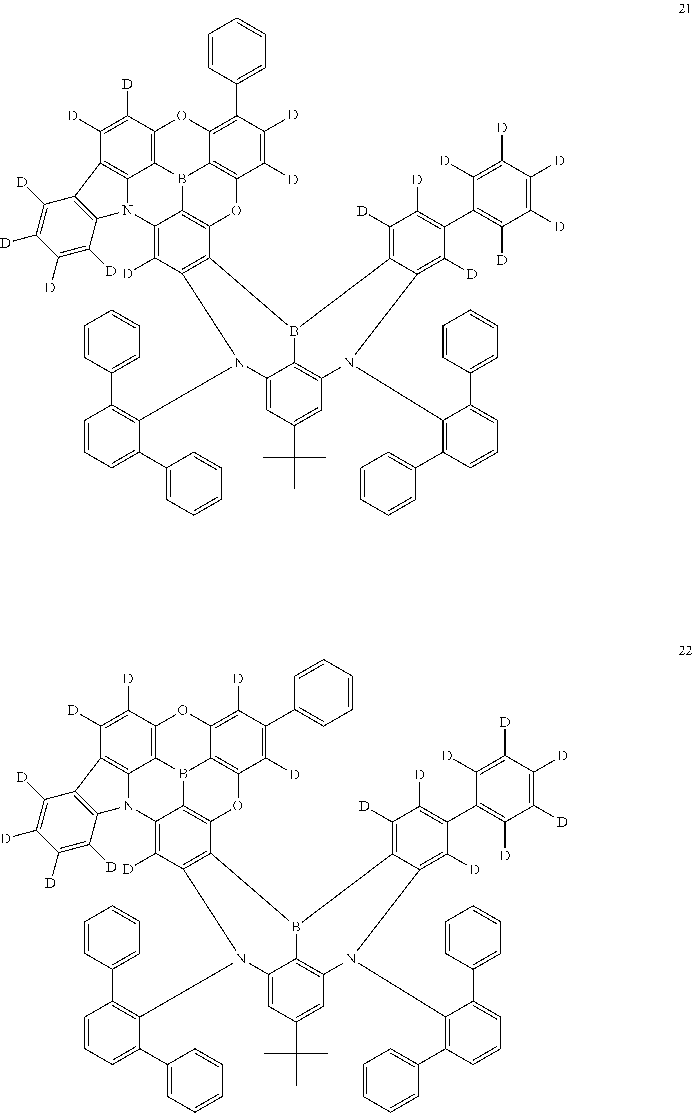 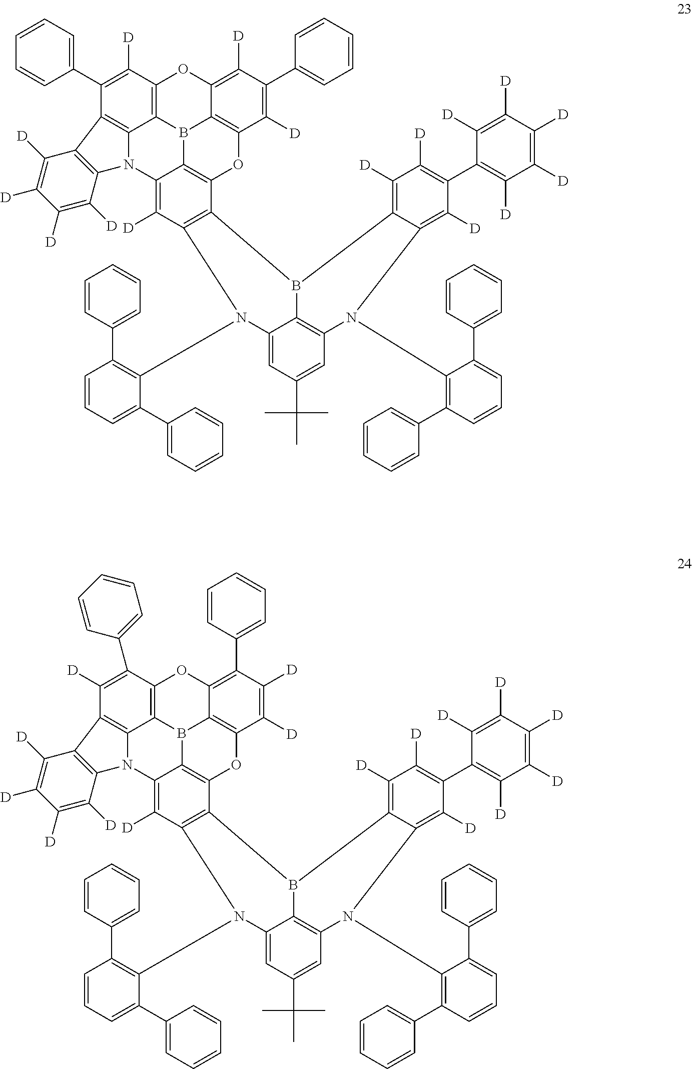 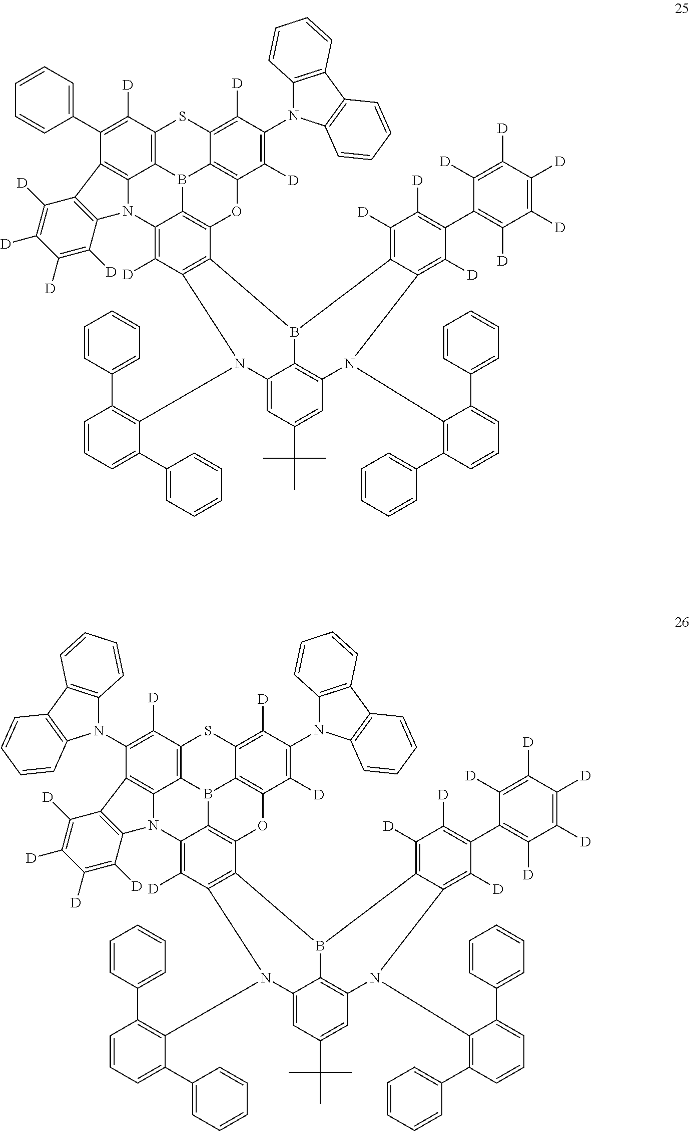 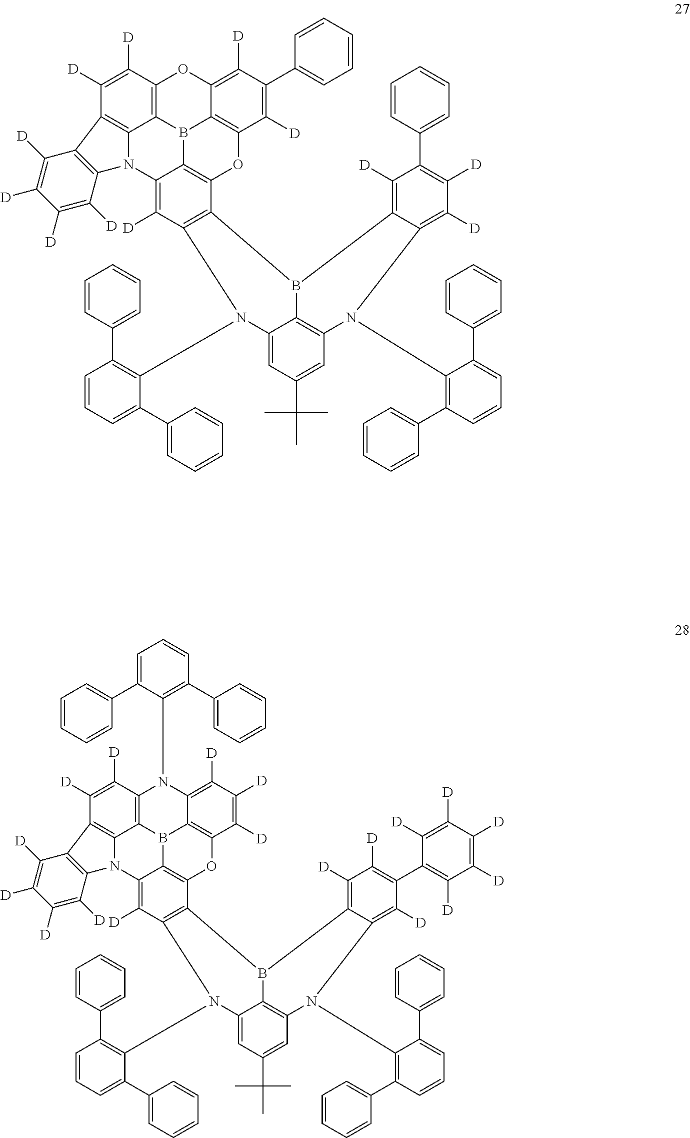 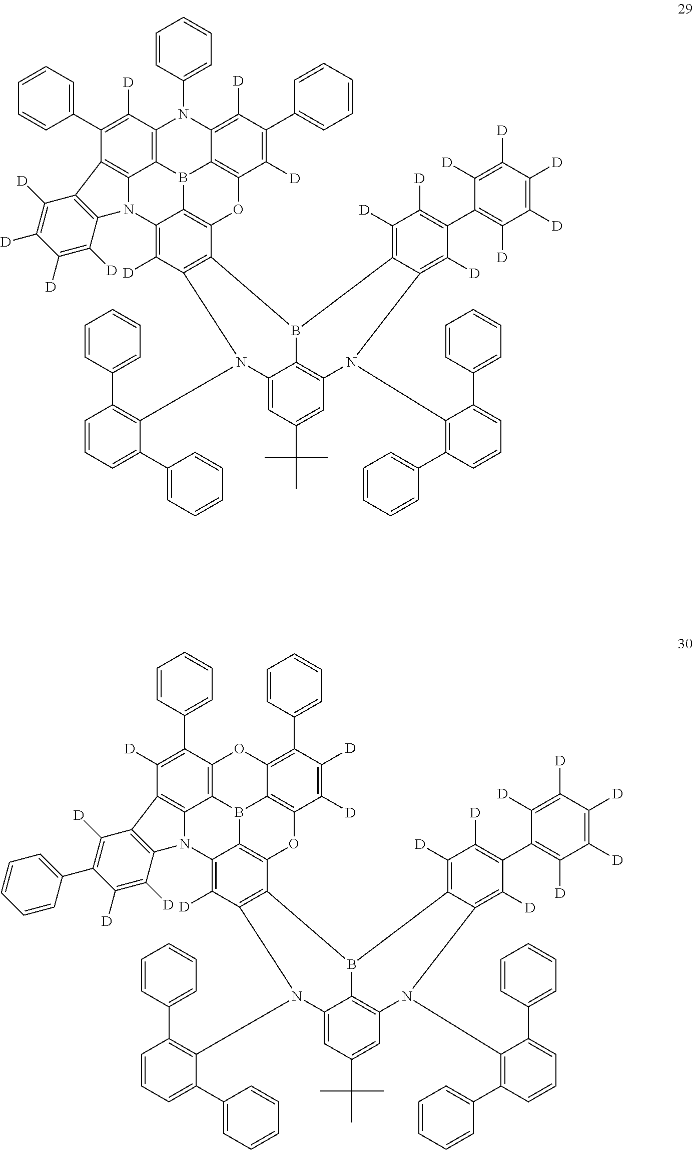 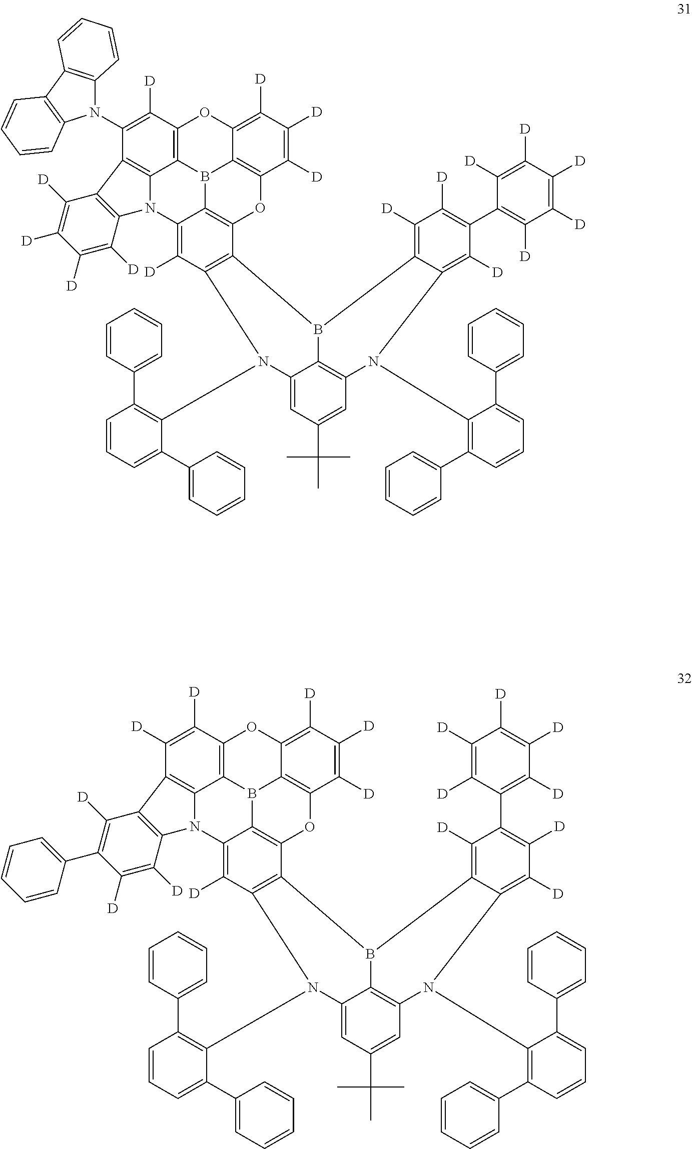 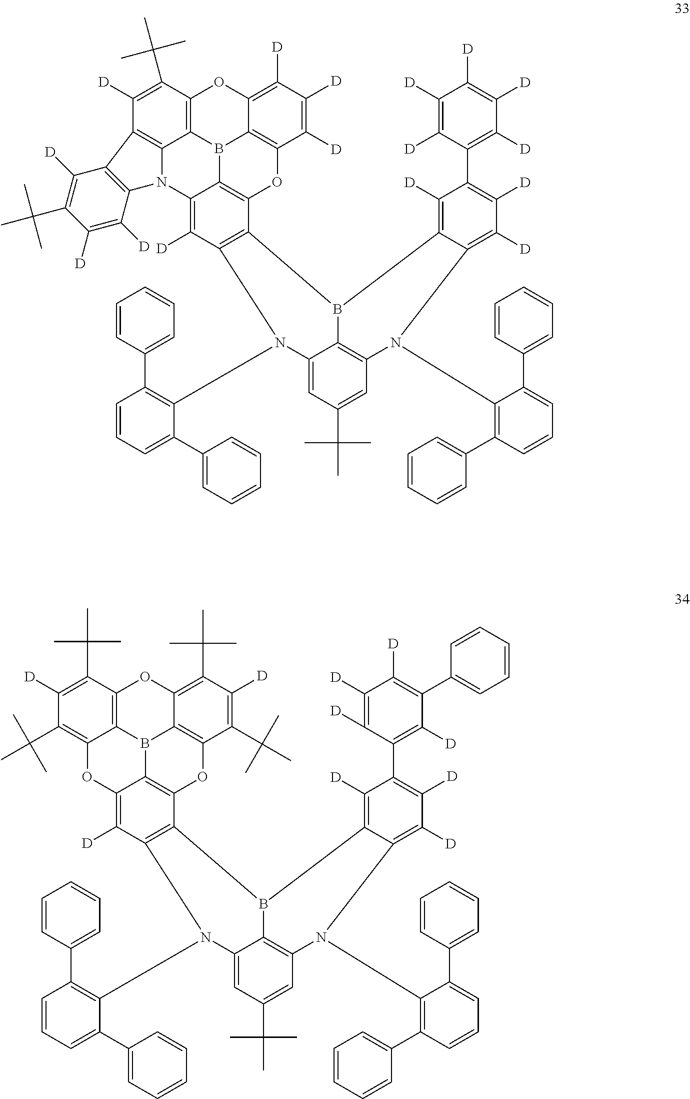 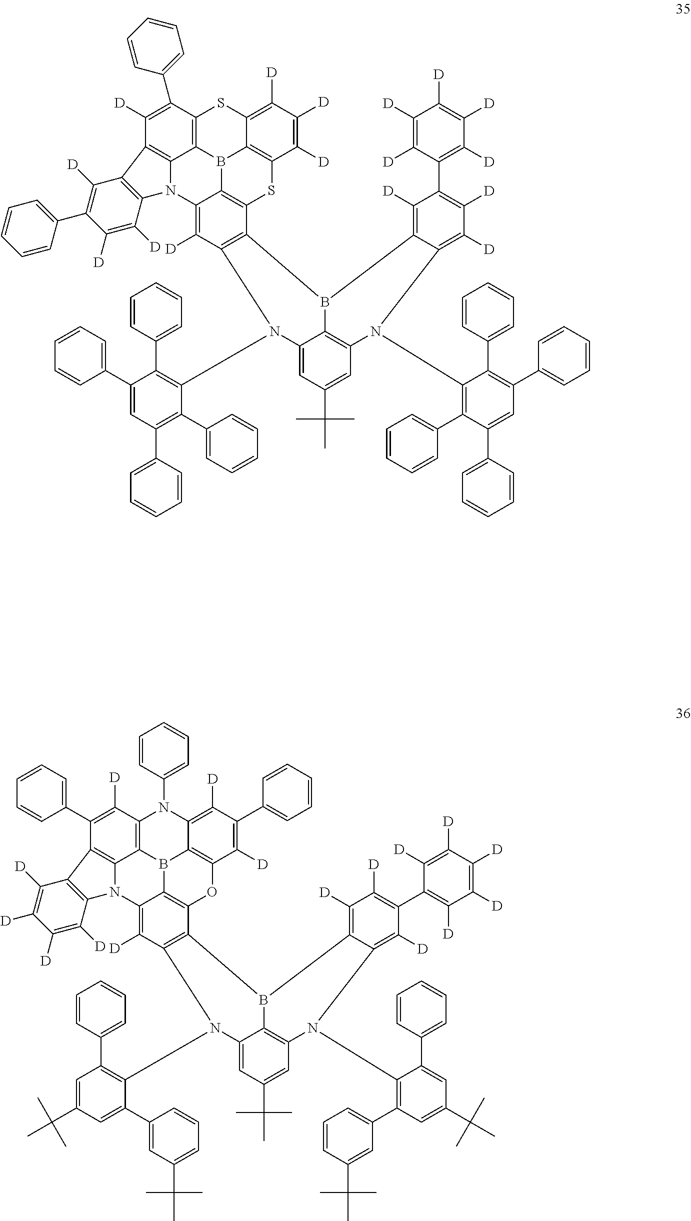 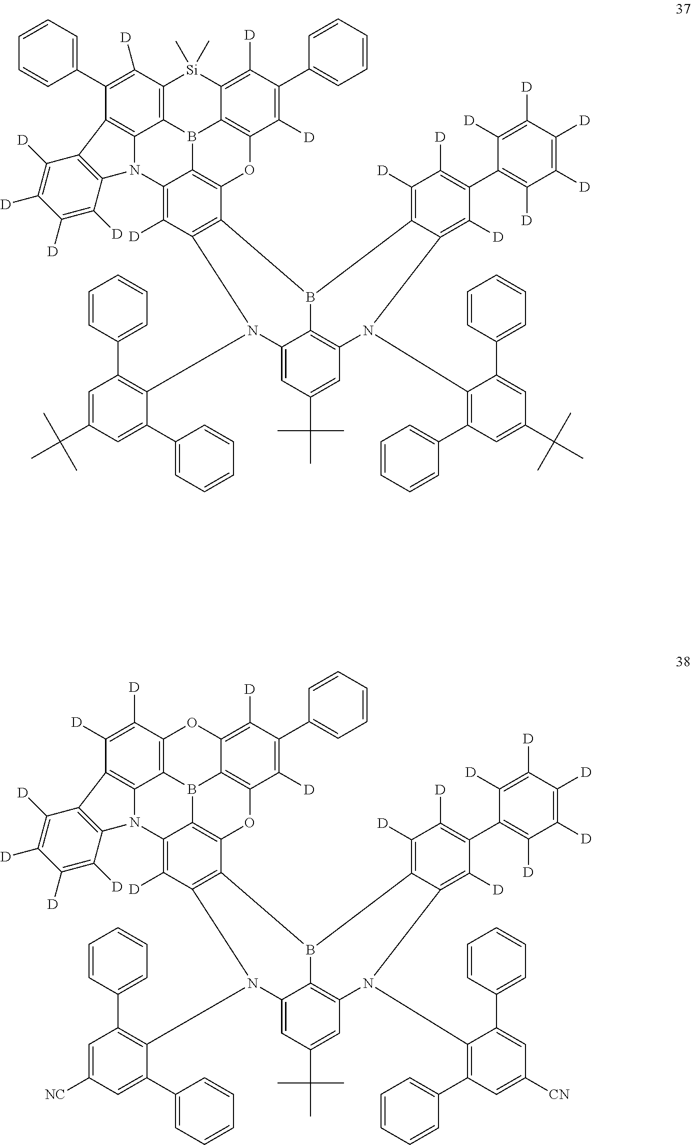 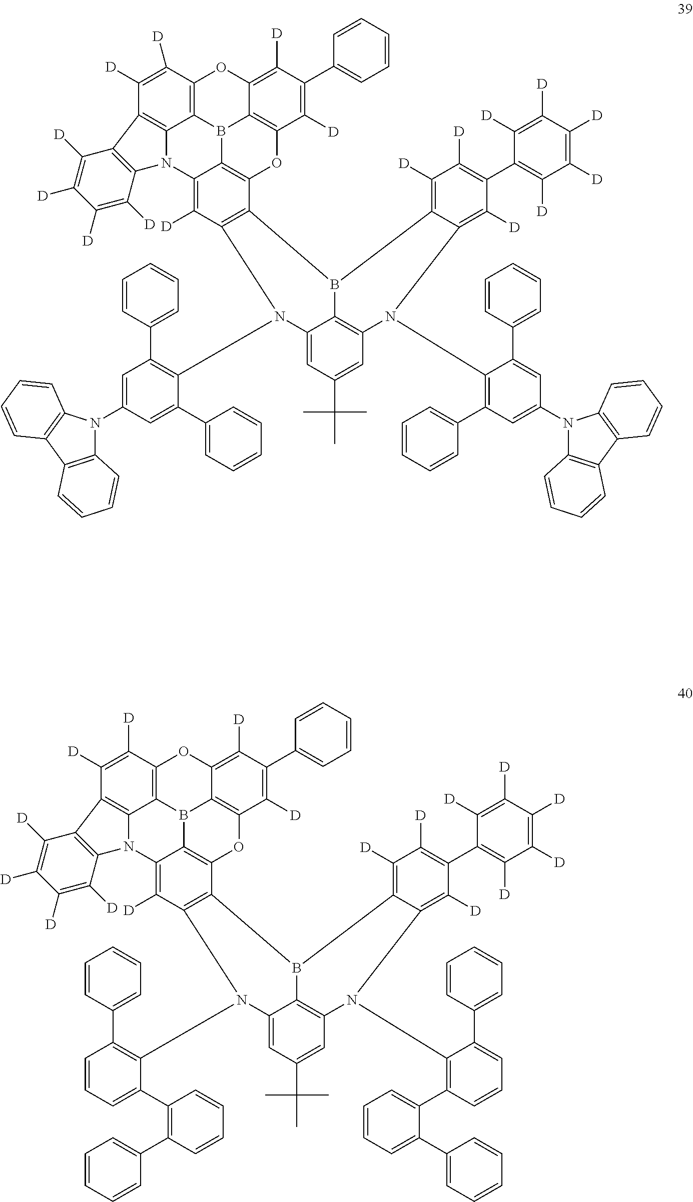 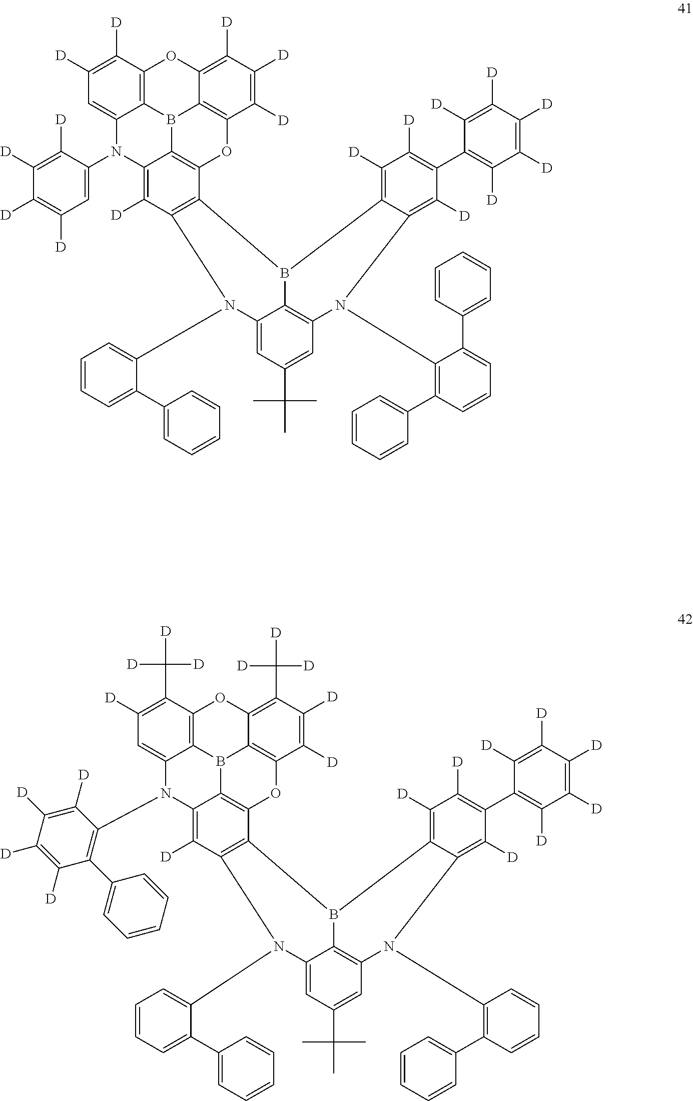 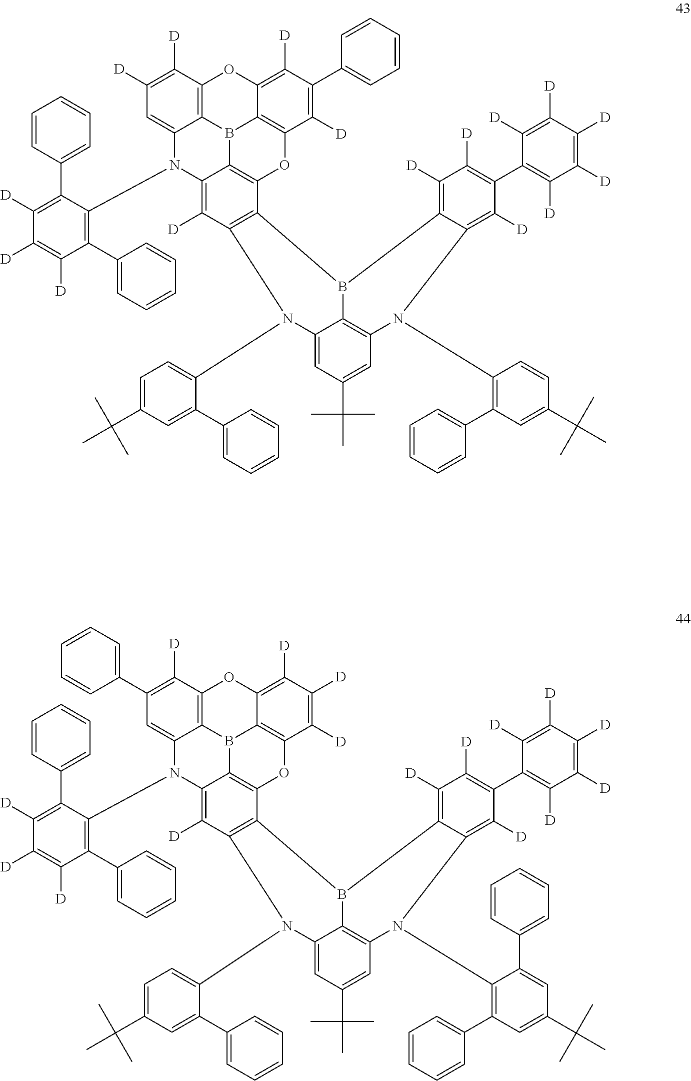 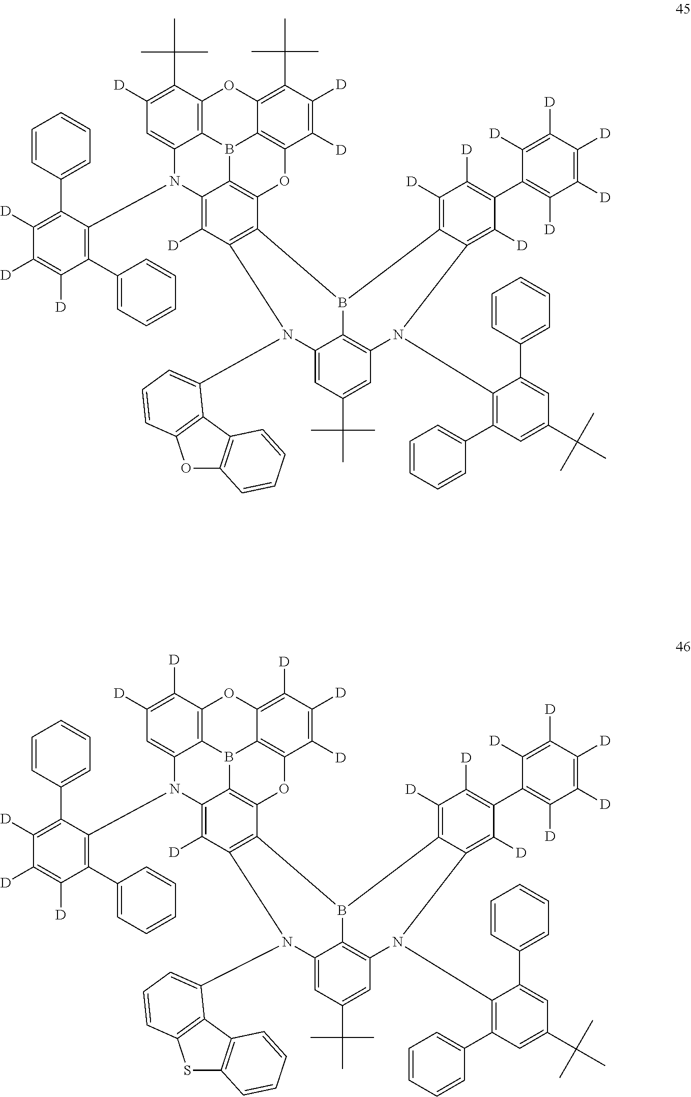 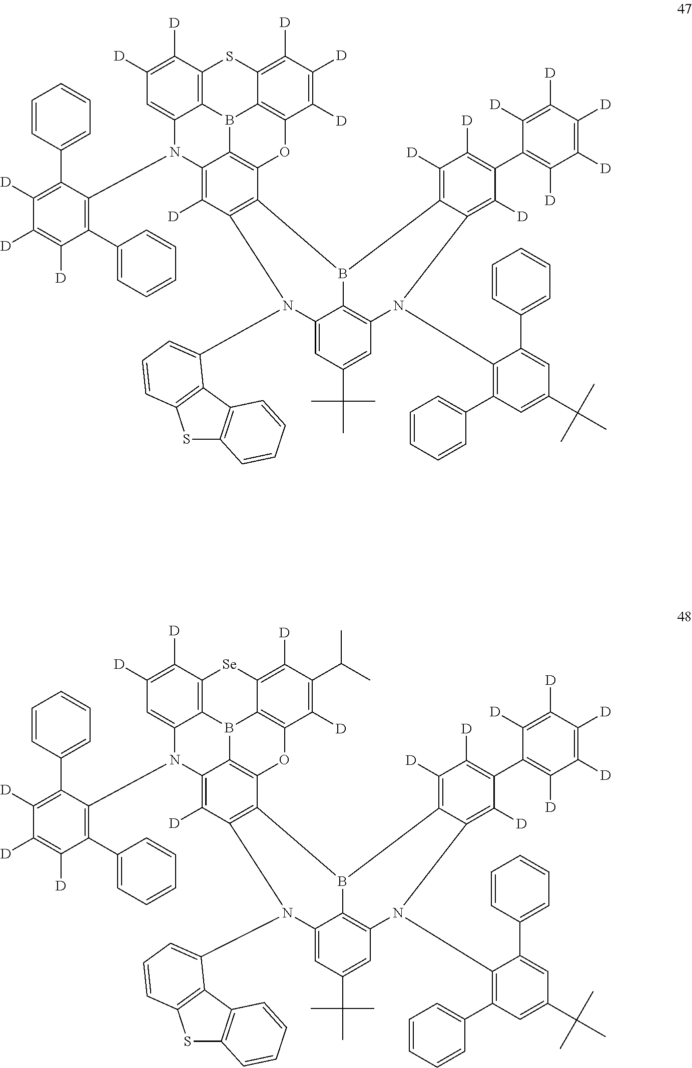 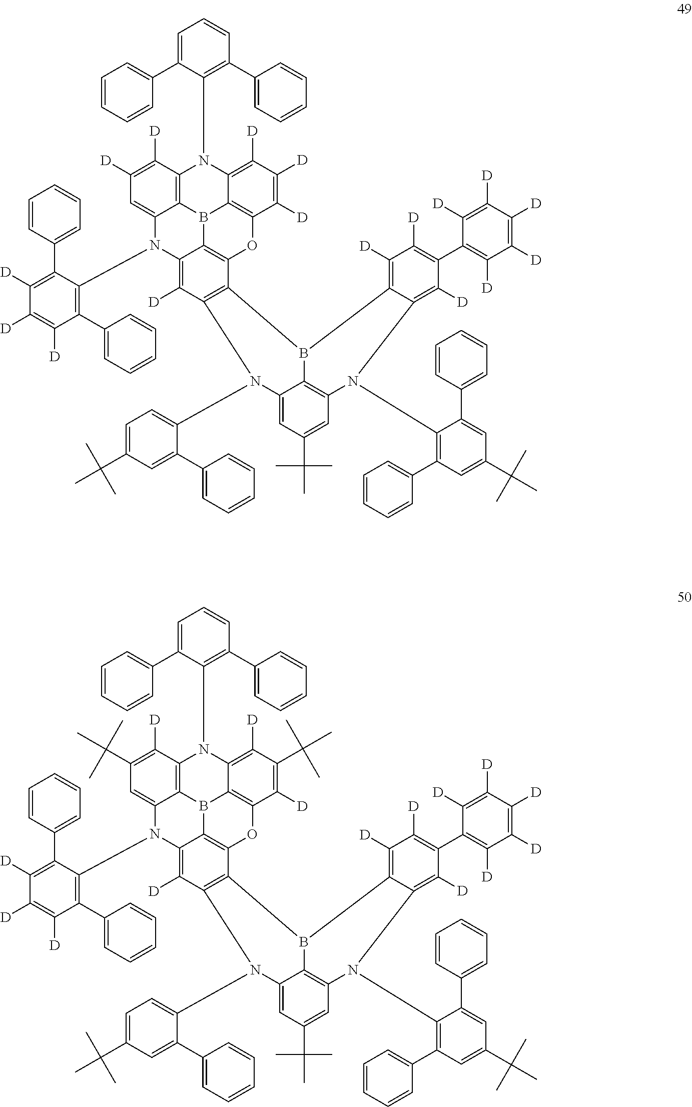 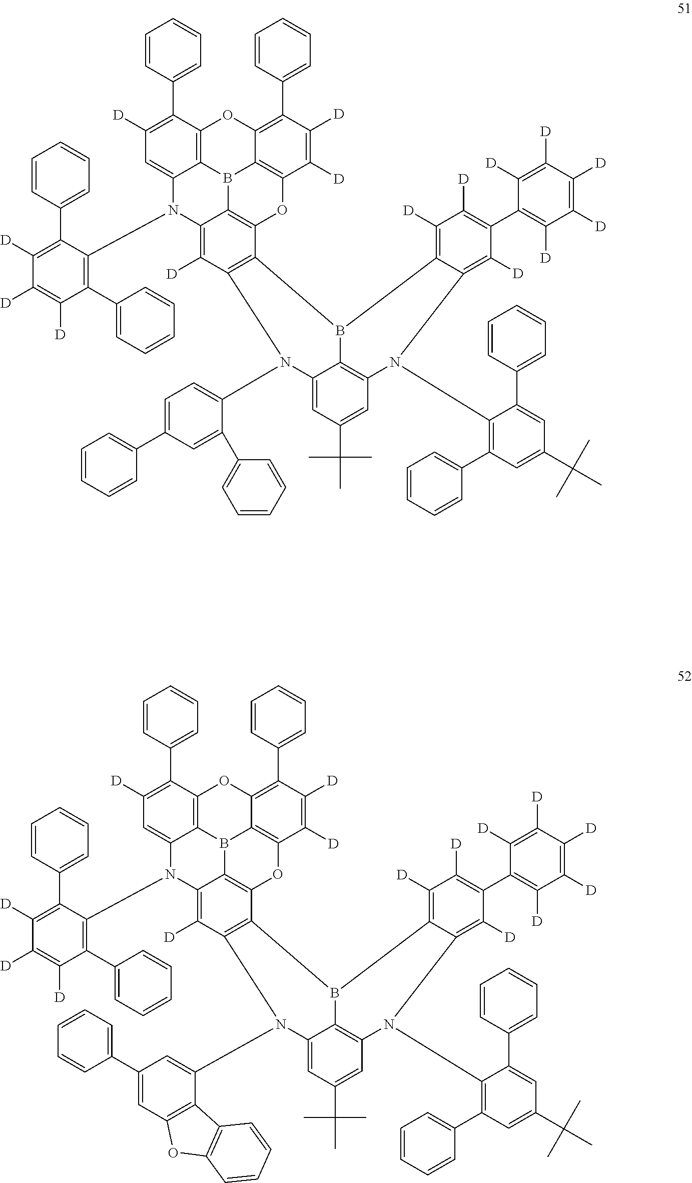 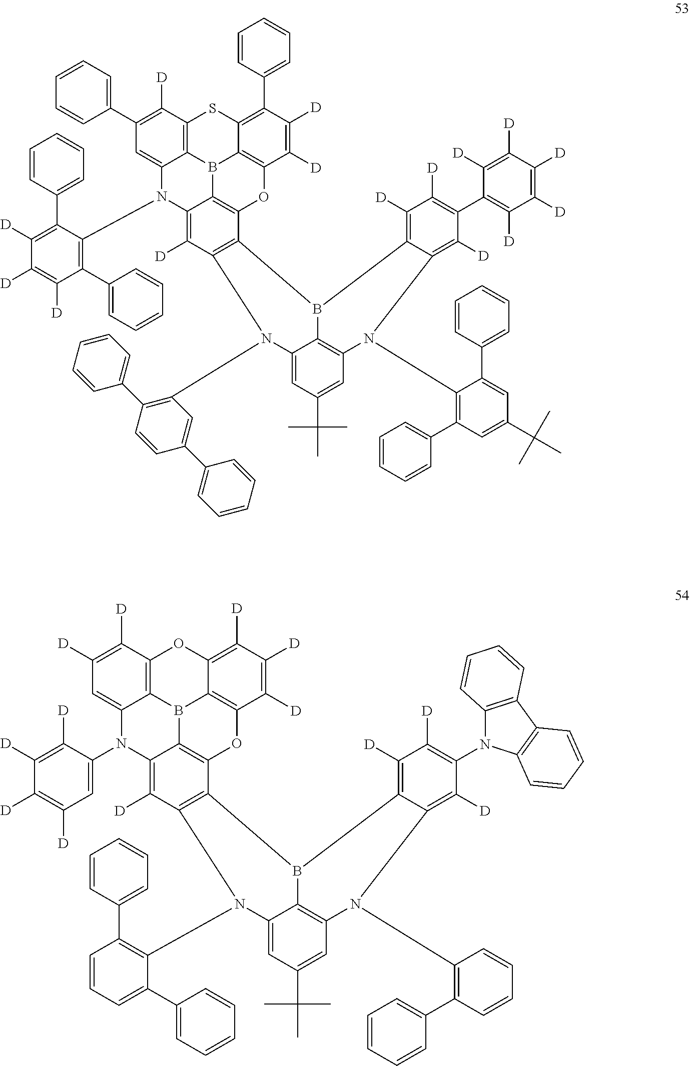 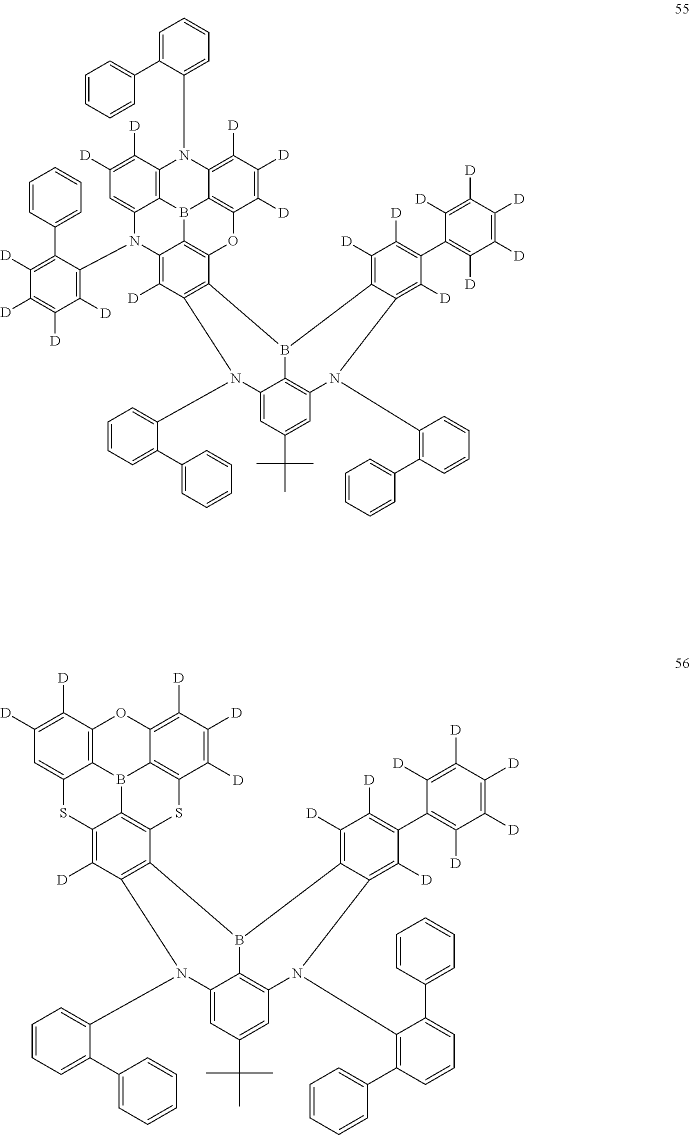 [SMILES: [2H]c1cc2c3c(c1[2H])N(c1ccccc1-c1ccccc1)c1c([2H])c([2H])c([2H])c4c1B3c1c(c3c(c([2H])c1N2c1c([2H])c([2H])c([2H])c([2H])c1-c1ccccc1)N(c1ccccc1-c1ccccc1)c1cc(C(C)(C)C)cc2c1B3c1c([2H])c([2H])c(-c3c([2H])c([2H])c([2H])c([2H])c3[2H])c([2H])c1N2c1ccccc1-c1ccccc1)O4 ; [2H]c1cc2c3c(c1[2H])Oc1c([2H])c([2H])c([2H])c4c1B3c1c(c([2H])c3c(c1S4)B1c4c(cc(C(C)(C)C)cc4N(c4c(-c5ccccc5)cccc4-c4ccccc4)c4c([2H])c(-c5c([2H])c([2H])c([2H])c([2H])c5[2H])c([2H])c([2H])c41)N3c1ccccc1-c1ccccc1)S2] 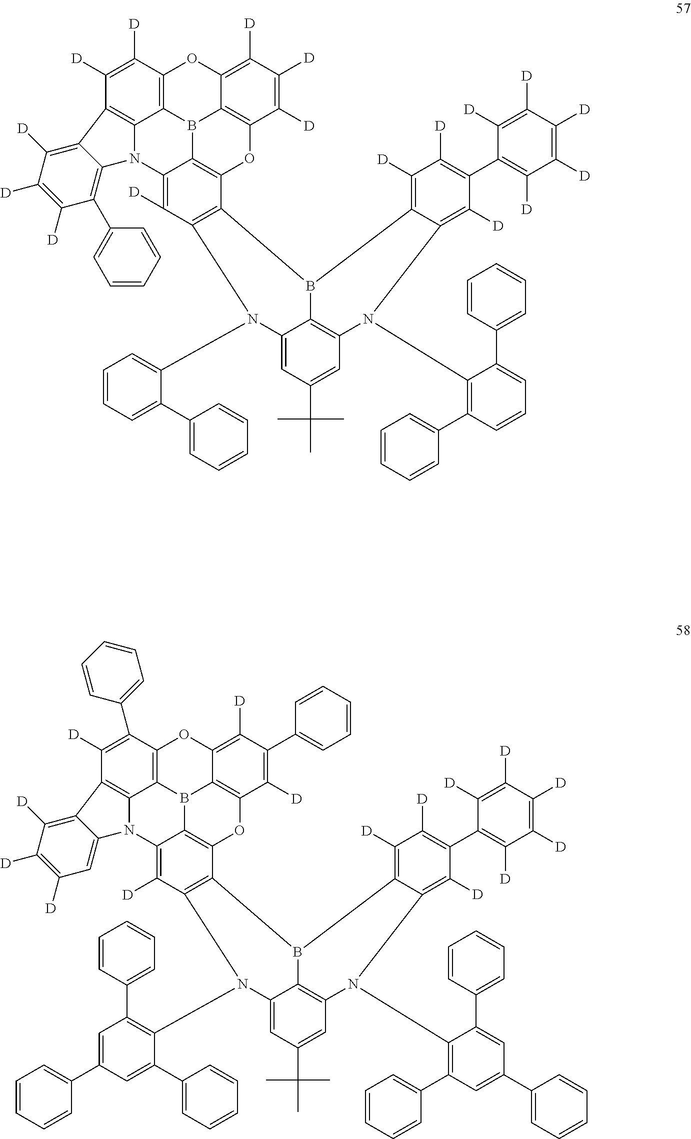 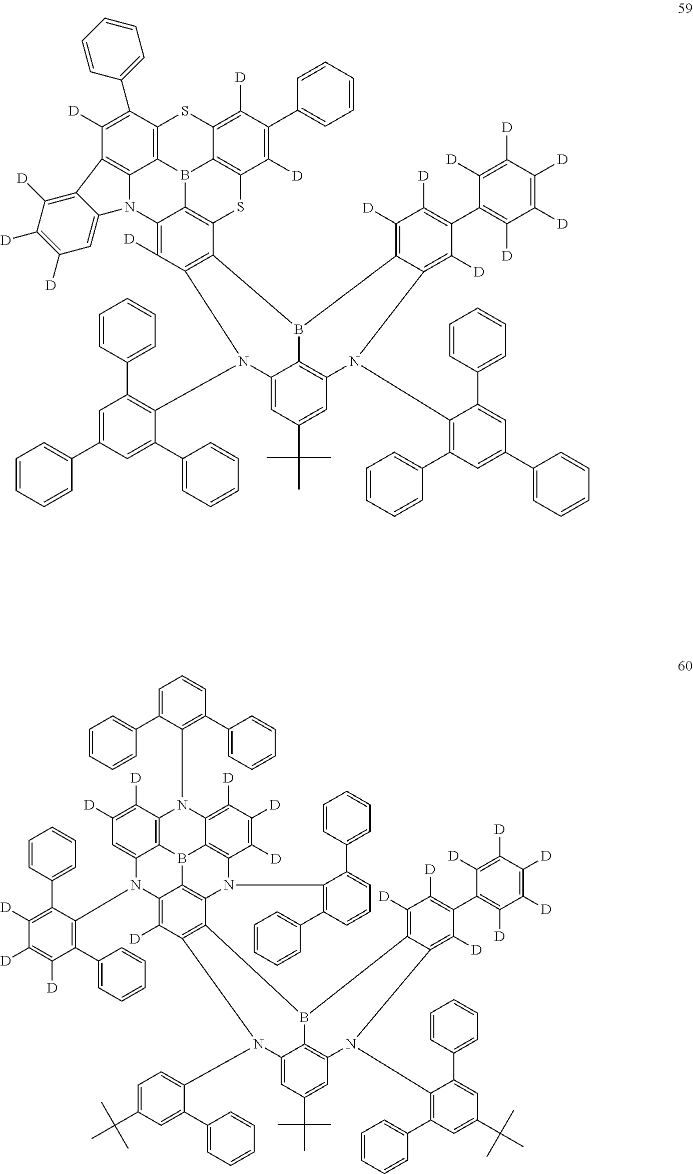 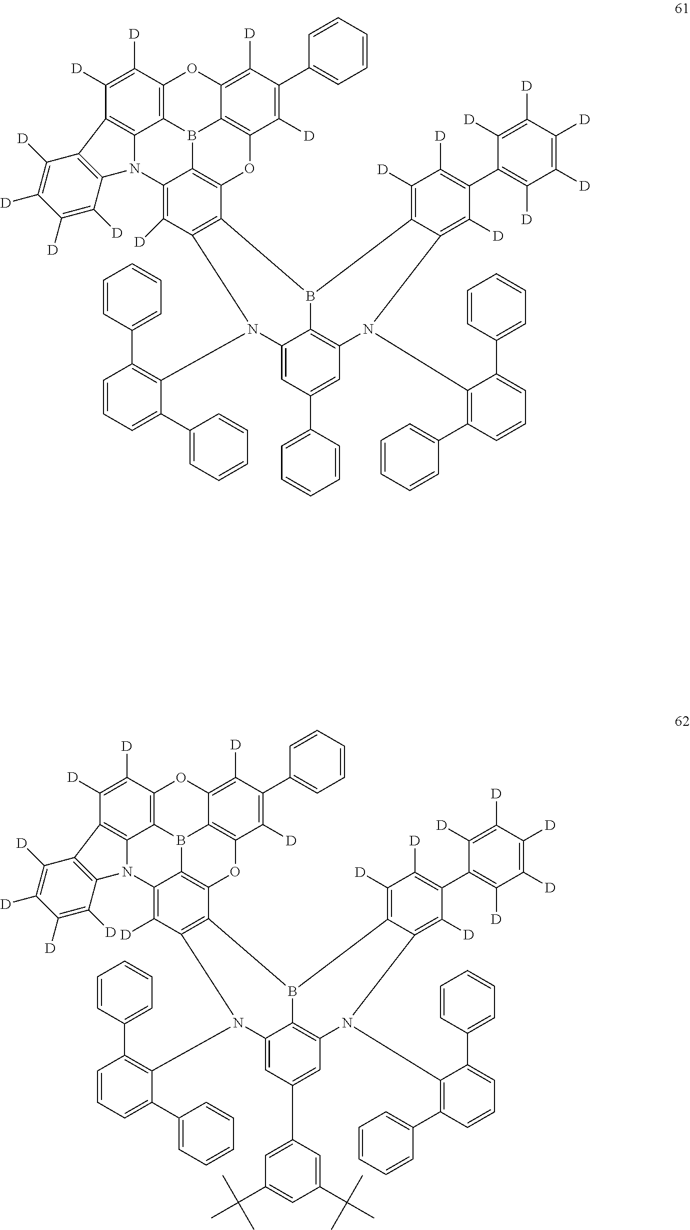 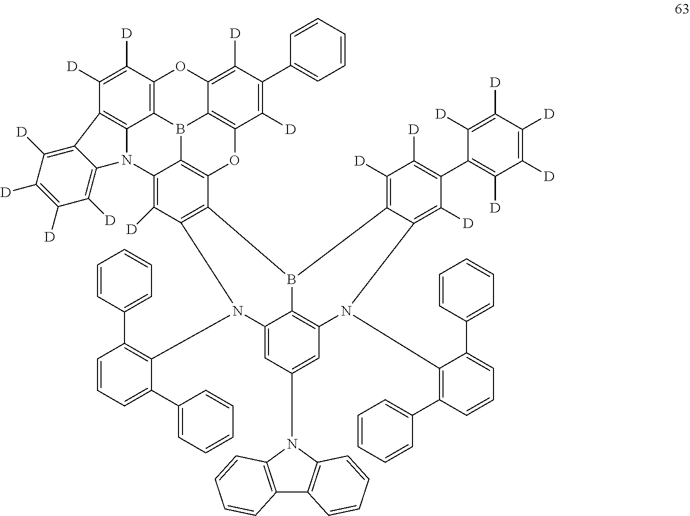 [SMILES: [2H]c1c([2H])c([2H])c(-c2c([2H])c([2H])c3c(c2[2H])N(c2c(-c4ccccc4)cccc2-c2ccccc2)c2cc(-n4c5ccccc5c5ccccc54)cc4c2B3c2c3c5c(c([2H])c2N4c2c(-c4ccccc4)cccc2-c2ccccc2)-n2c4c([2H])c([2H])c([2H])c([2H])c4c4c([2H])c([2H])c6c(c42)B5c2c(c([2H])c(-c4ccccc4)c([2H])c2O6)O3)c([2H])c1[2H]] 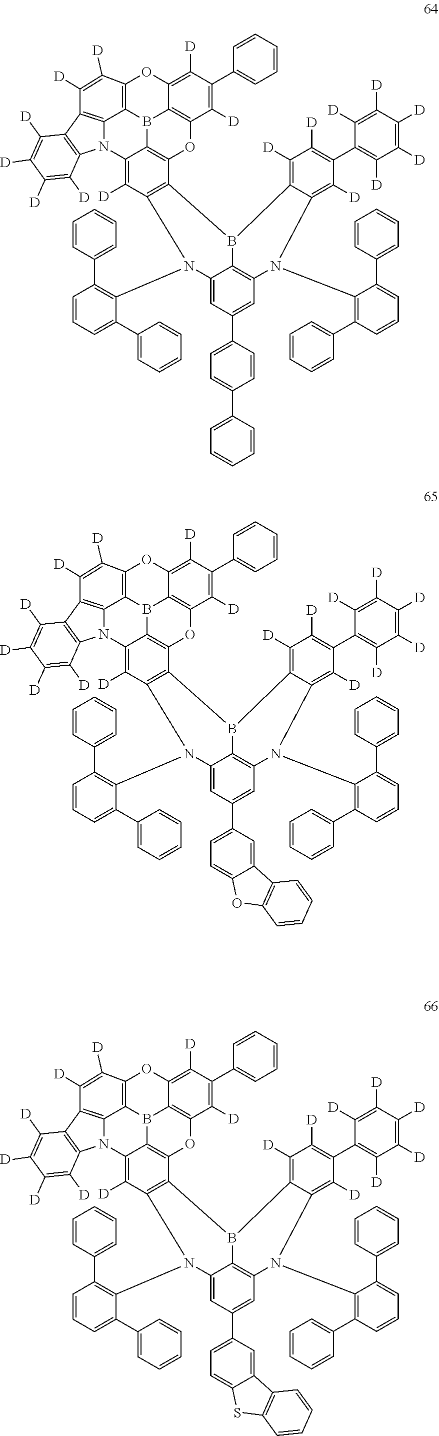 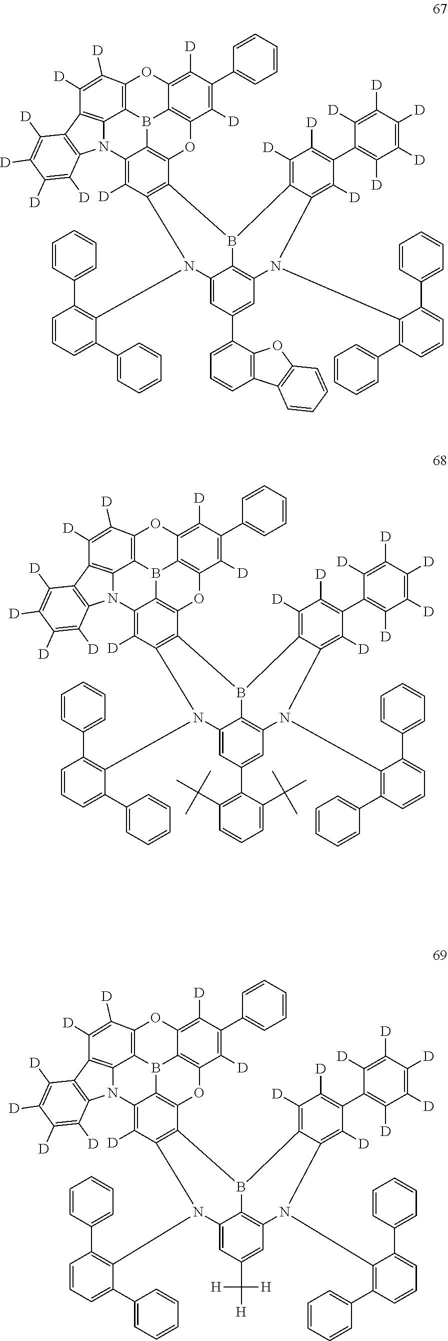 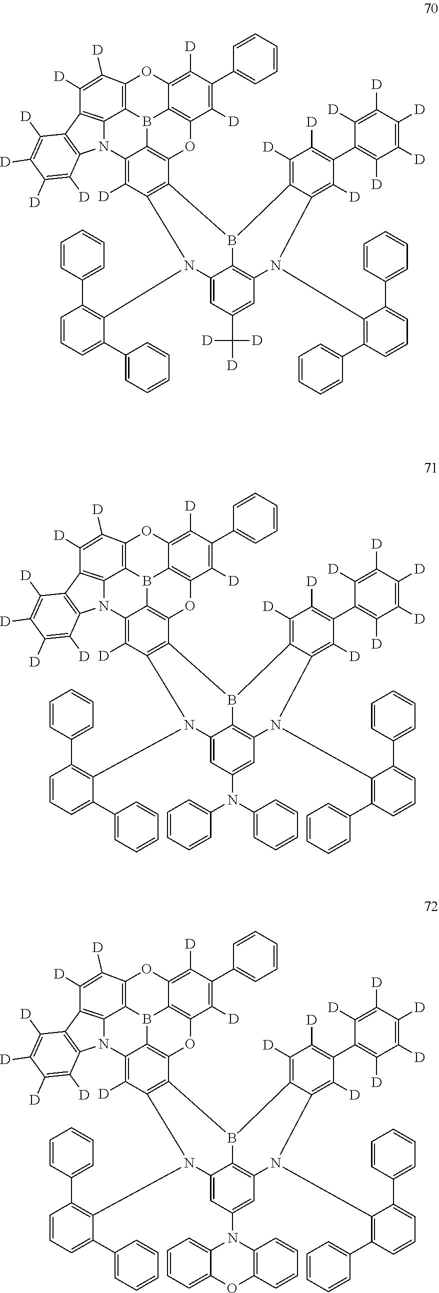 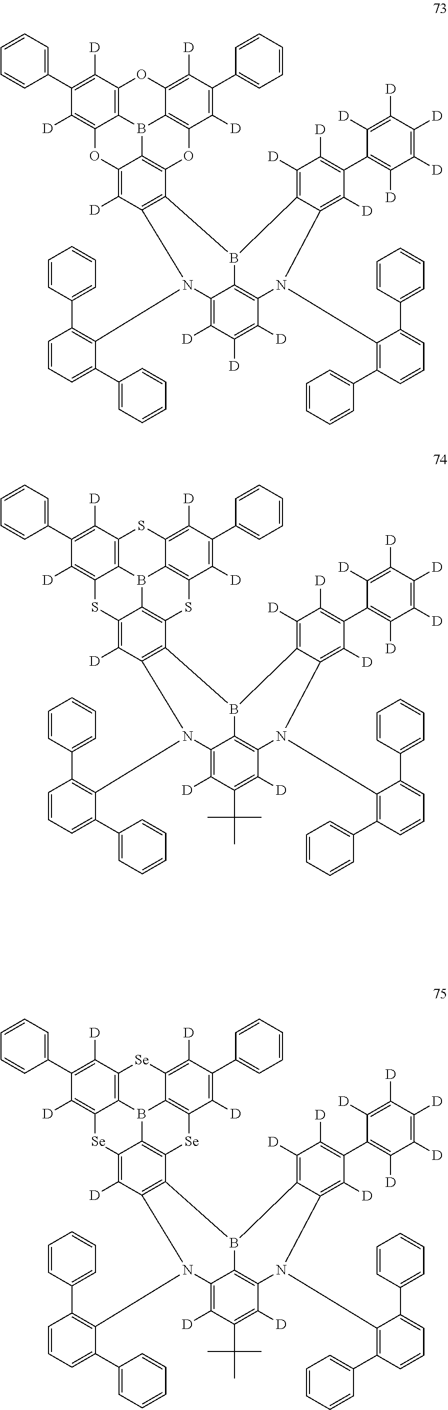 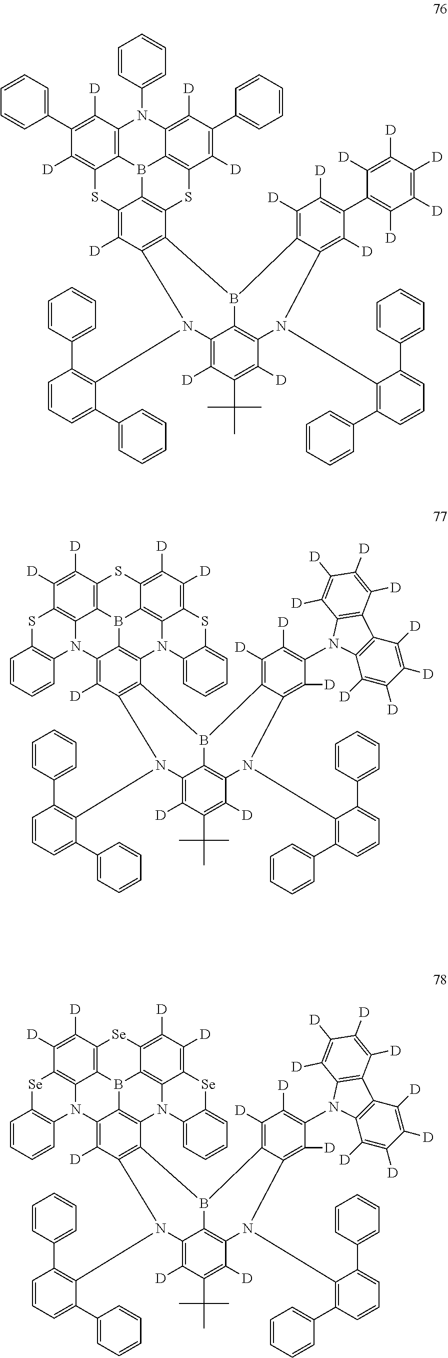 [chemical structure]
